SEQUENCE LISTING (158 sequences)

SEQ ID NO: 1 - protein, 475 aa, Chlamydomonas reinhardtii
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 2 - protein, 475 aa, Chlamydomonas reinhardtii
  VARIANT (28)..(28): D to H
MVPQTETKAGAGFKAGVKDYRLTYYTPHYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 3 - protein, 475 aa, Chlamydomonas reinhardtii
  VARIANT (31)..(31): V to K
MVPQTETKAGAGFKAGVKDYRLTYYTPDYVKRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 4 - protein, 475 aa, Chlamydomonas reinhardtii
  VARIANT (34)..(34): T to H
MVPQTETKAGAGFKAGVKDYRLTYYTPDYVVRDHDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 5 - protein, 475 aa, Chlamydomonas reinhardtii
  VARIANT (36)..(36): I to L
MVPQTETKAGAGFKAGVKDYRLTYYTPDYVVRDTDLLAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 6 - protein, 475 aa, Chlamydomonas reinhardtii
  VARIANT (68)..(68): T to Q
MVPQTETKAGAGFKAGVKDYRLTYYTPDYVVRDTDILAAFRMTPQPGVPPEECGAAVAAESSTGTWTQVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 7 - protein, 475 aa, Chlamydomonas reinhardtii
  VARIANT (68)..(68): T to S
MVPQTETKAGAGFKAGVKDYRLTYYTPDYVVRDTDILAAFRMTPQPGVPPEECGAAVAAESSTGTWTSVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 8 - protein, 475 aa, Chlamydomonas reinhardtii
  VARIANT (83)..(83): R to Q
MVPQTETKAGAGFKAGVKDYRLTYYTPDYVVRDTDILAAFRMTPQPGVPPEECGAAVAAESSTGTWTTVWTDGLTSLDRYKGQCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 9 - protein, 475 aa, Chlamydomonas reinhardtii
  VARIANT (83)..(83): R to H
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 10 - protein, 475 aa, Chlamydomonas reinhardtii
  VARIANT (88)..(88): E to S
MVPQTETKAGAGFKAGVKDYRLTYYTPDYVVRDTDILAAFRMTPQPGVPPEECGAAVAAESSTGTWTTVWTDGLTSLDRYKGRCYDISPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 11 - protein, 475 aa, Chlamydomonas reinhardtii
  VARIANT (87)..(87): I to Y
MVPQTETKAGAGFKAGVKDYRLTYYTPDYVVRDTDILAAFRMTPQPGVPPEECGAAVAAESSTGTWTTVWTDGLTSLDRYKGRCYDYEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 12 - protein, 475 aa, Chlamydomonas reinhardtii
  VARIANT (312)..(312): R to K
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFKVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 13 - protein, 475 aa, Chlamydomonas reinhardtii
  VARIANT (317)..(317): A to G
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKGLRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 14 - protein, 475 aa, Chlamydomonas reinhardtii
  VARIANT (320)..(320): M to L
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRLSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 15 - protein, 475 aa, Chlamydomonas reinhardtii
  VARIANT (355)..(355): E to P
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVPKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 16 - protein, 475 aa, Chlamydomonas reinhardtii
  VARIANT (358)..(358): R to Q
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDQSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 17 - protein, 475 aa, Chlamydomonas reinhardtii
  VARIANT (365)..(365): T to S
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFSQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 18 - protein, 475 aa, Chlamydomonas reinhardtii
  VARIANT (315)..(315): A to S
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLSKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 19 - protein, 475 aa, Chlamydomonas reinhardtii
  VARIANT (317)..(317): A to T
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKTLRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 20 - protein, 475 aa, Chlamydomonas reinhardtii
  VARIANT (6)..(6): E to G
  VARIANT (83)..(83): R to H
MVPQTGTKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 21 - protein, 475 aa, Chlamydomonas reinhardtii
  VARIANT (8)..(8): K to Y
  VARIANT (83)..(83): R to H
MVPQTETYAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 22 - protein, 475 aa, Chlamydomonas reinhardtii
  VARIANT (11)..(11): A to L
  VARIANT (83)..(83): R to H
MVPQTETKAGLGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 23 - protein, 475 aa, Chlamydomonas reinhardtii
  VARIANT (36)..(36): I to L
  VARIANT (83)..(83): R to H
MVPQTETKAGAGFKAGVKDYRLTYYTPDYVVRDTDLLAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 24 - protein, 475 aa, Chlamydomonas reinhardtii
  VARIANT (53)..(53): C to L
  VARIANT (83)..(83): R to H
MVPQTETKAGAGFKAGVKDYRLTYYTPDYVVRDTDILAAFRMTPQPGVPPEELGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 25 - protein, 475 aa, Chlamydomonas reinhardtii
  VARIANT (83)..(83): R to H
  VARIANT (87)..(87): I to L
MVPQTETKAGAGFKAGVKDYRLTYYTPDYVVRDTDILAAFRMTPQPGVPPEECGAAVAAESSTGTWTTVWTDGLTSLDRYKGHCYDLEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 26 - protein, 475 aa, Chlamydomonas reinhardtii
  VARIANT (83)..(83): R to H
  VARIANT (89)..(89): P to G
MVPQTETKAGAGFKAGVKDYRLTYYTPDYVVRDTDILAAFRMTPQPGVPPEECGAAVAAESSTGTWTTVWTDGLTSLDRYKGHCYDIEGVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 27 - protein, 475 aa, Chlamydomonas reinhardtii
  VARIANT (83)..(83): R to H
  VARIANT (93)..(93): E to H
MVPQTETKAGAGFKAGVKDYRLTYYTPDYVVRDTDILAAFRMTPQPGVPPEECGAAVAAESSTGTWTTVWTDGLTSLDRYKGHCYDIEPVPGHDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 28 - protein, 475 aa, Chlamydomonas reinhardtii
  VARIANT (83)..(83): R to H
  VARIANT (95)..(95): N to Q
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDQQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 29 - protein, 475 aa, Chlamydomonas reinhardtii
  VARIANT (83)..(83): R to H
  VARIANT (145)..(145): V to G
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYGKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 30 - protein, 475 aa, Chlamydomonas reinhardtii
  VARIANT (83)..(83): R to H
  VARIANT (168)..(168): G to P
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRPLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 31 - protein, 475 aa, Chlamydomonas reinhardtii
  VARIANT (83)..(83): R to H
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 32 - protein, 475 aa, Chlamydomonas reinhardtii
  VARIANT (83)..(83): R to H
  VARIANT (230)..(230): A to Y
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQYETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 33 - protein, 475 aa, Chlamydomonas reinhardtii
  VARIANT (83)..(83): R to H
  VARIANT (341)..(341): V to Q
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREQTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 34 - protein, 475 aa, Chlamydomonas reinhardtii
  VARIANT (83)..(83): R to H
  VARIANT (355)..(355): E to L
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVLKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 35 - protein, 475 aa, Chlamydomonas reinhardtii
  VARIANT (83)..(83): R to H
  VARIANT (355)..(355): E to Y
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVYKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 36 - protein, 475 aa, Chlamydomonas reinhardtii
  VARIANT (83)..(83): R to H
  VARIANT (355)..(355): E to Q
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVQKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 37 - protein, 475 aa, Chlamydomonas reinhardtii
  VARIANT (83)..(83): R to H
  VARIANT (355)..(355): E to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVSKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 38 - protein, 475 aa, Chlamydomonas reinhardtii
  VARIANT (83)..(83): R to H
  VARIANT (359)..(359): S to L
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRLRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 39 - protein, 475 aa, Chlamydomonas reinhardtii
  VARIANT (83)..(83): R to H
  VARIANT (359)..(359): S to G
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRGRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 40 - protein, 475 aa, Chlamydomonas reinhardtii
  VARIANT (83)..(83): R to H
  VARIANT (367)..(367): D to Q
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQQWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 41 - protein, 475 aa, Chlamydomonas reinhardtii
  VARIANT (83)..(83): R to H
  VARIANT (367)..(367): D to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQSWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 42 - protein, 475 aa, Chlamydomonas reinhardtii
  VARIANT (83)..(83): R to H
  VARIANT (392)..(392): E to D
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVDIFGDDACLQFGGGTLGHPWGNAPGAAANRVALEACTQARNEGRDLAREGGDVIRSACKWSPELAAACEVWKEIKFEFDTIDKL

SEQ ID NO: 43 - protein, 475 aa, Chlamydomonas reinhardtii
  VARIANT (83)..(83): R to H
  VARIANT (439)..(439): R to Y
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAYEGGDVIRSACKWSPELAAACEVWKEIKFEFDTIDKL

SEQ ID NO: 44 - protein, 475 aa, Chlamydomonas reinhardtii
  VARIANT (83)..(83): R to H
  VARIANT (450)..(450): K to Y
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACYWSPELAAACEVWKEIKFEFDTIDKL

SEQ ID NO: 45 - protein, 475 aa, Chlamydomonas reinhardtii
  VARIANT (83)..(83): R to H
  VARIANT (460)..(460): E to Q
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACQVWKEIKFEFDTIDKL

SEQ ID NO: 46 - protein, 475 aa, Chlamydomonas reinhardtii
  VARIANT (46)..(46): P to Q
  VARIANT (87)..(87): I to Y
MVPQTETKAGAGFKAGVKDYRLTYYTPDYVVRDTDILAAFRMTPQQGVPPEECGAAVAAESSTGTWTTVWTDGLTSLDRYKGRCYDYEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 47 - protein, 475 aa, Chlamydomonas reinhardtii
  VARIANT (87)..(87): I to Y
  VARIANT (89)..(89): P to G
MVPQTETKAGAGFKAGVKDYRLTYYTPDYVVRDTDILAAFRMTPQPGVPPEECGAAVAAESSTGTWTTVWTDGLTSLDRYKGRCYDYEGVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 48 - protein, 475 aa, Chlamydomonas reinhardtii
  VARIANT (87)..(87): I to Y
  VARIANT (255)..(255): V to K
MVPQTETKAGAGFKAGVKDYRLTYYTPDYVVRDTDILAAFRMTPQPGVPPEECGAAVAAESSTGTWTTVWTDGLTSLDRYKGRCYDYEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAKCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 49 - protein, 475 aa, Chlamydomonas reinhardtii
  VARIANT (87)..(87): I to Y
  VARIANT (272)..(272): G to S
MVPQTETKAGAGFKAGVKDYRLTYYTPDYVVRDTDILAAFRMTPQPGVPPEECGAAVAAESSTGTWTTVWTDGLTSLDRYKGRCYDYEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTSGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 50 - protein, 475 aa, Chlamydomonas reinhardtii
  VARIANT (11)..(11): A to S
  VARIANT (312)..(312): R to K
MVPQTETKAGSGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFKVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 51 - protein, 475 aa, Chlamydomonas reinhardtii
  VARIANT (312)..(312): R to K
  VARIANT (355)..(355): E to P
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFKVLAKALRMSGGDHLHSGTVVGKLEGEREVTLGFVDLMRDDYVPKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 52 - protein, 475 aa, Chlamydomonas reinhardtii
  VARIANT (30)..(30): V to L
  VARIANT (83)..(83): R to H
  VARIANT (200)..(200): T to S
MVPQTETKAGAGFKAGVKDYRLTYYTPDYLVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 53 - protein, 475 aa, Chlamydomonas reinhardtii
  VARIANT (83)..(83): R to H
  VARIANT (89)..(89): P to G
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIEGVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 54 - protein, 475 aa, Chlamydomonas reinhardtii
  VARIANT (83)..(83): R to H
  VARIANT (89)..(89): P to S
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIESVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 55 - protein, 475 aa, Chlamydomonas reinhardtii
  VARIANT (83)..(83): R to H
  VARIANT (94)..(94): D to H
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEHNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 56 - protein, 475 aa, Chlamydomonas reinhardtii
  VARIANT (83)..(83): R to H
  VARIANT (94)..(94): D to P
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEPNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 57 - protein, 475 aa, Chlamydomonas reinhardtii
  VARIANT (83)..(83): R to H
  VARIANT (95)..(95): N to H
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDHQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 58 - protein, 475 aa, Chlamydomonas reinhardtii
  VARIANT (83)..(83): R to H
  VARIANT (95)..(95): N to Q
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDQQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 59 - protein, 475 aa, Chlamydomonas reinhardtii
  VARIANT (83)..(83): R to H
  VARIANT (102)..(102): A to S
  VARIANT (200)..(200): T to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVSYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 60 - protein, 475 aa, Chlamydomonas reinhardtii
  VARIANT (83)..(83): R to H
  VARIANT (200)..(200): T to S
  VARIANT (249)..(249): E to D
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEDMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 61 - protein, 475 aa, Chlamydomonas reinhardtii
  VARIANT (6)..(6): E to G
  VARIANT (95)..(95): N to Q
MVPQTGTKAGAGFKAGVKDYRLTYYTPDYVVRDTDILAAFRMTPQPGVPPEECGAAVAAESSTGTWTTVWTDGLTSLDRYKGRCYDIEPVPGEDQQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 62 - protein, 475 aa, Chlamydomonas reinhardtii
  VARIANT (6)..(6): E to G
  VARIANT (145)..(145): V to G
MVPQTGTKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYGKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 63 - protein, 475 aa, Chlamydomonas reinhardtii
  VARIANT (36)..(36): I to L
  VARIANT (95)..(95): N to Q
MVPQTETKAGAGFKAGVKDYRLTYYTPDYVVRDTDLLAAFRMTPQPGVPPEECGAAVAAESSTGTWTTVWTDGLTSLDRYKGRCYDIEPVPGEDQQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 64 - protein, 475 aa, Chlamydomonas reinhardtii
  VARIANT (46)..(46): P to Q
  VARIANT (89)..(89): P to G
MVPQTETKAGAGFKAGVKDYRLTYYTPDYVVRDTDILAAFRMTPQQGVPPEECGAAVAAESSTGTWTTVWTDGLTSLDRYKGRCYDIEGVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 65 - protein, 475 aa, Chlamydomonas reinhardtii
  VARIANT (83)..(83): R to Q
  VARIANT (367)..(367): D to S
MVPQTETKAGAGFKAGVKDYRLTYYTPDYVVRDTDILAAFRMTPQPGVPPEECGAAVAAESSTGTWTTVWTDGLTSLDRYKGQCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQSWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 66 - protein, 475 aa, Chlamydomonas reinhardtii
  VARIANT (89)..(89): P to G
  VARIANT (359)..(359): S to G
MVPQTETKAGAGFKAGVKDYRLTYYTPDYVVRDTDILAAFRMTPQPGVPPEECGAAVAAESSTGTWTTVWTDGLTSLDRYKGRCYDIEGVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRGRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 67 - protein, 475 aa, Chlamydomonas reinhardtii
  VARIANT (355)..(355): E to P
  VARIANT (359)..(359): S to G
MVPQTETKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVPKDRGRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 68 - protein, 475 aa, Chlamydomonas reinhardtii
  VARIANT (6)..(6): E to G
  VARIANT (46)..(46): P to Q
  VARIANT (89)..(89): P to G
MVPQTGTKAGAGFKAGVKDYRLTYYTPDYVVRDTDILAAFRMTPQQGVPPEECGAAVAAESSTGTWTTVWTDGLTSLDRYKGRCYDIEGVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 69 - protein, 475 aa, Chlamydomonas reinhardtii
  VARIANT (6)..(6): E to G
  VARIANT (83)..(83): R to Q
  VARIANT (87)..(87): I to L
MVPQTGTKAGAGFKAGVKDYRLTYYTPDYVVRDTDILAAFRMTPQPGVPPEECGAAVAAESSTGTWTTVWTDGLTSLDRYKGQCYDLEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 70 - protein, 475 aa, Chlamydomonas reinhardtii
  VARIANT (6)..(6): E to G
  VARIANT (272)..(272): G to S
  VARIANT (355)..(355): E to P
MVPQTGTKAGAGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTSGFTANTSLAIYCRDNGLLLHIHRAMHAVIDRQRNHGIHFRVLAKALRMSGGDHLHSGTVVGKLEGEREVTLGFVDLMRDDYVPKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 71 - protein, 475 aa, Chlamydomonas reinhardtii
  VARIANT (11)..(11): A to L
  VARIANT (83)..(83): R to H
  VARIANT (355)..(355): E to P
MVPQTETKAGLGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVPKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 72 - protein, 475 aa, Chlamydomonas reinhardtii
  VARIANT (11)..(11): A to L
  VARIANT (87)..(87): I to Y
  VARIANT (355)..(355): E to P
MVPQTETKAGLGFKAGVKDYRLTYYTPDYVVRDTDILAAFRMTPQPGVPPEECGAAVAAESSTGTWTTVWTDGLTSLDRYKGRCYDYEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVPKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 73 - protein, 475 aa, Chlamydomonas reinhardtii
  VARIANT (11)..(11): A to L
  VARIANT (93)..(93): E to H
  VARIANT (359)..(359): S to L
MVPQTETKAGLGFKAGVKDYRLTYYTPDYVVRDTDILAAFRMTPQPGVPPEECGAAVAAESSTGTWTTVWTDGLTSLDRYKGRCYDIEPVPGHDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRLRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 74 - protein, 475 aa, Chlamydomonas reinhardtii
  VARIANT (11)..(11): A to S
  VARIANT (359)..(359): S to G
  VARIANT (367)..(367): D to Q
MVPQTETKAGSGFKAGVKDYRLTYYTPDYVVRDTDILAAFRMTPQPGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRGRGIYFTQQWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 75 - protein, 475 aa, Chlamydomonas reinhardtii
  VARIANT (46)..(46): P to Q
  VARIANT (95)..(95): N to Q
  VARIANT (355)..(355): E to Y
MVPQTETKAGAGFKAGVKDYRLTYYTPDYVVRDTDILAAFRMTPQQGVPPEECGAAVAAESSTGTWTTVWTDGLTSLDRYKGRCYDIEPVPGEDQQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVYKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 76 - protein, 475 aa, Chlamydomonas reinhardtii
  VARIANT (83)..(83): R to H
  VARIANT (200)..(200): T to S
  VARIANT (355)..(355): E to S
MVPQTETKAGAGFKAGVKDYRLTYYTPDYVVRDTDILAAFRMTPQPGVPPEECGAAVAAESSTGTWTTVWTDGLTSLDRYKGHCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFSKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVSKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 77 - protein, 475 aa, Chlamydomonas reinhardtii
  VARIANT (46)..(46): P to Q
  VARIANT (359)..(359): S to L
MVPQTETKAGAGFKAGVKDYRLTYYTPDYVVRDTDILAAFRMTPQQGVPPEECGAAVAAESSTGTWTTVWTDGLTSLDRYKGRCYDIEPVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRLRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 78 - protein, 475 aa, Chlamydomonas reinhardtii
  VARIANT (87)..(87): I to L
  VARIANT (89)..(89): P to G
  VARIANT (355)..(355): E to Q
MVPQTETKAGAGFKAGVKDYRLTYYTPDYVVRDTDILAAFRMTPQPGVPPEECGAAVAAESSTGTWTTVWTDGLTSLDRYKGRCYDLEGVPGEDNQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVQKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 79 - protein, 475 aa, Chlamydomonas reinhardtii
  VARIANT (93)..(93): E to H
  VARIANT (95)..(95): N to Q
MVPQTETKAGAGFKAGVKDYRLTYYTPDYVVRDTDILAAFRMTPQPGVPPEECGAAVAAESSTGTWTTVWTDGLTSLDRYKGRCYDIEPVPGHDQQYIAYVAYPIDLFEEGSVTNMFTSIVGNVFGFKALRALRLEDLRIPPAYVKTFVGPPHGIQVERDKLNKYGRGLLGCTIKPKLGLSAKNYGRAVYECLRGGLDFTKDDENVNSQPFMRWRDRFLFVAEAIYKAQAETGEVKGHYLNATAGTCEEMMKRAVCAKELGVPIIMHDYLTGGFTANTSLAIYCRDNGLLLHIHRAMHAVIDRQRNHGIHFRVLAKALRMSGGDHLHSGTVVGKLEGEREVTLGFVDLMRDDYVEKDRSRGIYFTQDWCSMPGVMPVASGGIHVWHMPALVEIFGDDACLQFGGGTLGHPWGNAPGAAANRVALEACTQARNEGRDLAREGGDVIRSACKWSPELAAACEVWKEIKFEFDTIDKL

SEQ ID NO: 80 - DNA, 1428 bp, Chlamydomonas reinhardtii
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 81 - DNA, 1428 bp, Chlamydomonas reinhardtii
  mutation (82)..(84)
atggttccacaaacagaaactaaagcaggtgctggattcaaagccggtgtaaaagactaccgtttaacatactacacacctcactacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 82 - DNA, 1428 bp, Chlamydomonas reinhardtii
  mutation (91)..(93)
atggttccacaaacagaaactaaagcaggtgctggattcaaagccggtgtaaaagactaccgtttaacatactacacacctgattacgtaaaaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 83 - DNA, 1428 bp, Chlamydomonas reinhardtii
  mutation (100)..(102)
atggttccacaaacagaaactaaagcaggtgctggattcaaagccggtgtaaaagactaccgtttaacatactacacacctgattacgtagtaagagatcacgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 84 - DNA, 1428 bp, Chlamydomonas reinhardtii
  mutation (106)..(108)
atggttccacaaacagaaactaaagcaggtgctggattcaaagccggtgtaaaagactaccgtttaacatactacacacctgattacgtagtaagagatactgatttattagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 85 - DNA, 1428 bp, Chlamydomonas reinhardtii
  mutation (202)..(204)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactcaagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 86 - DNA, 1428 bp, Chlamydomonas reinhardtii
  mutation (202)..(204)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggacttcagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 87 - DNA, 1428 bp, Chlamydomonas reinhardtii
  mutation (247)..(249)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcaatgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 88 - DNA, 1428 bp, Chlamydomonas reinhardtii
  mutation (247)..(249)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 89 - DNA, 1428 bp, Chlamydomonas reinhardtii
  mutation (262)..(264)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatctcaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 90 - DNA, 1428 bp, Chlamydomonas reinhardtii
  mutation (259)..(261)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgattacgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 91 - DNA, 1428 bp, Chlamydomonas reinhardtii
  mutation (934)..(936)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttcaaagttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 92 - DNA, 1428 bp, Chlamydomonas reinhardtii
  mutation (949)..(951)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaaggccttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 93 - DNA, 1428 bp, Chlamydomonas reinhardtii
  mutation (958)..(960)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtttatctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 94 - DNA, 1428 bp, Chlamydomonas reinhardtii
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttccaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 95 - DNA, 1428 bp, Chlamydomonas reinhardtii
  mutation (1072)..(1074)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccaaagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 96 - DNA, 1428 bp, Chlamydomonas reinhardtii
  mutation (1093)..(1095)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttctcacaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 97 - DNA, 1428 bp, Chlamydomonas reinhardtii
  mutation (943)..(945)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttagtaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 98 - DNA, 1428 bp, Chlamydomonas reinhardtii
  mutation (949)..(951)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaaactcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 99 - DNA, 1428 bp, Chlamydomonas reinhardtii
  mutation (16)..(18)
  mutation (247)..(249)
atggttccacaaacaggcactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 100 - DNA, 1428 bp, Chlamydomonas reinhardtii
  mutation (22)..(24)
  mutation (247)..(249)
atggttccacaaacagaaacttacgcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 101 - DNA, 1428 bp, Chlamydomonas reinhardtii
  mutation (31)..(33)
  mutation (247)..(249)
atggttccacaaacagaaactaaagcaggtttaggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 102 - DNA, 1428 bp, Chlamydomonas reinhardtii
  mutation (106)..(108)
  mutation (247)..(249)
atggttccacaaacagaaactaaagcaggtgctggattcaaagccggtgtaaaagactaccgtttaacatactacacacctgattacgtagtaagagatactgatttattagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 103 - DNA, 1428 bp, Chlamydomonas reinhardtii
  mutation (157)..(159)
  mutation (247)..(249)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaattaggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 104 - DNA, 1428 bp, Chlamydomonas reinhardtii
  mutation (247)..(249)
  mutation (259)..(261)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatttagaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 105 - DNA, 1428 bp, Chlamydomonas reinhardtii
  mutation (247)..(249)
  mutation (265)..(267)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaggcgttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 106 - DNA, 1428 bp, Chlamydomonas reinhardtii
  mutation (247)..(249)
  mutation (277)..(279)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtcacgacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 107 - DNA, 1428 bp, Chlamydomonas reinhardtii
  mutation (247)..(249)
  mutation (283)..(285)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagaccaacaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 108 - DNA, 1428 bp, Chlamydomonas reinhardtii
  mutation (247)..(249)
  mutation (433)..(435)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacggcaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 109 - DNA, 1428 bp, Chlamydomonas reinhardtii
  mutation (247)..(249)
  mutation (502)..(504)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtccacttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 110 - DNA, 1428 bp, Chlamydomonas reinhardtii
  mutation (247)..(249)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 111 - DNA, 1428 bp, Chlamydomonas reinhardtii
  mutation (247)..(249)
  mutation (688)..(690)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaatacgaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 112 - DNA, 1428 bp, Chlamydomonas reinhardtii
  mutation (247)..(249)
  mutation (1021)..(1023)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaacaaactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 113 - DNA, 1428 bp, Chlamydomonas reinhardtii
  mutation (247)..(249)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttttaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 114 - DNA, 1428 bp, Chlamydomonas reinhardtii
  mutation (247)..(249)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgtttacaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 115 - DNA, 1428 bp, Chlamydomonas reinhardtii
  mutation (247)..(249)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttcaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 116 - DNA, 1428 bp, Chlamydomonas reinhardtii
  mutation (247)..(249)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgtttcaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 117 - DNA, 1428 bp, Chlamydomonas reinhardtii
  mutation (247)..(249)
  mutation (1075)..(1077)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtttacgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 118 - DNA, 1428 bp, Chlamydomonas reinhardtii
  mutation (247)..(249)
  mutation (1075)..(1077)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtggccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 119 - DNA, 1428 bp, Chlamydomonas reinhardtii
  mutation (1075)..(1077)
  mutation (1099)..(1101)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaacaatggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 120 - DNA, 1428 bp, Chlamydomonas reinhardtii
  mutation (247)..(249)
  mutation (1099)..(1101)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaatcatggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 121 - DNA, 1428 bp, Chlamydomonas reinhardtii
  mutation (247)..(249)
  mutation (1174)..(1176)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgacatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 122 - DNA, 1428 bp, Chlamydomonas reinhardtii
  mutation (247)..(249)
  mutation (1315)..(1317)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgcttacgaaggtggcgacgtaattcgttcagcttgtaaatggtctccagaacttgctgctgcatgtgaagtttggaaagaaattaaattcgaatttgatactattgacaaactttaa

SEQ ID NO: 123 - DNA, 1428 bp, Chlamydomonas reinhardtii
  mutation (247)..(249)
  mutation (1348)..(1350)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgttactggtctccagaacttgctgctgcatgtgaagtttggaaagaaattaaattcgaatttgatactattgacaaactttaa

SEQ ID NO: 124 - DNA, 1428 bp, Chlamydomonas reinhardtii
  mutation (247)..(249)
  mutation (1378)..(1389)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtcaagtttggaaagaaattaaattcgaatttgatactattgacaaactttaa

SEQ ID NO: 125 - DNA, 1428 bp, Chlamydomonas reinhardtii
  mutation (136)..(138)
  mutation (259)..(261)
atggttccacaaacagaaactaaagcaggtgctggattcaaagccggtgtaaaagactaccgtttaacatactacacacctgattacgtagtaagagatactgatattttagctgcattccgtatgactccacaacaaggtgttccacctgaagaatgtggtgctgctgtagctgctgaatcttcaacaggtacatggactacagtatggactgacggtttaacaagtcttgaccgttacaaaggtcgttgttacgattacgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 126 - DNA, 1428 bp, Chlamydomonas reinhardtii
  mutation (259)..(261)
  mutation (265)..(267)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgattacgaaggcgttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 127 - DNA, 1428 bp, Chlamydomonas reinhardtii
  mutation (259)..(261)
  mutation (763)..(765)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgattacgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcaaaatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 128 - DNA, 1428 bp, Chlamydomonas reinhardtii
  mutation (259)..(261)
  mutation (814)..(816)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgattacgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacatcaggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 129 - DNA, 1428 bp, Chlamydomonas reinhardtii
  mutation (31)..(33)
  mutation (934)..(936)
atggttccacaaacagaaactaaagcaggttcaggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttcaaagttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 130 - DNA, 1428 bp, Chlamydomonas reinhardtii
  mutation (934)..(936)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttcaaagttcttgctaaagctcttcgtatgtctggtggtgaccaccttcactctggtactgttgtaggtaaactagaaggtgaacgtgaagttactctaggtttcgtagacttaatgcgtgatgactacgttccaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 131 - DNA, 1428 bp, Chlamydomonas reinhardtii
  mutation (88)..(90)
  mutation (247)..(249)
  mutation (598)..(600)
atggttccacaaacagaaactaaagcaggtgctggattcaaagccggtgtaaaagactaccgtttaacatactacacacctgattacttagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 132 - DNA, 1428 bp, Chlamydomonas reinhardtii
  mutation (247)..(249)
  mutation (265)..(267)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaggcgttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 133 - DNA, 1428 bp, Chlamydomonas reinhardtii
  mutation (247)..(249)
  mutation (265)..(267)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaatcagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 134 - DNA, 1428 bp, Chlamydomonas reinhardtii
  mutation (247)..(249)
  mutation (280)..(282)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaacacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 135 - DNA, 1428 bp, Chlamydomonas reinhardtii
  mutation (247)..(249)
  mutation (280)..(282)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaaccaaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 136 - DNA, 1428 bp, Chlamydomonas reinhardtii
  mutation (247)..(249)
  mutation (283)..(285)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagaccaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 137 - DNA, 1428 bp, Chlamydomonas reinhardtii
  mutation (247)..(249)
  mutation (283)..(285)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagaccaacaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 138 - DNA, 1428 bp, Chlamydomonas reinhardtii
  mutation (247)..(249)
  mutation (304)..(306)
  mutation (598)..(600)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtatcatacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 139 - DNA, 1428 bp, Chlamydomonas reinhardtii
  mutation (247)..(249)
  mutation (598)..(600)
  mutation (745)..(747)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagacatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 140 - DNA, 1428 bp, Chlamydomonas reinhardtii
  mutation (16)..(18)
  mutation (283)..(285)
atggttccacaaacaggcactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagaccaacaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 141 - DNA, 1428 bp, Chlamydomonas reinhardtii
  mutation (16)..(18)
  mutation (433)..(435)
atggttccacaaacaggcactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacggcaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 142 - DNA, 1428 bp, Chlamydomonas reinhardtii
  mutation (106)..(108)
  mutation (283)..(285)
atggttccacaaacagaaactaaagcaggtgctggattcaaagccggtgtaaaagactaccgtttaacatactacacacctgattacgtagtaagagatactgatttattagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagaccaacaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 143 - DNA, 1428 bp, Chlamydomonas reinhardtii
  mutation (136)..(138)
  mutation (265)..(267)
atggttccacaaacagaaactaaagcaggtgctggattcaaagccggtgtaaaagactaccgtttaacatactacacacctgattacgtagtaagagatactgatattttagctgcattccgtatgactccacaacaaggtgttccacctgaagaatgtggtgctgctgtagctgctgaatcttcaacaggtacatggactacagtatggactgacggtttaacaagtcttgaccgttacaaaggtcgttgttacgatatcgaaggcgttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 144 - DNA, 1428 bp, Chlamydomonas reinhardtii
  mutation (247)..(249)
  mutation (1099)..(1101)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcaatgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaatcatggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 145 - DNA, 1428 bp, Chlamydomonas reinhardtii
  mutation (265)..(267)
  mutation (1075)..(1077)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaggcgttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtggccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 146 - DNA, 1428 bp, Chlamydomonas reinhardtii
  mutation (1063)..(1065)
  mutation (1075)..(1077)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttccaaaagaccgtggccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 147 - DNA, 1428 bp, Chlamydomonas reinhardtii
  mutation (16)..(18)
  mutation (136)..(138)
  mutation (265)..(267)
atggttccacaaacaggcactaaagcaggtgctggattcaaagccggtgtaaaagactaccgtttaacatactacacacctgattacgtagtaagagatactgatattttagctgcattccgtatgactccacaacaaggtgttccacctgaagaatgtggtgctgctgtagctgctgaatcttcaacaggtacatggactacagtatggactgacggtttaacaagtcttgaccgttacaaaggtcgttgttacgatatcgaaggcgttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 148 - DNA, 1428 bp, Chlamydomonas reinhardtii
  mutation (16)..(18)
  mutation (247)..(249)
  mutation (259)..(261)
atggttccacaaacaggcactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcaatgttacgatttagaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 149 - DNA, 1428 bp, Chlamydomonas reinhardtii
  mutation (16)..(18)
  mutation (814)..(816)
  mutation (1063)..(1065)
atggttccacaaacaggcactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacatcaggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttccaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 150 - DNA, 1428 bp, Chlamydomonas reinhardtii
  mutation (31)..(33)
  mutation (247)..(249)
  mutation (1063)..(1065)
atggttccacaaacagaaactaaagcaggtttaggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttccaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 151 - DNA, 1428 bp, Chlamydomonas reinhardtii
  mutation (31)..(33)
  mutation (259)..(261)
  mutation (1063)..(1065)
atggttccacaaacagaaactaaagcaggtttaggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgattacgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttccaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 152 - DNA, 1428 bp, Chlamydomonas reinhardtii
  mutation (31)..(33)
  mutation (277)..(279)
  mutation (1075)..(1077)
atggttccacaaacagaaactaaagcaggtttaggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtcacgacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtttacgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 153 - DNA, 1428 bp, Chlamydomonas reinhardtii
  mutation (31)..(33)
  mutation (1075)..(1077)
  mutation (1099)..(1101)
atggttccacaaacagaaactaaagcaggttcaggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtggccgtggtatttacttcactcaacaatggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 154 - DNA, 1428 bp, Chlamydomonas reinhardtii
  mutation (136)..(138)
  mutation (283)..(285)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaacaaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagaccaacaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgtttacaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 155 - DNA, 1428 bp, Chlamydomonas reinhardtii
  mutation (247)..(249)
  mutation (598)..(600)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcactgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgacttttcaaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgtttcaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 156 - DNA, 1428 bp, Chlamydomonas reinhardtii
  mutation (136)..(138)
  mutation (1075)..(1077)
atggttccacaaacagaaactaaagcaggtgctggattcaaagccggtgtaaaagactaccgtttaacatactacacacctgattacgtagtaagagatactgatattttagctgcattccgtatgactccacaacaaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtttacgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 157 - DNA, 1428 bp, Chlamydomonas reinhardtii
  mutation (259)..(261)
  mutation (265)..(267)
  mutation (1063)..(1065)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatttagaaggcgttccgggtgaagacaaccaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttcaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

SEQ ID NO: 158 - DNA, 1428 bp, Chlamydomonas reinhardtii
  mutation (277)..(279)
  mutation (283)..(285)
atggttccacaaacagaaactaaagcaggtgctggattcaaagccggtgtaaaagactaccgtttaacatactacacacctgattacgtagtaagagatactgatattttagctgcattccgtatgactccacaaccaggtgttccacctgaagaatgtggtgctgctgtagctgctgaatcttcaacaggtacatggactacagtatggactgacggtttaacaagtcttgaccgttacaaaggtcgttgttacgatatcgaaccagttccgggtcacgaccaacaatacattgcttacgtagcttacccaatcgacttattcgaagaaggttcagtaactaacatgttcacttctattgtaggtaacgtattcggtttcaaagctttacgtgctctacgtcttgaagaccttcgtattccacctgcttacgttaaaacattcgtaggtcctccacacggtattcaggtagaacgtgacaaattaaacaaatatggtcgtggtcttttaggttgtacaatcaaacctaaattaggtctttcagctaaaaactacggtcgtgcagtttatgaatgtttacgtggtggtcttgactttactaaagacgacgaaaacgtaaactcacaaccattcatgcgttggcgtgaccgtttccttttcgttgctgaagctatttacaaagctcaagcagaaacaggtgaagttaaaggtcactacttaaacgctactgctggtacttgtgaagaaatgatgaaacgtgcagtatgtgctaaagaattaggtgtacctattattatgcacgactacttaacaggtggtttcacagctaacacttcattagctatctactgtcgtgacaacggtcttcttctacacatccaccgtgctatgcacgcggttattgaccgtcaacgtaaccacggtattcacttccgtgttcttgctaaagctcttcgtatgtctggtggtgaccaccttcactctggtactgttgtaggtaaactagaaggtgaacgtgaagttactctaggtttcgtagacttaatgcgtgatgactacgttgaaaaagaccgtagccgtggtatttacttcactcaagactggtgttcaatgccaggtgttatgccagttgcttcaggtggtattcacgtatggcacatgccagctttagttgaaatcttcggtgatgacgcatgtcttcagttcggtggtggtactctaggtcacccttggggtaacgctccaggtgctgcagctaaccgtgtagctcttgaagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaa

(19) United States
(12) Patent Application Publication
(10) Pub. No.: US 2019/0112617 A1
(43) Pub. Date: Apr. 18, 2019

(54) MODIFIED RUBISCO LARGE SUBUNIT PROTEINS

(71) Applicant: RENEW BIOPHARMA, INC., San Diego, CA (US)

(72) Inventors: Christopher YOHN, San Diego, CA (US); Yan POON, San Diego, CA (US); Daniel SANTOS, Novato, CA (US); Bryan O'NEILL, San Diego, CA (US)

(21) Appl. No.: 16/090,193

(22) PCT Filed: Mar. 30, 2017
(86) PCT No.: PCT/US2017/024870

§ 371 Date: Sep. 28, 2018

Related U.S. Application Data
(60) Provisional application No. 62/315,196, filed on Mar. 30, 2016.

(51) Int. Cl. C12N 15/82; C12N 9/88; C07K 14/415; C12N 15/52

(52) U.S. Cl. CPC C12N 15/8269; C12N 9/88; C12Y 401/01039; C12N 15/52; C07K 14/415

(57) ABSTRACT

Provided herein are modified Rubisco large subunit proteins and nucleid acids encoding such proteins, as well as photosynthetic organism transformed with said nucleic acids and expressing said proteins. In certain embodiments, photosynthetic organisms containing said modified Rubisco large subunit proteins exhibit increased biomass production.

### BACKGROUND

Ribulose-1,5-biphosphate carboxylase oxygenase, commonly known as RuBisCo, or more simply Rubisco, is an enzyme involved in the first step of carbon fixation by photosynthetic organisms and is considered to be the most abundant enzyme on Earth. Carbon fixation is the process by which photosynthetic organisms capture atmospheric carbon to produce high energy molecules such as glucose used to produce biomass.

In addition to being one of the most abundant, Rubisco is also one of the largest enzymes. The functional Rubisco enzyme is made up of a combination of large (55 kDa) and small (15 kDa) subunits. Four pairs of large subunits (rbcL) are capped on each end by four small subunits (rbcS), each of which interacts with three large subunits. The large subunit is encoded by chloroplast DNA while the small subunit, with a few exceptions, is encoded by nuclear DNA.

Despite its importance in photosynthesis, Rubisco is a very inefficient enzyme and is the rate limiting step in photosynthesis. Improvements in the efficiency of Rubisco and so photosynthesis would have major beneficial impacts. An improved ability of photosynthetic organism to fix carbon would allow more efficient production of biomass to meet the nutritional needs of humans and animals as well as for other uses. An increase ability to fix carbon would also be important in reducing the amount of atmospheric carbon which has been associated with climate change. The amino acid sequences of the large subunit is fairly conserved across species while the sequence of the small unit is more divergent. Thus, there is a need for improved Rubisco proteins. In addition, the conserved nature of the large subunit makes it a good target for improvement.

### SUMMARY

Provide herein is: 1 A transformed photosynthetic organism comprising an exogenous polynucleotide encoding a modified rbcL protein of any one of SEQ ID NO: 1; wherein said modification consists of at least one amino acid substitution in the loop at positions 25-35, the β-sheet at positions 83-89, the α-helix at positions 310-321 and the loop-helix-loop at positions 355-365; and wherein the transformed photosynthetic organism expresses the modified rbcL protein and produces increased biomass as compared to an untransformed photosynthetic organism of the same species. (2) A transformed photosynthetic organism comprising an exogenous polynucleotide encoding a modified rbcL protein of any one of SEQ ID NOs 2 to 79; wherein the transformed photosynthetic organism expresses the modified rbcL protein and produces increased biomass as compared to an untransformed photosynthetic organism of the same species. (3) A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein comprises at least one of the following modifications: a) a change from D to H at position 28; b) a change from V to K at position 31; c) a change from T to H at position 34; d) a change from I to L at position 36; e) a change from T to Q at position 68; f) a change from T to S at position 68; g). a change from R to Q at position 83; h) a change from R to H at position 83; i) a change from E to S at position 88; j) a change from I to Y at position 87; k) a change from R to K at position 312; l) a change from A to G at position 317; m) a change from M to L at position 320; n) a change from E to P at position 355; o) a change from R to Q at position 358; p) a change from T to S at position 365; q) a change from A to S at position 315; r) a change from A to T at position 317; s) a change from R to H at position 83 and a change from E to G at position 6; t) a change from R to H at position 83 and a change from K to Y at position 8; u) a change from R to H at position 83 and a change from A to L at position 11; v) a change from R to H at position 83 and a change from I to L at position 36; w) a change from R to H at position 83 and a change from C to L at position 53; x) a change from R to H at position 83 and a change from I to L at position 87; y) a change from R to H at position 83 and a change from P to G at position 89; z) a change from R to H at position 83 and a change from E to H at position 93; aa) a change from R to H at position 83 and a change from N to Q at position 95; ab) a change from R to H at position 83 and a change from V to G at position 145; ac) a change from R to H at position 83 and a change from G to P at position 168; ad) a change from R to H at position 83 and a change from T to S at position 200; ae) a change from R to H at position 83 and a change from A to Y at position 230; af) a change from R to H at position 83 and a change from V to Q at position 341; ag) a change from R to H at position 83 and a change from E to L at position 355; ah) a change from R to H at position 83 and a change from E to Y at position 355; ai) a change from R to H at position 83 and a change from E to Q at position 355; aj) a change from R to H at position 83 and a change from E to S at position 355; ak) a change from R to H at position 83 and a change from S to L at position 359; al) a change from R to H at position 83 and a change from S to G at position 359; am) a change from R to H at position 83 and a change from D to Q at position 367; an) a change from R to H at position 83 and a change from D to S at position 367; ao) a change from R to H at position 83 and a change from E to D at position 392; ap) a change from R to H at position 83 and a change from R to Y at position 439; aq) a change from R to H at position 83 and a change from K to Y at position 450; ar) a change from R to H at position 83 and a change from E to Q at position 460; as) a change from I to Y at position 87 and a change from P to Q at position 46; at) a change from I to Y at position 87 and a change from P to G at position 89; au) a change from I to Y at position 87 and a change from V to K at position 255; av) a change from I to Y at position 87 and a change from G to S at position 272; aw) a change from R to K at position 312 and a change from A to S at position 11; ax) a change from R to K at position 312 and a change from E to P at position 355; ay) a change from V to L at position 30, a change from R to H at position 83 and a change from T to S at position 200; az) a change from R to H at position 83, a change from P to G at position 89 and a change from T to S at position 200; ba) a change from a change from R to H at position 83, a change from P to S at position 89 and a change from T to S at position 200; bb) a change from R to H at position 83, a change from D to H at position 94 and a change from T to S at position 200; bc) a change from R to H at position 83, a change from D to P at position 94 and a change from T to S at position 200; bd) a change from R to H at position 83, a change from N to H at position 95 and a change from T to S at position 200; be) a change from R to H at position 83, a change from N to Q at position 95 and a change from T to S at position 200; bf) a change from R to H at position 83, a change from A to S at position 102 and a change from T to S at position 200; bg) a change from R to H at position 83, a change from T to S at position 200 and a change from E to D at position 249; bh) a change from E to G at position 6 and a change from N to Q at position 95; bi) a change from E to G at position 6 and a change from V to G at position 145; bj) a change from I to L at position 36 and a change from N to Q at position 95; bk) a change from P to Q at position 46 and a change from P to G at position 89; bl) a change from R to Q at position 83 and a change from D to S at position 367; bm) a change from P to G at position 89 and a change from S to G at position 359; bn) a change from E to P at position 355 and a change from S to G at position 359; bo) a change from E to G at position 6, a change from P to Q at position 46 and a change from P to G at position 89; bp) a change from E to G at position 6, a change from R to Q at position 83 and a change from I to L at position 87; bq) a change from a change from E to G at position 6, a change from G to S at position 272 and a change from E to P at position 355; br) a change from A to L at position 11, a change from R to H at position 83 and a change from E to P at position 355; bs) a change from A to L at position 11, a change from I to Y at position 87 and a change from E to P at position 355; bt) a change from A to L a position 11, a change from E to H at position 93 and change from S to L at position 359; bu) a change from A to S at position 11, a change from S to G at position 359 and a change from D to Q at position 367; bv) a change from P to Q at position 46, a change from N to Q at position 95 and a change from E to Y at position 355; bw) a change from R to H at position 83, a change from T to S at position 200 and a change from E to S at position 355; bx) a change from P to G at position 46 and a change from S to L at position 359; by) a change from I to L at position 87, a change from P to G at position 89, and a change from E to Q at position 355; and bz) a change from E to H at position 93 and a change from N to Q at position 95.

Also provided is: (4) A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein consists of a modification selected from: a) a change from D to H at position 28; b) a change from V to K at position 31; c) a change from T to H at position 34; d) a change from I to L at position 36; e) a change from T to Q at position 68; f) a change from T to S at position 68; g) a change from R to Q at position 83; h) a change from R to H at position 83; i) a change from E to S at position 88; j) a change from I to Y at position 87; k) a change from R to K at position 312; l) a change from A to G at position 317; m) a change from M to L at position 320; n) a change from E to P at position 355; o) a change from R to Q at position 358; p) a change from T to S at position 365; q) a change from A to S at position 315; r) a change from A to T at position 317; s) a change from R to H at position 83 and a change from E to G at position 6; t) a change from R to H at position 83 and a change from K to Y at position 8; u) a change from R to H at position 83 and a change from A to L at position 11; v) a change from R to H at position 83 and a change from I to L at position 36; w) a change from R to H at position 83 and a change from C to L at position 53; x) a change from R to H at position 83 and a change from I to L at position 87; y) a change from R to H at position 83 and a change from P to G at position 89; z) a change from R to H at position 83 and a change from E to H at position 93; aa) a change from R to H at position 83 and a change from N to Q at position 95; ab) a change from R to H at position 83 and a change from V to G at position 145; ac) a change from R to H at position 83 and a change from G to P at position 168; ad) a change from R to H at position 83 and a change from T to S at position 200; ae) a change from R to H at position 83 and a change from A to Y at position 230; af) a change from R to H at position 83 and a change from V to Q at position 341; ag) a change from R to H at position 83 and a change from E to L at position 355; ah) a change from R to H at position 83 and a change from E to Y at position 355; ai) a change from R to H at position 83 and a change from E to Q at position 355; aj) a change from R to H at position 83 and a change from E to S at position 355; ak) a change from R to H at position 83 and a change from S to L at position 359; al) a change from R to H at position 83 and a change from S to G at position 359; am) a change from R to H at position 83 and a change from D to Q at position 367; an) a change from R to H at position 83 and a change from D to S at position 367; ao) a change from R to H at position 83 and a change from E to D at position 392; ap) a change from R to H at position 83 and a change from R to Y at position 439; aq) a change from R to H at position 83 and a change from K to Y at position 450; ar) a change from R to H at position 83 and a change from E to Q at position 460; as) a change from I to Y at position 87 and a change from P to Q at position 46; at) a change from I to Y at position 87 and a change from P to G at position 89; au) a change from I to Y at position 87 and a change from V to K at position 255; av) a change from I to Y at position 87 and a change from G to S at position 272; aw) a change from R to K at position 312 and a change from A to S at position 11; ax) a change from R to K at position 312 and a change from E to P at position 355; ay) a change from V to L at position 30, a change from R to H at position 83 and a change from T to S at position 200; az) a change from R to H at position 83, a change from P to G at position 89 and a change from T to S at position 200; ba) a change from a change from R to H at position 83, a change from P to S at position 89 and a change from T to S at position 200; bb) a change from R to H at position 83, a change from D to H at position 94 and a change from T to S at position 200; bc) a change from R to H at position 83, a change from D to P at position 94 and a change from T to S at position 200; bd) a change from R to H at position 83, a change from N to H at position 95 and a change from T to S at position 200; be) a change from R to H at position 83, a change from N to Q at position 95 and a change from T to S at position 200; bf) a change from R to H at position 83, a change from A to S at position 102 and a change from T to S at position 200; bg) a change from R to H at position 83, a change from T to S at position 200 and a change from E to D at position 249; bh) a change from E to G at position 6 and a change from N to Q at position 95; bi) a change from E to G at position 6 and a change from V to G at position 145; bj) a change from I to L at position 36 and a change from N to Q at position 95; bk) a change from P to Q at position 46 and a change from P to G at position 89; bl) a change from R to Q at position 83 and a change from D to S at position 367; bm) a change from P to G at position 89 and a change from S to G at position 359; bn) a change from E to P at position 355 and a change from S to G at position 359; bo) a change from E to G at position 6, a change from P to Q at position 46 and a change from P to G at position 89; bp) a change from E to G at position 6, a change from R to Q at position 83 and a change from I to L at position 87; bq) a change from a change from E to G at position 6, a change from G to S at position 272 and a change from E to P at position 355; br) a change from A to L at position 11, a change from R to H at position 83 and a change from E to P at position 355; bs) a change from A to L at position 11, a change from I to Y at position 87 and a change from E to P at position 355; bt) a change from A to L a position 11, a change from E to H at position 93 and change from S to L at position 359; bu) a change from A to S at position 11, a change from S to G at position 359 and a change from D to Q at position 367; bv) a change from P to Q at position 46, a change from N to Q at position 95 and a change from E to Y at position 355; bw) a change from R to H at position 83, a change from T to S at position 200 and a change from E to S at position 355; bx) a change from P to G at position 46 and a change from S to L at position 359; by) a change from I to L at position 87, a change from P to G at position 89, and a change from E to Q at position 355; or bz) a change from E to H at position 93 and a change from N to Q at position 95.

In addition is provided (5) A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein comprises at least one of the following: a) an H at position 28; b) a K at position 31; c) an H at position 34; d) an L at position 36; e) a Q at position 68; f) an S at position 68; g) a Q at position 83; h) an H at position 83; i) an S at position 88; j) a Y at position 87; k) a K at position 312; l) a G at position 317; m) an L at position 320; n) a P at position 355; o) a Q at position 358; p) an S at position 365; q) an S at position 315; r) a T at position 317; s) an H at position 83 and a G at position 6; t) an H at position 83 and a Y at position 8; u) an H at position 83 and an L at position 11; v) an H at position 83 and an L at position 36; w) an H at position 83 and an L at position 53; x) an H at position 83 and an L at position 87; y) an H at position 83 and a G at position 89; z) an H at position 83 and an H at position 93; aa) an H at position 83 and a Q at position 95; ab) an H at position 83 and a G at position 145; ac) an H at position 83 and a P at position 168; ad) an H at position 83 and an S at position 200; ae) an H at position 83 and a Y at position 230; af) an H at position 83 and Q at position 341; ag) an H at position 83 and an L at position 355; ah) an H at position 83 and a Y at position 355; ai) an H at position 83 and a Q at position 355; aj) an H at position 83 and an S at position 355; ak) an H at position 83 and an L at position 359; al) an R to H at position 83 and a G at position 359; am) an H at position 83 and a Q at position 367; an) an H at position 83 and an S at position 367; ao) an H at position 83 and a D at position 392; a p) an H at position 83 and a Y at position 439; aq) an H at position 83 and a Y at position 450; ar) an H at position 83 and a Q at position 460; as) a Y at position 87 and a Q at position 46; at) a Y at position 87 and a G at position 89; au) a Y at position 87 and a K at position 255; av) a Y at position 87 and an S at position 272; aw) a K at position 312 and an S at position 11; ax) a K at position 312 and a P at position 355; ay) an L at position 30, an H at position 83 and an S at position 200; az) an H at position 83, a G at position 89 and an S at position 200; ba) an H at position 83, an S at position 89 and an S at position 200; bb) an H at position 83, an H at position 94 and an S at position 200; bc) an H at position 83, a P at position 94 and an S at position 200; bd) an H at position 83, an H at position 95 and an S at position 200; be) an H at position 83, a Q at position 95 and an S at position 200; bf) an H at position 83, an S at position 102 and an S at position 200; bg) an H at position 83, an S at position 200 and a D at position 249; bh) a G at position 6 and a Q at position 95; bi) a G at position 6 and G at position 145; bj) an L at position 36 and a Q at position 95; bk) a Q at position 46 and a G at position 89; bl) a Q at position 83 and an S at position 367; bm) a G at position 89 and a G at position 359; bn) a P at position 355 and a G at position 359; bo) a G at position 6, a Q at position 46 and a G at position 89; bp) a G at position 6, a Q at position 83 and an L at position 87; bq) a G at position 6, an S at position 272 and a P at position 355; br) an L at position 11, an H at position 83 and a P at position 355; bs) an L at position 11, a Y at position 87 and a P at position 355; bt) an L a position 11, an H at position 93 and an L at position 359; bu) an S at position 11, a G at position 359 and a Q at position 367; bv) a Q at position 46, a Q at position 95 and a Y at position 355; bw) an H at position 83, an S at position 200 and an S at position 355; bx) a G at position 46 and an L at position 359; by) an L at position 87, a G at position 89, and a Q at position 355; and bz) an H at position 93 and a Q at position 95.

Further provided is: (6) A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein consists of: a) an H at position 28; b) a K at position 31; c) an H at position 34; d) an L at position 36; e) a Q at position 68; f) an S at position 68; g) a Q at position 83; h) an H at position 83; i) an S at position 88; j) a Y at position 87; k) a K at position 312; l) a G at position 317; m) an L at position 320; n) a P at position 355; o) a Q at position 358; p) an S at position 365; q) an S at position 315; r) a Tat position 317; S) an H at position 83 and a G at position 6; t) an H at position 83 and a Y at position 8; u) an H at position 83 and an L at position 11; v) an H at position 83 and an L at position 36; w) an H at position 83 and an L at position 53; x) an H at position 83 and an L at position 87; y) an H at position 83 and a G at position 89; z) an H at position 83 and an H at position 93; aa) an H at position 83 and a Q at position 95; ab) an H at position 83 and a G at position 145; ac) an H at position 83 and a P at position 168; ad) an H at position 83 and an S at position 200; ae) an H at position 83 and a Y at position 230; af) an H at position 83 and Q at position 341; ag) an H at position 83 and an L at position 355; ah) an H at position 83 and a Y at position 355; ai) an H at position 83 and a Q at position 355; aj) an H at position 83 and an S at position 355; ak) an H at position 83 and an L at position 359; al) an R to H at position 83 and a G at position 359; am) an H at position 83 and a Q at position 367; an) an H at position 83 and an S at position 367; ao) an H at position 83 and a D at position 392; ap) an H at position 83 and a Y at position 439; aq) an H at position 83 and a Y at position 450; ar) an H at position 83 and a Q at position 460; as) a Y at position 87 and a Q at position 46; at) a Y at position 87 and a G at position 89; au) a Y at position 87 and a K at position 255; av) a Y at position 87 and an S at position 272; aw) a K at position 312 and an S at position 11; ax) a K at position 312 and a P at position 355; ay) an L at position 30, an H at position 83 and an S at position 200; az) an H at position 83, a G at position 89 and an S at position 200; ba) an H at position 83, an S at position 89 and an S at position 200; bb) an H at position 83, an H at position 94 and an S at position 200; bc) an H at position 83, a P at position 94 and an S at position 200; bd) an H at position 83, an H at position 95 and an S at position 200; be) an H at position 83, a Q at position 95 and an S at position 200; bf) an H at position 83, an S at position 102 and an S at position 200; bg) an H at position 83, an S at position 200 and a D at position 249; bh) a G at position 6 and a Q at position 95; bi) a G at position 6 and G at position 145; bj) an L at position 36 and a Q at position 95; bk) a Q at position 46 and a G at position 89; bl) a Q at position 83 and an S at position 367; bm) a G at position 89 and a G at position 359; bn) a P at position 355 and a G at position 359; bo) a G at position 6, a Q at position 46 and a G at position 89; bp) a G at position 6, a Q at position 83 and an L at position 87; bq) a G at position 6, an S at position 272 and a P at position 355; br) an L at position 11, an H at position 83 and a P at position 355; bs) an L at position 11, a Y at position 87 and a P at position 355; bt) an L a position 11, an H at position 93 and an L at position 359; bu) an S at position 11, a G at position 359 and a Q at position 367; bv) a Q at position 46, a Q at position 95 and a Y at position 355; bw) an H at position 83, an S at position 200 aid an S at position 355; bx) a G at position 46 and an L at position 359; by) an L at position 87, a G at position 89, and a Q at position 355; or bz) an H at position 93 and a Q at position 95.

Also provide is: (7) The transformed photosynthetic organism of any one of 3 to 6, wherein the transformed photosynthetic organism expresses the modified rbcL protein and produces increased biomass as compared to an untransformed photosynthetic organism of the same species. (8) The transformed photosynthetic organism of 1, 2 or 7 wherein the increase in biomass is measured by at least one of a competition assay, growth rate, carrying capacity, productivity or cell proliferation. (9) The transformed photosynthetic organism of 8, wherein the increase in biomass is measured by a competition assay. (10) The transformed photosynthetic organism of 9, wherein the competition assay is performed in a turbidostat. (11) The transformed photosynthetic organism of 1, 2 or 7, wherein the increase is shown by the transformed photosynthetic organism having a positive selection coefficient as compared to an untransformed organism of the same species. (12) The transformed photosynthetic organism of 11, wherein the positive selection coefficient is from 0.05 to 0.10, from 0.10 to 0.5, from 0.5 to 0.75, from 0.75 to 1.0, from 1.0 to 1.5, from 1.5 to 2.0, or from 2.0 to 3.0. (13) The transformed photosynthetic organism of 8, wherein the increased biomass is measured by growth rate. (14) The transformed photosynthetic organism of 13, wherein the transformed photosynthetic organism has an increased growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (15) The transformed photosynthetic organism of 8, wherein the increased biomass is measured by carrying capacity. (16) The transformed photosynthetic organism of 15, wherein the units of carrying capacity are mass per unit of volume or mass per unit of area. (17) The transformed photosynthetic organism of 8, where the increase biomass is measured by increased productivity. (18) The transformed photosynthetic organism of 17, wherein the units of productivity are grams per meter squared per day, mass per unit area such as tons per acre/hectare, or volume per unit area such as bushels per acre/hectare. (19) The transformed photosynthetic organism of 17 wherein the transformed photosynthetic organism has an increase in productivity as compared to an untransformed photosynthetic organism of the same species of from 5% to 25%, or from 25% to 50%, of from 50% to 75%, of from 75% to 100%, of from 100% to 150%, of from 150% to 200%, for from 200% to 300% or from 300% to 400%. (20) The transformed photosynthetic organism of any one of 1 to 6, wherein the transformed photosynthetic organism is a bacterium. (21) The transformed photosynthetic organism of 20, wherein the bacterium is a cyanobacterium. (22) The transformed photosynthetic organism of any one of 1 to 6, wherein the transformed photosynthetic organism is an alga. (23) The transformed photosynthetic organism of 22, wherein the alga is a microalga. (24) The transformed photosynthetic organism of 23, wherein the microalga is at least one of Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. (25) The transformed photosynthetic organism of 24, wherein the microalga is at least one of Chlamydomonas. reinhardtii, N. oceanica, N. salina, Dunaliella. salina, H. pluvalis, S. dimorphus, Dunaliella viridis, N. oculata, Dunaliella tertiolecta, S. Maximus, or A. Fusiformus. (26) The transformed photosynthetic organism of one of 1 to 6, wherein the transformed photosynthetic organism is a vascular plant. (27) The transformed photosynthetic organism of 26, wherein the vascular plant is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Oleo europaea), sesame, peanut (Arachis hypogaea), Arabidopsis sp., tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops or fruits. (28) The transformed photosynthetic organism of 2, wherein said exogenous polynucleotide is selected from the group consisting of SEQ ID NOs. 81-159. (29) A method for increasing biomass production in a photosynthetic organism comprising transforming the photosynthetic organism with an exogenous polynucleotide encoding a modified rbcL protein of SEQ ID NO: 1 to produce a transformed photosynthetic organism; wherein said modification to SEQ ID NO 1 comprises at least one amino acid substitution in the loop at positions 25-35, the β-sheet at positions 83-89, the α-helix at positions 310-321 and the loop-helix-loop at positions 355-365; and wherein the transformed photosynthetic organism expresses the modified rbcL protein and produces increased biomass as compared to an untransformed photosynthetic organism of the same species. (30) A method for increasing biomass production by a photosynthetic organism comprising transforming the photosynthetic organism with an exogenous polynucleotide encoding a modified rbcL protein of any one of SEQ ID NOs 2 to 79 to produce a transformed photosynthetic organism; wherein the transformed photosynthetic organism expresses the modified rbcL protein and produces increased biomass as compared to an untransformed photosynthetic organism of the same species.

Also provided is (31) A method for increasing biomass production by a photosynthetic organism comprising transforming the photosynthetic organism with an exogenous polynucleotide encoding a modified rbcL protein of SEQ ID NO. 1 or a protein having at least 95% sequence identity to SEQ ID NO. 1; wherein the transformed photosynthetic organism expresses the modified rbcL protein and produces increased biomass as compared to an untransformed photosynthetic organism of the same species; and wherein the modified rbcL protein comprises at least one of the following modifications: a) a change from D to H at position 28; b) a change from V to K at position 31; c) a change from T to H at position 34; d) a change from I to L at position 36; e) a change from T to Q at position 68; f) a change from T to S at position 68; g) a change from R to Q at position 83; h) a change from R to H at position 83; i) a change from E to S at position 88; j) a change from I to Y at position 87; k) a change from R to K at position 312; l) a change from A to G at position 317; m) a change from M to L at position 320; n) a change from E to P at position 355; o) a change from R to Q at position 358; p) a change from T to S at position 365; q) a change from A to S at position 315; r) a change from A to T at position 317; s) a change from R to H at position 83 and a change from E to G at position 6; t) a change from R to H at position 83 and a change from K to Y at position 8; u) a change from R to H at position 83 and a change from A to L at position 11; v) a change from R to H at position 83 and a change from I to L at position 36; w) a change from R to H at position 83 and a change from C to L at position 53; x) a change from R to H at position 83 and a change from I to L at position 87; y) a change from R to H at position 83 and a change from P to G at position 89; z) a change from R to H at position 83 and a change from E to H at position 93; aa) a change from R to H at position 83 and a change from N to Q at position 95; ab) a change from R to H at position 83 and a change from V to G at position 145; ac) a change from R to H at position 83 and a change from G to P at position 168; ad) a change from R to H at position 83 and a change from T to S at position 200; ae) a change from R to H at position 83 and a change from A to Y at position 230; af) a change from R to H at position 83 and a change from V to Q at position 341; ag) a change from R to H at position 83 and a change from E to L at position 355; ah) a change from R to H at position 83 and a change from E to Y at position 355; ai) a change from R to H at position 83 and a change from E to Q at position 355; aj) a change from R to H at position 83 and a change from E to S at position 355; ak) a change from R to H at position 83 and a change from S to L at position 359; al) a change from R to H at position 83 and a change from S to G at position 359; am) a change from R to H at position 83 and a change from D to Q at position 367; an) a change from R to H at position 83 and a change from D to S at position 367; ao) a change from R to H at position 83 and a change from E to D at position 392; ap) a change from R to H at position 83 and a change from R to Y at position 439; aq) a change from R to H at position 83 and a change from K to Y at position 450; ar) a change from R to H at position 83 and a change from E to Q at position 460; as) a change from I to Y at position 87 and a change from P to Q at position 46; at) a change from I to Y at position 87 and a change from P to G at position 89; au) a change from I to Y at position 87 and a change from V to K at position 255; av) a change from I to Y at position 87 and a change from G to S at position 272; aw) a change from R to K at position 312 and a change from A to S at position 11; ax) a change from R to K at position 312 and a change from E to P at position 355; ay) a change from V to L at position 30, a change from R to H at position 83 and a change from T to S at position 200; az) a change from R to H at position 83, a change from P to G at position 89 and a change from T to S at position 200; ba) a change from a change from R to H at position 83, a change from P to S at position 89 and a change from T to S at position 200; bb) a change from R to H at position 83, a change from D to H at position 94 and a change from T to S at position 200; bc) a change from R to H at position 83, a change from D to P at position 94 and a change from T to S at position 200; bd) a change from R to H at position 83, a change from N to H at position 95 and a change from T to S at position 200; be) a change from R to H at position 83, a change from N to Q at position 95 and a change from T to S at position 200; bf) a change from R to H at position 83, a change from A to S at position 102 and a change from T to S at position 200; bg) a change from R to H at position 83, a change from T to S at position 200 and a change from E to D at position 249; bh) a change from E to G at position 6 and a change from N to Q at position 95; bi) a change from E to G at position 6 and a change from V to G at position 145; bj) a change from I to L at position 36 and a change from N to Q at position 95; bk) a change from P to Q at position 46 and a change from P to G at position 89; bl) a change from R to Q at position 83 and a change from D to S at position 367; bm) a change from P to G at position 89 and a change from S to G at position 359; bn) a change from E to P at position 355 and a change from S to G at position 359; bo) a change from E to G at position 6, a change from P to Q at position 46 and a change from P to G at position 89; bp) a change from E to G at position 6, a change from R to Q at position 83 and a change from I to L at position 87; bq) a change from a change from E to G at position 6, a change from G to S at position 272 and a change from E to P at position 355; br) a change from A to L at position 11, a change from R to H at position 83 and a change from E to P at position 355; bs) a change from A to L at position 11, a change from I to Y at position 87 and a change from E to P at position 355; bt) a change from A to L a position 11, a change from E to H at position 93 and change from S to L at position 359; bu) a change from A to S at position 11, a change from S to G at position 359 and a change from D to Q at position 367; bv) a change from P to Q at position 46, a change from N to Q at position 95 and a change from E to Y at position 355; bw) a change from R to H at position 83, a change from T to S at position 200 and a change from E to S at position 355; bx) a change from P to G at position 46 and a change from S to L at position 359; by) a change from I to L at position 87, a change from P to G at position 89, and a change from E to Q at position 355; and bz) a change from E to H at position 93 and a change from N to Q at position 95.

Also provided is (32) A method for increasing biomass production by a photosynthetic organism comprising transforming the photosynthetic organism with an exogenous polynucleotide encoding a modified rbcL protein of SEQ ID NO. 1 or a protein having at least 95% sequence identity to SEQ ID NO. 1 to produce a transformed photosynthetic organism; wherein the transformed photosynthetic organism expresses the modified rbcL protein and produces increased biomass as compared to an untransformed photosynthetic organism of the same species; and wherein the modified rbcL protein consists of a modification selected from: a) a change from D to H at position 28; b) a change from V to K at position 31; c) a change from T to H at position 34; d) a change from I to L at position 36; e) a change from T to Q at position 68; f) a change from T to S at position 68; g) a change from R to Q at position 83; h) a change from R to H at position 83; i) a change from E to S at position 88; j) a change from I to Y at position 87; k) a change from R to K at position 312; l) a change from A to G at position 317; m) a change from M to L at position 320; n) a change from E to P at position 355; o) a change from R to Q at position 358; p) a change from T to S at position 365; q) a change from A to S at position 315; r) a change from A to T at position 317; s) a change from R to H at position 83 and a change from E to G at position 6; t) a change from R to H at position 83 and a change from K to Y at position 8; u) a change from R to H at position 83 and a change from A to L at position 11; v) a change from R to H at position 83 and a change from I to L at position 36; w) a change from R to H at position 83 and a change from C to L at position 53; x) a change from R to H at position 83 and a change from I to L at position 87; y) a change from R to H at position 83 and a change from P to G at position 89; z) a change from R to H at position 83 and a change from E to H at position 93; aa) a change from R to H at position 83 and a change from N to Q at position 95; ab) a change from R to H at position 83 and a change from V to G at position 145; ac) a change from R to H at position 83 and a change from G to P at position 168; ad) a change from R to H at position 83 and a change from T to S at position 200; ae) a change from R to H at position 83 and a change from A to Y at position 230; af) a change from R to H at position 83 and a change from V to Q at position 341; ag) a change from R to H at position 83 and a change from E to L at position 355; ah) a change from R to H at position 83 and a change from E to Y at position 355; ai) a change from R to H at position 83 and a change from E to Q at position 355; aj) a change from R to H at position 83 and a change from E to S at position 355; ak) a change from R to H at position 83 and a change from S to L at position 359; al) a change from R to H at position 83 and a change from S to G at position 359; am) a change from R to H at position 83 and a change from D to Q at position 367; an) a change from R to H at position 83 and a change from D to S at position 367; ao) a change from R to H at position 83 and a change from E to D at position 392; ap) a change from R to H at position 83 and a change from R to Y at position 439; aq) a change from R to H at position 83 and a change from K to Y at position 450; ar) a change from R to H at position 83 and a change from E to Q at position 460; as) a change from I to Y at position 87 and a change from P to Q at position 46; at) a change from I to Y at position 87 and a change from P to G at position 89; au) a change from I to Y at position 87 and a change from V to K at position 255; av) a change from I to Y at position 87 and a change from G to S at position 272; aw) a change from R to K at position 312 and a change from A to S at position 11; ax) a change from R to K at position 312 and a change from E to P at position 355; ay) a change from V to L at position 30, a change from R to H at position 83 and a change from T to S at position 200; az) a change from R to H at position 83, a change from P to G at position 89 and a change from T to S at position 200; ba) a change from a change from R to H at position 83, a change from P to S at position 89 and a change from T to S at position 200; bb) a change from R to H at position 83, a change from D to H at position 94 and a change from T to S at position 200; bc) a change from R to H at position 83, a change from D to P at position 94 and a change from T to S at position 200; bd) a change from R to H at position 83, a change from N to H at position 95 and a change from T to S at position 200; be) a change from R to H at position 83, a change from N to Q at position 95 and a change from T to S at position 200; bf) a change from R to H at position 83, a change from A to S at position 102 and a change from T to S at position 200; bg) a change from R to H at position 83, a change from T to S at position 200 and a change from E to D at position 249; bh) a change from E to G at position 6 and a change from N to Q at position 95; bi) a change from E to G at position 6 and a change from V to G at position 145; bj) a change from I to L at position 36 and a change from N to Q at position 95; bk) a change from P to Q at position 46 and a change from P to G at position 89; bl) a change from R to Q at position 83 and a change from D to S at position 367; bm) a change from P to G at position 89 and a change from S to G at position 359; bn) a change from E to P at position 355 and a change from S to G at position 359; bo) a change from E to G at position 6, a change from P to Q at position 46 and a change from P to G at position 89; bp) a change from E to G at position 6, a change from R to Q at position 83 and a change from I to L at position 87; bq) a change from a change from E to G at position 6, a change from G to S at position 272 and a change from E to P at position 355; br) a change from A to L at position 11, a change from R to H at position 83 and a change from E to P at position 355; bs) a change from A to L at position 11, a change from I to Y at position 87 and a change from E to P at position 355; bt) a change from A to L a position 11, a change from E to H at position 93 and change from S to L at position 359; bu) a change from A to S at position 11, a change from S to G at position 359 and a change from D to Q at position 367; bv) a change from P to Q at position 46, a change from N to Q at position 95 and a change from E to Y at position 355; bw) a change from R to H at position 83, a change from T to S at position 200 and a change from E to S at position 355; bx) a change from P to G at position 46 and a change from S to L at position 359; by) a change from I to L at position 87, a change from P to G at position 89, and a change from E to Q at position 355; or bz) a change from E to H at position 93 and a change from N to Q at position 95.

Provided herein is (33) A method for increasing biomass production by a photosynthetic organism comprising transforming the photosynthetic organism with an exogenous polynucleotide encoding a modified rbcL protein of SEQ ID NO. 1 or a protein having at least 95% sequence identity to SEQ ID NO. 1 to produce a transformed photosynthetic organism; wherein the transformed photosynthetic organism expresses the modified rbcL protein and produces increased biomass as compared to an untransformed photosynthetic organism of the same species; and wherein the modified rbcL protein comprises at least one of the following: a) an H at position 28; b) a K at position 31; c) an H at position 34; d) an L at position 36; e) a Q at position 68; f) an S at position 68; g) a Q at position 83; h) an H at position 83; i) an S at position 88; j) a Y at position 87; k) a K at position 312; l) a G at position 317; m) an L at position 320; n) a P at position 355; o) a Q at position 358; p) an S at position 365; q) an S at position 315; r) a T at position 317; s) an H at position 83 and a G at position 6; t) an H at position 83 and a Y at position 8; u) an H at position 83 and an L at position 11; v) an H at position 83 and an L at position 36; w) an H at position 83 and an L at position 53; x) an H at position 83 and an L at position 87; y) an H at position 83 and a G at position 89; z) an H at position 83 and an H at position 93; aa) an H at position 83 and a Q at position 95; ab) an H at position 83 and a G at position 145; ac) an H at position 83 and a P at position 168; ad) an H at position 83 and an S at position 200; ae) an H at position 83 and a Y at position 230; af) an H at position 83 and Q at position 341; ag) an H at position 83 and an L at position 355; ah) an H at position 83 and a Y at position 355; ai) an H at position 83 and a Q at position 355; aj) an H at position 83 and an S at position 355; ak) an H at position 83 and an L at position 359; al) an R to H at position 83 and a G at position 359; am) an H at position 83 and a Q at position 367; an) an H at position 83 and an S at position 367; ao) an H at position 83 and a D at position 392; ap) an H at position 83 and a Y at position 439; aq) an H at position 83 and a Y at position 450; ar) an H at position 83 and a Q at position 460; as) a Y at position 87 and a Q at position 46; at) a Y at position 87 and a G at position 89; au) a Y at position 87 and a K at position 255; av) a Y at position 87 and an S at position 272; aw) a K at position 312 and an S at position 11; ax) a K at position 312 and a P at position 355; ay) an L at position 30, an H at position 83 and an S at position 200; az) an H at position 83, a G at position 89 and an S at position 200; ba) an H at position 83, an S at position 89 and an S at position 200; bb) an H at position 83, an H at position 94 and an S at position 200; bc) an H at position 83, a P at position 94 and an S at position 200; bd) an H at position 83, an H at position 95 and an S at position 200; be) an H at position 83, a Q at position 95 and an S at position 200; bf) an H at position 83, an S at position 102 and an S at position 200; bg) an H at position 83, an S at position 200 and a D at position 249; bh) a G at position 6 and a Q at position 95; bi) a G at position 6 and G at position 145; bj) an L at position 36 and a Q at position 95; bk) a Q at position 46 and a G at position 89; bl) a Q at position 83 and an S at position 367; bm) a G at position 89 and a G at position 359; bn) a P at position 355 and a G at position 359; bo) a G at position 6, a Q at position 46 and a G at position 89; bp) a G at position 6, a Q at position 83 and an L at position 87; bq) a G at position 6, an S at position 272 and a P at position 355; br) an L at position 11, an H at position 83 and a P at position 355; bs) an L at position 11, a Y at position 87 and a P at position 355; bt) an L a position 11, an H at position 93 and an L at position 359; bu) an S at position 11, a G at position 359 and a Q at position 367; bv) a Q at position 46, a Q at position 95 and a Y at position 355; bw) an H at position 83, an S at position 200 and an S at position 355; bx) a G at position 46 and an L at position 359; by) an L at position 87, a G at position 89, and a Q at position 355; and bz) an H at position 93 and a Q at position 95.

Further provided is: (34) A method for increasing biomass production by a photosynthetic organism comprising transforming said photosynthetic organism with an exogenous polynucleotide encoding a modified rbcL protein of SEQ ID NO. 1 or a protein having at least 95% sequence identity to SEQ ID NO. 1 to produce a transformed photosynthetic organism; wherein the transformed photosynthetic organism expresses the modified rbcL protein and produces increased biomass as compared to an untransformed photosynthetic organism of the same species; and wherein the modified rbcL protein consists of: a) an H at position 28; b) a K at position 31; c) an H at position 34; d) an L at position 36; e) a Q at position 68; f) an S at position 68; g) a Q at position 83; h) an H at position 83; i) an S at position 88; j) a Y at position 87; k) a K at position 312; l) a G at position 317; m) an L at position 320; n) a P at position 355; o) a Q at position 358; p) an S at position 365; q) an S at position 315; r) a Tat position 317; s) an H at position 83 and a G at position 6; t) an H at position 83 and a Y at position 8; u) an H at position 83 and an L at position 11; v) an H at position 83 and an L at position 36; w) an H at position 83 and an L at position 53; x) an H at position 83 and an L at position 87; y) an H at position 83 and a G at position 89; z) an H at position 83 and an H at position 93; aa) an H at position 83 and a Q at position 95; ab) an H at position 83 and a G at position 145; ac) an H at position 83 and a P at position 168; ad) an H at position 83 and an S at position 200; ae) an H at position 83 and a Y at position 230; af) an H at position 83 and Q at position 341; ag) an H at position 83 and an L at position 355; ah) an H at position 83 and a Y at position 355; ai) an H at position 83 and a Q at position 355; aj) an H at position 83 and an S at position 355; ak) an H at position 83 and an L at position 359; al) an R to H at position 83 and a G at position 359; am) an H at position 83 and a Q at position 367; an) an H at position 83 and an S at position 367; ao) an H at position 83 and a D at position 392; ap) an H at position 83 and a Y at position 439; aq) an H at position 83 and a Y at position 450; ar) an H at position 83 and a Q at position 460; as) a Y at position 87 and a Q at position 46; at) a Y at position 87 and a G at position 89; au) a Y at position 87 and a K at position 255; av) a Y at position 87 and an S at position 272; aw) a K at position 312 and an S at position 11; ax) a K at position 312 and a P at position 355; ay) an L at position 30, an H at position 83 and an S at position 200; az) an H at position 83, a G at position 89 and an S at position 200; ba) an H at position 83, an S at position 89 and an S at position 200; bb) an H at position 83, an H at position 94 and an S at position 200; bc) an H at position 83, a P at position 94 and an S at position 200; bd) an H at position 83, an H at position 95 and an S at position 200; be) an H at position 83, a Q at position 95 and an S at position 200; bf) an H at position 83, an S at position 102 and an S at position 200; bg) an H at position 83, an S at position 200 and a D at position 249; bh) a G at position 6 and a Q at position 95; bi) a G at position 6 and G at position 145; bj) an L at position 36 and a Q at position 95; bk) a Q at position 46 and a G at position 89; bl) a Q at position 83 and an S at position 367; bm) a G at position 89 and a G at position 359; bn) a P at position 355 and a G at position 359; bo) a G at position 6, a Q at position 46 and a G at position 89; bp) a G at position 6, a Q at position 83 and an L at position 87; bq) a G at position 6, an S at position 272 and a P at position 355; br) an L at position 11, an H at position 83 and a P at position 355; bs) an L at position 11, a Y at position 87 and a P at position 355; bt) an L a position 11, an H at position 93 and an L at position 359; bu) an S at position 11, a G at position 359 and a Q at position 367; bv) a Q at position 46, a Q at position 95 and a Y at position 355; bw) an H at position 83, an S at position 200 and an S at position 355; bx) a G at position 46 and an L at position 359; by) an L at position 87, a G at position 89, and a Q at position 355; or bz) an H at position 93 and a Q at position 95. (35) The method of any one of 29 to 34, wherein the increase in biomass production is measured by at least one of a competition assay, growth rate, carrying capacity, productivity or cell proliferation. (36) The method of 35, wherein the increase in biomass production is measured by a competition assay. (37) The method of 36, wherein the competition assay is performed in a turbidostat. (38) The method of any one of 29 to 34, wherein the increase in biomass production is shown by the transformed photosynthetic organism having a positive selection coefficient as compared to an untransformed photosynthetic organism of the same species. (39) The method of 38, wherein the positive selection coefficient is from 0.05 to 0.10, from 0.10 to 0.5, from 0.5 to 0.75, from 0.75 to 1.0, from 1.0 to 1.5, from 1.5 to 2.0, or from 2.0 to 3.0. (40) The method of 35, wherein the increase in biomass production is measured by growth rate. (41) The method of 40, wherein the transformed photosynthetic organism has an increased growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%. (42) The method of 35, wherein the increase in biomass production is measured by carrying capacity. (43) The method of 42, wherein the units of carrying capacity are mass per unit of volume or mass per unit of area. (44) The method of 35, wherein the increase in biomass production is measured by increased productivity. (45) The method of 44, wherein the units of productivity are grams per meter squared per day, mass per unit area such as tons per acre/hectare, or volume per unit area such as bushels per acre/hectare. (46) The method of 4, wherein the transformed photosynthetic organism has an increase in productivity as compared to an untransformed photosynthetic organism of the same species of from 5% to 25%, or from 25% to 50%, of from 50% to 75%, of from 75% to 100%, of from 100% to 150%, of from 150% to 200%, for from 200% to 300% or from 300% to 400%. (47) The method of any one of 29 to 34, wherein the transformed photosynthetic organism is a bacterium. (48) The method of 47, wherein the bacterium is a cyanobacterium. (49) The method of any one of 29 to 34, wherein the transformed photosynthetic organism is an alga. (50) The method of 49, wherein the alga is a microalga. (51) The method of 50, wherein the microalga is at least one of Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. (52) The method of 51, wherein the microalga is at least one of Chlamydomonas. reinhardtii, N. oceanica, N. salina, Dunaliella. salina, H. pluvalis, S. dimorphus, Dunaliella viridis, N. oculata, Dunaliella tertiolecta, S. Maximus, or A. Fusiformus. (53) The method of one of 29 to 34, wherein the transformed photosynthetic organism is a vascular plant. (54) The method of 53, wherein the vascular plant is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, peanut (Arachis hypogaea), Arabidopsis sp., tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops or fruits.

Also provided is: (55) A modified rbcL protein of SEQ ID NO: 1 said modification to SEQ ID NO 1 comprising at least one amino acid substitution in the loop at positions 25-35, the β-sheet at positions 83-89, the α-helix at positions 310-321 and the loop-helix-loop at positions 355-365. And (56) a modified rbcL protein comprising any one of SEQ ID NOs 2 to 79.

Also provided is (57) A modified rbcL protein comprising SEQ ID NO. 1 or a protein having at least 95% sequence identity to SEQ ID NO. 1; said modification comprising at least one of the following: a) a change from D to H at position 28; b) a change from V to K at position 31; c) a change from T to H at position 34; d) a change from I to L at position 36; e) a change from T to Q at position 68; f) a change from T to S at position 68; g) a change from R to Q at position 83; h) a change from R to H at position 83; i) a change from E to S at position 88; j) a change from I to Y at position 87; k) a change from R to K at position 312; l) a change from A to G at position 317; m) a change from M to L at position 320; n) a change from E to P at position 355; o) a change from R to Q at position 358; p) a change from T to S at position 365; q) a change from A to S at position 315; r) a change from A to T at position 317; s) a change from R to H at position 83 and a change from E to G at position 6; t) a change from R to H at position 83 and a change from K to Y at position 8; u) a change from R to H at position 83 and a change from A to L at position 11; v) a change from R to H at position 83 and a change from I to L at position 36; w) a change from R to H at position 83 and a change from C to L at position 53; x) a change from R to H at position 83 and a change from I to L at position 87; y) a change from R to H at position 83 and a change from P to G at position 89; z) a change from R to H at position 83 and a change from E to H at position 93; aa) a change from R to H at position 83 and a change from N to Q at position 95; ab) a change from R to H at position 83 and a change from V to G at position 145; ac) a change from R to H at position 83 and a change from G to P at position 168; ad) a change from R to H at position 83 and a change from T to S at position 200; ae) a change from R to H at position 83 and a change from A to Y at position 230; af) a change from R to H at position 83 and a change from V to Q at position 341; ag) a change from R to H at position 83 and a change from E to L at position 355; ah) a change from R to H at position 83 and a change from E to Y at position 355; ai) a change from R to H at position 83 and a change from E to Q at position 355; aj) a change from R to H at position 83 and a change from E to S at position 355; ak) a change from R to H at position 83 and a change from S to L at position 359; al) a change from R to H at position 83 and a change from S to G at position 359; am) a change from R to H at position 83 and a change from D to Q at position 367; an) a change from R to H at position 83 and a change from D to S at position 367; ao) a change from R to H at position 83 and a change from E to D at position 392; ap) a change from R to H at position 83 and a change from R to Y at position 439; aq) a change from R to H at position 83 and a change from K to Y at position 450; ar) a change from R to H at position 83 and a change from E to Q at position 460; as) a change from I to Y at position 87 and a change from P to Q at position 46; at) a change from I to Y at position 87 and a change from P to G at position 89; au) a change from I to Y at position 87 and a change from V to K at position 255; av) a change from I to Y at position 87 and a change from G to S at position 272; aw) a change from R to K at position 312 and a change from A to S at position 11; ax) a change from R to K at position 312 and a change from E to P at position 355; ay) a change from V to L at position 30, a change from R to H at position 83 and a change from T to S at position 200; az) a change from R to H at position 83, a change from P to G at position 89 and a change from T to S at position 200; ba) a change from a change from R to H at position 83, a change from P to S at position 89 and a change from T to S at position 200; bb) a change from R to H at position 83, a change from D to H at position 94 and a change from T to S at position 200; bc) a change from R to H at position 83, a change from D to P at position 94 and a change from T to S at position 200; bd) a change from R to H at position 83, a change from N to H at position 95 and a change from T to S at position 200; be) a change from R to H at position 83, a change from N to Q at position 95 and a change from T to S at position 200; bf) a change from R to H at position 83, a change from A to S at position 102 and a change from T to S at position 200; bg) a change from R to H at position 83, a change from T to S at position 200 and a change from E to D at position 249; bh) a change from E to G at position 6 and a change from N to Q at position 95; bi) a change from E to G at position 6 and a change from V to G at position 145; bj) a change from I to L at position 36 and a change from N to Q at position 95; bk) a change from P to Q at position 46 and a change from P to G at position 89; bl) a change from R to Q at position 83 and a change from D to S at position 367; bm) a change from P to G at position 89 and a change from S to G at position 359; bn) a change from E to P at position 355 and a change from S to G at position 359; bo) a change from E to G at position 6, a change from P to Q at position 46 and a change from P to G at position 89; bp) a change from E to G at position 6, a change from R to Q at position 83 and a change from I to L at position 87; bq) a change from a change from E to G at position 6, a change from G to S at position 272 and a change from E to P at position 355; br) a change from A to L at position 11, a change from R to H at position 83 and a change from E to P at position 355; bs) a change from A to L at position 11, a change from I to Y at position 87 and a change from E to P at position 355; bt) a change from A to L a position 11, a change from E to H at position 93 and change from S to L at position 359; bu) a change from A to S at position 11, a change from S to G at position 359 and a change from D to Q at position 367; bv) a change from P to Q at position 46, a change from N to Q at position 95 and a change from E to Y at position 355; bw) a change from R to H at position 83, a change from T to S at position 200 and a change from E to S at position 355; bx) a change from P to G at position 46 and a change from S to L at position 359; by) a change from I to L at position 87, a change from P to G at position 89, and a change from E to Q at position 355; and bz) a change from E to H at position 93 and a change from N to Q at position 95.

Also provided is: (58) A modified rbcL protein comprising SEQ ID NO. 1 or a protein having at least 95% sequence identity to SEQ ID NO. 1; wherein the modification consists of: a) a change from D to H at position 28; b) a change from V to K at position 31; c) a change from T to H at position 34; d) a change from I to L at position 36; e) a change from T to Q at position 68; f) a change from T to S at position 68; g) a change from R to Q at position 83; h) a change from R to H at position 83; i) a change from E to S at position 88; j) a change from I to Y at position 87; k) a change from R to K at position 312; l) a change from A to G at position 317; m) a change from M to L at position 320; n) a change from E to P at position 355; o) a change from R to Q at position 358; p) a change from T to S at position 365; q) a change from A to S at position 315; r) a change from A to Tat position 317; s) a change from R to H at position 83 and a change from E to G at position 6; t) a change from R to H at position 83 and a change from K to Y at position 8; u) a change from R to H at position 83 and a change from A to L at position 11; v) a change from R to H at position 83 and a change from I to L at position 36; w) a change from R to H at position 83 and a change from C to L at position 53; x) a change from R to H at position 83 and a change from I to L at position 87; y) a change from R to H at position 83 and a change from P to G at position 89; z) a change from R to H at position 83 and a change from E to H at position 93; aa) a change from R to H at position 83 and a change from N to Q at position 95; ab) a change from R to H at position 83 and a change from V to G at position 145; ac) a change from R to H at position 83 and a change from G to P at position 168; ad) a change from R to H at position 83 and a change from T to S at position 200; ae) a change from R to H at position 83 and a change from A to Y at position 230; af) a change from R to H at position 83 and a change from V to Q at position 341; ag) a change from R to H at position 83 and a change from E to L at position 355; ah) a change from R to H at position 83 and a change from E to Y at position 355; ai) a change from R to H at position 83 and a change from E to Q at position 355; aj) a change from R to H at position 83 and a change from E to S at position 355; ak) a change from R to H at position 83 and a change from S to L at position 359; al) a change from R to H at position 83 and a change from S to G at position 359; am) a change from R to H at position 83 and a change from D to Q at position 367; an) a change from R to H at position 83 and a change from D to S at position 367; ao) a change from R to H at position 83 and a change from E to D at position 392; ap) a change from R to H at position 83 and a change from R to Y at position 439; aq) a change from R to H at position 83 and a change from K to Y at position 450; ar) a change from R to H at position 83 and a change from E to Q at position 460; as) a change from I to Y at position 87 and a change from P to Q at position 46; at) a change from I to Y at position 87 and a change from P to G at position 89; au) a change from I to Y at position 87 and a change from V to K at position 255; av) a change from I to Y at position 87 and a change from G to S at position 272; aw) a change from R to K at position 312 and a change from A to S at position 11; ax) a change from R to K at position 312 and a change from E to P at position 355; ay) a change from V to L at position 30, a change from R to H at position 83 and a change from T to S at position 200; az) a change from R to H at position 83, a change from P to G at position 89 and a change from T to S at position 200; ba) a change from a change from R to H at position 83, a change from P to S at position 89 and a change from T to S at position 200; bb) a change from R to H at position 83, a change from D to H at position 94 and a change from T to S at position 200; bc) a change from R to H at position 83, a change from D to P at position 94 and a change from T to S at position 200; bd) a change from R to H at position 83, a change from N to H at position 95 and a change from T to S at position 200; be) a change from R to H at position 83, a change from N to Q at position 95 and a change from T to S at position 200; bf) a change from R to H at position 83, a change from A to S at position 102 and a change from T to S at position 200; bg) a change from R to H at position 83, a change from T to S at position 200 and a change from E to D at position 249; bh) a change from E to G at position 6 and a change from N to Q at position 95; bi) a change from E to G at position 6 and a change from V to G at position 145; bj) a change from I to L at position 36 and a change from N to Q at position 95; bk) a change from P to Q at position 46 and a change from P to G at position 89; bl) a change from R to Q at position 83 and a change from D to S at position 367; bm) a change from P to G at position 89 and a change from S to G at position 359; bn) a change from E to P at position 355 and a change from S to G at position 359; bo) a change from E to G at position 6, a change from P to Q at position 46 and a change from P to G at position 89; bp) a change from E to G at position 6, a change from R to Q at position 83 and a change from I to L at position 87; bq) a change from a change from E to G at position 6, a change from G to S at position 272 and a change from E to P at position 355; br) a change from A to L at position 11, a change from R to H at position 83 and a change from E to P at position 355; bs) a change from A to L at position 11, a change from I to Y at position 87 and a change from E to P at position 355; bt) a change from A to L a position 11, a change from E to H at position 93 and change from S to L at position 359; bu) a change from A to S at position 11, a change from S to G at position 359 and a change from D to Q at position 367; bv) a change from P to Q at position 46, a change from N to Q at position 95 and a change from E to Y at position 355; bw) a change from R to H at position 83, a change from T to S at position 200 and a change from E to S at position 355; bx) a change from P to G at position 46 and a change from S to L at position 359; by) a change from I to L at position 87, a change from P to G at position 89, and a change from E to Q at position 355; or bz) a change from E to H at position 93 and a change from N to Q at position 95.

Also provided is: (59) A modified rbcL protein comprising SEQ ID NO. 1 or a protein having at least 95% sequence identity to SEQ ID NO. 1; wherein the modification to the rbcL protein comprises at least one of the following: a) an H at position 28; b) a K at position 31; c) an H at position 34; d) an L at position 36; e) a Q at position 68; f) an S at position 68; g) a Q at position 83; h) an H at position 83; i) an S at position 88; j) a Y at position 87; k) a K at position 312; l) a G at position 317; m) an L at position 320; n) a P at position 355; o) a Q at position 358; p) an S at position 365; q) an S at position 315; r) a T at position 317; s) an H at position 83 and a G at position 6; t) an H at position 83 and a Y at position 8; u) an H at position 83 and an L at position 11; v) an H at position 83 and an L at position 36; w) an H at position 83 and an L at position 53; x) an H at position 83 and an L at position 87; y) an H at position 83 and a G at position 89; z) an H at position 83 and an H at position 93; aa) an H at position 83 and a Q at position 95; ab) an H at position 83 and a G at position 145; ac) an H at position 83 and a P at position 168; ad) an H at position 83 and an S at position 200; ae) an H at position 83 and a Y at position 230; af) an H at position 83 and Q at position 341; ag) an H at position 83 and an L at position 355; ah) an H at position 83 and a Y at position 355; ai) an H at position 83 and a Q at position 355; aj) an H at position 83 and an S at position 355; ak) an H at position 83 and an L at position 359; al) an R to H at position 83 and a G at position 359; am) an H at position 83 and a Q at position 367; an) an H at position 83 and an S at position 367; ao) an H at position 83 and a D at position 392; ap) an H at position 83 and a Y at position 439; aq) an H at position 83 and a Y at position 450; ar) an H at position 83 and a Q at position 460; as) a Y at position 87 and a Q at position 46; at) a Y at position 87 and a G at position 89; au) a Y at position 87 and a K at position 255; av) a Y at position 87 and an S at position 272; aw) a K at position 312 and an S at position 11; ax) a K at position 312 and a P at position 355; ay) an L at position 30, an H at position 83 and an S at position 200; az) an H at position 83, a G at position 89 and an S at position 200; ba) an H at position 83, an S at position 89 and an S at position 200; bb) an H at position 83, an H at position 94 and an S at position 200; bc) an H at position 83, a P at position 94 and an S at position 200; bd) an H at position 83, an H at position 95 and an S at position 200; be) an H at position 83, a Q at position 95 and an S at position 200; bf) an H at position 83, an S at position 102 and an S at position 200; bg) an H at position 83, an S at position 200 and a D at position 249; bh) a G at position 6 and a Q at position 95; bi) a G at position 6 and G at position 145; bj) an L at position 36 and a Q at position 95; bk) a Q at position 46 and a G at position 89; bl) a Q at position 83 and an S at position 367; bm) a G at position 89 and a G at position 359; bn) a P at position 355 and a G at position 359; bo) a G at position 6, a Q at position 46 and a G at position 89; bp) a G at position 6, a Q at position 83 and an L at position 87; bq) a G at position 6, an S at position 272 and a P at position 355; br) an L at position 11, an H at position 83 and a P at position 355; bs) an L at position 11, a Y at position 87 and a P at position 355; bt) an L a position 11, an H at position 93 and an L at position 359; bu) an S at position 11, a G at position 359 and a Q at position 367; bv) a Q at position 46, a Q at position 95 and a Y at position 355; bw) an H at position 83, an S at position 200 and an S at position 355; bx) a G at position 46 and an L at position 359; by) an L at position 87, a G at position 89, and a Q at position 355; and bz) an H at position 93 and a Q at position 95.

Also provided is: (60) A modified rbcL protein comprising SEQ ID NO. 1 or a protein having at least 95% sequence identity to SEQ ID NO. 1; wherein the modification to the rbcL protein consists of: a) an H at position 28; b) a K at position 31; c) an H at position 34; d) an L at position 36; e) a Q at position 68; f) an S at position 68; g) a Q at position 83; h) an H at position 83; i) an S at position 88; j) a Y at position 87; k) a K at position 312; l) a G at position 317; m) an L at position 320; n) a P at position 355; o) a Q at position 358; p) an S at position 365; q) an S at position 315; r) a Tat position 317; s) an H at position 83 and a G at position 6; t) an H at position 83 and a Y at position 8; u) an H at position 83 and an L at position 11; v) an H at position 83 and an L at position 36; w) an H at position 83 and an L at position 53; x) an H at position 83 and an L at position 87; y) an H at position 83 and a G at position 89; z) an H at position 83 and an H at position 93; aa) an H at position 83 and a Q at position 95; ab) an H at position 83 and a G at position 145; ac) an H at position 83 and a P at position 168; ad) an H at position 83 and an S at position 200; ae) an H at position 83 and a Y at position 230; af) an H at position 83 and Q at position 341; ag) an H at position 83 and an L at position 355; ah) an H at position 83 and a Y at position 355; ai) an H at position 83 and a Q at position 355; aj) an H at position 83 and an S at position 355; ak) an H at position 83 and an L at position 359; al) an R to H at position 83 and a G at position 359; am) an H at position 83 and a Q at position 367; an) an H at position 83 and an S at position 367; ao) an H at position 83 and a D at position 392; ap) an H at position 83 and a Y at position 439; aq) an H at position 83 and a Y at position 450; ar) an H at position 83 and a Q at position 460; as) a Y at position 87 and a Q at position 46; at) a Y at position 87 and a G at position 89; au) a Y at position 87 and a K at position 255; av) a Y at position 87 and an S at position 272; aw) a K at position 312 and an S at position 11; ax) a K at position 312 and a P at position 355; ay) an L at position 30, an H at position 83 and an S at position 200; az) an H at position 83, a G at position 89 and an S at position 200; ba) an H at position 83, an S at position 89 and an S at position 200; bb) an H at position 83, an H at position 94 and an S at position 200; bc) an H at position 83, a P at position 94 and an S at position 200; bd) an H at position 83, an H at position 95 and an S at position 200; be) an H at position 83, a Q at position 95 and an S at position 200; bf) an H at position 83, an S at position 102 and an S at position 200; bg) an H at position 83, an S at position 200 and a D at position 249; bh) a G at position 6 and a Q at position 95; bi) a G at position 6 and G at position 145; bj) an L at position 36 and a Q at position 95; bk) a Q at position 46 and a G at position 89; bl) a Q at position 83 and an S at position 367; bm) a G at position 89 and a G at position 359; bn) a P at position 355 and a G at position 359; bo) a G at position 6, a Q at position 46 and a G at position 89; bp) a G at position 6, a Q at position 83 and an L at position 87; bq) a G at position 6, an S at position 272 and a P at position 355; br) an L at position 11, an H at position 83 and a P at position 355; bs) an L at position 11, a Y at position 87 and a P at position 355; bt) an L a position 11, an H at position 93 and an L at position 359; bu) an S at position 11, a G at position 359 and a Q at position 367; bv) a Q at position 46, a Q at position 95 and a Y at position 355; bw) an H at position 83, an S at position 200 and an S at position 355; bx) a G at position 46 and an L at position 359; by) an L at position 87, a G at position 89, and a Q at position 355; or bz) an H at position 93 and a Q at position 95.

Also provided is: (61) An isolated polynucleotide encoding any one of proteins SEQ ID NO. 2 to 79. (62) The isolated polynucleotide of 61, wherein said polynucleotide is any one of SEQ ID NO. 81 to 159.

### BRIEF DESCRIPTION OF THE DRAWINGS

FIG. 1 shows chloroplast transformation vector pSC179

FIG. 2 is a plot of variant frequency at the endpoint of primary screening.

FIG. 3A shows an exemplary variant having a selective advantage in a first secondary pool

FIG. 3B shows an exemplary variant having a selective advantage only in a primary pool

FIG. 4A, B show exemplary variants having a selective advantage in multiple environments

FIG. 5A shows an exemplary variant with a selection coefficient greater than 0 but not significantly different

FIG. 5B shows an exemplary variant without a value for s_{avg}

FIG. 6A shows an exemplary variant with a single significant selection coefficient

FIG. 6B shows an exemplary variant with evaluable data from a single pool

FIG. 7 shows vector pSE-3HP-K-tD2-GFP

FIG. 8 shows the results of microtiter plate culture with (A) or without (B) selection

FIG. 9 shows overlap PCR method used to regenerate variants.

FIG. 10A is an example of growth of 5 replicate well for one sample grown in MASM

FIG. 10B is an example of a growth curve using the information from FIG. 10A

FIG. 11 shows calculated s values for approximately two weeks of growth competition for some variant lines

FIG. 12 shows the selection coefficients for some lines versus a common competitor

FIGS. 13A, B, C show the results of competition with regenerated lines

FIGS. 14A, B, C show calculated growth rate differentials

FIGS. 15A, B are Western blots showing Rubisco protein levels

FIG. 16 shows relative rbcL transcript abundance

FIG. 17A shows median frequencies and interquartile ranges of all expected mutants in the SSM pool

FIG. 17B shows median frequencies and interquartile ranges of all expected mutants in the NNK library

FIG. 17C shows the frequency of single-mutant parental sequences in the SSM pool

FIG. 17D shows the frequency of single-mutant parental sequences in the NNK library

FIG. 18 shows the calculated Δs values relative to the mean of the wild type complemented strain for the top 26 lines

FIG. 19 is a Western blot showing exemplary Rubisco protein levels

FIG. 20 shows the distributions of mutant frequencies along with parental sequences for the SSM library

FIG. 21 shows the distribution sof selection coefficients as measure for all non-extinct variants in the SSM primary screen

FIG. 22 shows the distribution of selection coefficients as measured for all non-extenic variant in the triple combo primary screen

FIG. 23 shows an example of s_{avg} vs. sₛᵤₘ for all viable variants in the SSM094 primary screen

FIG. 24 shows calculated s values for two week of growth competition for original (A) and regenerated (B) lines.

FIG. 25 shows calculated s values for 16 validated variants competed en masse in turbidostats.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present inventive discovery.

As used in this specification and the appended claims, the singular forms “a”, “an” and “the” include plural reference unless the context clearly dictates otherwise.

An endogenous nucleic acid, nucleotide, polypeptide, or protein as described herein is defined in relationship to the host organism. An endogenous nucleic acid, nucleotide, polypeptide, or protein is one that naturally occurs in the host organism.

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

If an initial start codon (Met) is not present in any of the amino acid sequences disclosed herein, including sequences contained in the sequence listing, one of skill in the art would be able to include, at the nucleotide level, an initial ATG, so that the translated polypeptide would have the initial Met. If a start and/or stop codon is not present at the beginning and/or end of a coding sequence, one of skill in the art would know to insert an “ATG” at the beginning of the coding sequence and nucleotides encoding for a stop codon (any one of TAA, TAG, or TGA) at the end of the coding sequence. Any of the disclosed nucleotide sequences can be, if desired, fused to another nucleotide sequence that when operably linked to a “control element” results in the proper translation of the encoded amino acids (for example, a fusion protein). In addition, two or more nucleotide sequences can be linked by a short peptide, for example, a viral peptide.

Increased yield in higher plants can be manifested in phenotypes such as increased cell proliferation, increased organ or cell size and increased total plant mass. The phrases “an increase in biomass yield” and “an increase in biomass” are used interchangeably throughout the specification.

An increase in biomass yield can be defined by a number of growth measures, including, for example, a selective advantage during competitive growth, increased growth rate, increased carrying capacity, and/or increased culture productivity (as measured on a per volume or per area basis). For example, a competition assay can be between a transgenic strain and a wild-type strain, between several transgenic strains, or between several transgenic strains and a wild-type strain.

Disclosed herein are methods for increasing biomass of an organism by transforming a host cell or host organism with one or more of the nucleotides sequences encoding a Rubisco large subunit (rbcL) protein containing at least one of the mutations relative to SEQ ID NO. 1 disclosed herein. In some embodiments, a host cell is part of a multicellular organism. In other embodiments, a host cell is cultured as a unicellular organism. Host organisms can include any suitable host, for example, a microorganism. Microorganisms which are useful for the methods described herein include, for example, photosynthetic bacteria (e.g., cyanobacteria), algae and vascular plants.

Examples of host organisms that can be transformed with one or more of the polynucleotides or expressing one of the modified rbcL proteins disclosed herein include vascular and non-vascular organisms. The organism can be prokaryotic or eukaryotic. The organism can be unicellular or multicellular. A host organism is an organism comprising a host cell. In other embodiments, the host organism is photosynthetic. A photosynthetic organism is one that naturally photosynthesizes (e.g., an alga) or that is genetically engineered or otherwise modified to be photosynthetic. By way of example and not limitation, a non-vascular photosynthetic microalga species include C. reinhardtii, Nannochloropsis oceania, N. salina, D. salina, H. pluvalis, S. dimorphus, D. viridis, Chlorella sp., and D. tertiolecta.

In other embodiments the host organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, and peanut (Arachis hypogaea), as well as Arabidopsis, tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops and fruits.

The host cell can be prokaryotic. Examples of some prokaryotic organisms useful in the practice of the present disclosure include, but are not limited to, cyanobacteria (e.g., Synechococcus, Synechocystis, Athrospira, Gleocapsa, Oscillatoria, and, Pseudoanabaena).

In some embodiments, the host organism is eukaryotic (e.g. green algae, red algae, brown algae). In some embodiments, the algae is a green algae, for example, a Chlorophycean. The algae can be unicellular or multicellular. In some embodiments, eukaryotic microalgae, such as for example, a Chlamydomonas, Volvacales, Dunaliella, Nannochloropsis, Desmodesmus, Scenedesmus, Chlorella, or Hematococcus species, can be used in the disclosed methods. In more specific embodiments, the host cell is Chlamydomonas reinhardtii, Dunaliella salina, Haematococcus pluvialis, Nannochloropsis oceania, Nannochloropsis salina, Scenedesmus dimorphus, a Chlorella species, a Spirulina species, a Desmid species, Spirulina maximus, Arthrospira fusiformis, Dunaliella viridis, or Dunaliella tertiolecta.

In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

In some instances a host organism is vascular and photosynthetic. Examples of vascular plants include, but are not limited to, angiosperms, gymnosperms, rhyniophytes, or other tracheophytes. In other instances a host organism is non-vascular and photosynthetic. As used herein, the term “non-vascular photosynthetic organism,” refers to any macroscopic or microscopic organism, including, but not limited to, algae, cyanobacteria and photosynthetic bacteria, which does not have a vascular system such as that found in vascular plants. Examples of non-vascular photosynthetic organisms include bryophtyes, such as marchantiophytes or anthocerotophytes. In some instances the organism is a cyanobacteria. In some instances, the organism is algae (e.g., macroalgae or microalgae). The algae can be unicellular or multicellular algae.

In certain embodiments, the host cell is a plant. The term “plant” is used broadly herein to refer to a eukaryotic organism containing plastids, such as chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, and roots. A part of a plant useful for propagation includes, for example, seeds, fruits, cuttings, seedlings, tubers, and rootstocks.

Some of the host organisms useful in the disclosed embodiments are, for example, are extremophiles, such as hyperthermophiles, psychrophiles, psychrotrophs, halophiles, barophiles and acidophiles. Some of the host organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, a host cell expressing a protein of the present disclosure can be grown in a liquid environment which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 31., 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid environments.

An organism may be grown under conditions which permit photosynthesis, however, this is not a requirement (e.g., a host organism may be grown in the absence of light). In growth conditions where a host organism is not capable of photosynthesis (e.g., because of the absence of light), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or an organism-specific requirement. Organic carbon sources include any source of carbon which the host organism is able to metabolize including, but not limited to, acetate, simple carbohydrates (e.g., glucose, sucrose, and lactose), complex carbohydrates (e.g., starch and glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

Optimal growth of algal organisms occurs usually at a temperature of about 20° C. to about 25° C., although some organisms can still grow at a temperature of up to about 35° C. Active growth is typically performed in liquid culture. If the organisms are grown in a liquid medium and are shaken or mixed, the density of the cells can be anywhere from about 1 to 5×10⁸ cells/ml at the stationary phase. For example, the density of the cells at the stationary phase for Chlamydomonas sp. can be about 1 to 5×10⁷ cells/ml; the density of the cells at the stationary phase for Nannochloropsis sp. can be about 1 to 5×10⁸ cells/ml; the density of the cells at the stationary phase for Scenedesmus sp. can be about 1 to 5×10⁷ cells/ml; and the density of the cells at the stationary phase for Chlorella sp. can be about 1 to 5×10⁸ cells/ml. Exemplary cell densities at the stationary phase are as follows: Chlamydomonas sp. can be about 1×10⁷ cells/ml; Nannochloropsis sp. can be about 1×10⁸ cells/ml; Scenedesmus sp. can be about 1×10⁷ cells/ml; and Chlorella sp. can be about 1×10⁸ cells/ml. An exemplary growth rate may yield, for example, a two to twenty fold increase in cells per day, depending on the growth conditions. In addition, doubling times for organisms can be, for example, 5 hours to 30 hours. The organism can also be grown on solid media, for example, media containing about 1.5% agar, in plates or in slants.

One source of energy is fluorescent light that can be placed, for example, at a distance of about 1 inch to about two feet from the algae. Examples of types of fluorescent lights includes, for example, cool white and daylight. Bubbling with air or CO₂ improves the growth rate of the organism. Bubbling with CO₂ can be, for example, at 1% to 5% CO₂. If the lights are turned on and off at regular intervals (for example, 12:12 or 14:10 hours of light:dark) the cells of some organisms will become synchronized.

Long term storage of algae can be achieved by streaking them onto plates, sealing the plates with, for example, PARAFILM™, and placing them in dim light at about 10° C. to about 18° C. Alternatively, algae may be grown as streaks or stabs into agar tubes, capped, and stored at about 10° C. to about 18° C. Both methods allow for the storage of the organisms for several months.

For longer storage, the algae can be grown in liquid culture to mid to late log phase and then supplemented with a penetrating cryoprotective agent like DMSO or MeOH, and stored at less than −130° C. An exemplary range of DMSO concentrations that can be used is 5 to 8%. An exemplary range of MeOH concentrations that can be used is 3 to 9%.

Organisms can be grown on a defined minimal medium (for example, high salt medium (HSM), modified artificial sea water medium (MASM), or F/2 medium) with light as the sole energy source. In other instances, the organism can be grown in a medium (for example, tris acetate phosphate (TAP) medium), and supplemented with an organic carbon source.

Organisms, such as algae, can grow naturally in fresh water or marine water. Culture media for freshwater algae can be, for example, synthetic media, enriched media, soil water media, and solidified media, such as agar. Various culture media have been developed and used for the isolation and cultivation of fresh water algae and are described in Watanabe, M. W. (2005). Freshwater Culture Media. In R. A. Andersen (Ed.), Algal Culturing Techniques (pp. 13-20). Elsevier Academic Press. Culture media for marine algae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In some instances, organisms can be grown in containers wherein each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container. Culturing techniques for algae are well known to one of skill in the art and are described, for example, in Freshwater Culture Media. In R. A. Andersen (Ed.), Algal Culturing Techniques. Elsevier Academic Press.

Because photosynthetic organisms, for example, algae, require sunlight, CO₂ and water for growth, they can be cultivated in, for example, open ponds and lakes. However, these open systems are more vulnerable to contamination than a closed system. One challenge with using an open system is that the organism of interest may not grow as quickly as a potential invader. This becomes a problem when another organism invades the liquid environment in which the organism of interest is growing, and the invading organism has a faster growth rate and takes over the system. In addition, in open systems there is less control over water temperature, CO₂ concentration, and lighting conditions. The growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system are that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In these ponds, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors. Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

If the raceway pond is placed outdoors, there are several different ways to address the invasion of an unwanted organism. For example, the pH or salinity of the liquid in which the desired organism is in can be such that the invading organism either slows down its growth or dies. Also, chemicals can be added to the liquid, such as bleach, or a pesticide can be added to the liquid, such as glyphosate. In addition, the organism of interest can be genetically modified such that it is better suited to survive in the liquid environment. Any one or more of the above strategies can be used to address the invasion of an unwanted organism.

Alternatively, organisms, such as algae, can be grown in closed structures such as photobioreactors, where the environment is under stricter control than in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations. On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethlyene bag cultivation). A batch photobioreactor is set up with, for example, nutrients, an organism (for example, algae), and water, and the organism is allowed to grow until the batch is harvested. A continuous photobioreactor can be harvested, for example, either continually, daily, or at fixed time intervals.

High density photobioreactors are described in, for example, Lee, et al., Biotech. Bioengineering 44:1161-1167, 1994. Other types of bioreactors, such as those for sewage and waste water treatments, are described in, Sawayama, et al., Appl. Micro. Biotech., 41:729-731, 1994. Additional examples of photobioreactors are described in, U.S. Appl. Publ. No. 2005/0260553, U.S. Pat. Nos. 5,958,761, and 6,083,740. Also, organisms, such as algae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermentors. Additional methods of culturing organisms and variations of the methods described herein are known to one of skill in the art.

CO₂ can be delivered to any of the systems described herein, for example, by bubbling in CO₂ from under the surface of the liquid containing the organism. Also, sparges can be used to inject CO₂ into the liquid. Spargers are, for example, porous disc or tube assemblies that are also referred to as Bubblers, Carbonators, Aerators, Porous Stones and Diffusers. Nutrients that can be used in the systems described herein include, for example, nitrogen (in the form of NO₃⁻or NH₄⁺), phosphorus, and trace metals (Fe, Mg, K, Ca, Co, Cu, Mn, Mo, Zn, V, and B). The nutrients can come, for example, in a solid form or in a liquid form. If the nutrients are in a solid form they can be mixed with, for example, fresh or salt water prior to being delivered to the liquid containing the organism, or prior to being delivered to a photobioreactor.

Algae can be grown in large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at lease 5 square meters, at least 10 square meters, at least 200 square meters, at least 500 square meters, at least 1,500 square meters, at least 2,500 square meters, in area, or greater.

It should be recognized that the present disclosure is not limited to transgenic cells, organisms, and plastids containing polynucleotides and expressing modified rbcL proteins disclosed herein, but also encompasses such cells, organisms, and plastids transformed with additional nucleotide sequences encoding enzymes involved in fatty acid synthesis. Thus, some embodiments involve the introduction of one or more sequences encoding proteins involved in fatty acid synthesis in addition to a protein disclosed herein. For example, several enzymes in a fatty acid production pathway may be linked, either directly or indirectly, such that products produced by one enzyme in the pathway, once produced, are in close proximity to the next enzyme in the pathway. These additional sequences may be contained in a single vector either operatively linked to a single promoter or linked to multiple promoters, e.g. one promoter for each sequence. Alternatively, the additional coding sequences may be contained in a plurality of additional vectors. When a plurality of vectors are used, they can be introduced into the host cell or organism simultaneously or sequentially.

Additional embodiments provide a plastid, and in particular a chloroplast, transformed with a polynucleotide and expressing a modified rbcL protein of the present disclosure. The polynucleotide may be introduced into the genome of the plastid using any of the methods described herein or otherwise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga et al., Methods in Plant Molecular Biology, Cold Spring Harbor Laboratory Press, 1995; Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding any one or more of the proteins of the present disclosure. Methods for the assembly of artificial plastid genomes can be found in U.S. patent application Ser. No. 12/287,230 filed Oct. 6, 2008, published as U.S. Publication No. 2009/0123977 on May 14, 2009, and U.S. patent application Ser. No. 12/384,893 filed Apr. 8, 2009, published as U.S. Publication No. 2009/0269816 on Oct. 29, 2009, each of which is incorporated by reference in its entirety.

One or more polynucleotides of the present disclosure can also be modified such that the resulting amino acid is “substantially identical” to the unmodified or reference amino acid. A “substantially identical” amino acid sequence is a sequence that differs from a reference sequence by one or more conservative or non-conservative amino acid substitutions, deletions, or insertions, particularly when such a substitution occurs at a site that is not the active site (catalytic domains (CDs)) of the molecule and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). Conservative substitutions are those that substitute a given amino acid in a polypeptide by another amino acid of like characteristics. Examples of conservative substitutions are the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. In alternative aspects, these conservative substitutions can also be synthetic equivalents of these amino acids.

To generate a genetically modified host cell or organism, a polynucleotide, or a polynucleotide cloned into a vector, is introduced stably or transiently into a host cell, using established techniques, including, but not limited to, electroporation, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance.

A polynucleotide or recombinant nucleic acid molecule described herein, can be introduced into a cell (e.g., alga cell) using any method known in the art. A polynucleotide can be introduced into a cell by a variety of methods, which are well known in the art and selected, in part, based on the particular host cell. For example, the polynucleotide can be introduced into a cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann. Rev. Plant. Physiol. Plant Mol. Biol. 42:205-225, 1991).

In addition, polynucleotides encoding the modified rbcL proteins disclosed herein can be introduced into host cells, and in particular the chloroplasts of host cells, by polyethylene glycol (PEG) mediated transformation, or bacterially mediated or Agrobacterium mediated transformation. Methods for the transformation of chloroplasts are known to those of skill in the art and can be found, for example in Bock, Current Opinion in Biotechnol., 2014, 26:7-13; Wani et al., 2010, Current Genomics, 11:500-512; Wang et al., 2009, J. Genetics and Genomics, 36:387-398; and van Bel et al., 2001, Current Opin. Biotechnol., 12:144-149 and the references cited in each of these publications.

As discussed above, microprojectile mediated transformation can be used to introduce a polynucleotide into a cell (for example, as described in Klein et al., Nature 327:70-73, 1987). This method utilizes microprojectiles such as gold or tungsten, which are coated with the desired polynucleotide by precipitation with calcium chloride, spermidine or polyethylene glycol. The microprojectile particles are accelerated at high speed into a cell using a device such as the BIOLISTIC PD-1000 particle gun (BioRad; Hercules Calif.). Methods for the transformation using biolistic methods are well known in the art (for example, as described in Christou, Trends in Plant Science 1:423-431, 1996). Microprojectile mediated transformation has been used, for example, to generate a variety of transgenic plant species, including cotton, soybean, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto, Curr. Opin. Biotech. 5:158-162, 1994). The transformation of most dicotyledonous plants is possible with the methods described above. Transformation of monocotyledonous and dicotyledonous plants also can be transformed using, for example, biolistic methods as described above, bacterially mediated or Agrobacterium-mediated transformation, protoplast transformation, electroporation of partially permeabilized cells, introduction of DNA using glass fibers, glass bead agitation method, etc., as known in the art. Methods for biolistic transformation of algae are known in the art.

The basic techniques used for transformation and expression in photosynthetic microorganisms are similar to those commonly used for E. coli, Saccharomyces cerevisiae and other species. Transformation methods customized for a photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach, 1988, “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (See, for example, Sanford, Trends In Biotech. (1988) 6: 299-302, U.S. Pat. No. 4,945,050; electroporation (Fromm et al., Proc. Nat'l. Acad. Sci. (USA) (1985) 82: 5824-5828); use of a laser beam, electroporation, microinjection or any other method capable of introducing DNA into a host cell.

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad. Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves. Methods for the transformation of algal chloroplasts can be found in U.S. Patent Application Publication 2012/0252054 which is incorporated by reference in its entirety.

A further refinement in chloroplast transformation/expression technology that facilitates control over the timing and tissue pattern of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs. Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the protein can be modified for plastid targeting by employing plant cell nuclear transformation constructs wherein DNA coding sequences of interest are fused to any of the available transit peptide sequences capable of facilitating transport of the encoded enzymes into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of DNAs encoding the enzymes. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, or LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heijne et al. (1991) Plant Mol. Biol. Rep. 9: 104; Clark et al. (1989) J. Biol. Chem. 264: 17544; della-Cioppa et al. (1987) Plant Physiol. 84: 965; Romer et al. (1993) Biochem. Biophys. Res. Commun. 196: 1414; and Shah et al. (1986) Science 233: 478. Another transit peptide sequence is that of the intact ACCase from Chlamydomonas (genbank EDO96563, amino acids 1-33). The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present disclosure are not critical as long as delivery into a plastid is obtained. Proteolytic processing within the plastid then produces the mature enzyme. This technique has proven successful with enzymes involved in polyhydroxyalkanoate biosynthesis (Nawrath et al. (1994) Proc. Natl. Acad. Sci. USA 91: 12760), and neomycin phosphotransferase II (NPT-II) and CP4 EPSPS (Padgette et al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, α-carboxy-transferase, and plastid-targeted monocot multifunctional acetyl-CoA carboxylase (Mw, 220,000); plastidic subunits of the fatty acid synthase complex (e.g., acyl carrier protein (ACP), malonyl-ACP synthase, KASI, KASII, and KASIII); steroyl-ACP desaturase; thioesterases (specific for short, medium, and long chain acyl ACP); plastid-targeted acyl transferases (e.g., glycerol-3-phosphate and acyl transferase); enzymes involved in the biosynthesis of aspartate family amino acids; phytoene synthase; gibberellic acid biosynthesis (e.g., ent-kaurene synthases 1 and 2); and carotenoid biosynthesis (e.g., lycopene synthase).

In one embodiment, a transformation may introduce a nucleic acid into a plastid genome of the host cell (e.g., chloroplast). In another embodiment, a transformation may introduce a nucleic acid into the nuclear genome of the host cell. In still another embodiment, a transformation may introduce nucleic acids into both the nuclear genome and into a plastid genome.

Transformed cells can be plated on selective media following introduction of exogenous nucleic acids. This method may also comprise several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which (which chelates magnesium) is added to chelate toxic metals. Following the screening for clones with the proper integration of exogenous nucleic acids, clones can be screened for the presence of the encoded protein(s), products and/or phenotypes. Protein expression screening can be performed by Western blot analysis and/or enzyme activity assays. Transporter and/or product screening may be performed by any method known in the art, for example ATP turnover assay, substrate transport assay, HPLC or gas chromatography.

The expression of the polynucleotide can be accomplished by inserting a polynucleotide sequence (gene) encoding a modified rbcL protein disclosed herein into the chloroplast or nuclear genome of a microalgae. The modified cell can be made homoplasmic to ensure that the polynucleotide will be stably maintained in the chloroplast genome of all descendents. A cell is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein.

Construct, vector and plasmid are used interchangeably throughout the disclosure. Nucleic acids described herein, can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences.

In some embodiments, a polynucleotide of the present disclosure is cloned or inserted into an expression vector using cloning techniques known to one of skill in the art. The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2nd Ed., Cold Spring Harbor Press, (1989) and Ausubel et al., Short Protocols in Molecular Biology, 2nd Ed., John Wiley & Sons (1992). Vectors for plant transformation have been reviewed in Rodriguez et al. (1988) Vectors: A Survey of Molecular Cloning Vectors and Their Uses, Butterworths, Boston; Glick et al. (1993) Methods in Plant Molecular Biology and Biotechnology CRC Press, Boca Raton, Fla.; and Croy (1993) In Plant Molecular Biology Labfax, Hames and Rickwood, Eds., BIOS Scientific Publishers Limited, Oxford, UK.

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, and herpes simplex virus), PI-based artificial chromosomes, yeast plasmids, yeast artificial chromosomes, and any other vectors specific for specific hosts of interest (such as E. coli and yeast). Such vectors can include, for example, chromosomal, nonchromosomal and synthetic DNA sequences.

Numerous suitable expression vectors are known to those of skill in the art. The following vectors are provided by way of example; for bacterial host cells: pQE vectors (Qiagen), pBluescript plasmids, pNH vectors, lambda-ZAP vectors (Stratagene), pTrc99a, pKK223-3, pDR540, and pRIT2T (Pharmacia); for eukaryotic host cells: pXT1, pSG5 (Stratagene), pSVK3, pBPV, pMSG, pET21a-d(+) vectors (Novagen), and pSVLSV40 (Pharmacia). However, any other plasmid or other vector may be used so long as it is compatible with the host cell.

In some embodiments, the vector may comprise nucleotide sequences that are codon-biased for expression in the organism being transformed. In another embodiment, a gene of interest, for example, a biomass yield gene, may comprise nucleotide sequences that are codon-biased for expression in the organism being transformed. In addition, the nucleotide sequence of a tag may be codon-biased or codon-optimized for expression in the organism being transformed. A polynucleotide sequence may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased or chloroplast codon biased). In some embodiments, codon biasing occurs before mutagenesis to generate a polypeptide. In other embodiments, codon biasing occurs after mutagenesis to generate a polynucleotide. In yet other embodiments, codon biasing occurs before mutagenesis as well as after mutagenesis.

In some embodiments, a vector comprises a polynucleotide operably linked to one or more control elements, such as a promoter and/or a transcription terminator. Such polynucleotide may be heterologous with respect to the one or more control elements. The operably linked control element(s) and polynucleotide sequence are heterologous if not operably linked to each other in nature. A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the chloroplast) can be attached to the polynucleotide encoding a protein of interest. Such signals are well known in the art and have been widely reported.

In a vector, a nucleotide sequence of interest is operably linked to a promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control.

Promoters useful for the present disclosure may come from any source (e.g., viral, bacterial, fungal, protist, and animal) and may further include homologous, engineered or synthetic promoter sequences. The promoters contemplated herein can be specific to photosynthetic organisms, non-vascular photosynthetic organisms, and vascular photosynthetic organisms (e.g., algae, plants) and capable of driving expression of a sequence operably linked to such promoter in those organisms. In some instances, the nucleic acids above are inserted into a vector that comprises a promoter of a photosynthetic organism, e.g., algae. The promoter can be a constitutive promoter, tissue-specific promoter, developmental stage specific promoter, or an inducible promoter. A promoter typically includes necessary nucleic acid sequences near the start site of transcription, (e.g., a TATA element). Common promoters used in expression vectors include, but are not limited to, LTR or SV40 promoter, the E. coli lac or trp promoters, and the phage lambda PL promoter. Non-limiting examples of promoters are endogenous promoters such as the psbA and atpA promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression. Useful algal chloroplast promoters include, but are not limited to, the atpA, psbA, psbB, psbC, psbD, rbcL, 16S and psaA promoters. Useful algal nuclear promoters include, but are not limited to, arg7, nit1, tubulin, PsaD, Hsp70A, rbcS2 and Hsp70A/rbcS2 fusion (see Rasala, B. A., Lee, P. A., Shen, Z., Briggs, S. P., Mendez, M., & Mayfield, S. P. (2012). Robust Expression and Secretion of Xylanase1 in Chlamydomonas reinhardtii by Fusion to a Selection Gene and Processing with the FMDV 2A Peptide. PLoS ONE, 7(8), e43349. http://doi.org/10.1371/journal.pone.0043349).

A “constitutive” promoter is, for example, a promoter that is active under most environmental and developmental conditions. Constitutive promoters can, for example, maintain a relatively constant level of transcription.

An “inducible” promoter is a promoter that is active under controllable environmental or developmental conditions. For example, inducible promoters are promoters that initiate increased levels of transcription from DNA under their control in response to some change in the environment, e.g. the presence or absence of a nutrient or a change in temperature. Examples of inducible promoters/regulatory elements include, for example, a nitrate-inducible promoter (for example, as described in Bock et al, Plant Mol. Biol. 17:9 (1991)), or a light-inducible promoter, (for example, as described in Feinbaum et al, Mol Gen. Genet. 226:449 (1991); and Lam and Chua, Science 248:471 (1990)), or a heat responsive promoter (for example, as described in Muller et al., Gene 111: 165-73 (1992)).

In many embodiments, a polynucleotide of the present disclosure includes a nucleotide sequence, where the nucleotide sequence encoding the polypeptide is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (for example, as described in Guzman et al. (1995) J. Bacteriol. 177:4121-4130); a xylose-inducible promoter, e.g., Pxyl (for example, as described in Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

In particular reference to vascular plants, constitutive promoters include the CaMV 35S promoter (Odell et al. (1985) Nature 313: 810), the enhanced CaMV 35S promoter, the Figwort Mosaic Virus (FMV) promoter (Richins et al. (1987) NAR 20: 8451), the mannopine synthase (mas) promoter, the nopaline synthase (nos) promoter, and the octopine synthase (ocs) promoter. Useful inducible promoters include heat-shock promoters (Ou-Lee et al. (1986) Proc. Natl. Acad. Sci. USA 83: 6815; Ainley et al. (1990) Plant Mol. Biol. 14: 949), a nitrate-inducible promoter derived from the spinach nitrite reductase gene (Back et al. (1991) Plant Mol. Biol. 17: 9), hormone-inducible promoters (Yamaguchi-Shinozaki et al. (1990) Plant Mol. Biol. 15: 905; Kares et al. (1990) Plant Mol. Biol. 15: 905), and light-inducible promoters associated with the small subunit of RuBP carboxylase and LHCP gene families (Kuhlemeier et al. (1989) Plant Cell 1: 471; Feinbaum et al. (1991) Mol. Gen. Genet. 226: 449; Weisshaar et al. (1991) EMBO J. 10: 1777; Lam and Chua (1990) Science 248: 471; Castresana et al. (1988) EMBO J. 7: 1929; Schulze-Lefert et al. (1989) EMBO J. 8: 651).

Examples of useful tissue-specific, developmentally-regulated promoters include fruit-specific promoters such as the E4 promoter (Cordes et al. (1989) Plant Cell 1:1025), the E8 promoter (Deikman et al. (1988) EMBO J. 7: 3315), the kiwifruit actinidin promoter (Lin et al. (1993) PNAS 90: 5939), the 2A11 promoter (Houck et al., U.S. Pat. No. 4,943,674), and the tomato pZ130 promoter (U.S. Pat. Nos. 5,175,095 and 5,530,185); the β-conglycinin 7S promoter (Doyle et al. (1986) J. Biol. Chem. 261: 9228; Slighton and Beachy (1987) Planta 172: 356), and seed-specific promoters (Knutzon et al. (1992) Proc. Natl. Acad. Sci. USA 89: 2624; Bustos et al. (1991) EMBO J. 10: 1469; Lam and Chua (1991) J. Biol. Chem. 266: 17131; Stayton et al. (1991) Aust. J. Plant. Physiol. 18: 507). Fruit-specific gene regulation is discussed in U.S. Pat. No. 5,753,475. Other useful seed-specific promoters include, but are not limited to, the napin, phaseolin, zein, soybean trypsin inhibitor, 7S, ADR12, ACP, stearoyl-ACP desaturase, oleosin, Lasquerella hydroxylase, and barley aldose reductase promoters (Bartels (1995) Plant J. 7: 809-822), the EA9 promoter (U.S. Pat. No. 5,420,034), and the Bce4 promoter (U.S. Pat. No. 5,530,194). Useful embryo-specific promoters include the corn globulin 1 and oleosin promoters. Useful endosperm-specific promoters include the rice glutelin-1 promoter, the promoters for the low-pI α-amylase gene (Amy32b) (Rogers et al. (1984) J. Biol. Chem. 259: 12234), the high-pI α-amylase gene (Amy 64) (Khurseed et al. (1988) J. Biol. Chem. 263: 18953), and the promoter for a barley thiol protease gene (“Aleurain”) (Whittier et al. (1987) Nucleic Acids Res. 15: 2515). Plant functional promoters useful for preferential expression in seed plastids include those from plant storage protein genes and from genes involved in fatty acid biosynthesis in oilseeds. Examples of such promoters include the 5′ regulatory regions from such genes as napin (Kridl et al. (1991) Seed Sci. Res. 1: 209), phaseolin, zein, soybean trypsin inhibitor, ACP, stearoyl-ACP desaturase, and oleosin. Seed-specific gene regulation is discussed in EP 0 255 378 B1 and U.S. Pat. Nos. 5,420,034 and 5,608,152. Promoter hybrids can also be constructed to enhance transcriptional activity (Hoffman, U.S. Pat. No. 5,106,739), or to combine desired transcriptional activity and tissue specificity.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter; an araBAD promoter; in vivo regulated promoters, such as an ssaG promoter or a related promoter (for example, as described in U.S. Patent Publication No. 20040131637), a pagC promoter (for example, as described in Pulkkinen and Miller, J. Bacteriol., 1991: 173(1): 86-93; and Alpuche-Aranda et al., PNAS, 1992; 89(21): 10079-83), a nirB promoter (for example, as described in Harborne et al. (1992) Mol. Micro. 6:2805-2813; Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (for example, GenBank Accession Nos. AX798980, AX798961, and AX798183); a stationary phase promoter, e.g., a dps promoter, an spy promoter; a promoter derived from the pathogenicity island SPI-2 (for example, as described in WO96/17951); an actA promoter (for example, as described in Shetron-Rama et al. (2002) Infect. Immun. 70:1087-1096); an rpsM promoter (for example, as described in Valdivia and Falkow (1996). Mol. Microbiol. 22:367-378); a tet promoter (for example, as described in Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); and an SP6 promoter (for example, as described in Melton et al. (1984) Nucl. Acids Res. 12:7035-7056).

Non-limiting examples of suitable eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain, for example, one or more cloning sites such as a multiple cloning site, which can, but need not, be positioned such that a exogenous or endogenous polynucleotide can be inserted into the vector and operatively linked to a desired element.

The vector also can contain a prokaryote origin of replication (ori), for example, an E. coli ori or a cosmid ori, thus allowing passage of the vector into a prokaryote host cell, as well as into a plant chloroplast. Various bacterial and viral origins of replication are well known to those skilled in the art and include, but are not limited to the pBR322 plasmid origin, the 2u plasmid origin, and the SV40, polyoma, adenovirus, VSV, and BPV viral origins.

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype. A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (for example, as described in Giacomin, Plant Sci. 116:59-72, 1996; Scikantha, J. Bacteriol. 178:121, 1996; Gerdes, FEBS Lett. 389:44-47, 1996; and Jefferson, EMBO J. 6:3901-3907, 1997, fl-glucuronidase).

A selectable marker (or selectable gene) generally is a molecule that, when present or expressed in a cell, provides a selective advantage (or disadvantage) to the cell containing the marker, for example, the ability to grow in the presence of an agent that otherwise would kill the cell. The selection gene can encode for a protein necessary for the survival or growth of the host cell transformed with the vector. A selectable marker can provide a means to obtain, for example, prokaryotic cells, eukaryotic cells, and/or plant cells that express the marker and, therefore, can be useful as a component of a vector of the disclosure. The selection gene or marker can encode for a protein necessary for the survival or growth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin and paromycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad. Sci., USA 85:8047, 1988); mannose-6-phosphate isomerase which allows cells to utilize mannose (for example, as described in PCT Publication Application No. WO 94/20627); ornithine decarboxylase, which confers resistance to the ornithine decarboxylase inhibitor, 2-(difluoromethyl)-DL-ornithine (DFMO; for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:1241-1248, 1988), a mutant psbA, which confers resistance to atrazine (for example, as described in Smeda et al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, dtreptomycin, streptomycin, sulfonamide and sulfonylurea resistance in plants (for example, as described in Maliga et al., Methods in Plant Molecular Biology, Cold Spring Harbor Laboratory Press, 1995, page 39). The selection marker can have its own promoter or its expression can be driven by a promoter driving the expression of a polypeptide of interest. The promoter driving expression of the selection marker can be a constitutive or an inducible promoter.

Reporter genes greatly enhance the ability to monitor gene expression in a number of biological organisms. Reporter genes have been successfully used in chloroplasts of higher plants, and high levels of recombinant protein expression have been reported. In addition, reporter genes have been used in the chloroplast of C. reinhardtii. In chloroplasts of higher plants, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyltransferase (aadA, for example, as described in Svab and Maliga, Proc. Natl. Acad. Sci., USA 90:913-917, 1993), and the Aequorea victoria GFP (for example, as described in Sidorov et al., Plant J. 19:209-216, 1999) have been used as reporter genes (for example, as described in Heifetz, Biochemie 82:655-666, 2000). Each of these genes has attributes that make them useful reporters of chloroplast gene expression, such as ease of analysis, sensitivity, or the ability to examine expression in situ. Based upon these studies, other exogenous proteins have been expressed in the chloroplasts of higher plants such as Bacillus thuringiensis Cry toxins, conferring resistance to insect herbivores (for example, as described in Kota et al., Proc. Natl. Acad. Sci., USA 96:1840-1845, 1999), or human somatotropin (for example, as described in Staub et al., Nat. Biotechnol. 18:333-338, 2000), a potential biopharmaceutical. Several reporter genes have been expressed in the chloroplast of the eukaryotic green alga, C. reinhardtii, including aadA (for example, as described in Goldschmidt-Clermont, Nucl. Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 1993; and Ishikura et al., J. Biosci. Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet 263:404-410, 2000).

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001; Staub and Maliga, Plant Cell 4:39-45, 1992; and Kavanagh et al., Genetics 152:1111-1122, 1999, each of which is incorporated herein by reference). The entire chloroplast genome of C. reinhardtii is available to the public on the world wide web, at the URL “biology.duke.edu/chlamy_genome/-chloro.html” (see “view complete genome as text file” link and “maps of the chloroplast genome” link; J. Maul, J. W. Lilly, and D. B. Stern, unpublished results; revised Jan. 28, 2002; to be published as GenBank Acc. No. AF396929; and Maul, J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast. In this respect, the website containing the C. reinhardtii chloroplast genome sequence also provides maps showing coding and non-coding regions of the chloroplast genome, thus facilitating selection of a sequence useful for constructing a vector (also described in Maul, J. E., et al. (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlam-y/chloro/chlorol40.html”). In addition, the entire nuclear genome of C. reinhardtii is described in Merchant, S. S., et al., Science (2007), 318(5848):245-250, thus facilitating one of skill in the art to select a sequence or sequences useful for constructing a vector.

For expression of a modified rbcL protein in a host, an expression cassette or vector may be employed. The expression vector will comprise a transcriptional and translational initiation region, which may be inducible or constitutive, where the coding region is operably linked under the transcriptional control of the transcriptional initiation region, and a transcriptional and translational termination region. These control regions may be native to the gene, or may be derived from an exogenous source. Expression vectors generally have convenient restriction sites located near the promoter sequence to provide for the insertion of nucleic acid sequences encoding exogenous or endogenous proteins. A selectable marker operative in the expression host may be present. Vectors for plant transformation have been reviewed in Rodriguez et al. (1988) Vectors: A Survey of Molecular Cloning Vectors and Their Uses, Butterworths, Boston; Glick et al. (1993) Methods in Plant Molecular Biology and Biotechnology CRC Press, Boca Raton, Fla.; and Croy (1993) In Plant Molecular Biology Labfax, Hames and Rickwood, Eds., BIOS Scientific Publishers Limited, Oxford, UK.

The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

The description herein provides that host cells may be transformed with vectors. One of skill in the art will recognize that such transformation includes transformation with circular vectors, linearized vectors, linearized portions of a vector, or any combination of the above. Thus, a host cell comprising a vector may contain the entire vector in the cell (in either circular or linear form), or may contain a linearized portion of a vector of the present disclosure.

One of skill in the art will readily appreciate that minor differences in the sequence of the rbcL protein may exist between species such that the exact location of a given amino acid relative to the initial Methionine may vary. It is possible, however, to account for these slight differences by the use of sequence alignment. In this process, subject sequences are compared to a reference sequence, and the sequences aligned in manner as to minimize the mispairing between the sequences. By using this method, one of skill in the art can readily determine the equivalent position in the subject sequence relative to the reference sequence.

One example of an algorithm that is suitable for aligning nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between two sequences (for example, as described in Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. The output of the BLAST algorithm includes a graphic showing a nucleotide by nucleotide or amino acid by amino acid best fit alignment. Using this graphic, one of skill in the art can routinely determine the equivalent position in the subject and reference sequences. As used in this disclosure, such an equivalent position in a subject sequence is considered to “correspond to” the position identified in the reference sequence.

The following examples are intended to provide illustrations of the application of the present invention. The following examples are not intended to completely define or otherwise limit the scope of the invention.

### EXAMPLES

Experiments were conducted using a Rubisco mutant library screening for discovery of variants related to yield and stress. Highly diverse DNA libraries of over 26,000 Rubisco gene variants were screened through transformation and expression in a C. reinhardtii Rubisco knockout line followed by competitive growth in turbidostats. Clones from the pools of transgenic algae cells were sequenced at early and final time points. Variants present at the endpoint of the primary screen were then recombined into turbidostats as a secondary screen. Winning clones, characterized by an increased representation in these pools, were then used to identify the top candidates. Winning clones were validated in a process whereby the original transgenic lines were competed in growth competition assays in turbidostats head to head against a wild type analog. Transgenic lines were regenerated for the selected variants and these were assayed in turbidostats as well. Original lines were also analyzed by several methods including growth, photosynthetic, and biochemical assays.

Throughout the Examples the following terms and abbreviations are used:

s (selection coefficient): a measure of the relative fitness of a phenotype.

s_{avg}: the average of all s values for a set of replicates for a set.

sₛᵤₘ: the selection coefficient based on the sum of hits and totals for all replicates.

Δs_{avg}: the difference between s_{avg} of a winner and that of the control strain.

Sum_{BC}: the total number of reads associated with a barcoded amplicon in NGS

Sumₚₒₛ: the total number of error-free reads at a particular codon position in NGS

Region: a unique 7-amino acid segment of the rbcL protein or, alternatively, a unique 21-nucleotide segment of the rbcL gene.

Pool: a combination of 2 Regions (14 amino acids) used to divide SSM libraries into 34 distinct parts in the SSM library or a combination of 96 or 34 variants used to divide the Triple Combo library into 66 distinct parts.

Variant: a version of the rbcL protein derived from a mutagenic library, typically containing one or more point mutations from the native residue to one of 9 pre-defined amino acids or an algal strain expressing one of the altered versions of Rubisco.

TAP: A medium for growing algae containing acetate as a carbon source. Allows for mixotrophic or heterotrophic growth.

HSM: A medium for growing algae with no organic carbon source. It requires obligate photoautotrophic growth.

SSM: Site-saturation mutagenesis, a mechanism to generate systematic substitutions across a protein.

TC: Triple Combo, denoting Variant derived from three way combinations of top substitutions.

Table 1 shows the major components of the media used. The MASM used was modified from published formulations in that NH₄ was not used and NO₃ was the only nitrogen source.

TABLE 1

| Component | HSM | | mHSM | |
|---|---|---|---|---|
| NH₄Cl | 7.5 | mM | — | |
| KNO₃ | — | | 7.42 | mM |
| CaCl₂ | 0.35 | mM | 0.35 | mM |
| MgSO₄ | 0.4 | mM | 0.4 | mM |
| Potassium Phosphate sol'n | 1.35 | mM | 1.35 | mM |

| | |
|---|---|
| Trace elements | <<1 mM Zn, B, Mn, Fe, Co, Cu, Mo |

### Example 1. Single Point Mutations

### Screening

### Libraries

Libraries were generated based on a technique known as Site Saturation Mutagenesis (U.S. Pat. No. 5,830,650), where each amino acid in the C. reinhardtii Rubisco large subunit protein (rbcL) was substituted for 9 amino acids representing different classes of side-chain chemistries. These include the positively charged amino acids histidine (H) and lysine (K), negatively charged aspartic acid (D), polar neutral serine (S) and glutamine (Q), hydrophobic leucine (L) and tyrosine (Y), small flexible glycine (G), and small rigid proline (P).

In total 68 individual libraries were generated, each representing a unique 7-amino acid segment of the Rubisco protein referred to as a Region. Therefore the mutations at amino acid positions 1-7 were generated in the Region 1 library, mutations for amino acids 8-14 were generated in Region 2, and so forth. These 68 regions cover the entire Rubisco protein. To generate the mutations, DNA oligonucleotides covering portions of the rbcL gene were synthesized, each containing a single codon change to produce the desired amino acid substitution. Oligonucleotides for each region were ordered in a plate array according to the reference amino acid position and the respective substitution.

Each 63-mer oligonucleotide encompasses the 21 nucleotide region where the mutagenic codon resides, along with 21 nucleotides at both the 5′ and 3′ ends identical to the wild type sequence flanking that region. In addition, a single non-mutagenic oligonucleotide was designed for each region on the opposite DNA strand, such that 21 nucleotides of homology exist between each mutagenic oligonucleotide and its non-mutagenic counterpart.

Mutations were incorporated into the rbcL gene using an overlap PCR technique. Briefly, each mutagenic oligonucleotide was used as a primer in a PCR reaction with another primer at the opposite end of the gene, along with a template plasmid containing the wild type C. reinhardtii rbcL sequence. Primers are incorporated into the resulting PCR products, thereby introducing the mutagenic codon at one end of the amplicon. A second PCR is carried out in a similar fashion using the complimentary non-mutagenic oligonucleotide as a primer to amplify the portion of the gene not covered by the first reaction. In a third PCR, purified amplicons from each of the previous reactions are combined with primers that flank the rbcL gene to both amplify the assembled variant and add restriction endonuclease sites NdeI and SpeI to the 5′ and 3′ ends, respectively. Homology between the amplicons derived from the initial PCRs allows the entire gene to be assembled without the use of a traditional template.

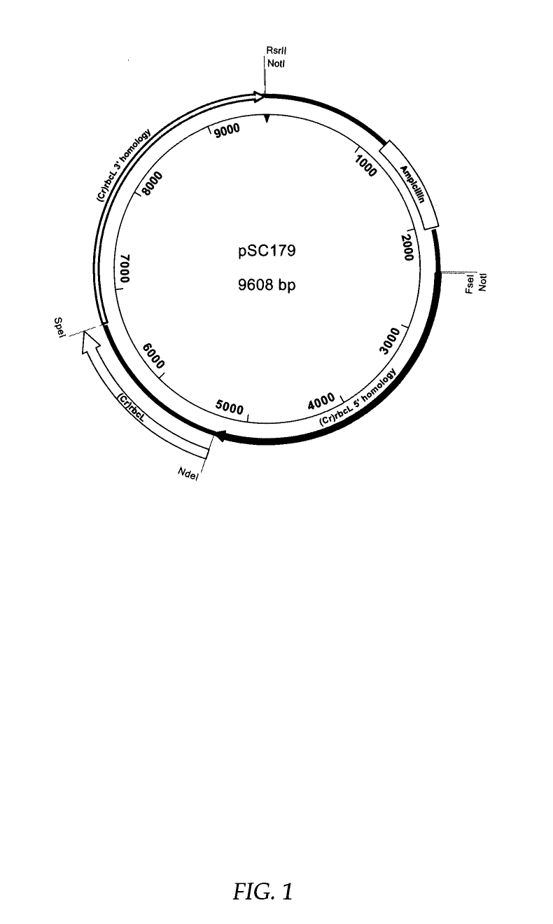

Full-length amplicons were digested with NdeI and SpeI, and ligated into the C. reinhardtii chloroplast transformation vector pSC179 (FIG. 1). The vector contains approximately 2.8 kb each of 5′ and 3′ flanking sequence homology to the rbcL locus in the C. reinhardtii chloroplast genome, as well as elements for propagation and selection in bacteria.

Once the libraries were ligated into the vector, they were transformed into bacteria for amplification and QC. Resulting bacterial colonies for each library (median n=2,400) were scraped into liquid cultures and plasmid DNA was purified the following day. The distribution of mutations in each library was determined by amplifying each mutagenic region with uniquely barcoded PCR primers, followed by Ion Torrent next-generation sequencing (NGS) described in further detail below. Traditional Sanger sequencing of variants in isolated bacterial colonies allowed for validation of the NGS approach. Each variant is reported as a percentage of the 7 amino acid region, and given a perfect distribution would be 1/63 or 1.6% for each variant. Actual variant percentages in the plasmid library ranged from 0%42.5%, with an average of 1.2% and median of 0.9%. While these wild type primers were removed from the original gene synthesis PCR, wild type sequences were present at varying levels (5.7%-60.2%) across the regions. These wild type sequences are likely derived from mis-priming via truncated mutagenic oligos in the overlap PCR or from original template carryover.

Based on the NGS sequencing results, all but 56 of the 4,266 expected mutants were detectable above the baseline noise level in the plasmid library. Note that the 9 substitutions of the start codon ATG were not present because that codon is part of the cloning site NdeI. The other variants that were not detected were simply not created during the PCR process or were below the level of detection for the sequencing method.

The NGS approach and Sanger sequencing of individual clones were compared to show that both gave essentially equivalent results and to select the better method for subsequent sequencing. For NGS, a set of primers with a unique barcode was designed for each region to produce an amplicon of ^{˜}240 bp. These 68 primer sets were used to amplify DNA from each of the 68 plasmid libraries. The 68 PCR products were combined and sequenced on an Ion Torrent 316 chip with 200 bp chemistry. Deconvoluted data for each barcode was then mapped to the reference rbcL sequence to determine the number of reads containing each of the 63 created variants. Sequencing errors due to insertions, deletions, early terminations, etc. were excluded from the analysis, and therefore the total number of sequences at each codon position varies across an amplicon. To normalize the counts for each variant, the raw number of reads was multiplied by the correction factor Sum_{BC}/Sumₚₒₛ, where Sum_{BC} is the total number of reads for the barcode (amplicon), and Sumₚₒₛ is the total number of error-free reads at that codon position. A noise threshold was established for NGS data as the max number of reads seen for a variant that is known to be nonexistent. In this case, the 9 variants for the Start codon (which was not mutagenized) were used. Across several Ion Torrent runs, the maximum number of counts seen for one of these variants was 21. Counts of 21 or below were considered noise and this was applied across all datasets.

Given an equal distribution of mutant sequences within a barcoded amplicon, the wild type codon will be present 6 out of 7 times in the region of interest, making up approximately 85% of the reads on a codon-by-codon basis. Therefore, the number of wild type sequences must be estimated with a different approach than the codon focused method used for variant counts. In each plasmid library, each nucleotide in a read was marked as “likely reference” if it was (i) identical to the reference sequence, (ii) called as a deletion, or (iii) not covered by that particular read. Reads containing 21/21 “likely reference” nucleotides in the region of interest were counted as wild type. Individual bacterial colonies each containing a single variant were also PCR amplified with gene specific primers and subsequently sequenced by the Sanger method. Shown here is a comparison of the Region 2 plasmid library using each sequencing method. The distribution of variants as measured by the two methods appears to be comparable though it is important to note that the Sanger data is based on 94 clonal sequences, while the NGS data has over 38,000 reads for this region. NGS was used for subsequent samples as it provides a more robust data set, often detecting variants that are too rare to be identified by the limited amount of Sanger sequencing that can be done. The main advantage of Sanger is that it allows for clonal isolation of particular variants while NGS does not. For this reason, some Sanger was still done in later stages.

### Primary Turbidostat Screening

A Rubisco knockout (ΔrbcL) algal strain was generated by transforming wild type C. reinhardtii cells via gold particle bombardment with a vector containing the kanamycin resistance gene aphA6 flanked by 5′ and 3′ homology to the rbcL locus. Since transformation occurs by homologous recombination in the C. reinhardtii chloroplast, selection of kanamycin resistant clones is indicative of rbcL displacement in one or more copies of the chloroplast genome. Continually passaging transformants on selective media resulted in homoplasmic knockout clones where no copies of rbcL were detectable by PCR. Since the knockout strain is non-photosynthetic it requires an organic carbon source such as acetate to grow. DNA from each rbcL variant library was transformed into the chloroplast genome of ΔrbcL C. reinhardtii cells via gold particle bombardment. Again, homologous recombination results in replacement at the rbcL locus, this time replacing the aphA6 kanamycin marker with rbcL variants. Selection for rbcL complementation was carried out on HSM agar, a minimal medium that necessitates obligate photoautotrophic growth. Any rbcL variants that were not present in these transformant lines were likely non-functional forms of Rubisco inactivated by the introduced amino acid substitution.

Transformed algal colonies for each region (median n=270) were scraped into flasks containing TAP media. 1-3 days later, cells were passaged to a new flask, and then inoculated into quadruplicate turbidostats 2-4 days later. The turbidostats were filled with HSM media and set to an OD₇₅₀ of approximately 0.3, which represents an early- to mid-log growth phase. Constant light of ^{˜}150 μEinstein was provided, with a constant stream of 0.2% CO₂ bubbling into the culture. Cultures were monitored at least daily for media replenishment, CO₂ delivery, culture settling, cell sticking, mechanical failure or any other issues. The cultures were grown under these optimal photoautotrophic conditions for three weeks. Samples were taken from the inoculum flasks and subsequently from each turbidostat at 7 day intervals, and single cells were sorted by fluorescence-activated cell sorting (FACS) into 96-well plates containing TAP media. Cell lysates were also prepared from each sample for DNA sequencing (NGS). After a week or more of growth, sorted strains were replicated onto solid media for longer-term recovery and isolation of transformed lines.

Sequencing and Analysis from Primary Turbidostat Screening

After 5-7 days of growth in 96-well plates, the individual sorted strains were used as template in a PCR reaction that amplified the rbcL gene based on gene specific primers. After ascertaining success in producing a single product from the reactions, the PCR products were treated for sequencing with Exonuclease I/Shrimp Alkaline Phosphatase (ExoSAP). These products were then sequenced via Sanger chemistry using a primer that reads into the region of interest. These clonally isolated and sequenced variants were used as a check on the NGS data, but were primarily for identification and isolation of particular clones of interest for validation work.

The distribution of mutants in each turbidostat was also determined by amplifying the region of interest from cell lysates with uniquely barcoded PCR primer sets, followed by Ion Torrent next-generation sequencing (similar to the plasmid library sequencing described above). Each set of replicates was combined after barcoding, i.e. 68 “A” replicates for one Ion Torrent chip, 68 “B” replicates for another chip, etc. for all four replicates and four Ion Torrent chips. Analysis of the NGS data from turbidostat algae samples was similar to that described earlier for the plasmid library analysis.

Sequences were analyzed in sets derived from each turbidostat replicate at beginning and ending timepoints, with the difference being baseline (time 0) datasets, which were analyzed per pool and then used as the starting point for each turbidostat replicate of that pool.

Hit counts and total sequences were used to calculate the ratio of each gene present in a given timepoint. These numbers were then be used to calculate a selection coefficient using the formula below (Lenski. Quantifying fitness and gene stability in microorganisms. Biotechnology (1991) vol. 15 pp. 173-92). Note that the selection coefficients used in this analysis do not conform strictly to some of the assumptions upon which the formula is based, in that this is not a single clone compared against a uniform population. Each clone is compared to the rest of the pool, which itself is made up of many other clones. However, within the experiment, the calculated selection coefficients provide a valid way to compare and rank potentially winning clones. Since wild type is also present in each pool, a selection coefficient can be calculated with wild type as a common competitor (i.e. the ratio (r) in the formula is the number of a variants divided by the number of wild type). However, this calculation is still influenced by the rest of the population and is not a true wild type-based selection. Additionally, the wild type count is based on a secondary comparison of the whole region to the reference rather than a codon by codon comparison as for the variants, a calculation that is not directly comparable and consistent with the method used for variant number counting.

ln(rₜ)=ln(r₀)+s·t

where r₀ is the ratio of hits for a given clone to hits for the remainder of the population at a starting time, rₜ is this ratio at time t and s is the selection coefficient (expressed in units of t⁻¹).

In many cases, a given sequence was identified at one time point but not detected in another time point (most commonly, a variant that existed at the initial time point but was selected against and was not detected at the endpoint). As the natural log of zero produces an error, assumptions were necessary in such cases. For any instance where the baseline was zero but the variant was detected at the endpoint, a value of 1 count was assigned to the baseline. For the more common case where the variant was detected at baseline but not at the endpoint (termed “extinct” variants), a value of 0.0001 was assigned to the endpoint resulting in a large negative selection coefficient, but avoiding the calculation error. Additionally, the noise threshold was applied to the dataset (i.e. 21 or less counts considered as noise). During the analysis, these assumptions were monitored to avoid consideration of artifactual data. As an example, if a clone was below the noise threshold in one timepoint and detected zero times in the other (therefore an assumed single hit), this could produce a rather large s value, negative or positive, depending on which timepoint had more total sequences. A winner, however, as not selected based on this type of data as a signal in the noise is not sufficient for accurate results.

The formula was used to estimate the length of time required for competition and the number of clones to analyze in order to reach a desired level of sensitivity. This estimate was based on Sanger sequencing prior to validation of NGS—use of NGS should give much more sensitivity and resolution. Assuming a 1/63 starting ratio, approximately 200 sequences at the endpoint and a sensitivity of 5% (i.e. 10 sequences out of 200), the time necessary to identify a clone with a selection coefficient of 0.0500 was calculated as follows:

ln(10/190)=ln(1/63)+0.0500d⁻¹·t days; t=23.9 days

Thus in the primary screen, an s value of approximately 0.05 should be detectable within 3 weeks of growth by sequencing approximately 200 clones. These calculated selection coefficients were then used to rank and select potential winning clones. See results section below for details.

### Secondary Turbidostat Screening

Variants present at the primary screening endpoint were recombined and subjected to a secondary screen. After 23-27 days of primary screening, flasks were prepared for each region by combining 15 mL of culture from each primary replicate turbidostat. All regions had four replicates running at the three week time point, except for Region 11 which had only three. Cultures from adjacent regions were then combined in equal volumes into new flasks using a sliding window of 8 regions, moving down four regions at a time. Each recombined culture is referred to as a Pool. In one strategy (A), Regions 1-8 were recombined into Pool 1, Regions 5-12 were recombined into Pool 2, and so forth for a total of 16 pools. These were grouped for analysis as Even and Odd to avoid overlap of regions, so that the Strategy A-Even pools contain Regions 5-68 and the Strategy A-Odd pools contain Regions 1-64. In a second strategy (B), 3 pools comprising adjacent regions from each ^{˜}⅓ of the protein were created in a similar fashion. In this second strategy, Regions 1-23 were recombined into Pool 17, Regions 24-46 into Pool 18, and Regions 47-68 into Pool 19. In total, each variant present at the primary screening endpoint was recombined into three distinct pools, except for variants from Regions 1-4 and 65-68, which were recombined into two distinct pools.

After 1 day of growth in flasks, each pool was inoculated into quadruplicate turbidostats. Additionally, single cells were sorted by FACS from each pool into 96-well plates and cell lysates were prepared for a baseline data point by NGS. The turbidostats were filled with HSM media and set to an OD₇₅₀ of approximately 0.3, which represents an early- to mid-log phase. Constant light of ^{˜}150 μEinstein was provided, with a constant stream of 0.2% CO₂ bubbling into the culture. Cultures were monitored at least daily for media replenishment, CO₂ delivery, culture settling, cell sticking, mechanical failure or any other issues. Samples were taken at 7 days and at 14 days, single cells were sorted by FACS into 96-well plates, and cell lysates were prepared for NGS. After a week or more of growth, sorted strains were replicated onto solid media for longer term recovery and isolation of transformed lines.

Sequencing and Analysis from Secondary Turbidostat Screening

Primer sets and amplicons were adjusted for the secondary screening. For Strategy A, 16 barcoded primer sets each amplify the 8 regions included in one of the 16 pools. These 16 amplicons were combined and sequenced on an Ion Torrent 316 chip (one for each of the four replicate sets). The three Strategy B pools were amplified as ^{˜}600 bp regions and each amplicon was sheared and barcoded during library creation. The three barcoded libraries were combined for each replicate set and run on Ion Torrent 318 chips (again, one for each of four replicate sets).

Analysis of the NGS data was as described earlier, with the exception that each barcode dataset covered 8 regions (Strategy A) or up to 23 regions (Strategy B) rather than the single region per barcode for the plasmid library and primary screening. Additionally, a higher noise threshold was used for the 318 Ion Torrent chips (i.e. 10-fold more data with the same error rate means a 10-fold higher noise level).

The calculated selection coefficients were then used to rank and select potential winning clones as proposed genes. See results below for details.

### Screening Results

### Primary Turbidostat Screening and Results

Two independent transformation waves, each consisting of 9 biolistic shots per region, provided the transformed Chlamydomonas lines for primary screening. After colonies had grown up on the transformation plates, they were counted and put into sets with an average of 383 (median=270) colonies in each. These sets represented the independent region variant clones that made up the pools for turbidostat screening.

Based on our experience with operating turbidostats, attrition was expected over the course of a multi-week experiment due to occasional equipment failure or culture crash. Therefore excess replicates were set up for screening. 68 pools were initially set up, one per region, and four replicate turbidostats were established per pool. The target screening time for the cultures was 3 weeks. During the three weeks of screening, only one turbidostat out of 272 failed to make the endpoint.

Use of NGS for data generation allowed for much more resolution over use of Sanger sequencing of individual clones. While Sanger data only provides data on the most prevalent variants in the population (and thus skews towards those variants that become dominant via a selective advantage), NGS allows sampling of nearly all variants in a pool. Thus those variants that are neutral or with a negative selection can be identified and characterized. Even those mutants that are present at the beginning of an experiment that go to zero (“extinct”) can be fairly reliably detected.

The primary screen pools had a relatively low diversity. Each pool had a maximum of 63 variants (7 amino acids×9 substitutions) with an actual average variant number per pool of 58. This also suggested that an average of 5 variants per pool did not complement the knockout rbcL strain. Because of this low diversity, the four replicates of each primary pool showed good reproducibility. Selection coefficient values derived from the primary screen, while relative only to variants within the region and some fraction of wild type, could be relied upon as a main criterion for selecting winners.

### Re-Rack for Secondary Turbidostat Screening

Given the relatively small number of variants in each pool during primary screening, coupled with the fact that the diversity should be even smaller and skewed towards winners at three weeks' time, secondary pools were created directly from the primary turbidostats. In Strategy A pools, this would produce a maximum diversity of 504 variants (8 regions×63 variants) with the actual average diversity of these 16 pools being 263 variants. For Strategy B pools, up to 1,449 variants (23 regions×63 variants) would be present with the actual diversity in these three secondary pools being 982 variants.

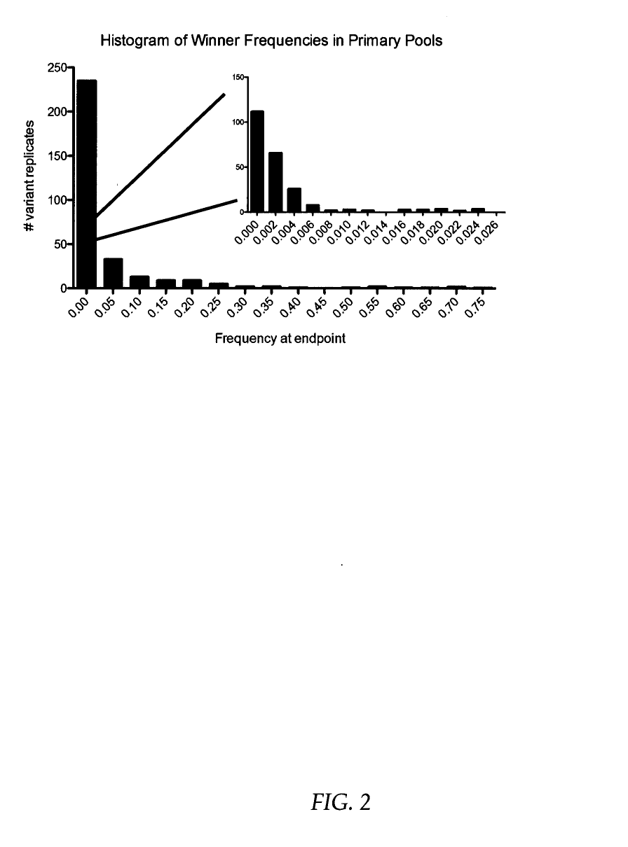

Once potential winning lines were identified from the NGS data, clones must be identified for subsequent Validation. Clonal algae samples from primary screen endpoints as well as secondary screen final timepoints were PCR amplified and sequenced. The liquid culture FACS plates were transferred to solid media at the time of sequencing. The colonies grown up on these plates were used to recover the strains for each potential winner. The strains were struck out for single colonies to ensure clonal isolation, then the rbcL gene was PCR amplified and sequenced to confirm the identity of each clone. These strains were also the isolates that were used in the Validation process. Many of the variants identified as winning lines were not in the set of sequenced clonal isolates. This was the main disadvantage of using NGS for data analysis. The sensitivity afforded by NGS leads to identification of winners that are at such a low frequency, even at the endpoint of screening, that hundreds or thousands of clones would have to be individually sequenced for each variant in order to clonally isolate the strain. This can be clearly seen in the plot of variant frequency at the endpoint of primary screening shown FIG. 2. This plot includes only the replicates for the ^{˜}100 proposed winning lines. Despite being winning lines many are still at low frequency in the primary pool endpoint—below Sanger detection levels. Any variant for which an algae clone cannot be identified will have to be regenerated before a complete Validation dataset can be generated. Given that integration of transgenes occurs by homologous recombination in the C. reinhardtii chloroplast, any variant that was detected in one pool was treated as equivalent to the same variant in any other pool. Additionally, this implies that regenerated lines should be equivalent to those isolated from the screen so that Validation with those lines will be comparable to work done with the original transformed and screened lines.

### Gene Selection

Selection coefficients were calculated for all variants in each replicate turbidostat, using the common baseline hit ratio for the pool and the final hit ratio for each replicate (column s in Table 2). The average of these replicates values was calculated as s_{avg}. In the example from primary screening given below, time is 21 days. As a demonstration, s for the first replicate in Table 2 (and highlighted in bold) is calculated as follows:

ln(rₜ)=ln(r₀)+s·t

ln(31429/(64322−31429))=ln(2218/(45188−2218))+s·21

ln(0.9555)=ln(0.0516)+s·21

s=0.1390

Example data for two variants at one position is given in Table 2 below. Position and original residue is anonymized, but actual data in presented.

TABLE 2

| | | Raw | | | | Corrected | Corrected | | | | | |
|---|---|---|---|---|---|---|---|---|---|---|---|---|
| Variant | Replicate | Counts | Sumₚₒₛ | Sum_{BC} | Correction | Counts | Sum_{BC} | Ratio | s | s_{avg} | stdev | CI |
| X999Q | Baseline | 1495 | 28573 | 42389 | 1.483 | 2218 | 45188 | 0.0516 | | | | |
| | A | 21297 | 39575 | 58403 | 1.475 | 31429 | 64322 | 0.9555 | 0.1390 | 0.1127 | 0.0384 | 0.0802 |
| | B | 6801 | 36513 | 53586 | 1.467 | 9981 | 53652 | 0.2285 | 0.0709 | | | |
| | C | 25910 | 44510 | 62868 | 1.412 | 36596 | 66339 | 1.2305 | 0.1510 | | | |
| | D | 11512 | 44395 | 63809 | 1.437 | 16546 | 65067 | 0.3410 | 0.0899 | | | |
| X999S | Baseline | 1427 | 28573 | 42389 | 1.483 | 2117 | 45188 | 0.0492 | | | | |
| | A | 924 | 39575 | 58403 | 1.475 | 1364 | 64322 | 0.0217 | −0.0390 | −0.0256 | 0.0761 | 0.1589 |
| | B | 330 | 36513 | 53586 | 1.467 | 484 | 53652 | 0.0091 | −0.0803 | | | |
| | C | 540 | 44510 | 62868 | 1.412 | 763 | 66339 | 0.0116 | −0.0686 | | | |
| | D | 10345 | 44395 | 63809 | 1.437 | 14869 | 65067 | 0.2962 | 0.0855 | | | |

As described previously, the counts for each variant were normalized across a given barcode. The raw count of reads in a given position (i.e. amino acid number) was multiplied by the correction factor Sum_{BC}/Sumₚₒₛ, where Sum_{BC} is the total number of reads for the barcode (amplicon), and Sumₚₒₛ is the total number of error-free reads at that codon position. The corrected Sum_{BC} for a barcode is calculated by summing all corrected counts for variants at all positions in a barcode, then adding the number of wild type counts as determined by the fraction of “likely reference” (see earlier description). For each s_{avg}, a 95% confidence internal (CI) was calculated to determine if the average was significantly higher than zero (one-sample, one-sided t test, p<0.05). In this example, the first Variant (indicated with an arbitrary starting amino acid and position of X999 substituted to Q) is statistically higher than zero as the average minus the CI is greater than zero.

A statistical test was applied in order to refine the number of variants considered for Selection. Each of the 4,266 possible variants had up to four separate s_{avg} measurements, each calculated from up to four replicates, resulting in over 60,000 s calculations. It was determined which of the four s_{avg} measurements (Primary, Secondary-Strategy A-Even and -Odd, Secondary-Strategy B) were statistically greater than zero. A one-sample, one-sided t test was used by calculating a 95% confidence interval (α=0.025, n=4) from the standard deviation followed by comparison of this CI to the average. Any s measurements with a CI less than the average were determined to be statistically greater than zero. Any variant with only a single replicate value in a given pool could not be tested for significance. This limited the pool of variants to only those where at least one s_{avg} measurement passed this test resulting in just over 500 variants.

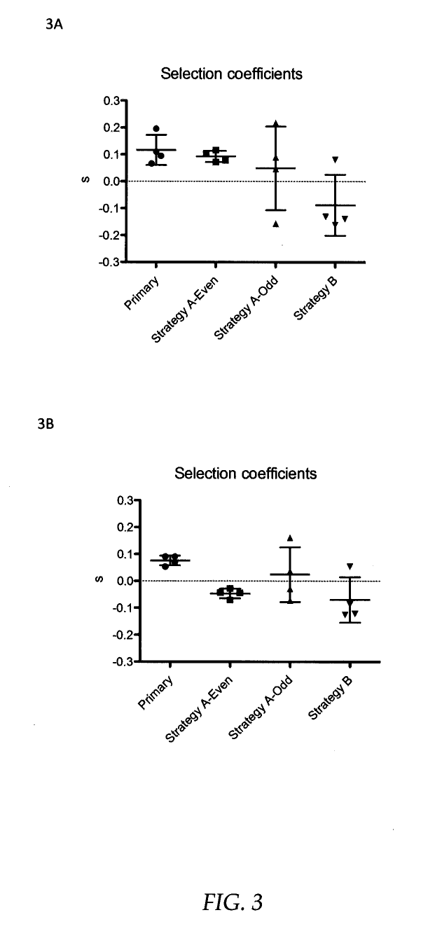

The first set of winners selected from this data was comprised of the variants that had high values of s_{avg} in the primary pools (Class 1). In this experiment, the primary screen had a low starting diversity (max 63 variants) and thus provided the most robust set of s measurements. While the relative selective advantage of a variant in one region of the protein relative to one in a different region cannot be directly determined from the primary screen, any that had a high value for s were presumably some of the most advantaged variants. Therefore any variants with a measured s_{avg} value of greater than 0.05 (and statistically greater than zero) were nominated as potential winners. Several, though not all, of these variants had a selective advantage in the secondary screens as well. Two examples are given in FIG. 3, FIG. 3A has a selective advantage in the first secondary pool, while the FIG. 3B shows an advantage only in the primary. A second small set of variants was added that showed any difference from zero in the primary pools (0<s_{avg}<0.05) and also showed a significant difference from zero in at least one of the secondary pools (Class 2).

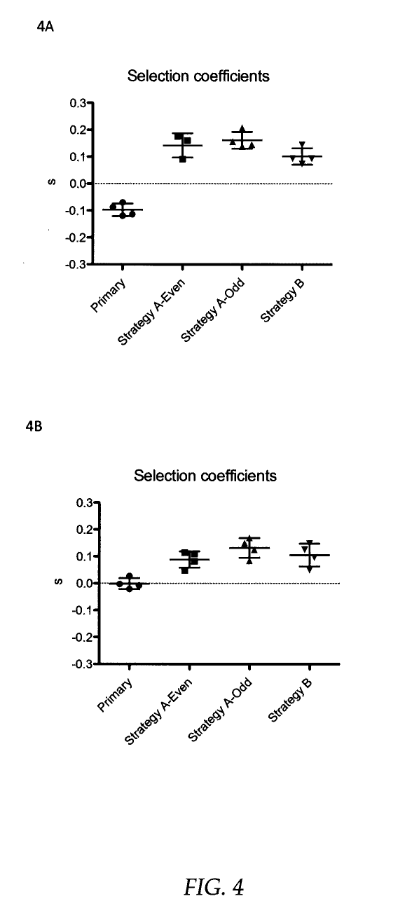

The secondary screening pools put many more variants (500 or more) into a single pool. This provides an opportunity to test variants from different regions against each other, but the higher diversity limits the resolution of the assay. The next class of variants (Class 3) showed a consistent selective advantage with s_{avg}>0.05 in all three of the secondary pools. This class included those potential winners that had a selective advantage no matter the environment in which they were screened. FIG. 4 shows two such variants. Despite a low or negative selective advantage in the primary pools, they both grew significantly better than the pool in all three secondary tests. In one case, a high frequency of wild type in the primary pool may have obscured the winning variant, while in the other case, three Class 1 winners were present in the same primary pool and could have interfered with this Class 3 winner.

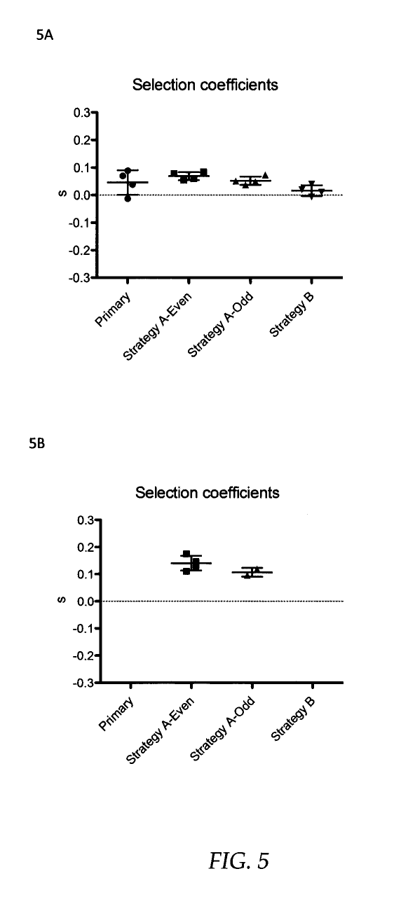

Just as the secondary pools were more diverse than the primary, within the secondary the Strategy B pools were the most diverse of all, with up to ^{˜}1,400 possible variants in each pool—one third of all variants created in the original libraries. This number of regions pushed against the limits of detection with the NGS approach. Because of this, more weight was given to the Strategy A pools where only 8 regions were combined at a time. Any variants with a statistically significant s_{avg} of greater than 0.05 in both Even and Odd Strategy A pools were also nominated to the winner list (Class 4). Looking at the Strategy B s_{avg} values for these variants showed that about ⅓ had a value significantly greater than zero but less than 0.05, ⅓ had a value greater than zero that was not statistically significant (FIG. 5A), and ⅓ did not have any value for s_{avg} (FIG. 5B). Typically this is due to an undetectable count number for several timepoints.

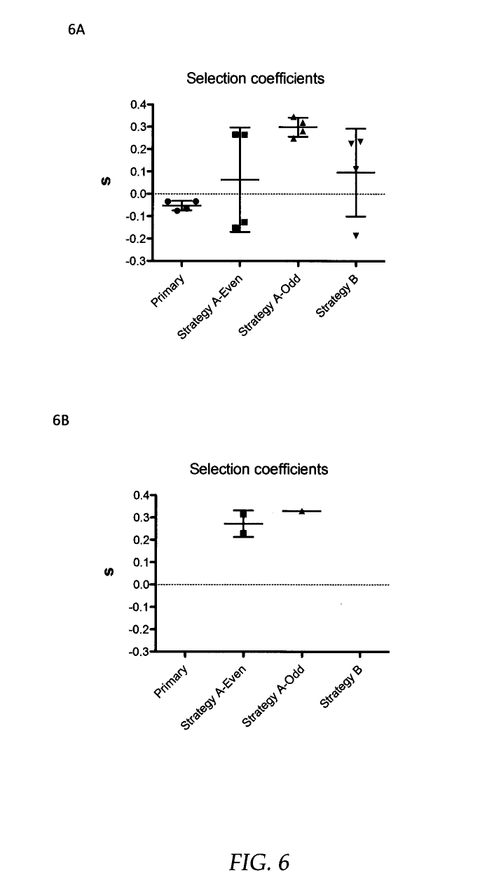

In some cases, a particular variant is masked in most of the pools due to the combination of genetics and environment found in those pools. Because of this phenomenon, winners were not selected based solely on a competitive advantage in multiple experiments. In fact, a winner could show an advantage in a single pool and not in any of the others in which it was screened. The final set of variants nominated to the winner list included those that showed a particularly strong selective advantage (s_{avg}>0.20) in any one secondary pool (Class 5). The example in FIG. 6A is very strong in Secondary Strategy A-Odd while more variable in the other secondary pools. The example FIG. 6B only had evaluable data for one pool (and a single replicate in another), but in that pool showed a very strong selective advantage.

The five classes are outlined in Table 3 below. For a variant to be included in a class, all columns must be true (e.g. Class 3 have s_{avg}>0.05 for all three secondary pools). The number of variants in each Class is also listed in the table. Note that a given variant was included in only one Class even if it qualified for more than one. That is, a number of Class 1 variants could also be considered Class 2 variants as they have at least one secondary pool s_{avg} greater than 0, or all Class 3 variants could also be considered Class 4 and several of them would qualify for Class 5. There are a total of 104 variants in Class 1-5.

TABLE 3

| | | Secondary | Secondary | Secondary | # Var- |
|---|---|---|---|---|---|
| Class | Primary | A-Even | A- Odd | B | iants |
| 1 | s_{avg} > 0.05 | — | — | — | 36 |
| 2 | s_{avg} > 0 | At least one s_{avg} > 0 | | | 5 |
| 3 | — | s_{avg} > 0.05 | s_{avg} > 0.05 | s_{avg} > 0.05 | 27 |
| 4 | — | s_{avg} > 0.05 | s_{avg} > 0.05 | — | 17 |

| | | | |
|---|---|---|---|
| 5 | — | At least one s_{avg} > 0.2 | 19 |

### Validation

Turbidostat Competitions with Primary Lines

Wild Type Analog Strain (Common Competitor).

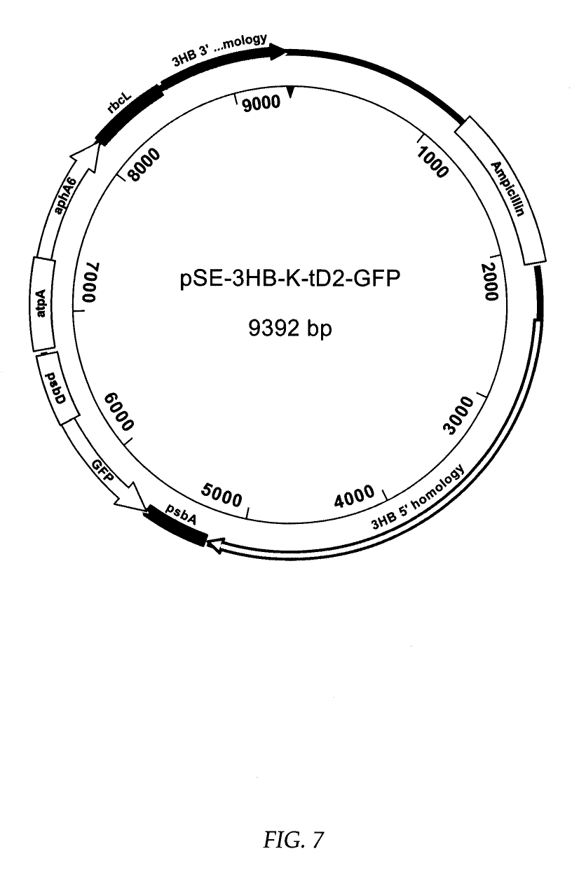

A simple method to determine the relative ratios of two strains in a turbidostat is to sort the population onto selective and non-selective media and count the number of viable colonies that form on each, provided that only one of the strains is capable of growing on the selective medium. Since Rubisco variants do not contain a selectable marker, a strain carrying a selectable marker was chosen as a common competitor for all Selected Variants, as well as wild type. The strain was generated by transforming wild type C. reinhardtii cells via gold particle bombardment with a plasmid containing the kanamycin resistance gene aphA6 driven by the atpA promoter and rbcL terminator, and GFP driven by the psbD promoter and psbA terminator, all flanked by homology to the chloroplast genome 3HB locus (see FIG. 7). This strain only expresses GFP and an antibiotic resistance gene from a neutral site in the chloroplast genome, and as a common competitor, the relative fitness of wild type and Selected Variants to this strain can be compared.

1:1 Turbidostate Competitions.

50 ml starter cultures of each original line winner were grown in TAP media to mid- to late-log phase in flasks. The kanamycin-resistant wild type analog strain was treated in the same manner though at larger scale. For inoculation into turbidostats OD₇₅₀ readings of wild type analog and winner cultures were taken and used to generate a solution containing wild type analog and Rubisco variant line at a ratio of 10:1 at a final OD₇₅₀ of approximately 0.5. 10 ml of this mixture was used to inoculate turbidostats with a final volume of 30 ml. Four replicate turbidostats were inoculated from each winner line. The turbidostats were filled with HSM media and set to an OD₇₅₀ of approximately 0.3, which represents an early- to mid-log growth phase. Constant light of ^{˜}150 μEinstein (μE) was provided, with a constant stream of 0.2% CO₂ bubbling into the culture.

A sample of the mixture used for turbidostat inoculation (time=0) was sorted for single colonies using FACS, then grown on both TAP media (permissive for winner and wild type analog) and TAP media containing 50 μg/ml kanamycin (permissive for wild type analog only). 384 events were analyzed for each media type. After 10-16 days of turbidostat growth, a sample was taken and used for the same sorting procedure.

After approximately one week of growth, photographs of sorted plates were taken by digital camera. Colony numbers on each plate were calculated using the colony counter plugin for ImageJ software. These colony numbers can then be used to calculate a selection coefficient using the formula provided previously.

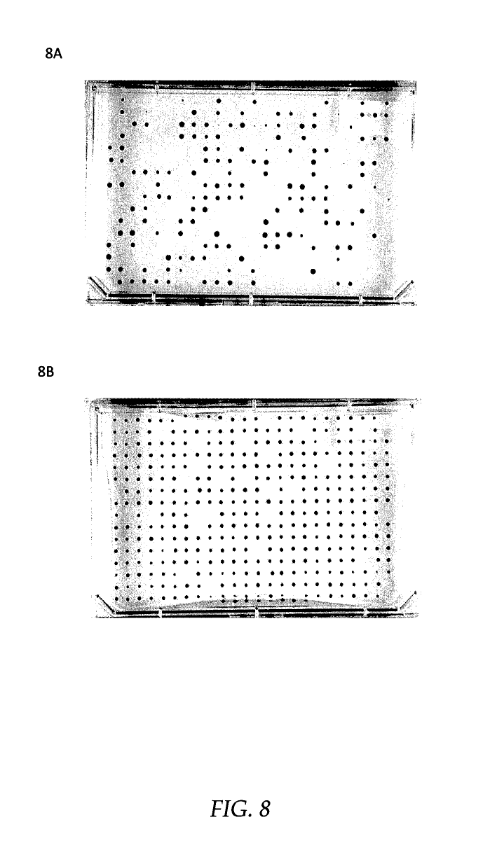

FIG. 8A shows an exemplary plate with selection and FIG. 8B is without selection. In this case, 151 colonies grew on selection and 354 grew without selection, implying that 203 are kanamycin-sensitive Rubisco variants (354−151=203). Based on these numbers, the ratio for this sample is calculated as:

rₜ=203/(354−203)=1.344

This value was calculated separately for each replicate turbidostat.

### Regeneration of Lines

Overlap PCR and Chloroplast Transformation.

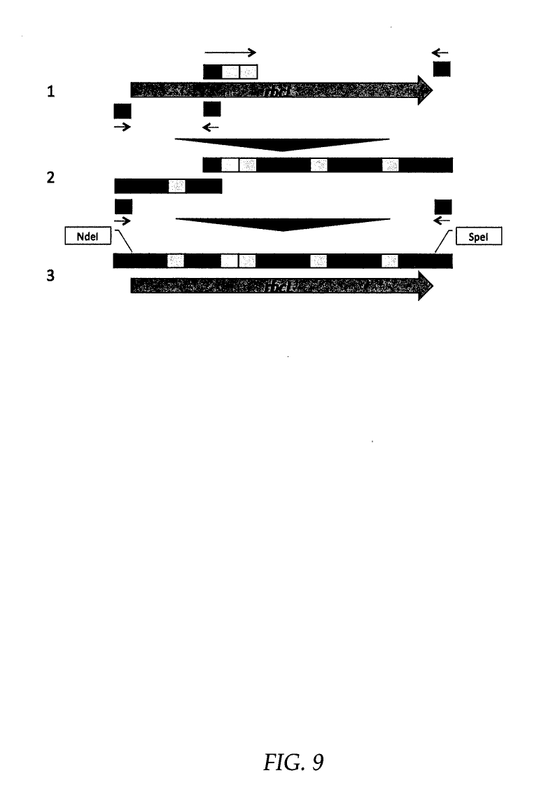

Variants were regenerated via the overlap PCR method used to generate the original variant libraries. Briefly, an oligonucleotide containing the mutation of interest was used as a primer in a PCR reaction with another primer at the opposite end of the gene, along with a template plasmid containing the wild type C. reinhardtii rbcL sequence. Primers are incorporated into the resulting PCR products, thereby introducing the mutagenic codon at one end of the amplicon. A second PCR is carried out in a similar fashion using the complimentary non-mutagenic oligonucleotide as a primer to amplify the portion of the gene not covered by the first reaction. In a third PCR, purified amplicons from each of the previous reactions are combined with primers that flank the rbcL gene to both amplify the assembled variant and add restriction endonuclease sites NdeI and SpeI to the 5′ and 3′ ends, respectively. Homology between the amplicons derived from the initial PCRs allows the entire gene to be assembled without the use of a traditional template (see FIG. 9).

Full-length amplicons were digested with NdeI and SpeI, and ligated into the C. reinhardtii chloroplast transformation vector pSC179 (see FIG. 1). The vector contains approximately 2.8 kb each of 5′ and 3′ flanking sequence homology to the rbcL locus in the C. reinhardtii chloroplast genome, as well as elements for propagation and selection in bacteria.

Ligation products were introduced to E. coli cells via electroporation, and plasmid DNA was isolated from individual colonies for sequence verification. Once a single bacterial colony containing the plasmid sequence of interest was identified it was scaled up in overnight culture for plasmid purification. DNA from each rbcL variant was transformed into the chloroplast genome of rbcLΔ C. reinhardtii cells via gold particle bombardment, and selection for rbcL complementation was carried out on HSM agar, a minimal medium that necessitates obligate photoautotrophic growth. Single colonies were isolated and scaled up on TAP agar for sequence verification.

Selection of transformed lines was carried out by restoration of photosynthesis, and photoautotrophic growth has been shown to drive transgenic strains toward a homoplasmic state in which every copy of the chloroplast genome contains the rbcL variant gene in place of aphA6. To this end clones were inoculated into single turbidostats filled with HSM media and set to an OD₇₅₀ of approximately 0.3, which represents an early- to mid-log growth phase. Constant light of ^{˜}150 μEinstein (μE) was provided, with a constant stream of 0.2% CO₂ bubbling into the culture. Cultures were grown under these photoautotrophic conditions for 3-4 days, or approximately 10-14 generations, prior to inoculating 1:1 competitions.

Turbidostat Competitions with Regenerated Lines

While regenerated lines were being driven to homoplasmicity in turbidostats, the wild type analog strain was scaled up in HSM media to OD₇₅₀ of approximately 0.3. A sample was taken from the regenerated line turbidostats and mixed with wild type analog cells at a ratio of 1:10 (calculated by OD₇₅₀), and the resulting mixture was inoculated into triplicate turbidostats. A sample of the mixture used for turbidostat inoculation was sorted using FACS onto both TAP media and TAP media containing 50 μg/ml kanamycin. 384 events were sorted onto each media type. Another sample was taken at the same time from residual culture in the inoculum turbidostats and sorted into 96-well plates containing 0.2 ml TAP medium in order to determine the approximate proportion of homoplasmic cells in the population.

After approximately one week of growth, photographs of sorted plates were taken by digital camera. Colony numbers on each plate were calculated using the colony counter plugin for ImageJ software. Selection coefficients were calculated as described above.

The 96-well plate cultures of the regenerated lines were also grown for approximately one week. Culture from each well was mixed in equal volume with Tris-EDTA buffer, and heated for 10 min at 98° C. to lyse the cells. For each lysate, 2 PCR reactions were performed: one with primers that amplify rbcL, and one with primers that amplify aphA6. The aphA6 PCR reaction is able to amplify the gene at an aphA6:rbcL ratio of 1:5000 after 35 cycles. The C. reinhardtii chloroplast is reported to contain approximately 80 copies of genomic DNA. Given the sensitivity described, any lysate that produced an rbcL band and no aphA6 band after 35 cycles of PCR was considered to be homoplasmic for the Rubisco variant gene.

Growth and Photosynthesis Assays with Primary Lines

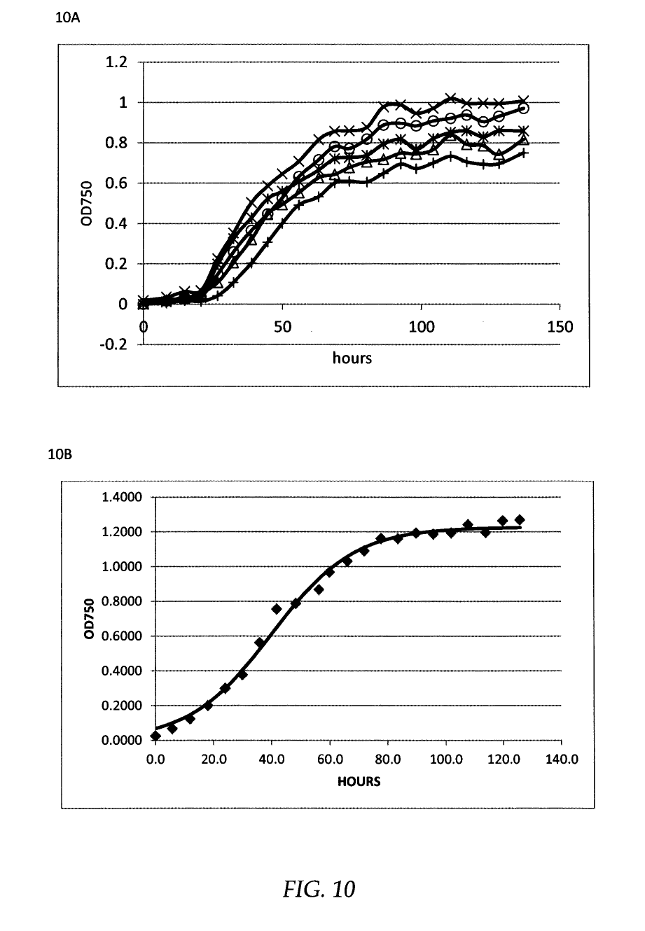

Cultures were grown to stationary phase in MASM, HSM, and TAP media. MASM and HSM are minimal medias with different nitrogen sources (NH₄ for HSM, NO₃ for MASM) while TAP contains an organic carbon source (acetate) and supports mixotrophic growth. Cultures were diluted to OD₇₅₀=0.2 and grown overnight. Overnight growth was followed by a second dilution to OD₇₅₀=0.02. These initial culture densities put the cells in lag or early log phase. At this point, 0.2 ml of each culture was added to a 96-well microtiter plate in randomized replicates. 96-well microtiter plates used in this assay contain opaque sides so that light exposure is equal across the entire plate, and a transparent base to allow OD acquisition in a 96-well plate reader. Plates were covered using a PDMS (poly dimethyl siloxane) lid in order to allow for gas exchange but minimize culture volume loss to evaporation. Covered plates were then set onto a shaker within a growth chamber supplied with 5% CO₂ or 0.04% CO₂ (air). Intermittent shaking was set to 25 seconds on at 1700 rpm, 1 second in each direction (CW/CCW) followed by 60 seconds off. Light incidence upon each plate lid was set to 130 μE. OD₇₅₀ was read every 6 hours for a maximum of 120 hours (until the cultures clearly enter stationary phase as evidenced by the leveling of the curve). The resulting OD₇₅₀ readings, which reflect culture growth, were plotted vs. time. Shown in FIG. 10A is an example of 5 replicate wells for one sample grown in MASM. The data are imported into a curve-fitting software package where a 3 parameter logistic function of the form

N(t)=K/(1+(K/N₀−1)·e^{(-r·t)})

is fit to the data. An example of this curve fit is shown in FIG. 10B. The 3 parameters are system specific and represent the carrying capacity (K), the maximal growth rate (r), and the initial density (Nₒ). Differentiating the logistic function yields a rate function; this function can be optimized and solved analytically. This solution for this optimization is equivalent to Kr/4, which is thus the peak theoretical productivity.

A subset of lines were assayed for growth characteristics in 1-liter column photobioreactors at two temperatures. Rubisco activity has been shown to be temperature sensitive in vivo; this assay was an attempt to find any temperature sensitivity changes caused by the variants. Cultures were scaled up in flasks containing MASM media and inoculated into column reactors in MASM at approximately 0.15-0.2 g/L. Each culture was inoculated into 6 replicate columns: 3 were run at 22° C., and 3 at 30° C. Culture pH was continually monitored and maintained at approximately 8.0 via CO₂ injection. Samples were taken daily from each column to determine biomass accumulation (dry weight per volume) and data was fit to a logistic function similar to that described above for microplate growth assays. Differences in growth rate between the two temperatures were calculated between each replicate at the lower temperature and each replicate at the higher temperature. These growth rate differentials were compared to determine any differences in temperature sensitive growth between the variants and wild type.

Fluorescence Induction and Relaxation (FIRe) Assay.

Lines were also assessed for photosynthetic characteristics using a Satlantic Fluorescence Induction and Relaxation (FIRe) fluorometer. Changes in Rubisco activity could have impacts upstream in the photosystems, especially at light saturating conditions where the dark reactions are typically rate limiting. The FIRe system relies on active stimulation and highly resolved detection of the induction and subsequent relaxation of chlorophyll fluorescence yields on micro- and millisecond time scales. Fluorescence induction can be used to calculate several photosynthetic parameters including the minimum (Fₒ) and maximum (Fₘ) fluorescence yields, the maximum quantum yield of photochemistry in PSII (Fᵥ/Fₘ), and the functional absorption cross-section of PSII (σ_{PSII}) (Gorbunov, M. Y., Kolber, Z. S., & Falkowski, P. G. (1999). Measuring photosynthetic parameters in individual algal cells by Fast Repetition Rate fluorometry. Photosynthesis Research, 62(2), 141-153).

Prior to sampling, cultures were grown in turbidostats for 2-4 days under the same conditions as those used in the screening process. Culture was then removed from the turbidostat, diluted in HSM media, and immediately placed into a cuvette for FIRe analysis. Each turbidostat was sampled 6 times.

Fluorescence was excited by radiation from blue (450 nm with 30 nm half-bandwidth) light-emitting diodes and recorded in the red spectral region (680 nm with 20 nm bandwidth). Electron transport rate (ETR), the number of electrons transported per second per reaction center, was calculated from the FIRe data output using the formula

ETR=PAR·σ_{PSII}·(ΔF′/Fₘ′)/(Fᵥ/Fₘ).

where PAR is the amount of photosynthetically active radiation (μE) and ΔF′/Fₘ′ is the quantum yield of photochemistry in PSII under ambient light. Subsequently ETRₘₐₓ was calculated by fitting a curve to ETR vs. PAR using TableCurve 2D software. Curves were fit using the formula

y=ETRₘₐₓ·(1−e^{(-x·α/ETRmax)})

where x is the PAR level, y is the electron transport rate calculated at each PAR, and a is the initial slope of the curve. The calculated ETRₘₐₓ and a from each measurement, together with σ_{PSII} and Fᵥ/Fₘ at PAR 0, were all exported to JMP for statistical analysis.
Biochemical Assays with Primary Lines

Western Blots.

Samples were taken from turbidostats and pelleted by centrifugation. Soluble protein was extracted from one pellet; mRNA from the other (see qPCR section below). Total soluble protein was extracted from the pellet using Bug Buster Protein Extraction Reagent (Nanogen) per manufacturer instructions. Samples were then normalized to 0.5 mg/ml using a DC Lowry Protein Assay kit (Pierce). A 3-point serial dilution of each normalized sample was made in Bug Buster Extraction Reagent. Samples were then denatured at 95° C., and equal volumes were loaded onto 12% polyacrylamide Bis-Tris gels (Bio-Rad). Following electrophoresis in MES running buffer, protein bands were transferred to a PVDF membrane. Membranes were incubated with a blocking solution followed by a rabbit anti-rbcL antibody (Agrisera). Finally, membranes were incubated with a HRP-conjugated goat anti-rabbit secondary antibody and developed using SuperSignal West Dura Extended Duration Substrate (Thermo Scientific). Average pixel intensity for each band was quantified using FluorChem software (Alpha Innotech).

Quantitative Real-Time PCT (qPCR).

RNA was extracted from algae pellets (see Western Blot section above) using Plant RNA Reagent (Invitrogen) following manufacturer instructions. Samples were then treated with DNAse and purified using RNeasy affinity columns (Qiagen). Double-stranded cDNA was generated from RNA using an iScript cDNA Synthesis kit (Bio-Rad). Each cDNA sample was used as a template in two PCR reactions: one with primers that amplify a short segment of the rbcL transcript, and one with primers that amplify a segment of the reference gene tufA, a chloroplast-encoded elongation factor with stable transcript levels under constant light conditions. All PCR reactions were set up with 6 replicates using iQ SYBR Green Supermix reagent and imaged in real time on a MyiQ thermocycler (Bio-Rad). Relative transcript levels were determined by the comparative C_{T} method (Livak and Schmittgen. Analysis of relative gene expression data using real-time quantitative PCR and the 2^{-ΔΔCT} method. Methods (2001) vol. 25 pp. 402-408), where tufA reactions were used as internal controls to determine the ΔC_{T} of corresponding rbcL reactions, and the wild type ΔC_{T} value was used as a calibrator to calculate ΔΔC_{T} for each primary line cDNA sample. For each sample the standard deviation of the difference (SD_{S}) between rbcL and tufA replicate C_{T} values was calculated to determine the upper and lower bounds of the error (see below).

SDₛ=(SD_{rbcL}²+SD_{tufA}²)^{1/2}

Relative transcript abundance(min,max)=2^{-ΔΔCT}(2^{-ΔΔCT-SDs},2^{-ΔΔCT+SDs})

### Validation Results

### Primary Line Competitions

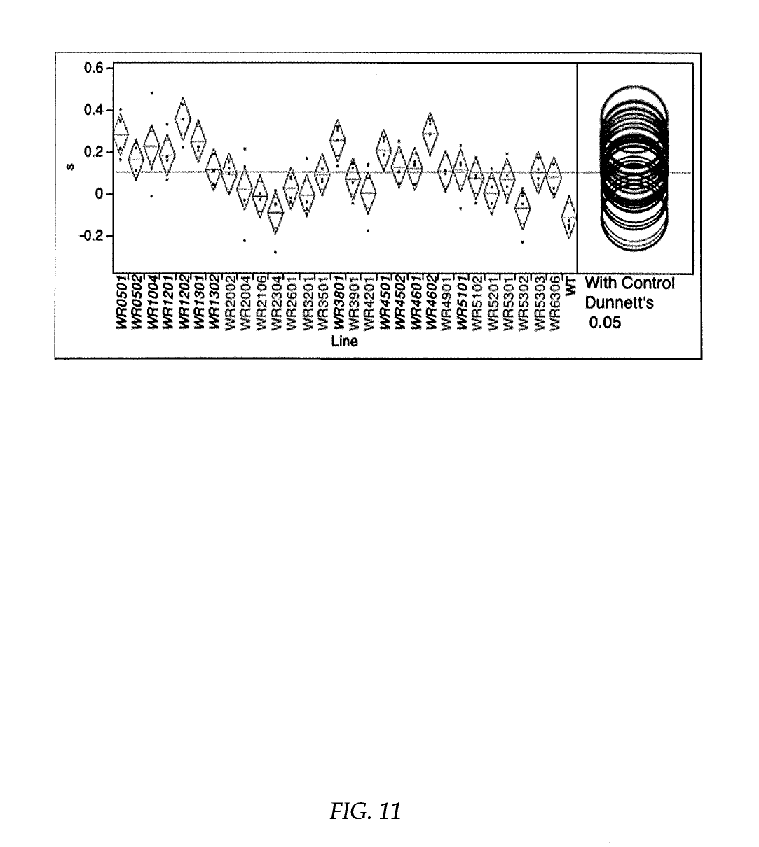

Lines were competed against a wild type analog in turbidostats. Variants were not recovered from the initial screening experiment were advanced directly into the cloning and regeneration steps. The calculated s values for approximately two weeks of growth competition for some of the lines are shown in the FIG. 11 and Table 4 below. It should be noted that in this experiment the wild type strain has a negative s value when competed against the wild type analog, and therefore all comparisons must be made to the wild type selection coefficient rather than the arbitrary s value of zero. 13 original lines had an average s value significantly greater than wild type (ANOVA with Dunnett's post test, p<0.05) versus the wild type analog. These are indicated by black text in the x-axis legend of the chart below. The remaining 16 failed to demonstrate a significantly increased competitive advantage over wild type by the Dunnett's test, though all have a mean s value higher than the mean value for wild type.

TABLE 4

| Level | p-Value | Mean | Std Dev |
|---|---|---|---|
| WR1202 | <.0001* | 0.3554 | 0.0966 |
| WR4602 | <.0001* | 0.2873 | 0.0739 |
| WR0501 | <.0001* | 0.2815 | 0.1111 |
| WR3801 | <.0001* | 0.2519 | 0.0847 |
| WR1301 | <.0001* | 0.2480 | 0.0658 |
| WR1004 | 0.0003* | 0.2258 | 0.2176 |
| WR4501 | 0.0007* | 0.2056 | 0.0701 |
| WR1201 | 0.0023* | 0.1819 | 0.1102 |
| WR0502 | 0.0060* | 0.1611 | 0.0831 |
| WR4502 | 0.0260* | 0.1263 | 0.0872 |
| WR4601 | 0.0357* | 0.1181 | 0.0507 |
| WR1302 | 0.0433* | 0.1129 | 0.0603 |
| WR5101 | 0.0460* | 0.1113 | 0.1282 |
| WR4901 | 0.0553 | 0.1062 | 0.0717 |
| WR5303 | 0.0675 | 0.1006 | 0.0563 |
| WR2002 | 0.0896 | 0.0923 | 0.0638 |
| WR3501 | 0.0980 | 0.0896 | 0.0318 |
| WR6306 | 0.1450 | 0.0773 | 0.0752 |
| WR5102 | 0.1636 | 0.0733 | 0.0898 |
| WR3901 | 0.1846 | 0.0692 | 0.0869 |
| WR5301 | 0.1927 | 0.0677 | 0.0979 |
| WR2601 | 0.5695 | 0.0226 | 0.0617 |
| WR2004 | 0.5947 | 0.0203 | 0.1933 |
| WR5201 | 0.7820 | 0.0027 | 0.0942 |
| WR4201 | 0.7868 | 0.0022 | 0.1607 |
| WR3201 | 0.8999 | −0.0109 | 0.1212 |
| WR2106 | 0.9271 | −0.0152 | 0.0815 |
| WR5302 | 1.0000 | −0.0704 | 0.1106 |
| WR2304 | 1.0000 | −0.0934 | 0.1295 |
| WT | 1.0000 | −0.1137 | 0.0694 |

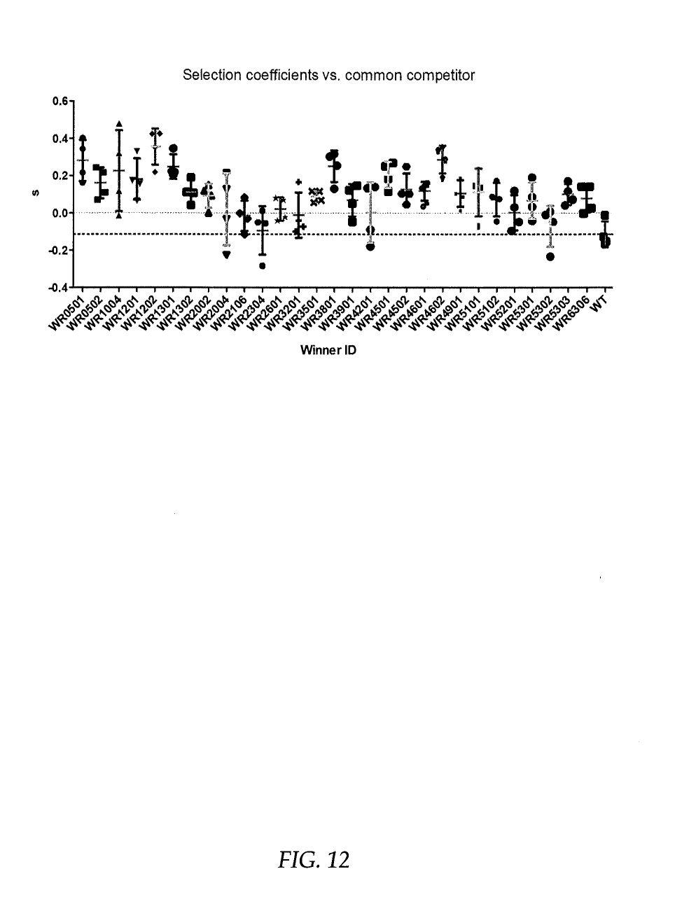

Another representation of the selection coefficients for the 29 original lines and wild type is shown in FIG. 12 with the mean of the wild type samples indicated with a dark dotted line for reference.

In some cases the number of kanamycin resistant colonies in the sorted samples was higher than the number of colonies on TAP plates containing no antibiotic. In this situation accurate s values were unable to be determined (as the natural log of a negative number gives an error). It is likely in these cases that the population in the turbidostat consists almost entirely of the wild type analog line and our sample size is not large enough to detect the relatively small number of Rubisco variant cells present. To allow calculation of s in cases where the number of colonies was higher on the kanamycin plates, the Rubisco variant colony number was manually adjusted to 1. This allows a calculation of s that will represent the minimum negative correct value.

### Regenerated Line Competitions

54 of the 103 selected variants lines were successfully regenerated. The remaining 49 variant plasmids did not yield viable colonies after two separate transformation efforts, each effort consisting of three biolistic shots. All regenerated lines were entered into competitions with the wild type analog in turbidostats as previously described. These lines were divided into three sets, shown below. In no case did any regenerated line show a statistically higher s value than wild type (ANOVA with Dunnett's post test, p<0.05). However, several anomalies in the setup and results suggested that these experimental results should be interpreted cautiously. This is described further below.

The variance in the replicates from the regenerated competition was higher than that for the original line competitions. One contributor to this was that triplicates were used for the regenerated lines while quadruplicates were used for original lines. Another possible cause for the low confidence results is that the screening setups varied. Due to the need to drive the regenerated lines to homoplasmicity, each strain was placed in a turbidostat for several days immediately prior to the competition setup. Original lines were already homoplasmic from the screening process and were taken directly from flasks into the competitions.

In a subset of the data the sorted clones that are the readout for the competition experiments did not grow as expected. 384 events were sorted onto TAP (permissive) media for each baseline (BL) and replicate (A-D or A-C). Normally, the vast majority (though typically not 100%) of the events are viable cells and produce a colony. In some cases a much smaller number of events produced viable cells. This suggested either that not all events were cells and/or that the sorted cells were differentially growing on the TAP plates. Either of these can skew the final result. Additionally, when a subset of colonies from low yielding Round 1 TAP plates were grown on TAP+Kanamycin (selective media), 100% of the colonies grew. This was not the expected result as the colonies sorted directly onto TAP+Kan indicated that some subset of cells were rbcL variants and therefore should not grow on the selective media.

When wild type is competed against the wild type analog used in these experiments, it typically gives a negative selection coefficient in the −0.1 to −0.2 range. In the round 2 set of regenerated lines, wild type gave a selection coefficient of +0.21 relative to the common competitor. Because of this, the entire dataset for these variants was not used.

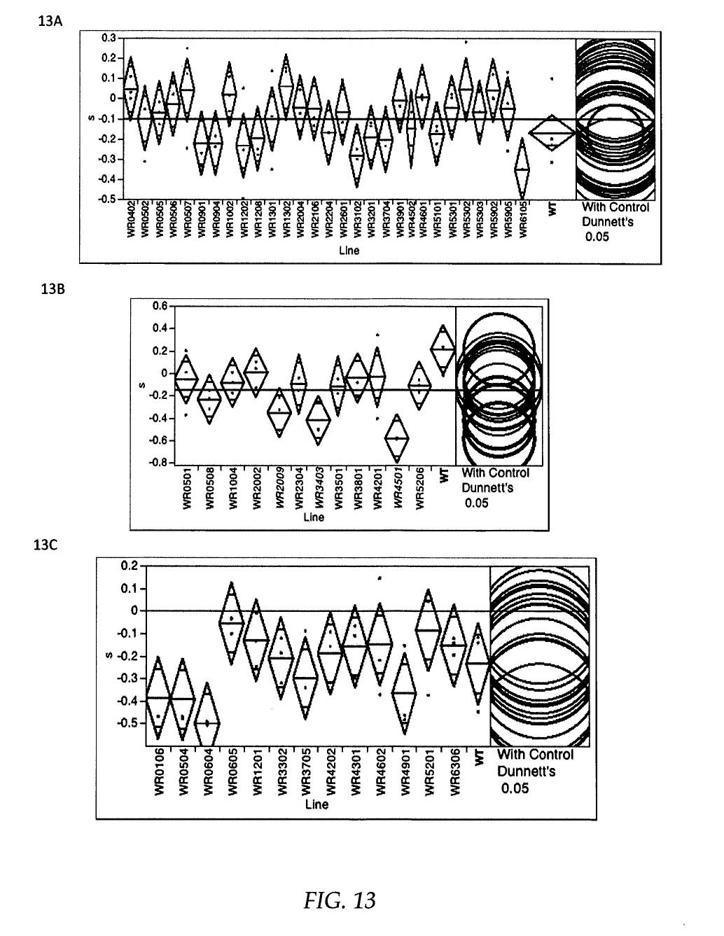

The results of the competition with the regenerated lines are shown in FIGS. 13A,B and C and Table 5, 6 and 7 which show the data points and means along with the ANOVA/Dunnett's post test for each of the three sets of regenerated lines, respectively.

TABLE 5

| Level | p-Value | Mean | Std Dev |
|---|---|---|---|
| WR1302 | 0.2731 | 0.0600 | 0.1426 |
| WR0402 | 0.3689 | 0.0461 | 0.0562 |
| WR0507 | 0.4051 | 0.0415 | 0.2572 |
| WR5302 | 0.3752 | 0.0453 | 0.2041 |
| WR5902 | 0.4062 | 0.0413 | 0.0691 |
| WR1002 | 0.5855 | 0.0204 | 0.0857 |
| WR4601 | 0.6992 | 0.0077 | 0.0119 |
| WR6105 | 0.6407 | −0.3533 | 0.0010 |
| WR3901 | 0.8523 | −0.0121 | 0.1124 |
| WR0506 | 0.9388 | −0.0286 | 0.1344 |
| WR2004 | 0.9808 | −0.0440 | 0.0779 |
| WR2106 | 0.9882 | −0.0492 | 0.1177 |
| WR5301 | 0.9820 | −0.0447 | 0.0989 |
| WR5905 | 0.9902 | −0.0511 | 0.1966 |
| WR3102 | 0.9932 | −0.2846 | 0.0283 |
| WR0505 | 0.9993 | −0.0720 | 0.0555 |
| WR2601 | 0.9990 | −0.0694 | 0.1434 |
| WR5303 | 0.9988 | −0.0679 | 0.1027 |
| WR1301 | 1.0000 | −0.1017 | 0.2442 |
| WR0502 | 1.0000 | −0.1033 | 0.1912 |
| WR1202 | 1.0000 | −0.2341 | 0.2745 |
| WR0901 | 1.0000 | −0.2231 | 0.1363 |
| WR0904 | 1.0000 | −0.2217 | 0.0298 |
| WR3704 | 1.0000 | −0.2074 | 0.1530 |
| WR1208 | 1.0000 | −0.1989 | 0.1152 |
| WR3201 | 1.0000 | −0.1937 | 0.1125 |
| WR2204 | 1.0000 | −0.1692 | 0.0020 |
| WR5101 | 1.0000 | −0.1747 | 0.0452 |
| WR4502 | 1.0000 | −0.1510 | 0.1170 |
| WT | 1.0000 | −0.1695 | 0.1488 |

TABLE 6

| Level | p-Value | Mean | Std Dev |
|---|---|---|---|
| WR4201 | 0.7003 | −0.0317 | 0.5260 |
| WR2002 | 0.7519 | 0.0054 | 0.1196 |
| WR2304 | 0.4345 | −0.0972 | 0.0777 |
| WR3801 | 0.5372 | −0.0410 | 0.1900 |
| WR0501 | 0.4772 | −0.0545 | 0.2915 |
| WR3501 | 0.3725 | −0.1147 | 0.0929 |
| WR1004 | 0.3583 | −0.0840 | 0.0911 |
| WR5206 | 0.2666 | −0.1114 | 0.0550 |
| WR0508 | 0.0562 | −0.2324 | 0.0820 |
| WR2009 | 0.0094* | −0.3514 | 0.1446 |
| WR3403 | 0.0032* | −0.4200 | 0.1429 |
| WR4501 | 0.0002* | −0.5824 | 0.0033 |
| WT | 1.0000 | 0.2123 | 0.2177 |

TABLE 7

| Level | p-Value | Mean | Std Dev |
|---|---|---|---|
| WR3302 | 1.0000 | −0.2107 | 0.1013 |
| WR4202 | 1.0000 | −0.1897 | 0.1159 |
| WR3705 | 0.9999 | −0.2986 | 0.1921 |
| WR4301 | 0.9991 | −0.1586 | 0.1219 |
| WR6306 | 0.9984 | −0.1537 | 0.0382 |
| WR4602 | 0.9974 | −0.1492 | 0.2651 |
| WR1201 | 0.9873 | −0.1311 | 0.1175 |
| WR4901 | 0.9353 | −0.3674 | 0.1844 |
| WR5201 | 0.8746 | −0.0856 | 0.2508 |
| WR0106 | 0.8629 | −0.3874 | 0.1420 |
| WR0504 | 0.8332 | −0.3940 | 0.1416 |
| WR0605 | 0.7315 | −0.0565 | 0.0396 |
| WR0604 | 0.2875 | −0.5006 | 0.0078 |
| WT | 1.0000 | −0.2351 | 0.1835 |

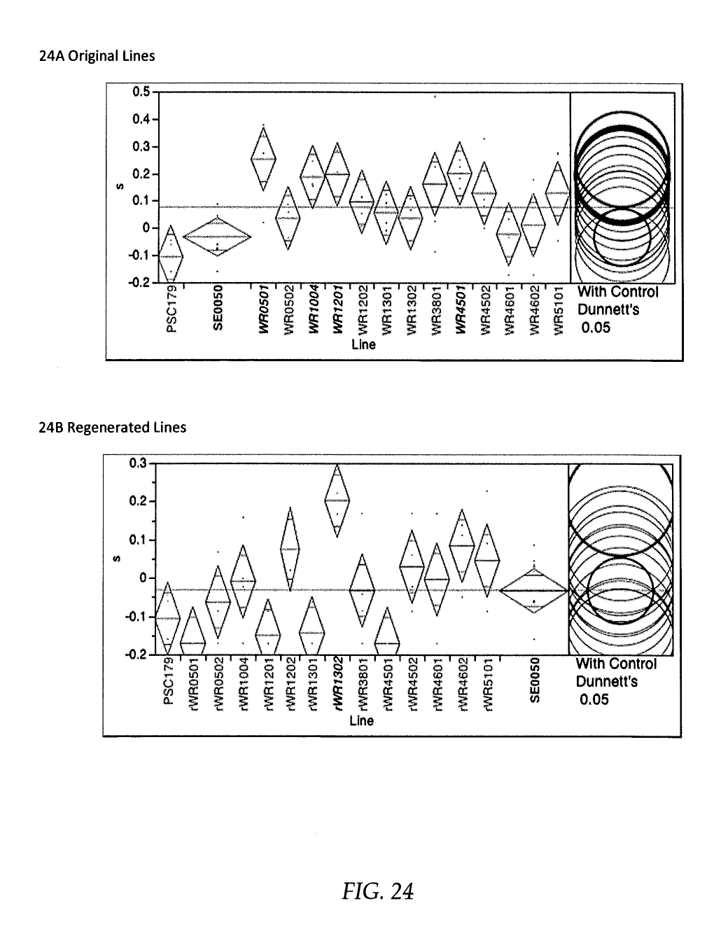

In another set of experiments, 13 original lines with significantly higher selection coefficients than wild type, along with the regenerated versions of those lines, were again competed 1:1 against a wild type analog strain in quadruplicate turbidostats. A knockout line complemented with the wild type Rubisco gene was also included in the experiment as a secondary control. The calculated s values for two weeks of growth competition are shown in FIGS. 24A and 24B and Table 8 below. It should be noted that in this experiment the wild type strain had a negative s value when competed against the wild type analog, and therefore all comparisons must be made to the wild type selection coefficient rather than the arbitrary s value of zero. 4 original lines and 1 regenerated line had average s values significantly greater than wild type (ANOVA with Dunnett's post test, p<0.05) versus the wild type analog. These are indicated by black text in the x-axis legend of in FIG. 24. The remaining 9 original lines failed to demonstrate a significantly increased competitive advantage over wild type by the Dunnett's test, though all have mean s values higher than the mean value for wild type. 7 regenerated lines had average s values above wild type, including 3 lines (WR1004, WR1202, WR5101) that were not previously validated with the initial competition experiments.

TABLE 8
Means Comparisons: Comparisons with a control using Dunnett's Method
Control Group = SE0050 (wild type)

| Level | p-Value | Mean | Std Dev |
|---|---|---|---|
| WR0501 | 0.0014 | 0.25207 | 0.1610 |
| WR0502 | 0.9858 | 0.03551 | 0.0524 |
| WR1004 | 0.0262 | 0.18649 | 0.0413 |
| WR1201 | 0.0172 | 0.19656 | 0.0773 |
| WR1202 | 0.5071 | 0.09588 | 0.0874 |
| WR1301 | 0.9053 | 0.05596 | 0.0616 |
| WR1302 | 0.9846 | 0.03616 | 0.0741 |
| WR3801 | 0.0702 | 0.16131 | 0.2503 |
| WR4501 | 0.0143 | 0.20081 | 0.0465 |
| WR4502 | 0.2164 | 0.12822 | 0.1412 |
| WR4601 | 1.0000 | −0.02198 | 0.1110 |
| WR4602 | 0.9998 | 0.01211 | 0.1669 |
| WR5101 | 0.2129 | 0.12876 | 0.1700 |
| rWR0501 | 0.1767 | −0.17128 | 0.0000 |
| rWR0502 | 1.0000 | −0.06359 | 0.0990 |
| rWR1004 | 1.0000 | −0.00945 | 0.1346 |
| rWR1201 | 0.3693 | −0.15036 | 0.0418 |
| rWR1202 | 0.6381 | 0.07448 | 0.0944 |
| rWR1301 | 0.4411 | −0.14444 | 0.0537 |
| rWR1302 | 0.0014 | 0.20166 | 0.0647 |
| rWR3801 | 1.0000 | −0.03282 | 0.1443 |
| rWR4501 | 0.1767 | −0.17128 | 0.0000 |
| rWR4502 | 0.9676 | 0.02946 | 0.1104 |
| rWR4601 | 1.0000 | −0.00364 | 0.1539 |
| rWR4602 | 0.3641 | 0.08437 | 0.0902 |
| rWR5101 | 0.8549 | 0.04571 | 0.1433 |
| PSC179 | 0.9763 | −0.10642 | 0.0620 |
| SE0050 | 1.0000 | −0.03322 | 0.1058 |

En Masse Competition.

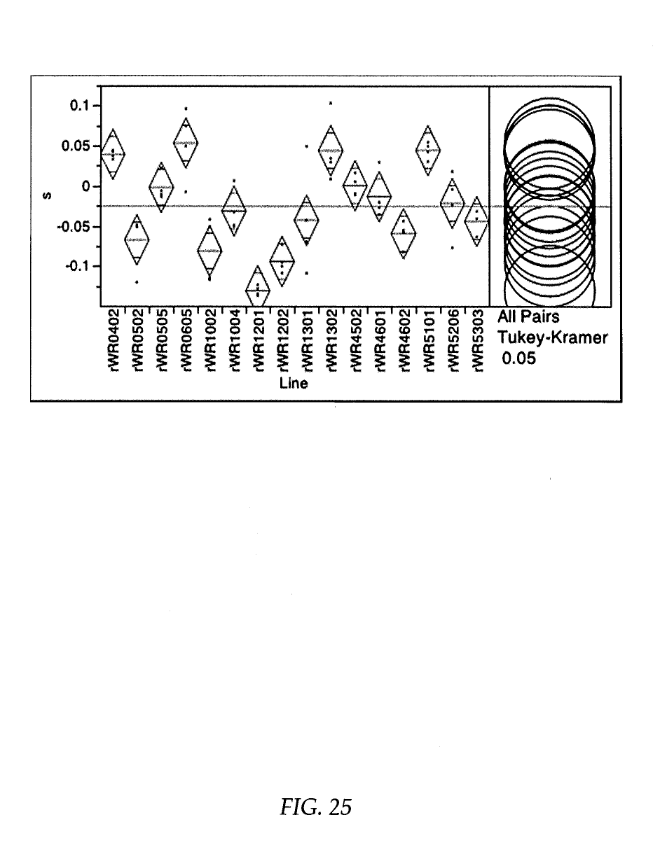

16 validated variants were competed en masse in turbidostats for three weeks. The calculated s values are shown in FIG. 25 and Table 9. An ANOVA with Tukey-Kramer HSD test was completed on the selection coefficient data. Levels not connected by the same letter in the table are significantly different. Mutations that were selected to be held constant in the next turn are marked with an asterisk.

TABLE 9

| Level | | | | | | | s_{avg} |
|---|---|---|---|---|---|---|---|
| rWR0605 | A | | | | | | 0.053169 |
| rWR5101 | A | B | | | | | 0.044251 |
| rWR1302* | A | B | | | | | 0.043971 |
| rWR0402 | A | B | | | | | 0.039126 |
| rWR4502* | A | B | C | | | | 0.000296 |
| rWR0505 | A | B | C | D | | | −0.00202 |
| rWR4601 | A | B | C | D | | | −0.01335 |
| rWR5206 | A | B | C | D | E | | −0.02173 |
| rWR1004 | | B | C | D | E | | −0.03153 |
| rWR1301 | | | C | D | E | | −0.04294 |
| rWR5303 | | | C | D | E | | −0.04434 |
| rWR4602* | | | C | D | E | F | −0.05962 |
| rWR0502 | | | C | D | E | F | −0.06756 |
| rWR1002 | | | | D | E | F | −0.08138 |
| rWR1202 | | | | | E | F | −0.09439 |
| rWR1201 | | | | | | F | −0.13076 |

43 regenerated lines of variants without original lines significantly better than wild type were competed 1:1 against the wild type analog strain in quadruplicate turbidostats. A knockout line complemented with the wild type Rubisco gene was also included in the experiment as a secondary control. The calculated s values for approximately two weeks of growth competition are shown in FIG. 26 and Table 10. 3 lines have an average s value significantly greater than wild type (ANOVA with Dunnett's post test, p<0.05) versus the wild type analog. These are indicated by black text in the x-axis legend of the FIG. 26. One additional line, while not significantly above wild type by Dunnett's, has all replicate s values above the wild type average. 40 regenerated lines failed to be validated by the criteria of all replicates having s values greater than the average wild type value. Of the 3 lines with all replicate s values above wild type, 2 (WR5206 and WR5303) have not been previously validated.

TABLE 10
Means Comparisons: Comparisons with a control using Dunnett's Method
Control Group = SE0050 (wild type)

| Level | p-Value | Mean | Std Dev |
|---|---|---|---|
| rWR0106 | 1.0000 | −0.1614 | 0.0000 |
| rWR0402 | 1.0000 | −0.1375 | 0.0000 |
| rWR0504 | 1.0000 | −0.1996 | 0.0000 |
| rWR0505 | 0.9626 | −0.0934 | 0.1161 |
| rWR0506 | 1.0000 | −0.1691 | 0.0934 |
| rWR0507 | 0.9802 | −0.0981 | 0.1229 |
| rWR0508 | 1.0000 | −0.1548 | 0.1081 |
| rWR0604 | 1.0000 | −0.1859 | 0.0000 |
| rWR0605 | 0.0125 | 0.0444 | 0.0734 |
| rWR0901 | 0.9976 | −0.1092 | 0.0585 |
| rWR0904 | 0.9989 | −0.1123 | 0.1068 |
| rWR1002 | 1.0000 | −0.1548 | 0.0000 |
| rWR1208 | 0.9922 | −0.1036 | 0.1484 |
| rWR2002 | 0.6306 | −0.0609 | 0.1019 |
| rWR2004 | 1.0000 | −0.1323 | 0.1670 |
| rWR2009 | 1.0000 | −0.1761 | 0.0000 |
| rWR2106 | 0.4281 | −0.0447 | 0.1247 |
| rWR2204 | 1.0000 | −0.1800 | 0.0000 |
| rWR2304 | 0.9981 | −0.2495 | 0.0000 |
| rWR2601 | 0.9293 | −0.0877 | 0.1727 |
| rWR2704 | 0.9920 | −0.2562 | 0.0000 |
| rWR3102 | 1.0000 | −0.1802 | 0.0753 |
| rWR3201 | 0.1922 | −0.0186 | 0.1723 |
| rWR3302 | 1.0000 | −0.1489 | 0.0000 |
| rWR3403 | 1.0000 | −0.2198 | 0.0000 |
| rWR3501 | 0.2981 | −0.0321 | 0.1719 |
| rWR3704 | 1.0000 | −0.2277 | 0.0000 |
| rWR3705 | 1.0000 | −0.1606 | 0.0000 |
| rWR3901 | 0.2557 | −0.3325 | 0.0000 |
| rWR4201 | 1.0000 | −0.1912 | 0.0000 |
| rWR4202 | 1.0000 | −0.1488 | 0.1177 |
| rWR4301 | 1.0000 | −0.1358 | 0.0000 |
| rWR4302 | 0.9509 | −0.2686 | 0.0000 |
| rWR4901 | 1.0000 | −0.1367 | 0.0000 |
| rWR5102 | 0.9997 | −0.2430 | 0.0075 |
| rWR5201 | 0.2990 | −0.0322 | 0.1194 |
| rWR5206 | 0.0766 | 0.0055 | 0.0501 |
| rWR5301 | 0.2291 | −0.0238 | 0.0869 |
| rWR5302 | 0.0242 | 0.0312 | 0.1617 |
| rWR5303 | 0.0001 | 0.1220 | 0.0973 |
| rWR5905 | 0.5489 | −0.0546 | 0.1095 |
| rWR6105 | 1.0000 | −0.2290 | 0.0000 |
| rWR6306 | 1.0000 | −0.1789 | 0.0770 |
| psc179 | 0.9964 | −0.2426 | 0.0000 |
| SE0050 | 1.0000 | −0.1799 | 0.1189 |

### Structural Information for Variants

In general these variants are clustered together on the protein structure and can be divided into four groups based on their locations: Loop 25-35, β-sheet 83-89, α-helix 310-321, and Other. It is interesting to note that 8 of the selected variants are present in one of two structural motifs. A β-strand from amino acid 83-89 contains four of these variants (R83Q, R83H, I87Y, E88S) while an α-helix from position 310-321 also contains four (R312K, A315S, A317G, M320L). Lending additional confidence to the validation of these lines, 6 of the 7 validated variants are within these structural elements (3 each of these sets of four).

### Growth and Biochemical Characteristics

Microtiter Plate Growth Assay.

Original lines were tested in microtiter plate growth assays using MASM, TAP and HSM media. All 29 original lines were tested in MASM and TAP (2 separate setup rounds were performed); the 13 original lines with significantly greater s versus a common competitor in turbidostats were also tested in HSM (1 setup round). All plates were set up in duplicate and incubated in 5% CO₂ and 0.04% CO₂ (air). In MASM and TAP media, full growth curves were obtained, while only linear growth rates were obtained in HSM. Growth rates were not obtained for cultures grown in HSM media without CO₂ enrichment, as no lines showed a significant increase in OD₇₅₀ over 66 hours. Logistic growth curves were fit to MASM and TAP growth data, and the calculated carrying capacities (K), maximum growth rates (r), and peak theoretical productivities (Kr/4) were compared to wild type replicates in the same plate. For HSM, only the growth rates were compared to the wild type growth rates. All comparisons were carried out using ANOVA with Dunnett's post test, p<0.05.

No line in MASM, TAP, or HSM showed a significant increase in any growth parameter in 5% CO₂, however 9 lines did show significant increases in peak theoretical productivity (Kr/4) and 11 lines showed significant increases in maximum growth rate (r) in MASM and/or TAP in 0.04% CO₂ in at least one round.

6 of 7 original lines that were validated by regeneration were different for at least one parameter, indicated by * in the summary table below. In both rounds 6 replicates were set up for each condition and randomly distributed across two plates. In the Table 11, below, + denotes a significant increase from wild type in 1 plate, ++ in 2 plates, and +++ in 3 plates (spanning two rounds).

TABLE 11

| | |
|---|---|
| MASM | TAP |

| Line | Kr/4 | r | Kr/4 | r |
|---|---|---|---|---|
| WR0502* | ++ | + | + | ++ |
| WR1301* | ++ | + | + | + |
| WR1004 | ++ | + | | + |
| WR2601 | + | | + | + |
| WR1201* | | | + | + |
| WR4201 | + | + | | |
| WR5201 | + | | + | |
| WR4601* | | | | +++ |
| WR4501 | ++ | | | |
| WR4502* | ++ | | | |
| WR3901 | | | | + |
| WR4602* | | + | | |
| WR5301 | | + | | |
| WR6306 | | + | | |

Column Growth Assay.

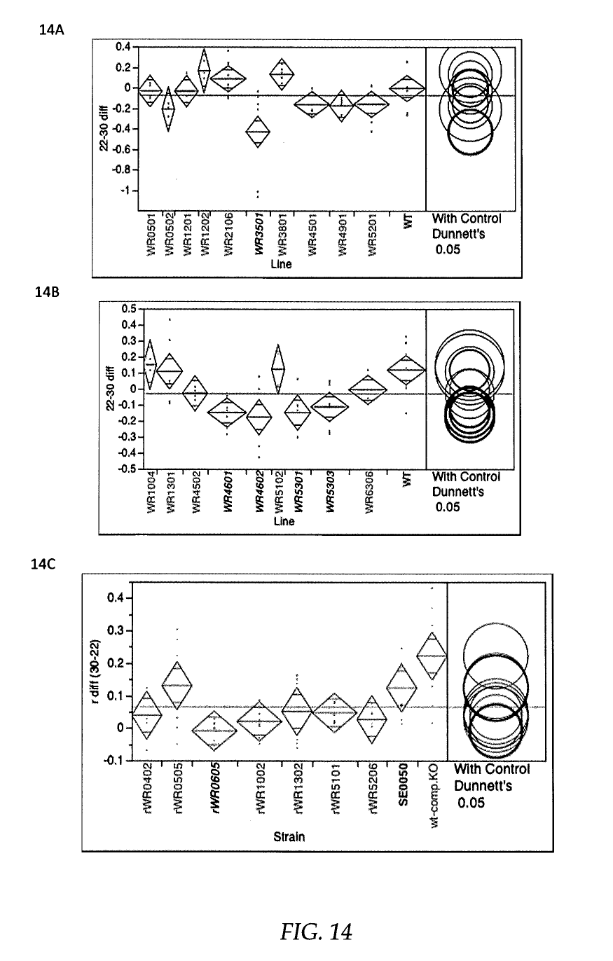

19 original lines were assayed for growth rate in column photobioreactors, including the 13 lines with significantly higher s than wild type against a common competitor in turbidostats. Two rounds of columns were set up, each including a wild type control. All samples were run at 22° C. and 30° C. in triplicate. No lines showed a significantly higher growth rate than wild type at either temperature, however 5 lines showed a significantly lower differential in growth rate between 22° C. and 30° C. as compared to wild type (ANOVA with Dunnett's post test, p<0.05), suggesting increased heat tolerance in those lines. Two of the significant lines (WR4601, WR4602) were validated by regenerated line turbidostat competitions. FIGS. 14A and B the calculated growth rate differentials for each setup. Lines with significantly lower differentials are indicated by black text in the x-axis legend.

In an additional experiment, 7 validated variants, along with a true wild type and a wild type-complemented knockout line, were assayed for growth rate in column photobioreactors. All samples were run at 22° C. and 30° C. in triplicate. No lines showed a significantly higher growth rate than wild type at either temperature; however 1 line (rWR0605) showed a significantly lower differential in growth rate between 22° C. and 30° C. as compared to wild type (ANOVA with Dunnett's post test, p<0.05), suggesting increased heat tolerance. FIG. 14C shows the calculated growth rate differentials. The line with a significantly lower differential is indicated by black text in the x-axis legend.

Fluorescence Induction and Relaxation (FIRe) Assay.

A subset of original lines including 13 with significantly greater s than wild type against a common competitor in turbidostats were assayed for multiple photosynthetic parameters using a Fluorescence Induction and Relaxation (FIRe) fluorometer. Four key parameters were analyzed: The electron transport rate at each PSII reaction center under light saturating conditions (ETRₘₐₓ); the rate of change of ETR during the transition from PAR 0 to actinic light (a); the functional absorption cross section of PSII at PAR 0 (σ_{PSII}); and the maximum quantum yield of photochemistry in PSII at PAR 0 (Fᵥ/Fₘ).

In order to characterize differences between Rubisco variants and wild type, an ANOVA with Tukey-Kramer HSD test was completed on each of the four FIRe datasets. This test is a single-step multiple comparison procedure and statistical test to find which means are significantly different from one another. The test compares the means of every sample to the means of every other sample; that is, it applies simultaneously to the set of all pairwise comparisons and identifies where the difference between two means is greater than the standard error would be expected to allow. Variation in growth conditions such as optical density can affect parameters measured by the FIRe instrument, and while culture conditions were carefully controlled by sampling from turbidostats, significant variability between biological replicates was occasionally observed. Therefore, variants were only considered to be significantly different from wild type if they were different from every wild type sample measured throughout the experiment for a given parameter. In the tables below, levels not connected by the same letter are significantly different.

TABLE 12
ETRₘₐₓ Data

| Level | | | | | | | Mean ETRₘₐₓ |
|---|---|---|---|---|---|---|---|
| WR3801 | A | | | | | | 162.97899 |
| WT11 | A | B | | | | | 156.85802 |
| WT10 | A | B | C | | | | 144.78856 |
| WR1301 | A | B | C | | | | 142.54087 |
| WT4 | A | B | C | | | | 141.59713 |
| WR0501 | A | B | C | D | | | 140.89596 |
| WR0502 | A | B | C | D | E | | 137.94656 |
| WT7 | A | B | C | D | E | | 136.27205 |
| WT5 | A | B | C | D | E | F | 135.25234 |
| WT3 | A | B | C | D | E | F | 132.54429 |
| WR1301-2 | A | B | C | D | E | F | 132.36616 |
| WR1202 | | B | C | D | E | F | 130.22673 |
| WT2 | | B | C | D | E | F | 128.60446 |
| WR1202-2 | | B | C | D | E | F | 127.94441 |
| WR1201 | | B | C | D | E | F | 126.54893 |
| WR1004 | | B | C | D | E | F | 125.73853 |
| WR2002 | | B | C | D | E | F | 125.13823 |
| WR4502 | | B | C | D | E | F | 124.66389 |
| WT1 | | | C | D | E | F | 124.33871 |
| WR4602 | | | C | D | E | F | 122.25236 |
| WR3201 | | | C | D | E | F | 122.07547 |
| WT6 | | | C | D | E | F | 121.32784 |
| WR5101 | | | C | D | E | F | 121.04656 |
| WT9 | | | C | D | E | F | 120.99460 |
| WR2304 | | | C | D | E | F | 120.87171 |
| WT8 | | | C | D | E | F | 119.95892 |
| WR4501 | | | C | D | E | F | 117.71173 |
| WR4601-2 | | | C | D | E | F | 115.56473 |
| WR4502-2 | | | C | D | E | F | 115.19107 |
| WR1302 | | | | D | E | F | 109.21182 |
| WR3501 | | | | | E | F | 107.12851 |
| WR4601 | | | | | | F | 103.37754 |

TABLE 13
Alpha (α) Data

| Level | | | | | | | | | | Mean α |
|---|---|---|---|---|---|---|---|---|---|---|
| WT2 | A | | | | | | | | | 1.2741273 |
| WT1 | A | B | | | | | | | | 1.2422882 |
| WR1202 | A | B | | | | | | | | 1.2396399 |
| WT9 | A | B | | | | | | | | 1.2376798 |
| WT8 | A | B | | | | | | | | 1.2279001 |
| WT6 | A | B | C | | | | | | | 1.2203373 |
| WR4502-2 | A | B | C | D | E | | | | | 1.2157143 |
| WT10 | A | B | C | D | | | | | | 1.2152914 |
| WT4 | A | B | C | D | E | F | | | | 1.2098003 |
| WT7 | A | B | C | D | E | F | | | | 1.2088857 |
| WR4502 | A | B | C | D | E | F | | | | 1.2072855 |
| WT5 | A | B | C | D | E | F | | | | 1.1920990 |
| WR1004 | A | B | C | D | E | F | | | | 1.1892039 |
| WR2002 | A | B | C | D | E | F | | | | 1.1823302 |
| WR0502 | A | B | C | D | E | F | | | | 1.1782374 |
| WR1201 | A | B | C | D | E | F | | | | 1.1692448 |
| WR4501 | | B | C | D | E | F | | | | 1.1530441 |
| WT3 | | B | C | D | E | F | | | | 1.1491220 |
| WR1302 | | B | C | D | E | F | | | | 1.1469121 |
| WR3501 | | B | C | D | E | F | | | | 1.1434294 |
| WR3801 | | B | C | D | E | F | | | | 1.1425063 |
| WR0501 | | B | C | D | E | F | G | | | 1.1243625 |
| WT11 | | | C | D | E | F | G | H | | 1.1052981 |
| WR3201 | | | | D | E | F | G | H | | 1.1006857 |
| WR5101 | | | | | E | F | G | H | | 1.0979012 |
| WR1202-2 | | | | | | F | G | H | I | 1.0908164 |
| WR4601-2 | | | | | | | G | H | I | 1.0172605 |
| WR2304 | | | | | | | G | H | I | 1.0164483 |
| WR4602 | | | | | | | G | H | I | 1.0104741 |
| WR1301-2 | | | | | | | | H | I | 0.9987615 |
| WR1301 | | | | | | | | H | I | 0.9882726 |
| WR4601 | | | | | | | | | I | 0.9769647 |

TABLE 14
Sigma (σ_{PSII}) Data

| Level | | | | | | | | | | Mean σ_{PSII} |
|---|---|---|---|---|---|---|---|---|---|---|
| WR0502 | A | | | | | | | | | 244.88333 |
| WR4601-2 | A | B | | | | | | | | 242.83333 |
| WR4601 | A | B | C | | | | | | | 232.06667 |
| WR0501 | | B | C | D | | | | | | 228.81667 |
| WR1201 | | B | C | D | | | | | | 228.18333 |
| WR3801 | | B | C | D | | | | | | 228.01667 |
| WR4502-2 | | B | C | D | | | | | | 227.23333 |
| WR1004 | | | C | D | E | | | | | 224.50000 |
| WR4501 | | | C | D | E | | | | | 224.31667 |
| WT7 | | | C | D | E | | | | | 224.23333 |
| WR3201 | | | C | D | E | F | | | | 222.60000 |
| WR1202 | | | C | D | E | F | G | | | 220.96667 |
| WT8 | | | C | D | E | F | G | | | 219.85000 |
| WT6 | | | C | D | E | F | G | | | 219.70000 |
| WT9 | | | C | D | E | F | G | H | | 218.83333 |
| WR5101 | | | C | D | E | F | G | H | | 218.10000 |
| WT5 | | | C | D | E | F | G | H | | 217.73333 |
| WR3501 | | | | D | E | F | G | H | I | 215.45000 |
| WR4502 | | | | D | E | F | G | H | I | 214.30000 |
| WT10 | | | | D | E | F | G | H | I | 214.14286 |
| WR2002 | | | | D | E | F | G | H | I | 213.85000 |
| WT2 | | | | D | E | F | G | H | I | 213.51667 |
| WT1 | | | | | E | F | G | H | I | 210.58333 |
| WT4 | | | | | E | F | G | H | I | 210.31667 |
| WR1301-2 | | | | | E | F | G | H | I | 209.58333 |
| WR1301 | | | | | E | F | G | H | I | 209.08333 |
| WR2304 | | | | | E | F | G | H | I | 208.56667 |
| WR1302 | | | | | | F | G | H | I | 206.95000 |
| WT3 | | | | | | | G | H | I | 205.85000 |
| WR1202-2 | | | | | | | | H | I | 203.55000 |
| WR4602 | | | | | | | | H | I | 202.98333 |
| WT11 | | | | | | | | | I | 200.26667 |

TABLE 15
Fᵥ/Fₘ Data

| Level | | | | | | | | | | Mean Fᵥ/Fₘ |
|---|---|---|---|---|---|---|---|---|---|---|
| WT6 | A | | | | | | | | | 0.60933333 |
| WT4 | A | | | | | | | | | 0.60916667 |
| WT1 | A | | | | | | | | | 0.60883333 |
| WT9 | A | | | | | | | | | 0.60666667 |
| WT11 | A | | | | | | | | | 0.60650000 |
| WT8 | A | B | | | | | | | | 0.60366667 |
| WT2 | A | B | C | | | | | | | 0.60166667 |
| WT3 | A | B | C | | | | | | | 0.60133333 |
| WT10 | A | B | C | D | | | | | | 0.59528571 |
| WR1202-2 | A | B | C | D | | | | | | 0.59500000 |
| WR2002 | A | B | C | D | E | | | | | 0.59283333 |
| WR4502 | A | B | C | D | E | F | | | | 0.58483333 |
| WR1202 | A | B | C | D | E | F | | | | 0.58416667 |
| WT7 | A | B | C | D | E | F | | | | 0.58333333 |
| WR5101 | A | B | C | D | E | F | | | | 0.58183333 |
| WR4602 | A | B | C | D | E | F | | | | 0.58016667 |
| WT5 | A | B | C | D | E | F | | | | 0.57416667 |
| WR1302 | A | B | C | D | E | F | | | | 0.57333333 |
| WR4502-2 | A | B | C | D | E | F | | | | 0.57100000 |
| WR3501 | A | B | C | D | E | F | G | | | 0.56866667 |
| WR4501 | | B | C | D | E | F | G | | | 0.56116667 |
| WR1301-2 | | | C | D | E | F | G | | | 0.55900000 |
| WR1201 | | | C | D | E | F | G | | | 0.55800000 |
| WR3801 | | | | D | E | F | G | | | 0.55516667 |
| WR1301 | | | | D | E | F | G | | | 0.55266667 |
| WR2304 | | | | | E | F | G | | | 0.54966667 |
| WR3201 | | | | | | F | G | H | | 0.54316667 |
| WR1004 | | | | | | | G | H | I | 0.52533333 |
| WR0501 | | | | | | | | H | I | 0.50400000 |
| WR0502 | | | | | | | | H | I | 0.50333333 |
| WR4601 | | | | | | | | | I | 0.49566667 |
| WR4601-2 | | | | | | | | | I | 0.48733333 |

In some cases strains that appeared as outliers for a particular parameter were re-run with a biological replicate (denoted with a −2 in the Tukey-Kramer tables above). 14 lines were identified that differed from wild type in at least one parameter; with 6 lines differing in two parameters and 1 line differing in three (all three parameters were different in separate biological replicates). No lines were identified that had significantly different ETRₘₐₓ than wild type. All lines with a different α were lower than wild type, all with a different σ_{PSII} were higher than wild type, and all with a different Fᵥ/Fₘ were lower than wild type. 6 of 7 original lines that were validated by regeneration were different for at least one parameter, indicated by * in the summary table below. Significant differences are denoted with a +, differences in both biological replicates with ++, and differences in one biological replicate but not the other with +/−.

TABLE 16

| Line | α (low) | σ_{PSII} (high) | Fᵥ/Fₘ (low) |
|---|---|---|---|
| WR4601* | ++ | ++ | ++ |
| WR1301* | ++ | | ++ |
| WR0501 | | + | + |
| WR0502* | | + | + |
| WR1201* | | + | + |
| WR3801 | | + | + |
| WR1004 | | | + |
| WR2304 | + | | + |
| WR3201 | | | + |
| WR3501 | | | + |
| WR4501 | | | + |
| WR4602* | + | | |
| WR1202 | +/− | | |
| WR4502* | | +/− | |

In additional experiments, 6 validated variants, along with a true wild type, a wild type-complemented knockout line, and 2 additional lines that had previously shown significant differences from WT (rWR1301, rWR4601) were assessed for photosynthetic characteristics using a Satlantic FIRe fluorometer. Four key parameters were analyzed: The electron transport rate at each PSII reaction center under light saturating conditions (ETRₘₐₓ); the rate of change of ETR during the transition from PAR 0 to actinic light (a); the functional absorption cross section of PSII at PAR 0 (σ_{PSII}); and the maximum quantum yield of photochemistry in PSII at PAR 0 (Fᵥ/Fₘ).

In order to characterize differences between Rubisco variants and wild type, an ANOVA with Tukey-Kramer HSD test was completed on each of the four FIRe datasets. Variation in growth conditions such as optical density can affect parameters measured by the FIRe instrument, and while culture conditions were carefully controlled, significant variability between biological replicates was occasionally observed. Therefore, variants were only considered to be significantly different from wild type if they were different from every wild type sample measured throughout the experiment for a given parameter. In the Tables 17, 18, 19 and 20, levels not connected by the same letter are significantly different.

TABLE 17
ETRₘₐₓ Data

| Level | | | | | | Mean ETRₘₐₓ |
|---|---|---|---|---|---|---|
| rWR1301 | A | | | | | 174.77078 |
| WT-comp. KO | | B | | | | 135.39168 |
| WT-comp. KO | | B | C | | | 123.85457 |
| rWR1002 | | B | C | D | | 114.40091 |
| rWR0505 | | B | C | D | | 112.92601 |
| rWR5303 | | B | C | D | | 110.58113 |
| rWR0402 | | | C | D | E | 103.40078 |
| rWR0605 | | | C | D | E | 98.99419 |
| rWR5206 | | | C | D | E | 98.63835 |
| rWR4601 | | | | D | E | 97.00687 |
| WT | | | | D | E | 95.19493 |
| WT | | | | | E | 82.41254 |

TABLE 18
α Data

| Level | | | | | Mean α |
|---|---|---|---|---|---|
| WT | A | | | | 1.51457 |
| rWR0402 | A | B | | | 1.483787 |
| WT | A | B | | | 1.482067 |
| rWR5303 | A | B | C | | 1.346916 |
| rWR5206 | A | B | C | | 1.342393 |
| rWR4601 | A | B | C | | 1.332534 |
| rWR1002 | | B | C | | 1.291824 |
| rWR0605 | | B | C | | 1.275719 |
| rWR0505 | | | C | | 1.264456 |
| WT-comp. KO | | | C | D | 1.200817 |
| rWR1301 | | | | D | 1.042624 |
| WT-comp. KO | | | | D | 1.031217 |

TABLE 19
Fᵥ/Fₘ Data

| Level | | | Mean Fᵥ/Fₘ |
|---|---|---|---|
| rWR5303 | A | | 0.678333 |
| rWR0605 | A | | 0.678 |
| rWR0402 | A | | 0.6755 |
| rWR1301 | A | | 0.6755 |
| rWR5206 | A | B | 0.6745 |
| WT-comp. KO | A | B | 0.6735 |
| WT | A | B | 0.673 |
| rWR4601 | A | B | 0.671833 |
| rWR0505 | A | | 0.6705 |
| WT-comp. KO | A | B | 0.665167 |
| rWR1002 | A | B | 0.665167 |
| WT | | B | 0.641833 |

TABLE 20
σ_{PSII} Data

| Level | | | | | | | Mean σ_{PSII} |
|---|---|---|---|---|---|---|---|
| WT | A | | | | | | 261.45 |
| rWR4601 | A | B | | | | | 253.7333 |
| rWR0402 | A | B | C | | | | 247.8167 |
| rWR1301 | A | B | C | | | | 247.8167 |
| rWR0605 | | B | C | D | | | 241.1833 |
| rWR5206 | | B | C | D | | | 240.7667 |
| rWR5303 | | | C | D | E | | 229.6833 |
| rWR0505 | | | | D | E | | 228.6417 |
| WT-comp. KO | | | | | E | | 220.2167 |
| WT-comp. KO | | | | | E | | 219.25 |
| rWR1002 | | | | | E | | 219.25 |
| WT | | | | | | F | 195.3 |

Western Blot.

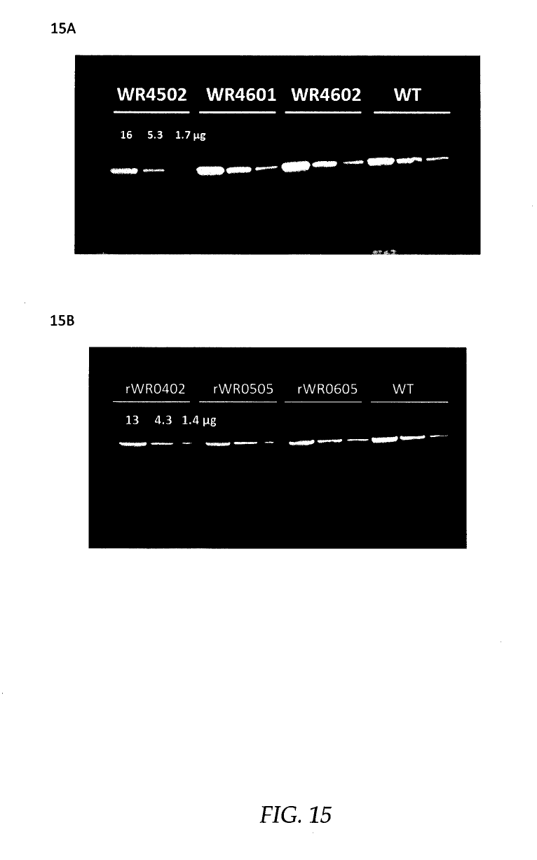

The 29 original lines were assayed for Rubisco protein levels by Western Blot. Ten protein gels were run in total, each with 2-3 original line protein samples and one wild type sample. Each sample was loaded at three dilutions (see example FIG. 15A). Spot densitometry was used to quantify the average pixel intensity (API) of each 52 kDa band after background correction, and a standard curve was generated to correlate pixel intensity to the amount of protein loaded at each point along the dilution series. APIs for diluted samples were multiplied by a dilution factor as determined by the standard curve resulting in 3 independent API measurements for each protein sample. The calculated API for each original line sample was compared to the wild type sample on its respective Western Blot. 8 samples had significantly lower mean API values than wild type and the remaining 21 samples had no significant difference from wild type based on ANOVA with Dunnett's post test (p<0.05). It is interesting to note that three of the lines with lower protein by Western are in the list of validated variants. The mean calculated intensities for each sample including the wild type samples from all Western Blots are shown below in Table 21.

TABLE 21

| Level | p-Value | Mean | Std Dev |
|---|---|---|---|
| WR4502 | 0.0013* | 11303.8 | 1992.7 |
| WR1202 | 0.0048* | 10537.7 | 4295.6 |
| WR3201 | 0.0053* | 13939.7 | 1022.1 |
| WR1201 | 0.0073* | 11156.9 | 1305.1 |
| WR3901 | 0.0073* | 15353.6 | 621.3 |
| WR1004 | 0.0242* | 10830.2 | 1967.2 |
| WR0502 | 0.0362* | 11355.9 | 3614.9 |
| WR2002 | 0.0377* | 12729.3 | 2242.6 |
| WR5301 | 0.0657 | 14216.0 | 2927.7 |
| WR4602 | 0.0735 | 19748.0 | 542.9 |
| WR2304 | 0.0783 | 9777.9 | 1966.1 |
| WR1301 | 0.1284 | 15100.3 | 818.1 |
| WR0501 | 0.2156 | 13685.4 | 2217.6 |
| WR6306 | 0.2433 | 18745.2 | 1021.3 |
| WR1302 | 0.3296 | 16354.8 | 1668.3 |
| WR2106 | 0.3522 | 12714.9 | 568.1 |
| WR4601 | 0.3556 | 18613.9 | 1039.9 |
| WR5101 | 0.4031 | 16793.7 | 3138.7 |
| WR5302 | 0.4673 | 18126.3 | 2919.0 |
| WR4201 | 0.544 | 19439.1 | 639.8 |
| WR5303 | 0.5688 | 19709.8 | 1153.5 |
| WR3801 | 0.5843 | 19294.0 | 2873.6 |
| WR4501 | 0.5929 | 19546.4 | 1859.8 |
| WR5201 | 0.7136 | 19261.4 | 3356.5 |
| WR2004 | 0.7333 | 18090.7 | 4596.4 |
| WR4901 | 0.7616 | 22193.7 | 3169.2 |
| WR3501 | 0.7763 | 19784.3 | 1950.9 |
| WR5102 | 0.9354 | 21359.6 | 3549.2 |
| WR2601 | 0.9969 | 17403.3 | 5497.0 |

Additionally, 5 validated variants, along with a true wild type, were assayed for Rubisco protein levels by Western Blot. Two protein gels were run in total, each with 2-3 variant samples and one wild type sample. Each sample was loaded at three dilutions (FIG. 15B). Spot densitometry was used to quantify the average pixel intensity (API) of each 52 kDa band after background correction, and a standard curve was generated to correlate pixel intensity to the amount of protein loaded at each point along the dilution series. APIs for diluted samples were multiplied by a dilution factor as determined by the standard curve resulting in 3 independent API measurements for each protein sample. The calculated API for each original line sample was compared to the wild type sample on its respective Western Blot. No sample showed a significant difference in intensity from wild type based on ANOVA with Dunnett's post test (p<0.05).

Quantitative Real-Time PCR (qPCR).

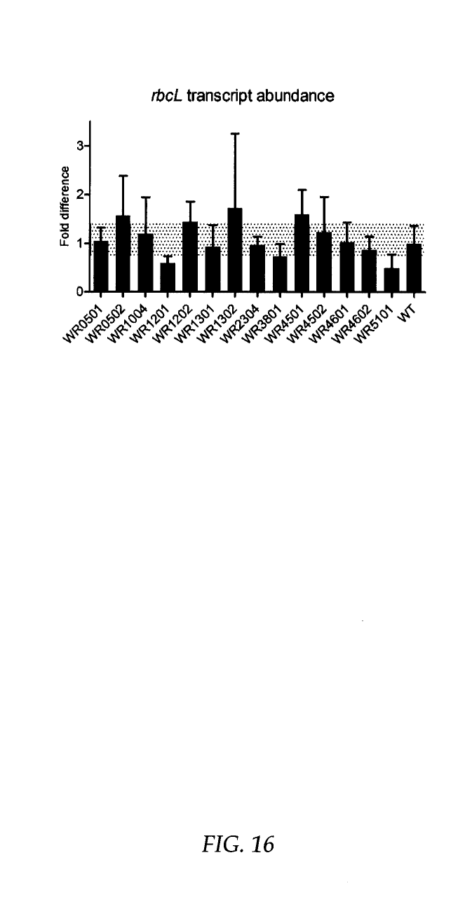

A subset of original lines including the 13 with significantly greater s than wild type in 1:1 turbidostat competitions and one line with no difference from wild type (WR2304) were assayed for rbcL transcript abundance using qPCR. Two plates of PCR were set up, each containing 7 original line samples and one wild type sample. The calculated relative transcript abundances within the set ranged from approximately 0.5-fold to 1.7-fold the wild type level, with most falling within the calculated error of wild type (see FIG. 16).

### Example 1 Validated Variants

The variant IDs and mutations are listed in the Table 22 below. It is interesting that all but 2 of the 16 validated variants cluster in four distinct regions of the rbcL structure: Loop 25-35, β-sheet 83-89, α-helix 310-321, and Loop-Helix-Loop 355-365.

Growth Characteristics.

43 regenerated lines were tested in microtiter plate growth assays using MASM, TAP and HSM media. The experiment was duplicated in two separate setup rounds. In each round, all plates were set up in duplicate and incubated in 5% CO₂ and 0.04% CO₂ (air). Each clone was assayed in quadruplicate for each condition (media, CO₂) in each round. In MASM and TAP media, full growth curves were obtained, while only linear growth rates were obtained in HSM. Growth rates were not obtained for cultures grown in HSM media without CO₂ enrichment, as no lines showed a significant increase in OD₇₅₀ over the duration of the experiment. Logistic growth curves were fit to MASM and TAP growth data, and the calculated carrying capacities (K), maximum growth rates (r), and peak theoretical productivities (Kr/4) were compared to wild type replicates in the same plate. For HSM (and in cases where logistic curves could not be fit), only the growth rates were compared to the wild type growth rates. All comparisons were carried out using ANOVA with Dunnett's post test, p<0.05.

39 of the 43 lines were significantly higher than wild type in at least one growth parameter under one or more conditions. The wild type-complemented knockout line was also included in the experiment, and outperformed wild type in MASM and TAP with enriched CO₂. 3 lines had significantly higher rates than both wild type and complemented knockout controls in TAP with no CO₂ enrichment (marked with * in Table 22), and 1 line had significantly higher productivity than both controls in MASM with no CO₂ enrichment (marked **). In the Table 22, + denotes a significant increase from wild type in 1 round, ++ in 2 rounds.

TABLE 22
Increased carrying capacity (K), productivity (Kr/4)
and/or growth rate (r) over wild type

| | 0.04% CO₂ | | | | 5% CO₂ | | | | | |
|---|---|---|---|---|---|---|---|---|---|---|
| | MASM | | | TAP | HSM | MASM | | TAP | | |
| VARIANT | K | Kr/4 | r | r | r | Kr/4 | r | K | Kr/4 | r |
| WT-Comp. | | | | | + | | + | | + | + |
| KO | | | | | | | | | | |
| rWR0106 | | | | | + | | | | | |
| rWR0402 | | | | | | | | | + | |
| rWR0504 | | | | | ++ | | | | + | + |
| rWR0505 | | | | | + | | | | | |
| rWR0506 | | | | | + | | | | + | + |
| rWR0507 | | | | | + | | | | | |
| rWR0508 | | | | | + | | | + | | |
| rWR0604* | + | | | + | | | | | | |
| rWR0605 | | | | + | + | | | | + | + |
| rWR0901 | | | | + | | | + | | + | + |
| rWR0904* | | | | + | | | | | | |
| rWR1002 | | | | | + | | | | + | + |
| rWR1208 | | | | | + | | + | | + | + |
| rWR2002 | | | | | | | + | | + | + |
| rWR2004 | | + | | + | ++ | | + | | + | + |
| rWR2009* | | | | ++ | | | | | | + |
| rWR2106 | | | | | + | | + | | + | + |
| rWR2304 | | | | | | | | | + | + |
| rWR2601 | | | | | | | + | | + | |
| rWR2704 | | | | | | | | | | + |
| rWR3102 | | | | | + | | | | | |
| rWR3201 | | | | | | | | | + | + |
| rWR3403 | + | + | | | | | | | | |
| rWR3501 | | | | | | | | | + | + |
| rWR3704 | | | | | + | | + | | + | + |
| rWR3705 | | | | | + | | | | + | |
| rWR3901 | | | | | | + | + | | + | + |
| rWR4201 | | | | | + | | | | + | + |
| rWR4301 | | | | | ++ | | | | | |
| rWR4302 | | | | | | + | | + | | |
| rWR4901** | + | ++ | + | | ++ | | | | + | + |
| rWR5102 | | | | + | + | + | | + | | |
| rWR5201 | | | | | | | | | + | + |
| rWR5206 | | + | | | + | | | | + | + |
| rWR5301 | | | | | | | | | + | |
| rWR5302 | | + | | | + | | + | | + | + |
| rWR5303 | | | | | + | | + | | ++ | ++ |
| rWR5905 | | | | | + | | + | | + | + |
| rWR6306 | | | | | ++ | | | | | |

### Example 1 Summary

Data for the 16 variants validated in the experiments of Example 1 are summarized in Table 23.

TABLE 23
Example 1 validated Rubisco variants

| Winner ID | Variant | Original Δs | Regenerated Δs | Element | K | Kr/4 | r | Low Δr, 22°-30° C. | FIRe | W. Blot low |
|---|---|---|---|---|---|---|---|---|---|---|
| WR0402 | D28H | | 0.2156, 0.0423 | L: 25-35 | | T | | | | |
| WR0502 | V31K | 0.2748, 0.0687 | 0.0662, −0.0304 | L: 25-35 | | M, T | | | S, F | Yes |
| WR0505 | T34H | | 0.0975, 0.0865 | L: 25-35 | | | H | | E, A | |
| WR0605 | I36L | | 0.1786, 0.2243 | | | T | T, H | Yes | A | |
| WR1002 | T68Q | | 0.1899, 0.0251 | | | T | T, H | | E, A | |
| WR1004 | T68S | 0.3395, 0.2197 | 0.0238 | | | M | M, T | | F | Yes |
| WR1201 | R83Q | 0.2956, 0.2298 | 0.1040, −0.1171 | β: 83-89 | | T | T | | S, F | Yes |
| WR1202 | R83H | 0.4691, 0.1291 | −0.0646, 0.0891 | β: 83-89 | | | | | | Yes |
| WR1301 | E88S | 0.3617, 0.0892 | 0.0678, −0.1112 | β: 83-89 | | M, T | M, T | | A, F | |
| WR1302 | I87Y | 0.2266, 0.0694 | 0.2295, 0.2349 | β: 83-89 | | | | | | |
| WR4502 | R312K | 0.2400, 0.1614 | 0.0185, 0.0627 | α: 312-321 | | M | M, T | | S | Yes |
| WR4601 | A317G | 0.2318, 0.0112 | 0.1771, 0.0296 | α: 312-321 | | | T | Yes | A, S, F | |
| WR4602 | M320L | 0.4010, 0.0453 | 0.0859, 0.1176 | α: 312-321 | | | M | Yes | A | |
| WR5101 | E355P | 0.2250, 0.1620 | −0.0052, 0.0789 | LHL: 355-365 | | | | | | |
| WR5206 | R358Q | | 0.1854 | LHL: 355-365 | | M, T | T, H | | | |
| WR5303 | T365S | 0.2143 | 0.1016, 0.3018 | LHL: 355-365 | | | | Yes | E | |

Table 23 KEY
Δs: selection coefficient(variant)-selection coefficient(wild type)
Element: (L) Loop; (β) beta sheet; (α) alpha helix; (LHL) loop-helix-loop
K, Kr/4, r: Significant microplate growth in: T = TAP, M = MASM, H = HSM
Low Δr, 22°-30° C.: column temp difference (significant = Yes)
FIRe: E = ETRₘₐₓ, A = alpha, S = sigma, F = Fᵥ/Fₘ for significant differences
W. Blot low: significant = Yes

### Example 2. Single- or Double-Point Mutations

### Screening

### Libraries

Two approaches were taken to generate mutagenic libraries. In one approach, Site Saturation Mutagenesis (U.S. Pat. No. 5,830,650) (SSM) was used to combine each of three single mutations validated in Example 1 (R83H, I87Y, and R312K) with a highly diverse set of second mutations evenly distributed across the protein. The result was three separate SSM libraries of approximately 4,200 double mutants, each with one of the previously identified advantageous mutations held constant. In a second approach, the residues of two structural elements containing several previously validated mutants (β-sheet 83-89 and α-helix 310-321) were mutated to all 20 amino acids using the degenerate codon NNK, both as single mutants and while holding one of four previously identified advantageous mutants in each element constant to generate double mutants. The result was five NNK libraries per structural element containing approximately 190-350 mutants each.

For SSM libraries, each amino acid in the C. reinhardtii RuBisCO large subunit protein (RBCL) was substituted for 9 amino acids representing different classes of side-chain chemistries. These include the positively charged amino acids histidine (H) and lysine (K), negatively charged aspartic acid (D), polar neutral serine (S) and glutamine (Q), hydrophobic leucine (L) and tyrosine (Y), small flexible glycine (G), and small rigid proline (P).

For each SSM constant mutation, 68 individual libraries were generated representing unique 7-amino acid segments of the RBCL protein referred to as Regions. Therefore the mutations at amino acid positions 1-7 were generated in the Region 1 library, mutations for amino acids 8-14 were generated in Region 2, and so forth. These 68 regions cover the entire protein. To generate the mutations, DNA oligonucleotides covering portions of the rbcL gene were synthesized, each containing a single codon change to produce the desired amino acid substitution. Oligonucleotides for each region were ordered in a plate array according to the reference amino acid position and the respective substitution.

Each 63-mer oligonucleotide encompassed the 21 nucleotide region where the mutagenic codon resides, along with 21 nucleotides at both the 5′ and 3′ ends identical to the parental (C. reinhardtii rbcL with R83H, I87Y, or R312K single point mutation) sequence flanking that region. In addition, a single non-mutagenic oligonucleotide was designed for each region on the opposite DNA strand, such that 21 nucleotides of homology exist between each mutagenic oligonucleotide and its non-mutagenic counterpart.

Oligonucleotides to generate NNK libraries were designed the same way, with the exception that the mutagenic portions were 21 or 36 nucleotides in length for the β-sheet and α-helix, respectively. For each codon in the mutagenic portion, a unique oligonucleotide was designed with the degenerate sequence NNK (N=any nucleotide; K=G or T) substituted for the wild type codon. The NNK sequence encodes 32 of the possible 64 codons, encompassing all 20 amino acids as well as a stop codon.

Mutations were incorporated into the rbcL gene using an overlap PCR technique. Briefly, each mutagenic oligonucleotide was used as a primer in a PCR reaction with another primer at the opposite end of the gene, along with a template plasmid containing the parental rbcL sequence. Primers are incorporated into the resulting PCR products, thereby introducing the mutagenic codon at one end of the amplicon. A second PCR is carried out in a similar fashion using the complementary non-mutagenic oligonucleotide as a primer to amplify the portion of the gene not covered by the first reaction. In a third PCR, purified amplicons from each of the previous reactions are combined with primers that flank the rbcL gene to both amplify the assembled variant and add the restriction endonuclease sites NdeI and SpeI to the 5′ and 3′ ends, respectively. Homology between the amplicons derived from the initial PCRs allows the entire gene to be assembled without the use of a traditional template.

Full-length amplicons were digested with NdeI and SpeI, and ligated into the C. reinhardtii chloroplast transformation vector pSC179 (FIG. 1). The vector contains approximately 2.8 kb each of 5′ and 3′ flanking sequence homology to the rbcL locus in the C. reinhardtii chloroplast genome, as well as elements for propagation and selection in bacteria.

Once the libraries were ligated into the vector, they were transformed into bacteria for amplification and QC. Resulting bacterial colonies for each library (median n>1,500) were scraped into liquid cultures and plasmid DNA was purified the following day. For SSM libraries, equimolar plasmid DNA was combined into Pools of 6-7 regions each, such that each SSM library was divided into 10 Pools. NNK libraries were separated based on the structural element and constant mutation (see Table 24 below).

TABLE 24

| SSM Pool | Regions | #AA's | Lib. Complexity | NNK Lib. | Region | #AA's | Lib. Complexity |
|---|---|---|---|---|---|---|---|
| 1 | 1-7 | 49 | 441 | β sheet | 2 | 7 | 224 |
| 2 | 8-14 | 49 | 441 | β + R83H | 2 | 7 | 192 |
| 3 | 15-21 | 49 | 441 | β + R83Q | 2 | 7 | 192 |
| 4 | 22-28 | 49 | 441 | β + I87Y | 2 | 7 | 192 |
| 5 | 29-35 | 49 | 441 | β + E88S | 2 | 7 | 192 |
| 6 | 36-42 | 49 | 441 | α helix | 7 | 12 | 384 |
| 7 | 43-49 | 49 | 441 | α + R312K | 7 | 12 | 352 |
| 8 | 50-56 | 49 | 441 | α + A315S | 7 | 12 | 352 |
| 9 | 57-62 | 42 | 378 | α + A317G | 7 | 12 | 352 |
| 10 | 63-68 | 42 | 378 | α + M320L | 7 | 12 | 352 |

The distribution of mutations in each library was determined by amplifying each mutagenic portion of the gene with uniquely barcoded PCR primers, followed by Ion Torrent next-generation sequencing (NGS). Dual sets of primers with unique barcodes were designed for each Pool/structural element to produce amplicons of ^{˜}200 bp (not including adapters or barcodes). The primer sets for each amplicon were identical with the exception that the adapter/barcode on the Forward primer of one set was attached to the Reverse primer of the other set, and vice-versa, to allow for bi-directional sequencing. These primer sets were used to amplify DNA from each of the plasmid libraries. PCR products from each library were combined and sequenced on an Ion Torrent 318 chip with 200 bp chemistry. Deconvoluted data for each barcode was then mapped to the reference rbcL sequence to determine the number of reads containing each of the expected variants. Each barcode comprised reads from both directions; Forward reads were used from the first codon of the mutagenic region to the mid-point of the amplicon, and Reverse reads were used from the mid-point of the amplicon to the last codon of the mutagenic region. Sequencing errors due to insertions, deletions, early terminations, etc. were excluded from the analysis, and therefore the total number of sequences at each codon position varies across an amplicon. To normalize the counts for each variant, the raw number of reads was multiplied by the correction factor Sum_{BC}/Sumₚₒₛ, where Sum_{BC} is the total number of reads for the barcode (amplicon), and Sumₚₒₛ is the total number of error-free reads at that codon position. A noise threshold was established for NGS data as the maximum observed frequency for a variant that is known to be nonexistent in the library. In this case, the 9 variants for the Start codon (which was not mutagenized) were used. In the plasmid libraries the maximum frequency observed for one of these variants was 7×10⁻⁵, and therefore all frequencies of 7×10⁻⁵ or below were considered noise (i.e. not distinguishable from zero).

Given an equal distribution of mutant sequences within an SSM barcode, the parental (reference) codon will be present 48 out of 49 times in the Pool of interest, making up approximately 98% of the reads on a codon-by-codon basis. Therefore, the number of parental sequences must be estimated with a different approach than the codon focused method used for variant counts. In the plasmid libraries, each nucleotide in a read was marked as “likely reference” if it was (i) identical to the reference sequence, (ii) called as a deletion, or (iii) not covered by that particular read. Reads containing 49/49 “likely reference” nucleotides in the region of interest were counted as parental sequences.

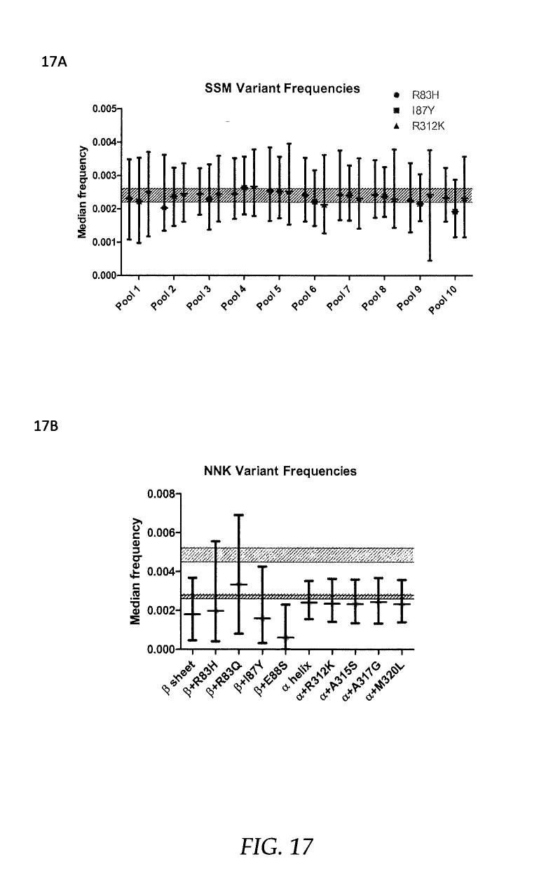

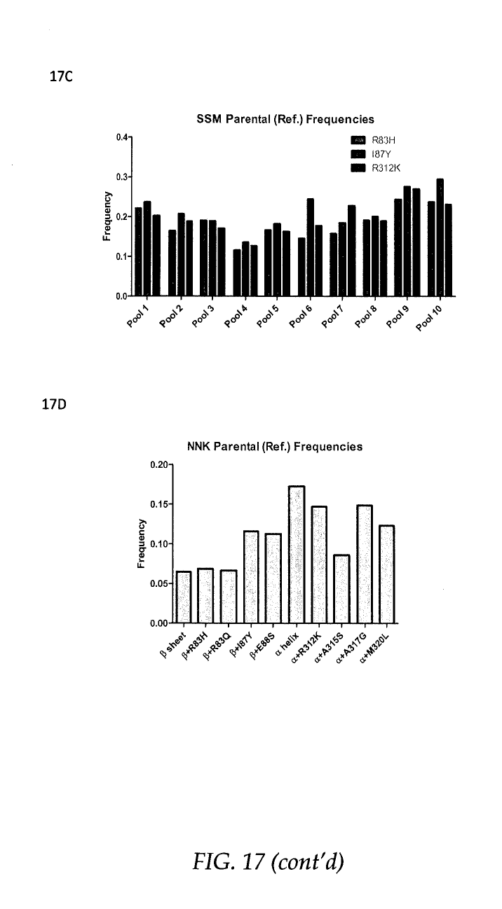

The distributions of mutant frequencies, along with parental sequences, are shown in FIG. 17. For each SSM Pool or NNK library the median frequencies and interquartile ranges of all expected mutants are shown in the FIGS. 17A and B. Given a perfect distribution within the SSM libraries, each variant would represent 1/441 (0.22%) or 1/378 (0.26%) of the sequences in Pools 1-8 or Pools 9-10, respectively. Perfect distribution of the NNK libraries would be 1/224 (0.45%) for β single mutants, 1/192 (0.52%) for β double mutants, 1/384 (0.26%) for a single mutants, and 1/352 (0.28%) for a double mutants. These ranges are indicated by the shaded areas on each graph. Actual variant percentages in the plasmid libraries ranged from 0%-25.9% for SSM, and 0%-26.6% for NNK. Although all oligonucleotides encoding the parental sequences were removed from the original gene synthesis PCR, single-mutant parental sequences were present at varying levels (6.5%-29.5%) across the libraries as shown in FIGS. 17C and D. Parental sequences are likely derived from mis-priming via truncated mutagenic oligos in the overlap PCR or from original template carryover.

Based on the NGS sequencing results, 98.3% and 98.6% of the expected mutants were detectable above the baseline noise level in the SSM and NNK plasmid libraries, respectively. Note that the 9 substitutions of the start codon ATG were not present in the SSM libraries because that codon is part of the cloning site NdeI. The other variants that were not detected were simply not created during the PCR process or are below the level of detection for the sequencing method.

### Primary Turbidostat Screening

A RuBisCO large subunit knockout (ΔrbcL) algal strain was generated by transforming the chloroplast genome of wild type C. reinhardtii cells via gold particle bombardment with a vector containing the kanamycin resistance gene aphA6 flanked by 5′ and 3′ homology to the rbcL locus. Since transformation of the chloroplast genome occurs by homologous recombination in the C. reinhardtii chloroplast, selection of kanamycin resistant clones is indicative of rbcL displacement in one or more copies of the chloroplast genome. Continually passaging transformants on selective media resulted in homoplasmic knockout clones where no copies of rbcL were detectable by PCR. Since the knockout strain is non-photosynthetic it requires an organic carbon source such as acetate to grow. DNA from each rbcL variant library was transformed into the chloroplast genome of ΔrbcL C. reinhardtii cells via gold particle bombardment. Again, homologous recombination results in replacement at the rbcL locus, this time replacing the aphA6 kanamycin marker with rbcL variants. Selection for rbcL complementation was carried out on HSM agar, a minimal medium that necessitates obligate photoautotrophic growth. Any rbcL variants that are not present in these transformant lines are likely non-functional forms of RuBisCO inactivated by the introduced amino acid substitution.

Transformed algal colonies for each SSM Pool were counted, and approximately one third of the colonies were scraped into three separate flasks containing TAP media. Pools were divided into three Subpools in order to decrease the complexity in each competition, as well as to create varied environments for mutants to compete in. Transformed algal colonies for the NNK libraries were scraped together into flasks en masse. Median coverage for the SSM Pools, SSM Subpools, and NNK libraries was 7.2, 2.4, and 12.9-fold, respectively.

One to three days following flask inoculation, cells were passaged to new flasks, and then inoculated into quadruplicate turbidostats two to four days later. The turbidostats were filled with HSM media and set to an OD₇₅₀ of approximately 0.3, which represents an early- to mid-log growth phase. Constant light of ^{˜}150 μEinstein was provided, with a constant stream of 0.2% CO₂ bubbling into the culture. Cultures were monitored at least daily for media replenishment, CO₂ delivery, culture settling, cell sticking, mechanical failure or any other issues. The cultures were grown under these optimal photoautotrophic conditions for four weeks. Samples were taken from the inoculum flasks and subsequently from each turbidostat at 7 day intervals starting at day 14, and single cells were sorted by fluorescence-activated cell sorting (FACS) into 96-well plates containing TAP media. Cell lysates were also prepared from each sample for DNA sequencing (NGS). After a week or more of growth, sorted strains were replicated onto solid media for longer-term recovery and isolation of transformed lines.

Sequencing and Analysis from Primary Turbidostat Screening

After 5-7 days of growth in 96-well plates, the individual sorted strains were used as template in a PCR reaction that amplified the rbcL gene based on gene specific primers. After ascertaining success in producing a single product from the reactions, the PCR products were treated for sequencing with Exonuclease I/Shrimp Alkaline Phosphatase (ExoSAP). These products were then sequenced via Sanger chemistry using a primer that reads into the region of interest. These clonally isolated and sequenced variants are used as a check on the NGS data, but are primarily for the identification and isolation of particular clones of interest for the validation process.

The distribution of mutants in each turbidostat was determined by amplifying the region of interest from cell lysates with uniquely barcoded PCR primer sets, followed by Ion Torrent next-generation sequencing (similar to the plasmid library sequencing described above). Barcoded amplicons from the baseline inoculums and all final replicates were combined together on separate Ion Torrent 318 chips, such that the baseline, final “A” replicates, final “B” replicates, and so forth were sequenced together. Analysis of the NGS data from turbidostat algae samples was similar to that described earlier for the plasmid library analysis, except that the non-existent variants used to determine the noise threshold were based on the TAG (premature Stop) codons created in the NNK libraries, which would be lethal mutations in vivo. The maximum ratio observed for one of these variants in algae was 0.002, and therefore all ratios of 0.002 or below were considered noise (i.e. not distinguishable from zero).

Sequences were analyzed from each turbidostat replicate at beginning and ending time points, with the difference being baseline (time 0) data, which was analyzed per set and then used as the starting point for each turbidostat replicate of that set. Since NNK libraries may contain synonymous mutants encoded by different codons, the total number of counts for each amino acid was calculated by summing the counts for individual codons, where applicable, prior to calculating ratios.

Hit counts and total sequences were used to calculate the ratio of each variant present in a given time point. These numbers can then be used to calculate a selection coefficient using the formula provided previously. Note that the selection coefficients used in this analysis do not conform strictly to some of the assumptions upon which the formula is based, in that this is not a single clone compared against a uniform population. Each clone to was compared to the rest of the population, which itself is made up of many other clones. However, within the experiment, the calculated selection coefficients provide a valid way to compare and rank potentially winning clones.

In many cases, a given sequence was identified at one time point but not detected in another time point. As the natural log of zero produces an error, assumptions were necessary in such cases. For any instance where the baseline was zero (i.e. below the noise threshold ratio of 0.002) but the variant was detected at the endpoint, a value of 1/c was assigned to the baseline ratio, where c equals the number of colonies that were scraped into the inoculum flask. For the more common case where the variant was detected at baseline but not at the endpoint (termed “extinct” variants), a value of 0.0001 was assigned to the endpoint ratio resulting in a large negative selection coefficient, but avoiding the calculation error. During the analysis, these assumptions were monitored to avoid consideration of artifactual data.

In order to successfully identify and recover winning clones from the primary screen, a formula was used to estimate the length of time required for competition and the number of clones to analyze in order to reach a desired level of sensitivity. Assuming the minimum 1-in-441 starting ratio for SSM libraries, approximately 200 sequences at the endpoint and a sensitivity of 1% (i.e. 2 sequences out of 200), it was possible to calculate the time necessary to identify a clone with a selection coefficient of 0.05 as follows:

$$ln ( \frac{2}{198} ) = ln ( \frac{1}{440} ) + 0.05 {d}^{- 1} \cdot t d ; t = 29.8 d$$

Thus in the primary screen, an s value of approximately 0.05 should be detectable within ^{˜}4 weeks of growth by sequencing approximately 200 clones. It is important to note that the above calculations are based on Sanger sequencing for recovery of winning clones; NGS sequencing has much higher sensitivity (^{˜}0.2%) so less time is required to identify winners by NGS. In addition, this calculation assumes 100% viability of all variants in the library; the true number of variants capable of complementing the knockout will be lower, which further reduces the amount of time required to isolate winning clones.

### Secondary Turbidostat Screening

For every variant detected by NGS in the primary screen, selection coefficients were calculated using the common baseline hit ratio and the final hit ratio for each replicate turbidostat (sᵣₑₚ). The average of these sᵣₑₚ values is calculated as s_{avg}. An alternative selection coefficient was also calculated for each variant by summing the final hits and the sum of total sequences for all replicates and using that as the final ratio for s calculation (sₛᵤₘ). The top 247 isolated variants based on s_{avg} and sₛᵤₘ were subjected to a head-to-head (1:1) turbidostat growth assay against a common competitor strain as a secondary screen.

In Example 1, the common competitor strain was a kanamycin-resistant (kan^{R}) C. reinhardtii wild type analog. While the ratio of kan^{R} to rbcL variant can be effectively determined by FACS-sorting a population for single cells followed by replica plating onto selective and permissive media, the process is labor intensive and relatively low throughput. An improved assay was developed which takes advantage of flow cytometry to determine the relative ratios of fluorescent and non-fluorescent strains in a population, allowing for increased resolution (^{˜}10 X) over sorting and replica plating. The common competitor strain for this assay was generated by transforming wild type C. reinhardtii cells with a plasmid containing codon-optimized Venus (GFP variant) and zeocin-resistance genes under control of a strong constitutive nuclear promoter. The clone selected to be the common competitor for the assay was shown to have stable fluorescence, and while it slightly outperforms wild type (s_{avg}=0.03), all lines including wild type were evaluated relative to it. Several turbidostat experiments competing the Venus⁺ strain against various non-fluorescent strains were run to demonstrate that ratios calculated by flow cytometry on a Millipore Guava EasyCyte are equivalent to those obtained by FACS-sorting and replica plating onto TAP/TAP+zeocin.

Winning clones from primary screening, along with the Venus⁺ strain, single-mutant parental clones, wild type, and ΔrbcL complemented with wild type rbcL, were inoculated into flasks containing TAP media. After 2-4 days of growth in flasks, cells were passaged to new flasks and grown for 1 additional day. Cell concentrations were then normalized by OD₇₅₀ and winners (and controls) were mixed in equal volume with the Venus⁺ strain and inoculated into triplicate turbidostats. The turbidostats were filled with HSM media and set to an OD₇₅₀ of approximately 0.3, which represents an early- to mid-log phase. Constant light of ^{˜}150 μEinstein was provided, with a constant stream of 0.2% CO₂ bubbling into the culture. Cultures were monitored at least daily for media replenishment, CO₂ delivery, culture settling, cell sticking, mechanical failure or any other issues. Samples were taken twice over the course of 5 days, typically on or around days 2 and 4. At each time point, samples were run on the Guava instrument to determine the relative ratio of Venus⁺ and rbcL variant in each population. The amount of growth media consumed between time points was also recorded to determine the approximate number of generations each turbidostat had gone through.

Analysis from Secondary Turbidostat Screening

Using Guava CytoSoft software, gates were applied to each flow cytometry run to differentiate non-green fluorescent cells from the Venus⁺ strain. The winner ratio was calculated for each sample as:

$$r = \frac{M 1}{M 2}$$

where M1 is the number of non-fluorescent counts in gate M1 (red channel), and M2 is the number of fluorescent counts in gate M2 (blue channel). Both strains fluoresce in the red channel due to the presence of chlorophyll.

The selection coefficient equation, ln(rₜ)=ln(r₀)+st, is in the form of a line y=b+mx, where the selection coefficient (s) is equivalent to the slope (m) of the natural log of the ratio over time. Alternatively, selection coefficients can be calculated by plotting ln(rₜ) vs. the number of generations. While turbidostats maintain optical density within a relatively narrow range, slight variances in density can affect the growth rate of a turbidostat's population, thereby varying the number of generations produced by replicate turbidostats. In order to control for this effect, media consumption between Guava samplings was used to calculate the number of generations at each time point, and selection coefficients were calculated in units of generations⁻¹ using the equation above but substituting r₀, rₜ, and t for the ratio (M1/M2) at the first Guava sampling, the ratio at the second sampling, and the number of generations between samplings, respectively. The calculated selection coefficients were then used to rank and select potential winning clones as Proposed Genes. See results below for details.

### Screening Results

### Primary Turbidostat Screening Results

Depending on transformation efficiency, 1-3 transformation rounds, each consisting of 54 biolistic shots per SSM Pool or NNK library, provided the transformed Chlamydomonas lines for primary screening. After colonies had grown up on the transformation plates, they were counted and put into sets with an average of 1,390 (SSM Subpool) or 3,410 (NNK library) colonies in each. These sets served as the inoculum cultures for primary turbidostat screening.

Based on previous experience with operating turbidostats, attrition was expected over the course of a multi-week experiment due to occasional equipment failure or culture crash. Therefore excess replicates were set up for screening. 100 sets were initially set up, one per SSM Subpool (90) or NNK library (10), and four replicate turbidostats were established per set (see figure below for SSM example). The target screening time for the cultures was four weeks, however in the event that a turbidostat failed to make the four week endpoint but samples were still collected for NGS at week two or week three, the turbidostat was still included in the analysis. Of the 400 primary screen turbidostats, 368 made it to the four week endpoint, 10 made it to week three, 1 made it to week two, and 21 failed before NGS samples were taken and therefore were excluded from the analysis. Every set was represented by at least 3 replicates in the NGS analysis.

Use of NGS for data generation allowed for much more resolution over use of Sanger sequencing of individual clones. While Sanger data only provides data on the most prevalent variants in the population (and thus skews towards those variants that become dominant via a selective advantage), NGS allows sampling of nearly all variants in a pool. Thus those variants that are neutral or with a negative selection can be identified and characterized. Even those mutants that are present at the beginning of an experiment that go to zero (“extinct”) can be fairly reliably detected.

The primary screen turbidostats had relatively low starting diversities, and consequently, high starting parental frequencies ranging from 24.9% to 92.6%. Each SSM Pool had a theoretical maximum of 441 variants (49 amino acids×9 substitutions) with actual average variant numbers per Pool of 45 (R83H), 32 (I87Y), and 20 (R312K). The numbers of variants per Subpool were roughly 3-fold lower. The NNK libraries had theoretical maximums of 217 and 372 variants for the β-sheet and α-helix, respectively; actual average variant numbers were 57 ((3) and 60 (a). This suggests that on average, 92% of the SSM variants and 79% of the NNK variants either did not complement the ΔrbcL strain or were below our detection limit. Because of this low diversity, the four replicates of each primary pool showed good reproducibility. Selection coefficient values derived from the primary screen, while relative only to variants within the Subpool/library and some fraction of parental variants, can be relied upon as a main criterion for selecting winners.

### Secondary Turbidostat 1:1 Competition Results

For every variant detected by NGS in the primary screen, selection coefficients were calculated using the common baseline hit ratio and the final hit ratio for each replicate turbidostat (sᵣₑₚ). The average of these sᵣₑₚ values is calculated as s_{avg}. To prevent large negative sᵣₑₚ values from excessively lowering s_{avg}, thereby masking good performance in other replicates, an alternative selection coefficient was also calculated for each variant by summing the final hits and the sum of total sequences for all replicates and using that as the final ratio for s calculation (sₛᵤₘ). Comparing all of the s_{avg} values for the replicates with the sₛᵤₘ value on the summed replicates gives significant positive correlations, suggesting that either measure would be useful for selecting winners. Given that the two selection coefficient calculations are not perfectly correlated (R=0.75), both were used to ensure all winners were identified.

Approximately 1,200 variants were identified at the endpoint of the primary screen. To select the top candidates, a cutoff was applied if s_{avg} and sₛᵤₘ were less than 0.02. This narrowed the list to 296 variants from the SSM libraries and 59 from NNK, for a total of 355 with s_{avg} and/or sₛᵤₘ>0.02. Given limited resources to run 1:1 turbidostat competitions, this list was further ranked by the number of replicates each variant had with sᵣₑₚ>0.100, 0.075, and 0.050. The remaining variants with no sᵣₑₚ>0.050 were sorted by descending s_{avg}. The list was then ranked from 1 to 355.

The top 247 variants on the list that were successfully isolated from FACS sorting were scaled up for 1:1 turbidostat competitions (211 from SSM and 36 from NNK). In 54 cases, a top variant was not identified by Sanger sequencing the FACS-sorted clones, or a variant was identified but was contaminated and therefore not scaled up. Thus, the actual ranking of variants that advanced to the secondary screen ranged from 1 to 301. If a top SSM variant was identified in more than one Subpool, the clone was isolated from the Subpool with the highest s_{avg} whenever possible. Likewise, top NNK variant clones were preferentially chosen based on the codon with the highest s_{avg}.

Four rounds of approximately 61 winners each, along with wild type and wild type-complemented knockout strains, were set up. The SSM parental variants were included in three separate setup rounds; the NNK parentals in one. No data was generated for the A317G parental line; one line in the secondary screen had this parental mutation. Each winner (or control) was mixed 1:1 with the Venus⁺ strain and inoculated into triplicate turbidostats. All competitions were run for at least 5 days, and samples were taken twice during that time. For each winner and control, Guava ratios were used to calculate sᵣₑₚ and s_{avg} values relative to the Venus⁺ strain.

The s_{avg} for each winner in the secondary screen was compared to the s_{avg} values for wild type, wild type-complemented knockout, and the parental line controls (where applicable) to generate three Δs_{avg} values, where Δs_{avg}(winner)=s_{avg}(winner)−s_{avg}(control). Δs_{avg}>0 implied that a winner line would outperform the control if they were competed head-to-head. Wild type and the complemented knockout were run in all setup rounds, and therefore winner lines were compared to the wild type/wild type-complemented controls in their respective round. Parental controls were not run in every round, therefore s_{avg}(parent) values were generated by averaging the sᵣₑₚ values across all rounds in which they were run.

### Gene Selection

Data from both the primary and secondary turbidostat screening experiments was used to determine the top 98 variants for Selection. In the primary screen, selection coefficients were calculated for all variants in each replicate turbidostat using the common baseline hit ratio for the set and the final hit ratio for each replicate (column sᵣₑₚ next page). The average of these replicate s values is calculated as s_{avg}. An alternative selection coefficient was also calculated for each variant by summing the final hits and the sum of total sequences for all replicates and using that as the final ratio for s calculation (column sₛᵤₘ). In the example from primary screening given below, the endpoint is at 28 days. As a demonstration, s for the first replicate in Table 25 (and highlighted in bold text) is calculated as follows:

$$ln ( {r}_{t} ) = ln ( {r}_{0} ) + s \cdot t$$

$$ln ( \frac{493}{54856 - 493} ) = ln ( \frac{142}{36652 - 142} ) + s \cdot 28$$

$$ln ( 0.0091 ) = ln ( 0.0039 ) + s \cdot 28$$

$$s = 0.0303$$

TABLE 25
Example data for two variants at one position. Positions and original residues are anonymized but actual data is presented.

| | | Raw | | | | Corrected | | | | | | Final | Final | |
|---|---|---|---|---|---|---|---|---|---|---|---|---|---|---|
| Variant | Replicate | Counts | Sumₚₒₛ | Sum_{BC} | Corr. | Counts | Ratio | Sᵣₑₚ | S_{avg} | stdev | CI | Hit Sum | Total Sum | sₛᵤₘ |
| X999H | Baseline | 136 | 35127 | 36652 | 1.043 | 142 | 0.0039 | | | | | | | |
| | A | 474 | 52747 | 54856 | 1.040 | 493 | 0.0091 | 0.0303 | 0.0392 | 0.0078 | 0.0163 | 2187 | 185536 | 0.0400 |
| | B | 750 | 50172 | 53976 | 1.076 | 807 | 0.0152 | 0.0486 | | | | | | |
| | C | 347 | 32740 | 34254 | 1.046 | 363 | 0.0107 | 0.0362 | | | | | | |
| | D | 502 | 40678 | 42450 | 1.044 | 524 | 0.0125 | 0.0417 | | | | | | |
| X999S | Baseline | 85 | 35127 | 36652 | 1.043 | 89 | 0.0024 | | | | | | | |
| | A | 9 | 52747 | 54856 | 1.040 | 9 | 0.0001 | −0.1139 | −0.0813 | 0.0653 | 0.1364 | 228 | 185536 | −0.0239 |
| | B | 193 | 50172 | 53976 | 1.076 | 208 | 0.0038 | 0.0167 | | | | | | |
| | C | 15 | 32740 | 34254 | 1.046 | 16 | 0.0001 | −0.1139 | | | | | | |
| | D | 11 | 40678 | 42450 | 1.044 | 11 | 0.0001 | −0.1139 | | | | | | |

As described earlier, the counts for each variant were normalized across a given barcode. The raw count of reads in a given position (i.e. amino acid number) was multiplied by the correction factor Sum_{BC}/Sumₚₒₛ, where Sum_{BC} is the total number of reads for the barcode (amplicon), and Sumₚₒₛ is the total number of error-free reads at that codon position. A one-sample, one-sided t-test was employed by calculating a 95% confidence interval (CI, α=0.025) from the standard deviation followed by comparison of this CI to the average. Any s measurements with a CI less than the average were determined to be statistically greater than zero. In this example, the first variant (indicated with an arbitrary starting amino acid and position of X999 substituted to H) is statistically higher than zero as the average minus the CI is greater than zero.

As previously described, the top 247 variants from primary screening were advanced to head-to-head turbidostat competitions against a wild type analog as a secondary screen. Selection coefficients (sᵣₑₚ and s_{avg}) were calculated for each winner as described above, with the exception that the baseline readings were taken individually from each replicate turbidostat 2-3 days following inoculation, and the number of generations between readings was used in place of elapsed time to control for differences in growth rates. Therefore, all s_{avg} and sₛᵤₘ values reported from the primary screen are in units of days⁻¹, while s_{avg} values reported from the secondary screen are in units of generations⁻¹. While a positive selection coefficient relative to another strain is indicative of a selective advantage regardless of the unit used, the magnitude of s values from the primary screen cannot be directly compared to those from secondary.

Following the secondary screen, all winners have up to 5 selection coefficient measurements to consider: two from primary screening (s_{avg}, sₛᵤₘ), and two or three from secondary screening (Δs_{avg}WT, Δs_{avg}WT-Comp, and Δs_{avg}Parent where applicable). Winners were sorted according to which outperformed all three controls in the secondary screen, followed by which outperformed wild type and wild type-complemented, followed by which outperformed wild type-complemented only, and finally by primary s_{ag}. In several cases, the same variant was present with two or more parental mutations (e.g. X999Q with R83H and I87Y parental mutations). In order to avoid proposing the same non-parental mutation for Selection more than once, all duplicate variants below the top variant as described by the ranking scheme in the previous sentence were removed from the list, leaving 203 unique variants to categorize for proposal.

The first set of winners selected from this data was comprised of 30 variants that outperformed the parental, wild type, and wild type-complemented controls (all Δs_{avg}>0) in the secondary screen (Class 1).

Class 2 is comprised of 18 variants that outperformed the wild type and wild-type complemented controls (Δs_{avg}WT>0, Δs_{avg}WT-Comp>0) in the secondary screen. These lines have a consistent growth advantage over wild type controls in secondary competition experiments.

The primary screen used in this experiment provided a robust dataset that gave a reliable indicator of variant performance, though of course in the context of the mixed variant population it competes against. Thus the next two classes rely on primary data for nomination of variants with the secondary screen as a filter to remove those that are major underperformers in 1:1 competitions. As described in the previously, 95% confidence intervals were utilized to determine whether the primary screen s_{avg} values were significantly greater than zero (p<0.05). Class 3 consists of 21 variants that outperformed the wild type-complemented control in the secondary screen and had s_{avg} values significantly >0 in the primary screen.

Class 4 is comprised of 19 variants that had s_{avg} and/or sₛᵤₘ values>0.075 in the primary screen and outperformed the wild type-complemented control in the secondary screen. While these variants did not demonstrate consistent performance across all primary screen replicates to pass the statistical test, they were selected for strongly enough in one or more replicate(s) to yield high average or sum selection coefficients.

Class 5 consists of 7 variants that strongly outperformed the wild type-complemented control in the secondary screen (Δs_{avg}WT-Comp>0.05). While these lines did not meet the higher threshold of performing better than true wild type in this secondary screen, their performance against this control strain was high enough to warrant inclusion for validation.

As discussed earlier, many of the amino acid substitutions found in winning variants were present in multiple winning lines from different libraries (and therefore with different parental substitutions). Most of these are included in Classes 1-5 above. Class 6 is composed of the remaining 3 variants that were each represented by three or more libraries (with different parental sequences) in the secondary screen and outperformed the wild type-complemented control. By definition, these variants had s_{avg} and/or sₛᵤₘ values>0.02 in the primary screen with multiple other mutations, at least one of which was able to outperform the complemented knockout line in a head-to-head growth competition.

The six classes are outlined in Table 26, below. For a variant to be included in a class, all columns must be true. The number of variants in each class is also listed in the table. Note that a given variant was included in only one class even if it qualified for more than one (e.g. all Class 1 variants could also be considered Class 2 variants). There are a total of 98 variants in Classes 1-6.

TABLE 26

| Class | Primary | Sec. Δs_{avg}Parent | Sec. Δs_{avg}WT | Sec. Δs_{avg}WT-Comp | # Variants |
|---|---|---|---|---|---|
| 1. | | >0 | >0 | >0 | 30 |
| 2. | | | >0 | >0 | 18 |
| 3. | s_{avg} > 0 (p < 0.05) | | | >0 | 21 |
| 4. | s_{avg} \| sₛᵤₘ > 0.075 | | | >0 | 19 |
| 5. | | | | >0.05 | 7 |
| 6. | s_{avg} \| sₛᵤₘ > 0.02 (×3 Lib's) | | | >0 | 3 |

### Validation

Turbidostat Competitions with Primary Lines

Wild Type Analog Strain (Common Competitor).

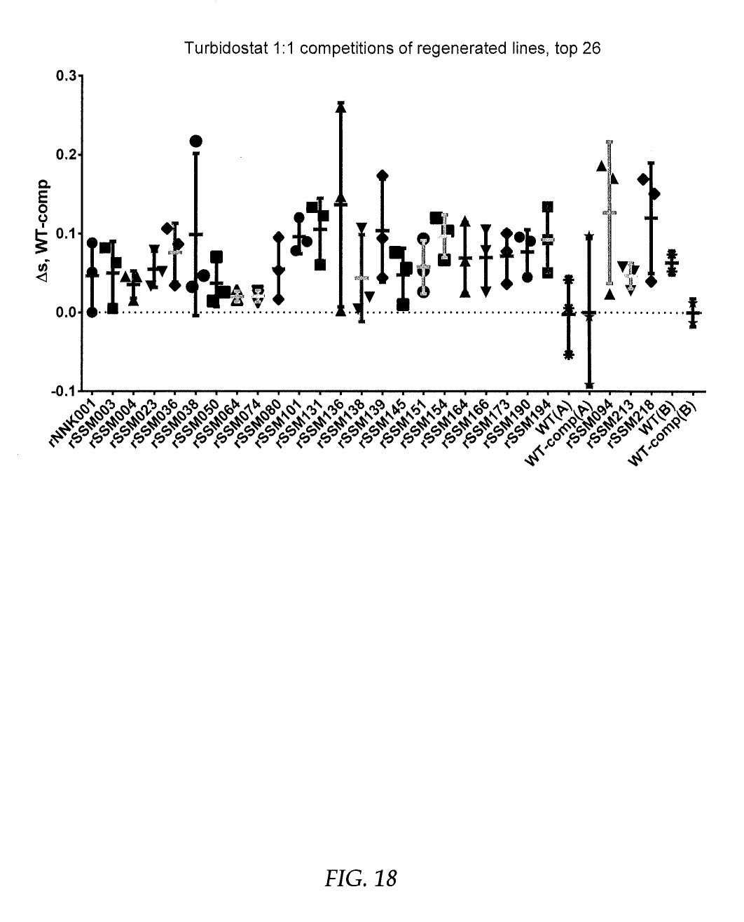

In Example 1, the common competitor strain was a kanamycin-resistant (kan^{R}) C. reinhardtii wild type analog. While the ratio of kan^{R} to rbcL variant can be effectively determined by FACS-sorting a population for single cells followed by replica plating onto selective and permissive media, the process is labor intensive and relatively low throughput. An improved assay was developed which takes advantage of flow cytometry to determine the relative ratios of fluorescent and non-fluorescent strains in a population, allowing for increased resolution (^{˜}10 X) over sorting and replica plating. The common competitor strain for this assay was generated by transforming wild type C. reinhardtii cells with a plasmid containing codon-optimized Venus (GFP variant) and zeocin-resistance genes under control of a strong constitutive nuclear promoter (see FIG. 18).

The clone selected to be the common competitor for the assay was shown to have stable fluorescence, and while it slightly outperformed wild type (s_{avg}=0.03), all lines including wild type were evaluated relative to it. Several turbidostat experiments competing the Venus⁺ strain against various non-fluorescent strains were run to demonstrate that ratios calculated by flow cytometry on a Millipore Guava EasyCyte were equivalent to those obtained by FACS-sorting and replica plating onto TAP/TAP+zeocin.

99 clones along with the Venus⁺ strain, single-mutant parental clones, wild type, and ΔrbcL complemented with wild type rbcL, were inoculated into flasks containing HSM media. One clone (SSM205) was not recovered from the original lines, but a regenerated line was created and tested. After 2-4 days of growth in flasks, cells were passaged to new flasks and grown for 1 additional day. Cell concentrations were then normalized by OD₇₅₀ and winners (and controls) were mixed in equal volume with the Venus⁺ strain and inoculated into triplicate turbidostats. Note that a previous test with control strains yielded no significant differences between turbidostat competitions started at initial ratios of 10:90 and 50:50. The turbidostats were filled with HSM media and set to an OD₇₅₀ of approximately 0.3, which represents an early- to mid-log phase. Constant light of ^{˜}150 μEinstein was provided, with a constant stream of 0.2% CO₂ bubbling into the culture. Cultures were monitored at least daily for media replenishment, CO₂ delivery, culture settling, cell sticking, mechanical failure or any other issues. Samples were taken twice over the course of 8 days, typically on or around days 2 and 8. At each time point, samples were run on the Guava instrument to determine the relative ratio of Venus⁺ and RuBisCO variant in each population. The amount of growth media consumed between time points was also recorded to determine the approximate number of generations for each turbidostat culture.

Analysis from Turbidostat Screening.

Using Guava CytoSoft software, gates were applied to each flow cytometry run to differentiate non-green fluorescent cells from the Venus⁺ strain (a GFP-expressing common competitor). The winner ratio was calculated as described above using the formula:

$$r = \frac{M 1}{M 2}$$

The selection coefficient equation, ln(rₜ)=ln(r₀)+st, is in the form of a line y=b+mx, where the selection coefficient (s) is equivalent to the slope (m) of the natural log of the ratio over time (generally days). Alternatively, selection coefficients can be calculated by plotting ln(rₜ) vs. the number of generations. While turbidostats maintain optical density within a relatively narrow range, slight variances in density can affect the growth rate of a turbidostat population, resulting in a variable number of generations for replicate turbidostats. In order to control for this effect, media consumption between Guava samplings was used to calculate the number of generations at each time point, and selection coefficients were calculated in units of generations⁻¹ using the equation above but substituting r₀, rₜ, and t for the ratio (M1/M2) at the first Guava sampling, the ratio at the second sampling, and the number of generations between samplings, respectively. The calculated selection coefficients were then used to rank and select potential winning clones as Validated Genes.

Turbidostat En Masse Competitions with Primary Lines.

En Masse Turbidostat Competition.

5 ml starter cultures of the 99 original lines were grown in HSM media to mid- to late-log phase in 24 deep-well blocks. Each culture was diluted to OD₇₅₀ of 0.3 with HSM media, and then mixed in equal volumes to generate an inoculum where each line represents about 1/99^{th} of the total population. This inoculum was then used to inoculate each of twelve replicate turbidostats. The turbidostats were run with HSM media at an OD₇₅₀ of approximately 0.3, which represents an early- to mid-log growth phase. Constant light of ^{˜}150 μEinstein (μE) was provided, with a constant stream of 0.2% CO₂ bubbling into the culture.

A sample of the mixture used for turbidostat inoculation (time=0) was sorted for single cells using FACS into 96-well plates containing liquid TAP media. 576 events were analyzed from the inoculum culture. After 20 days of turbidostat growth another sample was taken and used for the same sorting procedure. 96 events were analyzed from each turbidostat at the 20 day time point.

After FACS sorting, the individual strains were grown for approximately 1 week and then used as template in a PCR reaction that amplified the rbcL gene. After ascertaining success in producing a single product from the reactions, the PCR products were treated for sequencing with Exonuclease I/Shrimp Alkaline Phosphatase (ExoSAP). These products were then sequenced via Sanger chemistry (by outside vendors) using 4 separate primers that together cover the entire gene.

Sequences were analyzed in 6 sets of paired replicates. Sanger reads for each amplicon were assembled into contigs using Sequencher software (Gene Codes Corporation). Consensus sequences for each contig were then exported and aligned to the wild type reference sequence. The number of hits for each of the single or double mutant codons was counted for each set.

Hit counts and total sequences were used to calculate the ratio of each variant present in a given timepoint. These numbers were then used to calculate a selection coefficient as described previously.

Regeneration of Lines

Overlap PCR and Chloroplast Transformation.

Most of the regenerated mutations were cloned directly out of the original lines by PCR from genomic DNA. For the few clones that were not recovered from the original lines, mutations were incorporated into the rbcL gene using an overlap PCR technique. Briefly, each mutagenic oligonucleotide was used as a primer in a PCR reaction with another primer at the opposite end of the gene, along with a template plasmid containing the parental rbcL sequence. Primers are incorporated into the resulting PCR products, thereby introducing the mutagenic codon at one end of the amplicon. A second PCR is carried out in a similar fashion using the complementary non-mutagenic oligonucleotide as a primer to amplify the portion of the gene not covered by the first reaction. In a third PCR, purified amplicons from each of the previous reactions are combined with primers that flank the rbcL gene to both amplify the assembled variant and add the restriction endonuclease sites NdeI and SpeI to the 5′ and 3′ ends, respectively. Homology between the amplicons derived from the initial PCRs allows the entire gene to be assembled without the use of a traditional template.

Full-length amplicons were digested with NdeI and SpeI, and ligated into the C. reinhardtii chloroplast transformation vector pSC179 (FIG. 1). The vector contains approximately 2.8 kb each of 5′ and 3′ flanking sequence homology to the rbcL locus in the C. reinhardtii chloroplast genome, as well as elements for propagation and selection in bacteria.

Ligation products were introduced to E. coli cells via electroporation, and plasmid DNA was isolated from individual colonies for sequence verification. Once a single bacterial colony containing the plasmid sequence of interest was identified it was scaled up in overnight culture for plasmid purification. DNA from each rbcL variant was transformed into the chloroplast genome of rbcLΔ C. reinhardtii cells via gold particle bombardment, and selection for rbcL complementation was carried out on HSM agar, a minimal medium that necessitates obligate photoautotrophic growth. Single colonies were isolated and scaled up on TAP agar for sequence verification.

Selection of transformed lines was carried out by restoration of photosynthesis, and photoautotrophic growth has been shown to drive transgenic strains toward a homoplasmic state in which every copy of the chloroplast genome contains the rbcL variant gene in place of aphA6. To this end clones were inoculated into single turbidostats filled with HSM media and set to an OD₇₅₀ of approximately 0.3, which represents an early- to mid-log growth phase. Constant light of ^{˜}150 μEinstein (μE) was provided, with a constant stream of 0.2% CO₂ bubbling into the culture. Cultures were grown under these photoautotrophic conditions for 3-4 days, or approximately 10-14 generations, prior to inoculating 1:1 competitions.

For 11 of the 100 Selected Variants, all replicate selection coefficients were below the WT analog in the 1:1 turbidostat growth competition validation of the original lines and were therefore dropped from further validation work. These 11 Selected Variants were not regenerated, nor were they tested in the turbidostat competitions of regenerated lines, the MGRA and the Western Blot assays. In all, 89 of the Selected Variants were successfully regenerated. All of the regenerated lines were sequence confirmed by Sanger sequencing of isolated clones (by definition).

Turbidostat Competitions with Regenerated Lines.

1:1 Wild Type Competitions.

Regenerated lines were driven to homoplasmicity by growth in single turbidostats for 3-4 days under photoautotrophic conditions as described. PCR was conducted on a daily basis to monitor the homoplasmic development. Briefly, culture from each variant was mixed in equal volume with Tris-EDTA buffer, and heated for 10 min at 98° C. to lyse the cells. For each lysate, 2 PCR reactions were performed: one with primers that amplify rbcL, and one with primers that amplify aphA6. The aphA6 PCR reaction is able to amplify the gene at an aphA6:rbcL ratio of 1:5000 after 35 cycles. The C. reinhardtii chloroplast is reported to contain approximately 80 copies of genomic DNA. Given the sensitivity described, any lysate that produced an rbcL PCR product and no aphA6 PCR product after 35 cycles of PCR was considered to be homoplasmic for the RuBisCO variant gene.

Once a regenerated rbcL variant was confirmed homoplasmic by PCR, it was scaled up in individual flasks of HSM and then mixed with wild type analog (a GFP-expressing common competitor, the Venus⁺ strain) cells at a ratio of 50:50 and normalized to an OD₇₅₀ of 0.3. The resulting mixture was inoculated into triplicate turbidostats, along with relevant parental and wide type control strains. Media consumption was measured by weighing the media bottles twice daily with replacement whenever necessary. Non-fluorescent cell count from the RuBisCO variants and fluorescent cell count from the GFP-expressing wild type analog (i.e., the Venus⁺ strain) were conducted as described with the Guava flow cytometer. Selection coefficients were calculated as described above.

Microplate Growth Rate Assay (MGRA) with Primary Lines.

A total of 88 Selected Variants isolated from the original turbidostat screen were analyzed by a high-throughput 96-well plate-based assay. Briefly, cultures were grown to stationary phase in HSM and mHSM (modified HSM), which are minimal media with different nitrogen sources (NH₄ for HSM, NO₃ for mHSM). Cultures were diluted to OD₇₅₀=0.2 and grown overnight. Overnight growth was followed by a second dilution to OD₇₅₀=0.02. These initial culture densities put the cells in lag or early log phase. At this point, 0.2 ml of each culture was added to a 96-well microtiter plate in randomized replicates. 96-well microtiter plates used in this assay contain opaque sides so that light exposure is equal across the entire plate, and a transparent base to allow OD acquisition in a 96-well plate reader. Plates were covered using a PDMS (poly dimethyl siloxane) lid in order to allow for gas exchange but minimize culture volume loss to evaporation. Covered plates were then set onto a shaker within a growth chamber supplied with CO₂. Intermittent shaking was set to 25 seconds on at 1700 rpm, 1 second in each direction (CW/CCW) followed by 60 seconds off. Strains were evaluated at two CO₂ levels (5% and 0.2%) and two light conditions (180 μE and 90 μE) in the ambient temperature boxes (^{˜}26° C.). Additionally, strains were grown at 5% CO₂ and 180 μE light in a temperature controlled box at two temperatures (22° C. and 32° C.) to evaluate thermo-stability of the variants. Both HSM and mHSM media were tested in quadruplicate plates in the ambient temperature boxes, whereas HSM medium was used in the temperature controlled box in quadruplicate plates. Thus, a total of 10 growth conditions were evaluated combining various parameters (light/CO₂/temperature/growth media). See Table 27, below.

TABLE 27

| | | | | |
|---|---|---|---|---|
| Temp | Light | [CO₂] | Media | Box |

| | | | | | |
|---|---|---|---|---|---|
| ambient | 180 | μE | 5% | HSM | A |
| ambient | 180 | μE | 5% | mHSM | A |
| ambient | 180 | μE | 0.2% | HSM | A |
| ambient | 180 | μE | 0.2% | mHSM | A |
| ambient | 90 | μE | 5% | HSM | B |
| ambient | 90 | μE | 5% | mHSM | B |
| ambient | 90 | μE | 0.2% | HSM | B |
| ambient | 90 | μE | 0.2% | mHSM | B |
| 32° C. | 180 | μE | 5% | HSM | Controlled |
| 22° C. | 180 | μE | 5% | HSM | Controlled |

OD₇₅₀ was read every 6 hours for a maximum of 120 hours (until the cultures clearly exit logarithmic phase as evidenced by the leveling of the curve). Orientation and location of microplates in the boxes were randomized after each plate reading. The resulting OD₇₅₀ readings, which reflect culture growth, were plotted vs. time. Primary analysis was performed on OD₇₅₀ growth data using a linear region finding algorithm that seeks to maximize the product of Rsq and slope in a regression analysis. Using this model, the growth rate can be estimated by the slope of the graph.

The assay was conducted for all microplates grown in ambient temperature boxes. For microplates grown in the temperature controlled boxes, a further process was employed to calculate difference between Slope₂₂ and Slope₃₂ with the formula,

s₍₂₂₋₃₂₎²=s₂₂²+s₃₂²

where propagation of error was used to obtain variance of the difference, followed with ANOVA Dunnett's post test. Differences in growth rate between the two temperatures were also calculated between each replicate at the lower temperature and each replicate at the higher temperature. These growth rate differentials were compared to determine any differences in temperature sensitive growth between the variants and wild type.
Western Blot Assay with Primary Lines.

Approximately 3 ml of samples of each original line were taken from a 24-well plate at early log phase (OD₇₅₀=0.2), and pelleted by centrifugation. Total soluble protein was extracted from the pellet using Bug Buster Protein Extraction Reagent (Nanogen) per manufacturer instructions. The amount of total protein was determined with albumin (BSA) standards using a Pierce BCA Protein Assay Kit (Thermo Scientific). Samples were then normalized to 1.5 mg/ml and a 3-point serial dilution (15 μg, 7.5 μg and 3.75 μg of total protein) of each normalized sample was made in Bug Buster Extraction Reagent (Novagen). Samples were then denatured at 95° C., and equal volumes were loaded onto 12% polyacrylamide Bis-Tris gels (Bio-Rad), along with 0.5 μg of purified RuBisCO protein (Agrisera). Following electrophoresis in MES running buffer, protein bands were transferred to a PVDF membrane. Membranes were incubated with a blocking solution followed by a rabbit anti RuBisCO large subunit antibody (Agrisera) and rabbit anti ribosomal L30 antibody (Agrisera). Finally, membranes were incubated with an HRP-conjugated goat anti-rabbit secondary antibody (Agrisera) and developed using SuperSignal West Dura Extended Duration Substrate (Thermo Scientific). Average pixel intensity (API) for each band was quantified using FluorChem software (Alpha Innotech). Using the ROUT method in GraphPad Prism, an outlier analysis was performed on the API ratio between L30 and the purified RuBisCO control (L30/ctrl), which should be consistent across all samples and gels. Then the API ratio between RuBisCO and ribosomal L30 (RBCL/L30) was calculated for each sample at each dilution and was used to compare relative levels of RuBisCO protein abundance across all the gels.

### Validation Results

### Primary Line Competitions.

Turbidostat 1:1 Competition.

99 original lines were successfully isolated from algae clones derived from screening. All 99 lines were competed against a wild type analog in turbidostats. The remaining variant (SSM205) was not recovered from the initial screening experiment and was therefore advanced directly into the cloning and regeneration steps.

Two rounds of 49 and 50 winners each, along with wild type and wild type-complemented knockout strains, were set up. One of the SSM parental variants, R83H, was included in the first setup round because all of the variants were from the same parental background. For SSM libraries, each amino acid in the C. reinhardtii RuBisCO large subunit protein (RBCL) was substituted for 9 amino acids representing different classes of side-chain chemistries. These include the positively charged amino acids histidine (H) and lysine (K), negatively charged aspartic acid (D), polar neutral serine (S) and glutamine (Q), hydrophobic leucine (L) and tyrosine (Y), small flexible glycine (G), and small rigid proline (P). All three SSM parental variants (R83H, I87Y, R312K) were included in the second round because the variants were from a mix of parents. The NNK parentals (E88S and R83Q) were only included in the first round because all of the NNK variants were run in the first round. For NNK libraries, a unique oligonucleotide was designed with the degenerate sequence NNK (N=any nucleotide; K=G or T) substituted for the wild type codon. The NNK sequence encodes 32 of the possible 64 codons, encompassing all 20 amino acids as well as a stop codon. Each winner (or control) was mixed 1:1 with the Venus⁺ strain and inoculated into triplicate turbidostats. All competitions were run for at least 8 days, and samples were taken twice during that time. For each winner and control, Guava ratios were used to calculate sᵣₑₚ and s_{avg} values relative to the Venus⁺ strain.

The selection coefficient, s, of each replicate in the validation 1:1 competition was compared to the s_{avg} values for wild type, wild type-complemented knockout, and the parental line controls (where applicable) to generate three Δs_{avg} values, where Δs_{avg}(winner)=s_{avg}(winner)−s_{avg}(control). Δs_{avg}>0 implies that a winner line would outperform the control if they were competed head-to-head. Wild type and the complemented knockout were run in all setup rounds, and therefore winner lines were compared to the wild type/wild type-complemented controls in their respective round. Parental controls were run in their respective rounds, and therefore winner lines were compared to the parental controls in their respective round.

It should be noted that in these experiments the wild type and wild type complemented strains both have negative Δs_{avg} values when competed against the wild type analog, and therefore all comparisons are made to the wild type complemented selection coefficient rather than the arbitrary s value of zero. Two of the original lines (SSM094 and SSM088) have an average Δs value significantly greater than both the wild type complemented (p<0.01) and wild type (p<0.05) (ANOVA with Dunnett's post test). Two additional variants, SSM120 and NNK061, have an average Δs value significantly greater than the wild type complemented (p<0.05). There are 15 variants that have a mean Δs value higher than the mean value for both wild type and the wild type complemented. There are 55 variants that have a mean Δs value higher than the mean value for wild type complemented alone, including 22 variants that outperform the wild type complemented for all replicates (see Table 28 below).

TABLE 28

| Variant | Mean Δs | Std Dev | p-Value |
|---|---|---|---|
| SSM094 | 0.182 | 0.063 | <.0001* |
| SSM088 | 0.174 | 0.055 | <.0001* |
| SSM120 | 0.088 | 0.078 | 0.0444* |
| NNK061 | 0.087 | 0.059 | 0.0471* |
| SSM138 | 0.079 | 0.020 | 0.0992 |
| SSM043 | 0.076 | 0.040 | 0.1307 |
| WT | 0.075 | 0.062 | 0.0458* |
| SSM155 | 0.075 | 0.032 | 0.1406 |
| SSM036 | 0.075 | 0.060 | 0.1468 |
| NNK069 | 0.069 | 0.035 | 0.2334 |
| SSM273 | 0.068 | 0.044 | 0.2438 |
| SSM057 | 0.065 | 0.013 | 0.292 |
| SSM003 | 0.056 | 0.040 | 0.5255 |
| SSM125 | 0.054 | 0.016 | 0.5667 |
| SSM064 | 0.052 | 0.024 | 0.6337 |
| SSM080 | 0.052 | 0.026 | 0.6376 |
| SSM101 | 0.051 | 0.033 | 0.6633 |
| SSM146 | 0.046 | 0.021 | 0.7802 |
| SSM145 | 0.044 | 0.021 | 0.8464 |
| SSM191 | 0.043 | 0.010 | 0.8532 |
| SSM136 | 0.035 | 0.019 | 0.9692 |
| SSM172 | 0.033 | 0.012 | 0.982 |
| SSM154 | 0.015 | 0.007 | 1 |
| WT-comp | 0.000 | 0.015 | 1 |

Turbidostat En Masse Competition.

The original line versions of all 99 Selected Variants were competed en masse in turbidostats for three weeks. 51 of the 99 variants were identified in the final sort sequencing. Two of those 51 variants were not identified in the baseline sequencing and were assumed to have a starting ratio of 1/99. SSM209 was dominant in this experiment and largely took over in all replicate turbidostats.

### Regenerated Line Competitions.

1:1 Competition Data.

Of the 100 Selected Variants, 11 lines showed a lower selection coefficient in all replicates relative to the wild type complemented control. These 11 were not advanced further in the validation process. The remaining 89 variants were successfully regenerated. Two of the regenerated lines (rSSM191 and rSSM209) were not driven to the homoplasmic state within weeks (vs. days for a typical line) and therefore were not processed for 1:1 turbidostat competitions. The remaining 87 rbcL variants were competed 1:1 against a wild type analog strain in triplicate turbidostats as previously described. Also included in the experiment as controls were wild type, wild type complemented and parental strains upon which the Selected Variants were built. In order to compare results across experiments for each variant, Δs values were calculated by subtracting the s_{avg} value of the wild type complemented strain from the s value of each replicate. The calculated Δs values, relative to the mean of the wild type complemented strain, are shown in FIG. 18, where Δs=0 represents the average performance of wild type complemented control versus the common competitor. There are 54 variants with a mean Δs value higher than 0. Among them, 26 variants have a Δs, WT-comp>0 in all the three replicates analyzed. It is important to note that, while the wild type tends to have a higher selection coefficient than that of the wild type complemented strain, in one of the regenerated line 1:1 competition runs, the s values of wild type dropped to the same level of the wild type complemented. In all, 23 of the 54 lines were also identified as winners over the wild type (i.e., Δs, WT>0) in 1:1 competitions of regenerated lines.

Table 29 shows data for the top 26 regenerated lines where all replicates were above the wild type complemented s_{avg}. Two of the regenerated lines, rSSM094 (which is also a significant winner during the original line competitions) and rSSM136, have average Δs values significantly greater than the wild type complemented control (ANOVA with Dunnett's post test, p<0.05) versus the wild type analog. While the remaining 24 lines did not demonstrate a significantly increased competitive advantage over wild type or wild type complemented strains, all have mean s values and all replicate s values higher than the mean value for wild type complemented control.

TABLE 29

| Variant | Mean Δs | Std Dev | p-Value |
|---|---|---|---|
| rSSM136 | 0.137 | 0.129 | 0.0158* |
| rSSM094 | 0.127 | 0.090 | 0.0330* |
| rSSM218 | 0.120 | 0.070 | 0.0524 |
| rSSM131 | 0.105 | 0.039 | 0.1342 |
| rSSM139 | 0.104 | 0.065 | 0.1469 |
| rSSM038 | 0.099 | 0.103 | 0.1967 |
| rSSM154 | 0.097 | 0.027 | 0.2202 |
| rSSM101 | 0.096 | 0.022 | 0.2268 |
| rSSM194 | 0.092 | 0.042 | 0.2773 |
| rSSM190 | 0.077 | 0.028 | 0.5442 |
| rSSM036 | 0.076 | 0.037 | 0.5684 |
| rSSM173 | 0.071 | 0.032 | 0.6582 |
| rSSM166 | 0.070 | 0.040 | 0.6929 |
| rSSM164 | 0.069 | 0.045 | 0.7034 |
| rSSM151 | 0.058 | 0.034 | 0.8981 |
| rSSM080 | 0.055 | 0.039 | 0.9294 |
| rSSM023 | 0.055 | 0.023 | 0.9315 |
| rSSM003 | 0.050 | 0.040 | 0.9692 |
| rSSM145 | 0.048 | 0.034 | 0.9813 |
| rNNK001 | 0.047 | 0.044 | 0.9853 |
| rSSM213 | 0.047 | 0.016 | 0.9853 |
| rSSM138 | 0.044 | 0.055 | 0.9929 |
| rSSM050 | 0.037 | 0.030 | 0.9992 |
| rSSM004 | 0.035 | 0.017 | 0.9996 |
| WT | 0.024 | 0.050 | 1 |
| rSSM074 | 0.021 | 0.007 | 1 |
| rSSM064 | 0.020 | 0.007 | 1 |
| WT-comp | 0.000 | 0.067 | 1 |

### Validated Variants

There were 34 Selected Variants that had a mean selection coefficient above that of the wild type complemented strain (i.e., Δs, WT-comp>0) during 1:1 competitions of both original and regenerated lines. Since the 34 variants repeatedly demonstrated growth advantage over wild type and/or wild type complemented control strain in turbidostat experiments, and sometimes across all replicates, they were considered validated.

### Growth and Biochemical Characteristics.

Microplate Growth Rate Assay.

The 88 original lines were analyzed in microtiter plates as described above to test whether mutants that have high s in turbidostats have improved growth characteristics in another format. Assaying RuBisCO variants in changing temperature, CO₂, and light conditions provided an indication that changes to the RuBisCO protein were responsible for increased yield over wild type.

As carbon fixation and overall Rubisco function are known to be impacted by temperature, the growth performance of the Selected Variants was assayed at two temperatures to give an indication of the potential temperature dependence of any predicted yield increase. The OD_{750 nm} vs. time data was analyzed using a Linear Region Algorithm comparing slopes (growth rates) across strains by temperature. Only one strain, NNK061, resulted in a statistically significantly greater slope than WT-complemented at 32° C. To further investigate any improvement from the mutants, the differences between the slopes at 22° C. and 32° C. were analyzed using WT-complemented as a control. First, a t-test was performed comparing the slopes from 22° C. and 32° C. directly for each strain. The t-test showed no statistical difference in growth rate at the two temperatures for both WT and WT-comp, therefore any strain with a significant negative value would indicate an improvement in growth at 32° C. This test resulted in 4 strains showing statistically significant improvement at 32° C. Another analysis was performed using a difference measure, Diff(22-32), between the two temperatures. These differences were compared to WT-comp, and resulted in the same 4 strains demonstrating a high temperature improvement. A third approach used combinatorial differences between 22° C. and 32° C., leading to two strains (SSM155 and NNK061) that were also identified in the previous two analyses. A list of variants that exhibit significant growth improvement at 32° C. vs. 22° C. is summarized in Tables 30 and 31.

TABLE 30

| 32° C. | t-test | Diff(22-32) | Comb_diff |
|---|---|---|---|
| NNK061 | NNK061 | NNK061 | NNK061 |
| | SSM155 | SSM155 | SSM155 |
| | SSM203 | SSM203 | |
| | SSM252 | SSM252 | |
| | E88S | E88S | E88S |
| | R83H | R83H | R83H |
| | R312K | R312K | R312K |

TABLE 31

| | | Std | Std | |
|---|---|---|---|---|
| Strain | Diff(22-32) | Dev_Diff | Err_Diff | p-Value |
| E88S | −0.0053 | 0.0012 | 0.0004 | <.0001 |
| R83H | −0.0048 | 0.0015 | 0.0006 | <.0001 |
| R312K | −0.0047 | 0.0028 | 0.0011 | <.0001 |
| SSM155 | −0.0037 | 0.0033 | 0.0013 | 0.0003 |
| NNK061 | −0.0027 | 0.0037 | 0.0014 | 0.0048 |
| SSM203 | −0.0018 | 0.0013 | 0.0005 | 0.034 |
| SSM252 | −0.0016 | 0.0034 | 0.0013 | 0.049 |
| WT | 0.0021 | 0.0029 | 0.0011 | 1 |
| WT-comp* | 0.0033 | 0.0041 | 0.0016 | 1 |

A summary of statistically significantly different slopes (growth rates) from the linear finding algorithm for strains compared with wild type in ambient temperature boxes is presented in the Table 32. Cells marked with “Hi” indicate statistically significantly higher slopes compared with wild type complemented control. No variants from the 0.2% CO₂ conditions were found to have a significantly improved growth.

TABLE 32

| % CO₂, Light | 0.2,180 | 0.2,180 | 0.2,90 | 0.2,90 | 5,180 | 5,180 | 5,90 | 5,90 |
|---|---|---|---|---|---|---|---|---|
| Media | HSM | mHSM | HSM | mHSM | HSM | mHSM | HSM | mHSM |
| NNK001 | | | | | | Hi | | |
| NNK003 | | | | | Hi | | | |
| SSM030 | | | | | | Hi | | |

Western Blot.

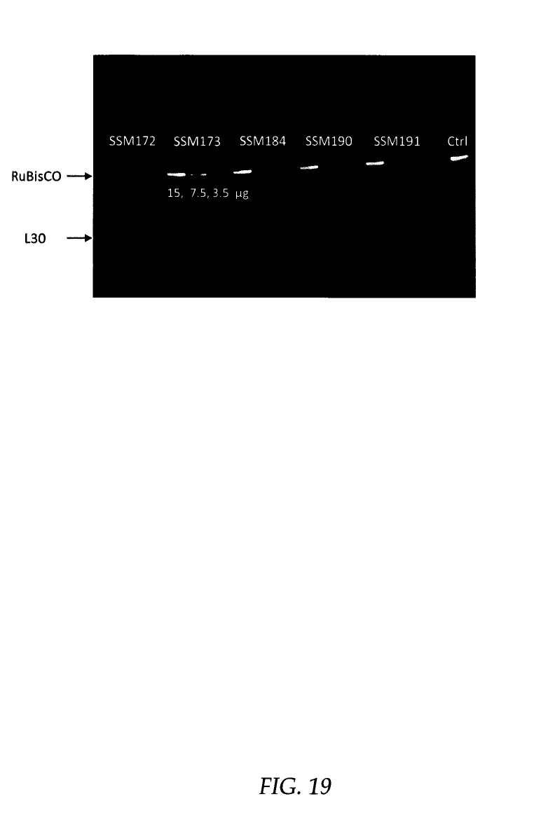

The 88 original lines were assayed for RuBisCO protein levels by Western Blot. The number “88” was the result of subtracting the 11 lines with a lower selection coefficient in all replicates, and 1 line that was not recovered, from the original 100 Selected Variants. 18 protein gels were run in total, each with 5 original line protein samples and one purified RuBisCO control. Each sample was loaded at three dilutions (see example FIG. 19). Spot densitometry was used to quantify the average pixel intensity (API) of each 52 kDa band after background correction, and the ratios between the RuBisCO and the L30 bands were calculated for each dilution. The calculated API ratio for each original line sample was compared to each other as well as to the wild type complemented strain. 12 samples had significantly lower mean API ratios than the wild type complemented strain based on ANOVA with Dunnett's post test (p<0.05). It is interesting to note that 7 of the lines with lower protein by Western are in the list of validated variants. Some apparent outliers (e.g., those with extremely low L30/RBCL ratios) not filtered by ROUT analysis were manually removed. The RuBisCO/L30 API ratios for each RuBisCO variant from all Western Blots, after filtering the outliers, are shown in Table 33.

TABLE 33

| Variant | Mean RbcL/L30 | Std Dev | p-Value |
|---|---|---|---|
| SSM049 | 0.594 | 0.334 | 0.0053* |
| SSM143 | 0.670 | 0.431 | 0.0066* |
| SSM136 | 0.741 | 0.200 | 0.0080* |
| SSM139 | 0.834 | 0.744 | 0.0103* |
| SSM137 | 0.926 | 0.443 | 0.0132* |
| SSM138 | 0.970 | 0.127 | 0.0148* |
| SSM094 | 0.978 | 0.304 | 0.0152* |
| SSM050 | 1.097 | 0.678 | 0.0206* |
| SSM057 | 1.100 | 0.433 | 0.0208* |
| SSM142 | 1.171 | 0.725 | 0.0248* |
| SSM166 | 1.380 | 0.678 | 0.0411* |
| SSM125 | 1.387 | 0.531 | 0.0417* |
| WT-comp | 5.499 | 2.616 | 1.0000 |

### Validation Summary

Based on the process of wild type competition and regeneration of transgenic lines, 34 Selected Variants were validated as having a competitive growth advantage due to expression of the RuBisCO variant. Additional confirmatory data was generated from physiological and biochemical assays of the Selected Variants. The validated variants and a summary of the data are listed in the Table 34.

TABLE 34

| | | | Expt 2 | Exp 1 | En Masse | Original | Regenerated | Low Δr, | | |
|---|---|---|---|---|---|---|---|---|---|---|
| Winner ID | Constant | Var | Cat | Winner | Winner | Δs | Δs | 22°-32° C. | r | W. Blot low |
| NNK061 | N/A | A315S | 2 | | | 0.087 | 0.053 | Yes | Yes | |
| NNK069 | N/A | A317T | 3 | A317G | | 0.069 | 0.009 | | | |
| | | | | (WR4601) | | | | | | |
| SSM003 | R83H | E6G | 1 | | | 0.056 | 0.050 | | | |
| SSM004 | R83H | K8Y | 1 | | | 0.007 | 0.035 | | | |
| SSM006 | R83H | A11L | 1 | | | 0.067 | 0.139 | | | |
| SSM023 | R83H | I36L | 3 | I36L | | 0.001 | 0.055 | | | |
| | | | | (WR0605) | | | | | | |
| SSM031 | R83H | C53L | 5 | | | 0.038 | 0.004 | | | |
| SSM036 | R83H | I87L | 1 | I87Y | | 0.075 | 0.076 | | | |
| | | | | (WR1302) | | | | | | |
| SSM042 | R83H | P89G | 3 | | | 0.007 | 0.026 | | | |
| SSM049 | R83H | E93H | 1 | | | 0.006 | 0.059 | | | Yes |
| SSM064 | R83H | N95Q | 2 | | | 0.052 | 0.020 | | | |
| SSM080 | R83H | V145G | 2 | | | 0.052 | 0.055 | | | |
| SSM088 | R83H | G168P | 1 | | Yes | 0.174 | 0.001 | | | |
| SSM094 | R83H | T200S | 4 | | | 0.182 | 0.127 | | | Yes |
| SSM101 | R83H | A230Y | 1 | | | 0.051 | 0.096 | | | |
| SSM131 | R83H | V341Q | 1 | | | 0.034 | 0.105 | | | |
| SSM136 | R83H | E355L | 3 | | | 0.035 | 0.137 | | | Yes |
| SSM137 | R83H | E355Y | 1 | | | 0.030 | 0.040 | | | Yes |
| SSM138 | R83H | E355Q | 1 | | | 0.079 | 0.044 | | | Yes |
| SSM139 | R83H | E355S | 3 | | | 0.000 | 0.104 | | | Yes |
| SSM145 | R83H | S359L | 1 | | | 0.044 | 0.048 | | | |
| SSM146 | R83H | S359G | 3 | | | 0.046 | 0.071 | | | |
| SSM154 | R83H | D367Q | 4 | | | 0.015 | 0.097 | | | |
| SSM155 | R83H | D367S | 4 | | | 0.075 | 0.019 | Yes | | |
| SSM165 | R83H | E392D | 1 | | Yes | 0.076 | 0.042 | | | |
| SSM166 | R83H | R439Y | 1 | | | 0.021 | 0.070 | | | Yes |
| SSM169 | R83H | K450Y | 5 | | | 0.037 | 0.011 | | | |
| SSM172 | R83H | E460Q | 4 | | | 0.033 | 0.011 | | | |
| SSM184 | I87Y | P46Q | 2 | | | 0.033 | 0.053 | | | |
| SSM190 | I87Y | P89G | 2 | | Yes | 0.108 | 0.077 | | | |
| SSM215 | I87Y | V255K | 2 | | | 0.006 | 0.026 | | | |
| SSM218 | I87Y | G272S | 2 | | | 0.089 | 0.120 | | | |
| SSM252 | R312K | A11S | 4 | | | 0.018 | 0.012 | Yes | | |
| SSM296 | R312K | E355P | 6 | E355P | Yes | 0.074 | 0.029 | | | |
| | | | | (WR5101) | | | | | | |

KEY
Constant: parental variant
Var: identified variant
Expt 2 Cat: category from Example 2 screening
Expt 1 Winner: Example 1 validated winner at same position
En Masse Winner: dominant in final sort of en masse turbidostats
Δs: selection coefficient (variant)-selection coefficient (wild type complemented)
Low Δr, 22-32° C.: microplate growth advantage at 32° C. (significant = Yes)
r: microplate growth rate (slope) (significant = Yes)
W. Blot low: significant = Yes

### Example 3

### Screening

In this experiment, two approaches were taken to generate mutagenic libraries. In one approach, Site Saturation Mutagenesis (SSM) (U.S. Pat. No. 5,830,650) was used to combine the top double mutation validated in Example 2 (R83H, T200S, aka SSM094) with a highly diverse set of third mutations evenly distributed across the protein. The result was a SSM library of approximately 4,200 triple mutants, with the two previously identified advantageous mutations held constant.

In a second approach, the top 27 amino acid substitutions from Examples 1 and 2 were systematically combined to create a comprehensive library of double and triple mutations across the protein. These top 27 mutations represent 18 different amino acid positions across the protein and were selected as follows. First, the top validated variants from Example 2 were chosen. Next, the top novel mutations from validated variants from Example 2 were chosen. Finally validated variants with mutations at sites of structural interest (e.g. putative RuBisCO activase interactions), as determined by structural analysis, were chosen. Five amino acid positions chosen were validated with multiple substitutions; therefore all of the substitutions found at that amino acid were included. A mock mutation of the start codon (methionine to methionine) served as a 28^{th} site to include all possible double mutants. See the Table 35 for a list of all amino acid residues mutated and the respective selection criteria. The result was a triple combo library of 2906 mutants.

TABLE 35
Triple Combo Mutations

| Amino | | Substi- | | |
|---|---|---|---|---|
| Acid | Original | tution | Final | |
| position | AA | AA | Mutation | Selection Criteria |
| 1 | M | M | M1M | |
| 6 | E | G | E6G | Top Variant Validated Ex 2 |
| 11 | A | S | A11S | Top Variant Validated Ex 2 |
| 11 | | L | A11L | |
| 28 | D | H | D28H | Top Variant Validated Ex 1 |
| 36 | I | L | I36L | Top Variant Validated Ex 1 |
| 46 | P | Q | P46Q | RuBisCo structural interest |
| 83 | R | Q | R83Q | Top Variant Validated Ex 1 |
| | | H | R83H | |
| 87 | I | Y | I87Y | Top Variant Validated EX 1 |
| | | L | I87L | |
| 89 | P | G | P89G | RuBisCo structural interest |
| 93 | E | H | E93H | RuBisCo structural interest |
| 95 | N | Q | N95Q | Top Variant Validated Ex 2 |
| 145 | V | G | V145G | Top Variant Validated Ex 2 |
| 200 | T | S | T200S | Top Variant Validated Ex 2 |
| 230 | A | Y | A230Y | Top Variant Validated Ex 2 |
| 272 | G | S | G272S | Top Variant Validated Ex 2 |
| 312 | R | K | R312K | Top Variant Validated Ex 1 |
| 355 | E | Q | E355Q | Top Variant Validated Ex 2 |
| | | P | E355P | |
| | | Y | E355Y | |
| | | S | E355S | |
| | | L | E355L | |
| 359 | S | G | S359G | Top Variant Validated Ex 2 |
| | S | L | S359L | |
| 367 | D | Q | D367Q | Top Variant Validated Ex 2 |
| | | S | D367S | |

For SSM libraries, each amino acid in the C. reinhardtii RuBisCO large subunit protein (RBCL) was substituted for 9 amino acids representing different classes of side-chain chemistries (as in previous Examples). These include the positively charged amino acids histidine (H) and lysine (K), negatively charged aspartic acid (D), polar neutral serine (S) and glutamine (Q), hydrophobic leucine (L) and tyrosine (Y), small flexible glycine (G), and small rigid proline (P).

For the SSM094 parental sequence, 68 individual libraries were generated representing unique 7-amino acid segments of the RBCL protein referred to as Regions. Therefore the mutations at amino acid positions 1-7 were generated in the Region 1 library, mutations for amino acids 8-14 were generated in Region 2, and so forth. These 68 regions cover the entire protein. To generate the mutations, DNA oligonucleotides covering portions of the rbcL gene were synthesized, each containing a single codon change to produce the desired amino acid substitution. Oligonucleotides for each region were ordered in a plate array according to the reference amino acid position and the respective substitution.

Each 63-mer oligonucleotide encompasses the 21 nucleotide region where the mutagenic codon resides, along with 21 nucleotides at both the 5′ and 3′ ends identical to the parental sequence (C. reinhardtii rbcL with R83H T200S double point mutation) flanking that region. In addition, a single non-mutagenic oligonucleotide was designed for each region on the opposite DNA strand, such that 21 nucleotides of homology exist between each mutagenic oligonucleotide and its non-mutagenic counterpart.

Mutations were incorporated into the rbcL gene using an overlap PCR technique. Briefly, each mutagenic oligonucleotide was used as a primer in a PCR reaction with another primer at the opposite end of the gene, along with a template plasmid containing the parental rbcL sequence. Primers are incorporated into the resulting PCR products, thereby introducing the mutagenic codon at one end of the amplicon. A second PCR is carried out in a similar fashion using the complementary non-mutagenic oligonucleotide as a primer to amplify the portion of the gene not covered by the first reaction. In a third PCR, purified amplicons from each of the previous reactions are combined with primers that flank the rbcL gene to both amplify the assembled variant and add the restriction endonuclease sites NdeI and SpeI to the 5′ and 3′ ends, respectively. Homology between the amplicons derived from the initial PCRs allows the entire gene to be assembled without the use of a traditional template.

For construction of the Triple Combo variant library a simple PERL script was used to create all possible three variant combinations of the 27 selected substitutions. In order to also create all double combinations, M1M was used as one of the input “substitutions” so that any triple combo including M1M was essentially a double mutant. Given that some positions had more than one possible substitution, some combinations of three variants are not possible (e.g. E355P and E355S cannot be combined).

For the SSM library, full-length amplicons were digested with NdeI and SpeI, and ligated into the C. reinhardtii chloroplast transformation vector pSC179 (FIG. 1). The vector contains approximately 2.8 kb each of 5′ and 3′ flanking sequence homology to the rbcL locus in the C. reinhardtii chloroplast genome, as well as elements for propagation and selection in bacteria.

Once the SSM libraries were ligated into the vector, they were transformed into bacteria for amplification and QC. Resulting bacterial colonies for each library (median n>1,500) were scraped into liquid cultures and plasmid DNA was purified the following day. For the SSM library, equimolar plasmid DNA was combined into Pools of 2 regions each, such that each SSM library was divided into 34 Pools. The Triple Combo library Pools were determined by randomly selecting 96 mutants in a lottery fashion to create 30 Pools. Those 30 Pools were then systematically randomized to create an additional 30 secondary Pools. The same process was used to create 6 additional Pools of 34 mutants each for a final total of 66 Pools (see Table 36). Thus each TC variant was screened twice in two independent, randomized Pools.

TABLE 36

| SSM | | | Library |
|---|---|---|---|
| Pool | Regions | #AA's | Complexity |
| 1 | 1-2 | 13 | 117 |
| 2-33 | 3-66 | 14 | 126 |
| 34 | 67-68 | 13 | 117 |

| Triple | |
|---|---|
| Combo | Library |
| Pool | Complexity |
| 1-60 | 96 |
| 61-66 | 34 |

The distribution of mutations in the SSM library was determined by amplifying each mutagenic portion of the gene with uniquely barcoded PCR primers, followed by Ion Torrent next-generation sequencing (NGS). Dual sets of primers with unique barcodes were designed for each Pool to produce amplicons of ^{˜}200 bp (not including adapters or barcodes). The primer sets for each amplicon were identical with the exception that the adapter/barcode on the Forward primer of one set was attached to the Reverse primer of the other set, and vice-versa, to allow for bi-directional sequencing. These primer sets were used to amplify DNA from each of the plasmid libraries. PCR products from each library were combined and sequenced on an Ion Torrent 318 chip with 200 bp chemistry. Deconvoluted data for each barcode was then mapped to the reference rbcL sequence to determine the number of reads containing each of the expected variants. Each barcode comprised reads from both directions; Forward reads were used from the first codon of the mutagenic region to the mid-point of the amplicon, and Reverse reads were used from the mid-point of the amplicon to the last codon of the mutagenic region. Sequencing errors due to insertions, deletions, early terminations, etc. were excluded from the analysis, and therefore the total number of sequences at each codon position varies across an amplicon. To normalize the counts for each variant, the raw number of reads was multiplied by the correction factor Sum_{BC}/Sumₚₒₛ, where Sum_{BC} is the total number of reads for the barcode (amplicon), and Sumₚₒₛ is the total number of error-free reads at that codon position. A noise threshold was established for NGS data as the maximum observed frequency for a variant that is known to be nonexistent in the library. In this case, the 9 variants for the Start codon (which was not mutagenized) were used.

Given an equal distribution of mutant sequences within an SSM barcode, the parental (reference) codon will be present 13 out of 14 times in the Pool of interest, making up approximately 93% of the reads on a codon-by-codon basis. Therefore, the number of parental sequences must be estimated with a different approach than the codon focused method used for variant counts. In the plasmid libraries, each nucleotide in a read was marked as “likely reference” if it was (i) identical to the reference sequence, (ii) called as a deletion, or (iii) not covered by that particular read. Reads containing 14/14 “likely reference” nucleotides in the region of interest were counted as parental sequences.

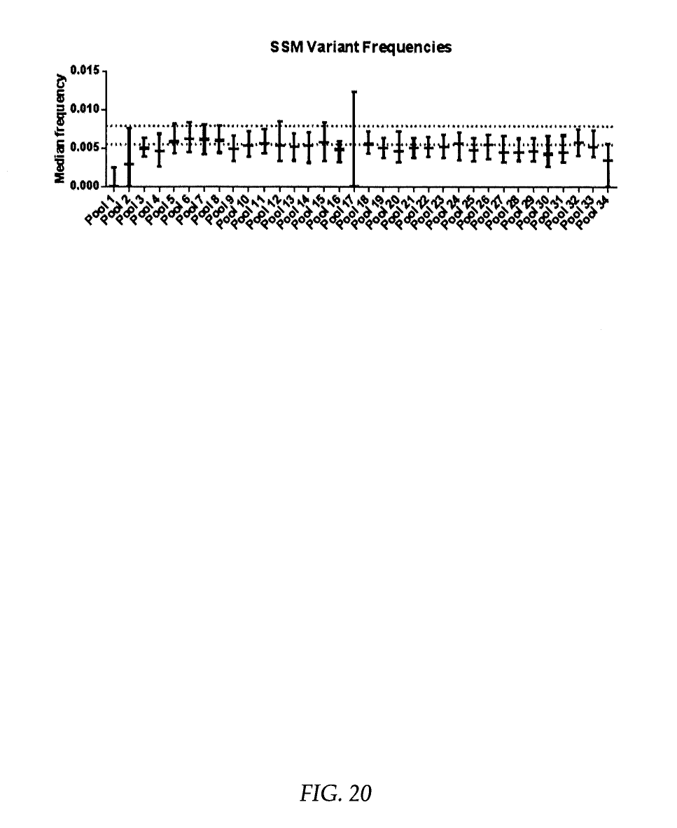

The distributions of mutant frequencies, along with parental sequences for the SSM library, are shown in FIG. 20. For each SSM Pool the median frequencies and interquartile ranges of all expected mutants are shown in the top graph. Given a perfect distribution within the SSM libraries, each variant would represent 1/126 (0.79%) or 1/117 (0.854%) of the sequences in Pools 2-32 or Pools 1, 34. These ranges are indicated by the dotted lines.

Based on the NGS sequencing results, 89% of the expected mutants were detectable above the baseline noise level in the SSM plasmid library. Note that the 9 substitutions of the start codon ATG were not present in the SSM libraries because that codon is part of the cloning site NdeI. The other variants that were not detected were simply not created during the PCR process or are below the level of detection for the sequencing method. Additionally, Pools 1 and 17 had very low sequence counts for windows 2 and 34 respectively which brought down the overall percentage of expected mutants detected. Parental sequence was present at 29% on average across all SSM libraries.

### Primary Turbidostat Screening.

A RuBisCO large subunit knockout (ΔrbcL) algal strain was generated by transforming the chloroplast genome of wild type C. reinhardtii cells via gold particle bombardment with a vector containing the kanamycin resistance gene aphA6 flanked by 5′ and 3′ homology to the rbcL locus. Since transformation of the chloroplast genome occurs by homologous recombination in the C. reinhardtii chloroplast, selection of kanamycin resistant clones is indicative of rbcL displacement in one or more copies of the chloroplast genome. Continually passaging transformants on selective media resulted in homoplasmic knockout clones where no copies of rbcL were detectable by PCR. Since the knockout strain is non-photosynthetic it requires an organic carbon source such as acetate to grow. DNA from each rbcL variant library was transformed into the chloroplast genome of ΔrbcL C. reinhardtii cells via gold particle bombardment. Again, homologous recombination results in replacement at the rbcL locus, this time replacing the aphA6 kanamycin marker with rbcL variants. Selection for rbcL complementation was carried out on HSM agar, a minimal medium that necessitates obligate photoautotrophic growth. Any rbcL variants that are not present in these transformant lines are likely non-functional forms of RuBisCO inactivated by the introduced amino acid substitution.

Transformed algal colonies for each SSM and Triple Combo Pool were counted, and colonies necessary for 3-5× coverage of the mutants were scraped into flasks containing TAP media en masse. One to three days following flask inoculation, cells were passaged to new flasks, and then inoculated into quadruplicate (SSM) or triplicate (Triple Combo) turbidostats two to four days later. The turbidostats were filled with HSM media and set to an OD₇₅₀ of approximately 0.3, which represents an early- to mid-log growth phase. Light of ^{˜}150 μEinstein was provided for 16 hours a day with 8 hours of dark (diurnal cycling), with a constant stream of 0.2% CO₂ bubbling into the culture. Cultures were monitored at least daily for media replenishment, CO₂ delivery, culture settling, cell sticking, mechanical failure or any other issues. The cultures were grown under these optimal photoautotrophic conditions for four weeks. Samples were taken from the inoculum flasks and subsequently from each turbidostat at 7 day intervals starting at day 14, and single cells were sorted by fluorescence-activated cell sorting (FACS) into 96-well plates containing TAP media. Cell lysates were also prepared from each sample for DNA sequencing. After a week or more of growth, sorted strains were replicated onto solid media for longer-term recovery and isolation of transformed lines.

SSM Sequencing and Analysis from Primary Turbidostat Screening

After 5-7 days of growth in 96-well plates, the individual sorted strains were used as template in a PCR reaction that amplified the rbcL gene based on gene specific primers. After ascertaining success in producing a single product from the reactions, PCR products were treated for sequencing with Exonuclease I/Shrimp Alkaline Phosphatase (ExoSAP) and sequenced via Sanger chemistry. For the SSM library a single primer reading into the region of interest was used. These clonally isolated and sequenced variants are used as a check on the NGS data, but are primarily for the identification and isolation of particular clones of interest for the secondary screening and validation processes.

For the SSM library, the distribution of mutants in each turbidostat was determined by amplifying the region of interest from cell lysates with uniquely barcoded PCR primer sets, followed by Ion Torrent next-generation sequencing (similar to the plasmid library sequencing described above). Barcoded amplicons from the baseline inoculums and all final replicates were combined together on separate Ion Torrent 318 chips, such that the baseline, final “A” replicates, final “B” replicates, and so forth were sequenced together. Analysis of the NGS data from turbidostat algae samples was similar to that described earlier for the plasmid library analysis. Based on Example 2 analysis of detection of “non-existent” STOP codons, all ratios of 0.002 or below were considered noise (i.e. not distinguishable from zero).

Sequences were analyzed from each turbidostat replicate at beginning and ending time points, with the difference being baseline (time 0) data, which was analyzed per set and then used as the starting point for each turbidostat replicate of that set.

Hit counts and total sequences were used to calculate the ratio of each variant present in a given time point. These numbers can then be used to calculate a selection coefficient using the formula given previously. As explained previously, the selection coefficients used in this analysis do not conform strictly to some of the assumptions upon which the formula is based, in that this is not a single clone compared against a uniform population. Each clone is compared to the rest of the population, which itself is made up of many other clones. However, within the experiment, the calculated selection coefficients provide a valid way to compare and rank potentially winning clones.

In many cases, a given sequence was identified at one time point but not detected in another time point. As the natural log of zero produces an error, assumptions were necessary in such cases. When analyzing the SSM library, any instance where the baseline was zero (i.e. below the noise threshold ratio of 0.002) but the variant was detected at the endpoint, a value of 1/c was assigned to the baseline ratio, where c equals the number of colonies that were scraped into the inoculum flask. For the more common case where the variant was detected at baseline but not at the endpoint (termed “extinct” variants), a value of 0.0001 was assigned to the endpoint ratio resulting in a large negative selection coefficient, but avoiding the calculation error. During the analysis, these assumptions were monitored to avoid consideration of artifactual data.

In order to successfully identify and recover winning clones from the primary screen, a formula was used to estimate the length of time required for competition and the number of clones to analyze in order to reach a desired level of sensitivity. Assuming the minimum 1-in-126 starting ratio for SSM libraries, approximately 200 sequences at the endpoint and a sensitivity of 2.5% (i.e. 5 sequences out of 200), the time necessary to identify a clone with a selection coefficient of 0.05 can be calculated as follows:

$$ln ( \frac{5}{195} ) = ln ( \frac{1}{126} ) + 0.05 {d}^{- 1} \cdot t d ; t = 23.4 d$$

Thus in the primary screen, an s value of approximately 0.05 d⁻¹ should be detectable within ^{˜}3 weeks of growth by sequencing approximately 200 clones. Based on this and our previous experience, a 4 week timeline was used in this Example.

Triple Combo Sequencing and Analysis from Primary Turbidostat Screening.

After 5-7 days of growth in 96-well plates, the individual sorted strains were used as template in a PCR reaction that amplified the rbcL gene based on gene specific primers. After ascertaining success in producing a single product from the reactions, PCR products were treated for sequencing with Exonuclease I/Shrimp Alkaline Phosphatase (ExoSAP) and sequenced via Sanger chemistry. For the Triple Combo library two primers covering the mutation window across the gene were used. These clonally isolated and sequenced variants are used as a check on the Pac Bio data, but are primarily for the identification and isolation of particular clones of interest for the secondary screening and validation processes.

The sequences for Triple Combo library mutations were determined by Pac Bio sequencing. Ion Torrent technology results in an average read length of 350-400 bp. In the Triple Combo library, mutations were distributed across the entire gene. To accurately determine which mutations were present in a specific line, Pac Bio sequencing was utilized because of its ability to read the entire length of the gene. Each Pool was amplified across the full length of the gene with unique non-symmetrical barcodes on the 5′ and 3′ ends for a total amplicon length of 1550 bps following the same baseline, “A” “B” etc. replicates as the Ion Torrent sequencing.

Data from the Pac Bio sequencing was analyzed as follows. Briefly, demultiplexed CCS (circular consensus sequences) were aligned to the rbcL reference sequence, followed by a repair step for any indels that converted to the wild type reference sequence. Subsequently, all nucleotide substitutions relative to the reference were listed. For each sequence, this list of SNPs was compared to all possible nucleotide substitutions based on the 27 known amino acid substitutions. For each sequence, accepted (i.e. known) amino acid changes were detailed and compiled by pool. Coupled with the total number of sequences per Pool, frequencies and ratios for each possible triple combo sequence were calculated and used for selection coefficient calculations.

Hit counts and total sequences were used to calculate the ratio of each variant present at a given time point. The same selection coefficient calculation as above was used. In many cases, a given sequence was identified at one time point but not detected in another time point. As the natural log of zero produces an error, assumptions were necessary in such cases. When analyzing the TC library, any instance where the baseline was zero but the variant was detected at the endpoint, a value of 1/c was assigned to the baseline ratio, where c equals the number of colonies that were scraped into the inoculum flask. During the analysis, these assumptions were monitored to avoid consideration of artifactual data. For the PacBio data, the number of reads from the baseline samples varied significantly, with some entire Pools having zero sequenced baseline variants. Therefore the entire baseline was calculated using the 1/c minimum positive ratio, a theoretical baseline ratio of each mutant based on the number of colonies transformed per Pool. This will result in the “minimum positive” s calculation for any variant detected in a final replicate pool. This also means that no “extinct” variants were detected, as by definition extinct variants have an assumed value at the endpoint, and any variant with assumed values at baseline and endpoint were excluded from analysis.

The same formula was used to estimate the length of time required for competition. Assuming the minimum 1-in-96 for the Triple Combo Pools 1-60, approximately 200 sequences at the endpoint and a sensitivity of 2.5% (i.e. 5 sequences out of 200), it was found that 4 weeks was more than sufficient for screening. For Pools 61-66 assuming the minimum 1-in-34, it was calculated that 2 weeks provides 1.5% sensitivity (i.e. 3 out of 200).

### Secondary Turbidostat Screening.

For every variant detected by NGS in the primary screen, selection coefficients were calculated using the common baseline hit ratio and the final hit ratio for each replicate turbidostat (sᵣₑₚ). The average of these sᵣₑₚ values is calculated as s_{avg}. An alternative selection coefficient was also calculated for each variant by summing the final hits and the sum of total sequences for all replicates and using that as the final ratio for s calculation (sₛᵤₘ). The top 264 isolated variants based on s_{avg} and sₛᵤₘ were subjected to a head-to-head (1:1) turbidostat growth assay against a common competitor strain as a secondary screen.

As in the previous Example, a common competitor strain was used. This strain is a wild type C. reinhardtii with a plasmid containing codon-optimized Venus (GFP variant) and zeocin-resistance genes under control of a strong constitutive nuclear promoter. The clone selected to be the common competitor for the assay was shown to have stable fluorescence. While the strain typically slightly outperforms wild type (s_{avg}=0.03), all lines including wild type will be evaluated relative to it.

Winning clones from primary screening, along with the Venus⁺ strain, the double mutant parental clone (SSM094), wild type, and ΔrbcL complemented with wild type rbcL, were inoculated into flasks containing TAP media. After 2-4 days of growth in flasks, cells were passaged to new flasks and grown for 1 additional day. Cell concentrations were then normalized by OD₇₅₀ and winning clones (and controls) were mixed in equal volume with the Venus⁺ strain and inoculated into triplicate turbidostats. The turbidostats were filled with HSM media and set to an OD₇₅₀ of approximately 0.3, which represents an early- to mid-log phase. Light of ^{˜}150 μEinstein was provided for 16 hours a day with 8 hours of dark (diurnal cycling), with a constant stream of 0.2% CO₂ bubbling into the culture. Cultures were monitored at least daily for media replenishment, CO₂ delivery, culture settling, cell sticking, mechanical failure or any other issues. Samples were taken three times over the course of 7 days, typically on or around days 2, 4 and 6. At each time point, samples were run on the Guava easyCyte flow cytometer (EMD Millipore; Billerica, Mass.) to determine the relative ratio of Venus⁺ and rbcL variant in each population. The amount of growth media consumed between time points was also recorded to determine the approximate number of generations each turbidostat had gone through.

Analysis from Secondary Turbidostat Screening.

Using Guava CytoSoft software, gates were applied to each flow cytometry run to differentiate non-green fluorescent cells from the Venus⁺ strain. The winner ratio was calculated for each sample as described previously. Also as described previously, the selection coefficient equation, ln(rₜ)=ln(r₀)+st, is in the form of a line y=b+mx, where the selection coefficient (s) is equivalent to the slope (m) of the natural log of the ratio over time. Alternatively, selection coefficients can be calculated by plotting ln(rₜ) vs. the number of generations. While turbidostats maintain optical density within a relatively narrow range, slight variances in density can affect the growth rate of a turbidostat's population, thereby varying the number of generations produced by replicate turbidostats. In order to control for this effect, media consumption between Guava samplings was used to calculate the number of generations at each time point, and selection coefficients were calculated in units of generations⁻¹ using the previously described equation but substituting r₀, rₜ, and t for the ratio (M1/M2) at the first Guava sampling, the ratio at the subsequent samplings, and the number of generations between samplings, respectively. The calculated selection coefficients were then used to rank and select potential winning clones as Proposed Genes. See results below for details.

### Screening Results

### SSM Primary Turbidostat Screening Results.

Depending on transformation efficiency, 1-3 transformation rounds, each consisting of 5 biolistic shots per SSM Pool, provided the transformed Chlamydomonas lines for primary screening. After colonies had grown up on the transformation plates, they were counted and put into sets with an average of 821 (SSM) colonies in each. These sets served as the inoculum cultures for primary turbidostat screening.

Based on our experience with operating turbidostats, attrition is expected over the course of a multi-week experiment due to occasional equipment failure or culture crash. Therefore excess replicates were set up for screening. 34 sets were initially set up, one per SSM Pool. The SSM library had four replicate turbidostats established per set. The target screening time for the cultures was four weeks, however in the event that a turbidostat failed to make the four week endpoint, but samples were still collected for NGS at week two or week three, the turbidostat was still included in the analysis. Of the 136 SSM library primary screen turbidostats, 135 made it to the four week endpoint, and one failed before NGS samples were taken and therefore was excluded from the analysis. Every set was represented by at least 3 replicates in the NGS analysis.

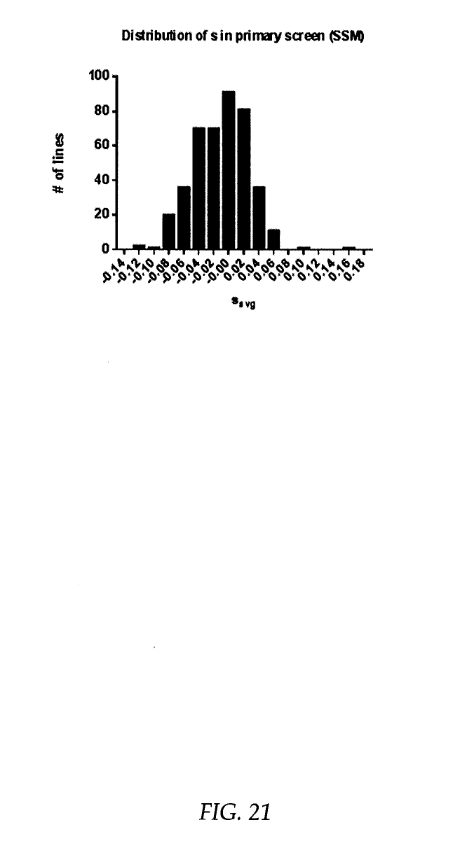

Use of NGS for data generation allowed for much more resolution over use of Sanger sequencing of individual clones. While Sanger data only provides data on the most prevalent variants in the population (and thus skews towards those variants that become dominant via a selective advantage), NGS allows sampling of nearly all variants in a pool. Thus those variants that are neutral or with a negative selection can be identified and characterized. Even those mutants that are present at the beginning of an experiment that go to zero (“extinct”) can be fairly reliably detected. FIG. 21 shows the distribution of selection coefficients as measured for all non-extinct variants in the SSM primary screen.

Each SSM Pool had a theoretical maximum of 126 variants (14 amino acids×9 substitutions) with actual average variant numbers per Pool of 17. This suggests that on average 86% of the SSM variants either did not complement the ΔrbcL strain or were below our detection limit. Selection coefficient values derived from the primary screen, while relative only to variants within the library and some fraction of parental variants, can be relied upon as a main criterion for selecting winning clones.

### Triple Combo Primary Turbidostat Screening Results.

Depending on transformation efficiency, 1-3 transformation rounds, each consisting of 5 biolistic shots per SSM Pool, provided the transformed Chlamydomonas lines for primary screening. After colonies had grown up on the transformation plates, they were counted and put into sets with an average of 587 Triple Combo Pool colonies in each. These sets served as the inoculum cultures for primary turbidostat screening.

The 66 Pools were set up in triplicate. The target screening time for Pools 1 through 60 was four weeks, however in the event that a turbidostat failed to make the four week endpoint but samples were still collected for NGS at week three, the turbidostat was still included in the analysis. Pools 61-66, which had considerably less diversity, had a target screening time of 2 weeks. Of the 198 Triple Combo library primary screen turbidostats, 196 made it to the completion week endpoint, and 2 failed before NGS samples were taken and therefore were excluded from the analysis. All Pools were represented in the analysis.

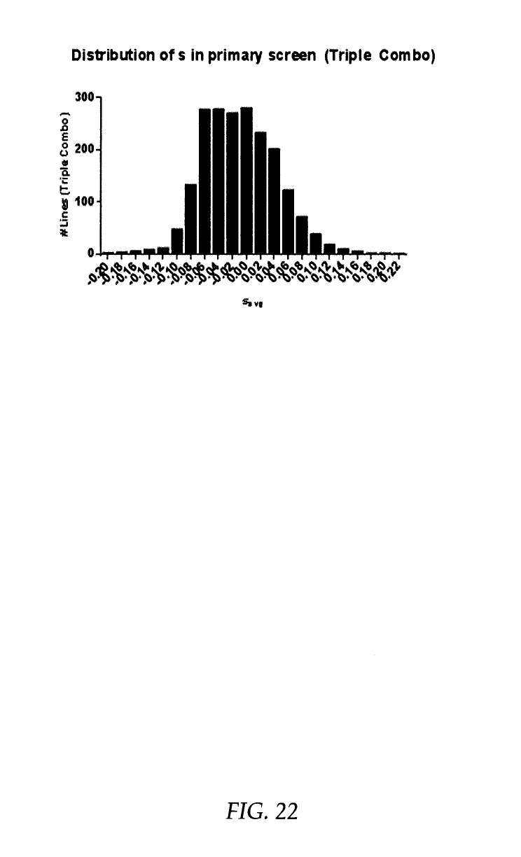

Use of Pac Bio next-generation sequencing for data generation allowed for much more resolution over use of Sanger sequencing of individual clones. While Sanger data only provides data on the most prevalent variants in the population (and thus skews towards those variants that become dominant via a selective advantage), NGS allows sampling of nearly all variants in a pool. Thus those variants that are neutral or with a negative selection can be identified and characterized. FIG. 22 shows the distribution of selection coefficients as measured for all non-extinct variants in the Triple combo primary screen. The Triple Combo library had a theoretical maximum of 96 or 34 variants per Pool.

Due to variable PacBio sequencing of the baseline complementation rates for this library were not determined. Selection coefficient values derived from the primary screen, while relative only to variants within the library and some fraction of parental variants, can be relied upon as a main criterion for selecting winners.

### Secondary Turbidostat 1:1 Competition Results.

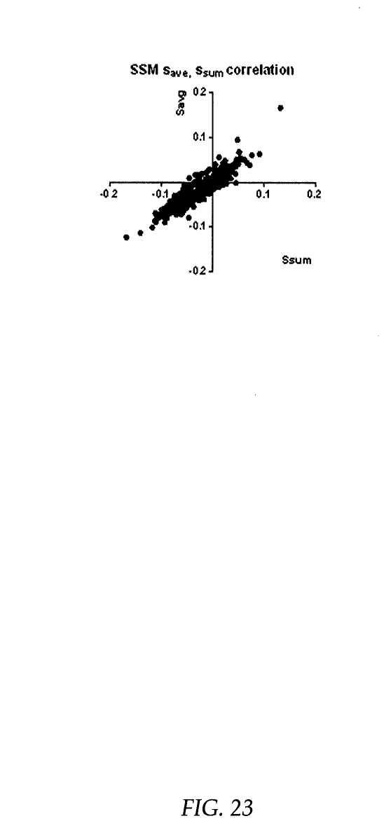

For every variant detected by NGS in the primary screen, selection coefficients were calculated using the common baseline hit ratio for the SSM library or the minimum positive ratio for the Triple Combo library and the final hit ratio for each replicate turbidostat (sᵣₑₚ). The average of these sᵣₑₚ values is calculated as s_{avg}. To prevent large negative sᵣₑₚ values from excessively lowering s_{avg}, thereby masking good performance in other replicates, an alternative selection coefficient was also calculated for each variant by summing the final hits and the sum of total sequences for all replicates and using that as the final ratio for s calculation (sₛᵤₘ). Comparing all of the s_{avg} values for the replicates with the sₛᵤₘ value on the summed replicates gives significant positive correlations, suggesting that either measure would be useful for selecting winning clones. FIG. 23 shows an example of s_{avg} vs. sₛᵤₘ for all viable variants in the SSM094 primary screen. Given that the two selection coefficient calculations are not perfectly correlated (R=0.85), both were used to ensure all winning clones are identified.

Approximately 420 variants were identified at the endpoint of the primary screen of the SSM library. To select the top candidates, a cutoff was applied if s_{avg} and sₛᵤₘ were less than 0. This narrowed the list to 190 variants from the SSM libraries.

The top 155 SSM variants on the list that were successfully isolated from FACS sorting were scaled up for 1:1 turbidostat competitions. In 35 cases, a top variant was not identified by Sanger sequencing of the FACS-sorted clones, or a variant was identified but was contaminated and therefore not scaled up. Thus, the actual ranking of variants that advanced to the secondary screen ranged from 1 to 164.

Approximately 1440 variants were identified at the endpoint of the Triple Combo library primary screen. To select the top candidates, a cutoff was applied if the variant was only found in one replicate turbidostat or if the s_{avg} and sₛᵤₘ were less than 0.05. This narrowed the list to 198 variants. This list was further ranked to reflect variants that did well in both TC Pools in which it competed versus variants that did well in one Pool but not both.

The top 109 variants on the list that were successfully isolated from FACs sorting were scaled up for 1:1 turbidostat competitions. In 89 cases, a top variant was not identified by Sanger sequencing the FACS-sorted clones, or a variant was identified but was contaminated and therefore not scaled up. Thus, the actual ranking of variants that advanced to the secondary screen ranged from 1 to 197. If a top TC variant was identified in both Pools, the clone was isolated from the Pool with the highest s_{avg} whenever possible. There were multiple Pools where many winner variants were identified but could not be found by Sanger sequencing. This is likely an artifact of the Pac Bio data/analysis, particularly the need to use the minimum positive ratio for the baseline calculation.

In summary, the top 155 SSM and 109 Triple Combo variants were set up, along with wild-type and wild type-complemented knockout strains, in 5 rounds of approximately 35 winning clones each and one round of 90 winning clones. The Example 2 winner and Example 3 SSM parent of SSM094 (R83H T200S) was also set up as a control for every round. Each winner (or control) was mixed 1:1 with the Venus⁺ strain and inoculated into triplicate turbidostats; the controls were inoculated into quadruplicate turbidostats. All competitions were run for at least 7 days, and samples were taken thrice during that time. For each winner and control, Guava ratios were used to calculate sᵣₑₚ and s_{ag} values relative to the Venus⁺ strain.

The s_{avg} for each winner in the secondary screen was compared to the s_{avg} values for wild type, wild type-complemented knockout, and the SSM094 control to generate three Δs_{avg} values, where Δs_{avg}(winner)=s_{avg}(winner)−s_{avg}(control). Δs_{avg}>0 implies that a winner line would outperform the control if they were competed head-to-head. Wild type and the complemented knockout were run in all setup rounds, and therefore winner lines were compared to the wild type/wild type-complemented controls in their respective round.

### Gene Selection.

Data from both the primary and secondary turbidostat screening experiments was used to select the top 97 variants for Selection. In the primary screen, selection coefficients were calculated for all variants in each replicate turbidostat using the common baseline hit ratio for the set and the final hit ratio for each replicate (column sᵣₑₚ Table 32). The average of these replicate s values is calculated as s_{avg}. An alternative selection coefficient was also calculated for each variant by summing the final hits and the sum of total sequences for all replicates and using that as the final ratio for s calculation (column sₛᵤₘ). In the example from primary screening given below, the endpoint is at 28 days. As a demonstration, s for the first replicate in the table on the next page (and highlighted in bold text) is calculated as follows:

$$ln ( {r}_{t} ) = ln ( {r}_{0} ) + s \cdot t$$

$$ln ( \frac{493}{54856 - 493} ) = ln ( \frac{142}{36652 - 142} ) + s \cdot 28$$

$$ln ( 0.0091 ) = ln ( 0.0039 ) + s \cdot 28$$

$$s = 0.0303$$

Example data for two variants at one position is given in Table 37. Positions and original residues are anonymized but actual data is presented.

TABLE 37

| | | Raw | | | | Corrected | | | | | | Final | Final | |
|---|---|---|---|---|---|---|---|---|---|---|---|---|---|---|
| Variant | Replicate | Counts | Sumₚₒₛ | Sum_{BC} | Corr. | Counts | Ratio | Sᵣₑₚ | S_{avg} | stdev | CI | Hit Sum | Total Sum | sₛᵤₘ |
| X999H | Baseline | 136 | 35127 | 36652 | 1.043 | 142 | 0.0039 | | | | | | | |
| | A | 474 | 52747 | 54856 | 1.040 | 493 | 0.0091 | 0.0303 | 0.0392 | 0.0078 | 0.0163 | 2187 | 185536 | 0.0400 |
| | B | 750 | 50172 | 53976 | 1.076 | 807 | 0.0152 | 0.0486 | | | | | | |
| | C | 347 | 32740 | 34254 | 1.046 | 363 | 0.0107 | 0.0362 | | | | | | |
| | D | 502 | 40678 | 42450 | 1.044 | 524 | 0.0125 | 0.0417 | | | | | | |
| X9995 | Baseline | 85 | 35127 | 36652 | 1.043 | 89 | 0.0024 | | | | | | | |
| | A | 9 | 52747 | 54856 | 1.040 | 9 | 0.0001 | −0.1139 | −0.0813 | 0.0653 | 0.1364 | 228 | 85536 | −0.0239 |
| | B | 193 | 50172 | 53976 | 1.076 | 208 | 0.0038 | 0.0167 | | | | | | |
| | C | 15 | 32740 | 34254 | 1.046 | 16 | 0.0001 | −0.1139 | | | | | | |
| | D | 11 | 40678 | 42450 | 1.044 | 11 | 0.0001 | −0.1139 | | | | | | |

As described above, the counts for each variant were normalized across a given barcode. The raw count of reads in a given position (i.e. amino acid number for the SSM library) was multiplied by the correction factor Sum_{BC}/Sumₚₒₛ, where Sum_{BC} is the total number of reads for the barcode (amplicon), and Sumₚₒₛ is the total number of error-free reads at that codon position. All variants with an assumed ratio of 0.0001 were considered extinct (indicated by underlining). A one-sample, one-sided t-test was employed by calculating a 95% confidence interval (CI, α=0.025) from the standard deviation followed by comparison of this CI to the average. Any measurements with a CI less than the average were determined to be statistically greater than zero. In this example, the first variant (indicated with an arbitrary starting amino acid and position of X999 substituted to H) is statistically higher than zero as the average minus the CI is greater than zero.

For the Triple Combo libraries, total reads per barcode and raw counts per variant were used directly as the correction factor was not necessary.

As previously described, the top 264 variants from primary screening were advanced to head-to-head turbidostat competitions against a wild type analog as a secondary screen. Selection coefficients (sᵣₑₚ and s_{avg}) were calculated for each winning clone in the primary screen as described above. For the secondary screen, the number of generations between readings was used in place of elapsed time to control for differences in growth rates. Therefore, all s_{avg} and sₛᵤₘ values reported from the primary screen are in units of days⁻¹, while s_{avg} values reported from the secondary screen are in units of generations⁻¹. While a positive selection coefficient relative to another strain in the primary screen is indicative of a selective advantage regardless of the unit used, the magnitude of s values from the primary screen cannot be directly compared to those from secondary.

Following the secondary screen, all winning clones have up to 7 selection coefficient measurements to consider: two (SSM) or four (TC) from primary screening (s_{avg}, sₛᵤₘ), and two or three from secondary screening (Δs_{avg}WT, Δs_{avg}WT-Comp, and Δs_{avg}SSM094). Winning clones were sorted according to which outperformed all three controls in the secondary screen, followed by which outperformed wild type and wild type-complemented, followed by which outperformed wild type-complemented only.

The first set of winning clones selected from this data was comprised of 22 variants that outperformed the Example 2 winner SSM094, wild type, and wild type-complemented controls (all Δs_{avg}>0) in the secondary screen (Class 1). The focus of this effort was to show added yield improvements by combining new mutations with previously validated ones; the most direct measure of which is to compare the performance of a TCP double or triple-mutant variant or a SSM triple mutant to the double mutant Example 2 winner (Δs_{avg}SSM094).

Class 2 is comprised of 29 variants that outperformed the wild type and wild-type complemented controls (Δs_{avg}WT>0, Δs_{avg}WT-Comp>0) in the secondary screen. These lines have a consistent growth advantage over wild type controls in secondary competition experiments.

The primary screen used in this Example provided a robust dataset that gave a reliable indicator of variant performance, though of course in the context of the mixed variant population it competed against. Thus the next class relied on primary data for nomination of variants with the secondary screen as a filter to remove those that are major underperformers in 1:1 competitions. As described in the previously, 95% confidence intervals were utilized to determine whether the primary screen s_{ag} values were significantly greater than zero (p<0.05). Class 3 consists of 8 variants that outperformed the wild type-complemented control in the secondary screen and had s_{avg} values significantly >0 in the primary screen.

Class 4 consists of 38 variants that strongly outperformed the wild type-complemented control in the secondary screen (Δs_{avg}WT-Comp>0). While these lines did not meet the higher threshold of performing better than true wild type in this secondary screen, their performance against this control strain and their performance in the primary screen were high enough to warrant inclusion for further validation.

The four classes are outlined in the Table 38. For a variant to be included in a class, all columns must be true. The number of variants in each class is also listed in the table. Note that a given variant was included in only one class even if it qualified for more than one (e.g. all Class 1 variants could also be considered Class 2 variants). There are a total of 97 variants in Classes 1-4.

TABLE 38

| Class | Primary | Sec. Δs_{avg}Parent | Sec. Δs_{avg}WT | Sec. Δs_{avg}WT-Comp | # Variants |
|---|---|---|---|---|---|
| 1. | | >0 | >0 | >0 | 22 |
| 2. | | | >0 | >0 | 29 |
| 3. | s_{avg} > 0 (p < 0.05) | | | >0 | 8 |
| 4. | | | | >0 | 38 |

### Validation

Turbidostat Competitions with Selected Variants.

Regeneration of Lines.

SSM variants were cloned directly out of the original lines by PCR from genomic DNA. Full-length amplicons were digested with NdeI and SpeI, and ligated into the chloroplast transformation vector pSC179 (FIG. 1). The vector contains approximately 2.8 kb each of 5′ and 3′ flanking sequence homology to the rbcL locus in the C. reinhardtii chloroplast genome, as well as elements for propagation and selection in bacteria.

Ligation products were introduced to E. coli cells via electroporation, and plasmid DNA was isolated from individual colonies for sequence verification. Once a single bacterial colony containing the plasmid sequence of interest was identified it was scaled up in overnight culture for plasmid purification.

Variants from the Triple Combo library were previously cloned into the pSC179 vector. Glycerol stocks containing these variant vectors were plated onto the proper selection media and then scaled up in overnight culture for plasmid purification.

DNA from each rbcL variant was transformed into the chloroplast genome of rbcLΔ C. reinhardtii cells via gold particle bombardment. Selection for rbcL complementation was carried out on HSM solid media, a minimal medium that necessitates obligate photoautotrophic growth. Single colonies were isolated and replica plated on TAP solid medium for sequence verification.

Selection of transformed lines was carried out by restoration of photosynthesis, and photoautotrophic growth has been shown to drive transgenic strains toward a homoplasmic state in which every copy of the chloroplast genome contains the rbcL variant gene in place of aphA6. Clones were inoculated into single turbidostats filled with HSM media and set to an OD₇₅₀ of approximately 0.3. Constant light of ^{˜}150 μEinstein (μA) was provided, with a constant stream of 0.2% CO₂ bubbling into the culture. Cultures were grown under photoautotrophic conditions for 3-4 days and subsequently plated on solid HSM media to isolate single colonies.

PCR was performed to confirm homoplasmy. Cell lysate was used in a multiplex PCR reaction with one set of primers that amplify rbcL, and one set of primers that amplify aphA6. Control multiplex PCR reactions showed the aphA6 PCR reaction is able to amplify the gene at an aphA6:rbcL ratio of 1:100 after 35 cycles. The C. reinhardtii chloroplast is reported to contain approximately 80 copies of genomic DNA. Given the sensitivity described, any lysate that produced an rbcL PCR product and no aphA6 PCR product after 35 cycles of PCR was considered to be homoplasmic for the RuBisCO variant gene.

Wild Type Analog Strain (Common Competitor).

In Example 1, the common competitor strain was a kanamycin-resistant (kan^{R}) C. reinhardtii wild type analog. While the ratio of kan^{R} to rbcL variant can be effectively determined by FACS-sorting a population for single cells followed by replica plating onto selective and permissive media, the process is labor intensive and relatively low throughput. An improved assay was developed in Example 2, which takes advantage of flow cytometry to determine the relative ratios of fluorescent and non-fluorescent strains in a population. The common competitor strain for this assay was generated by transforming wild type C. reinhardtii cells with a plasmid containing codon-optimized YFP and zeocin-resistance genes under control of a strong constitutive nuclear promoter. This line was again used for the experiments described in this Example.

Confirmed homoplasmic lines were grown in 1 ml of TAP media to saturation in 96-well deep-well blocks. The cultures were then acclimated to HSM media by diluting back 1:10 in 50 ml flasks. Cultures were grown two days in HSM media prior to inoculation in turbidostats. The wild type and YFP strain were treated in the same manner though at larger scale. Selected Variant lines were normalized by OD₇₅₀ and mixed at a ratio of 1:1 with the YFP strain in triplicate. Each turbidostat was filled with HSM to a final volume of 35 ml. Cultures were grown under a constant stream of 0.2% CO₂ and a 16H/8H light-dark diurnal cycle. A light intensity of ^{˜}150 μE/m² was provided during the 16H phase of the cycle.

Starting on the day of setup (day 0), each turbidostat was sampled for FACS and the corresponding media bottle was weighed to approximate the number of generations. FACS was performed on the Guava easyCyte flow cytometer to calculate the relative ratios of the Selected Gene and YFP strain in each turbidostat. Data were collected every other day through day 10.

Using Guava CytoSoft software, gates were applied to each flow cytometry run to differentiate non-green fluorescent cells from the Venus strain (a YFP-expressing common competitor). The winner ratio was calculated as previously described.

En Masse Turbidostat Competition.

5 ml starter cultures of the 96 Selected Variants were grown in HSM media to mid- to late-log phase in 24 deep-well blocks. Each culture was diluted to OD₇₅₀ of 0.3 with HSM media, and then mixed in equal volumes to generate an inoculum where each line represents about 1/96^{th} of the total population. This inoculum was then used to inoculate each of eight replicate turbidostats. The turbidostats were run with HSM media at an OD₇₅₀ of approximately 0.3, which represents an early- to mid-log growth phase. Constant light of ^{˜}150 μEinstein (μE) was provided, with a constant stream of 0.2% CO₂ bubbling into the culture.

A sample of the mixture used for turbidostat inoculation (time=0) was sorted for single cells using FACS into 96-well plates containing liquid TAP media. 768 events were analyzed from the inoculum culture. After 14 days of turbidostat growth another sample was taken and used for the same sorting procedure. 384 events were analyzed from each turbidostat at the 14 day time point.

After FACS sorting, the individual strains were grown for approximately 1 week and then used as template in a PCR reaction that amplified the rbcL gene. After ascertaining success in producing a single product from the reactions, the PCR products were treated for sequencing with Exonuclease I/Shrimp Alkaline Phosphatase (ExoSAP). These products were then sequenced via Sanger chemistry using 4 separate primers that together cover the entire gene.

Sanger reads for each amplicon were assembled into contigs using Sequencher software (Gene Codes Corporation). Consensus sequences for each contig were then exported and aligned to the wild type reference sequence. The number of hits for each of the single, double or triple mutant codons was counted for each set.

Hit counts and total sequences were used to calculate the ratio of each variant present in a given timepoint. These numbers were then used to calculate a selection coefficient as previously described.

### Characterization Assays.

Microplate Growth Rate Assay.

Selected Variants were analyzed by a high-throughput 96-well plate-based assay. Briefly, cultures were grown to stationary phase in TAP and acclimated to HSM and mHSM prior to the start of the experiment. Cultures were diluted to OD₇₅₀=0.2 and grown overnight. Overnight growth was followed by a second dilution to OD₇₅₀=0.05. These initial culture densities put the cells in lag or early log phase. At this point, 200 μl of each culture was added to a 96-well microtiter plate in randomized replicates. 96-well microtiter plates used in this assay contain opaque sides and a transparent base so that light exposure is equal across the entire plate. Plates were sealed using a PDMS lid in order to allow for gas exchange but minimize culture volume loss to evaporation. Sealed plates were then grown in 3 different conditions—ambient temperature (25° C.) supplied with 5% CO₂, ambient temperature supplied with 1% CO₂, and 32° C. supplied with 5% CO₂. Intermittent shaking was set to occur for 15 s/min at 1700 rpm. Light incidence upon each plate lid was 140-150 μE. OD₇₅₀ was read at approximately 6 hour intervals for a maximum of 96 hours. The resulting OD₇₅₀ readings, which reflect culture growth, were plotted vs. time. A linear selection algorithm was used to determine the growth rate (see results).

Photosynthesis Yield Assay.

Selected Variants were also assessed for photosynthetic quantum yield using the FluorCAM 800MF (Photon Systems Instruments; Brno, Czech Republic). The FluorCAM works by exposing cultures to pulses of saturating light, which briefly suppresses photochemical yield and induces maximal fluorescence yield. The FluorCAM specializes in the quick and reliable assessment of the effective quantum yield of photochemical energy conversion in photosynthesis. Samples were grown in TAP media to saturation in 96-well deep-well blocks. Cultures were acclimated in minimal media—HSM and mHSM—by 1:10 dilution in deep-well blocks. Blocks were incubated in a CO₂ controlled growth box under constant light of 80-100 μE for two days prior to screening. Samples were screened in triplicate in 96-well clear-bottom, white microplates. Wild type C. reinhardtii was included as a control. Samples were dark adapted ten minutes prior to imaging. The minimum fluorescence signal (F₀) and the maximal yield (Fₘ) were measured and the photosynthesis yield (Y=Fᵥ/Fₘ) was calculated. Analysis was performed with FluorCam7 software.

Protein quantitation by ELISA. Cultures for Selected Variants were grown to mid-logarithmic phase in HSM and harvested by centrifugation at OD₇₅₀=0.4. Samples were flash frozen and stored at −20° C. prior to cell lysis. Cell pellets were lysed in 1 ml of 1×PBS pH 7.4 containing 1× BugBuster protein extraction reagent (EMD Millipore) and cOmplete ULTRA protease inhibitor (Roche). Samples were incubated for 15 minutes at room temperature followed by sonication for 15 seconds at 30% amplitude. Lysates were cleared by centrifugation at 14,000 rpm for 30 minutes. 7K MWCO Zeba Spin Desalting Columns (Thermo Scientific) were used to remove detergents and buffer exchange samples into 1×PBS pH 7.4. Total protein concentration was determined with the BCA protein assay (Thermo Scientific) and samples were normalized to a final total protein concentration equal to 300 μg/ml.

RuBisCO standards were prepared from a 2.5 mg/ml purified protein stock and a 1:2 serial dilution of 1 μg/ml RBCL protein was performed. NUNC MaxiSorp Immuno plates (Thermo Scientific) were coated with 100 μl of RBCL standard in duplicate and 100 μl of each Selected Variant and control (SE0050 (WT), 179 complement, and SSM094) in replicates of six. Plates were sealed and incubated at 4° C. overnight.

The following day, plates were washed once with 1×PBS+0.05% Tween-20 pH 7.4 and blocked with PBS Starting Block (Thermo Scientific) for 30 minutes at room temperature. After plates were washed once with 1×PBST, 100 μl of anti-RBCL IgY (1/25,000; Genway) was added to RBCL standards and one-half of the Selected Variant wells. 100 μl of anti-L30 antibody (1/20,000; Agrisera) was added to the remaining Selected Variant wells and the plates were incubated for 1 hour at room temperature. After washing the plates three times in 1×PBST, 100 μl of horseradish peroxidase-conjugated secondary antibody was added to the appropriate wells and incubated 1 hour at room temperature. Goat-anti-IgY HRP (1/50,000; Genway) was used against the anti-RBCL antibody and goat-anti-rabbit HRP (1/50,000; Agrisera) was used against the anti-L30 antibody. After washing the plates three times in 1×PBST, 100 μl of 1 Step Ultra TMB substrate (Thermo Scientific) was added to each well. Plates were incubated for 15 minutes at room temperature. The reaction was quenched with 100 μl 2N sulfuric acid and the absorbance at 450 nm was measured. The ratio of RBCL signal-to-L30 signal was calculated for all samples and compared to the wild type complemented strain.

### Validation Results

### Original Line Competitions.

All Selected Variants were competed in 1:1 competitions during the screening process. These results were used as a secondary screen to determine the Selection of specific variants. For details, see the screening report and final selected variant report from May 2014. One line (wTC110) did not successfully advance to regeneration because a confirmed homoplasmic and axenic line was not available in a timely manner.

### Turbidostat En Masse Competition.

The 96 Selected Variants were competed en masse in turbidostats for two weeks. 95 of the 96 variants were identified in the final sort sequencing. Three of those 95 variants were not identified in the baseline sequencing and were assumed to have a starting ratio of 1/96. 42 Selected Variants had a positive s_{avg} value. A one-sample, one-sided t-test was employed by calculating a 95% confidence interval (CI, α=0.025) from the standard deviation followed by comparison of this CI to the average. Any s measurements with a CI less than the average were determined to be statistically greater than zero. 17 lines passed this statistical test.

### Regenerated Line Competitions.

Regenerated lines for 95 Selected Variants entered into competitions via a common competitor in turbidostats. Selection coefficients were calculated from the regenerated lines based on their performance against the YFP common competitor strain as previously described. Replicates with R² values below 0.6 for the line fit of ln(rₜ) vs. generations were not included in s calculations. In order to compare results across experiments for each variant, Δs values were calculated by subtracting the s_{avg} value of the wild type complemented strain from the s value of each replicate. Thirteen variants also have all Δsᵣₑₚ greater than 0 with four of those having all three replicates reported. The values in Table 39 below reflect the Δs_{avg} for each Selected Variant compared with the three controls—SE0050 wild type, wild type complemented, and SSM094. As values were determined by subtracting the s_{avg} value of the control strain from the calculated s value of each Selected Variant replicate.

TABLE 39

| Sapphire ID | Δs_{avg}(wt) | Δs_{avg}(comp) | Δs_{avg}(SSM094) |
|---|---|---|---|
| SE0050 | N/A | 0.0765 | 0.0335 |
| 179comp | −0.0962 | N/A | −0.0429 |
| SSM094 | −0.0532 | 0.0429 | N/A |
| SSM315 | −0.103 | −0.0265 | −0.0694 |
| SSM318 | −0.0078 | −0.0369 | −0.0798 |
| SSM320 | −0.0341 | −0.0117 | −0.0546 |
| SSM324 | −0.209 | −0.1326 | −0.1755 |
| SSM326 | −0.0678 | 0.0087 | −0.0343 |
| SSM331 | −0.0872 | −0.0108 | −0.0537 |
| SSM332 | −0.1341 | −0.0577 | −0.1006 |
| SSM333 | −0.1672 | −0.0907 | −0.1336 |
| SSM334 | −0.2119 | −0.2723 | −0.3152 |
| SSM335 | 0.002 | −0.1603 | −0.2032 |
| SSM336 | −0.0838 | −0.2305 | −0.2734 |
| SSM337 | −0.092 | −0.0155 | −0.0584 |
| SSM338 | −0.1275 | −0.0511 | −0.094 |
| SSM339 | −0.2902 | −0.2137 | −0.2566 |
| SSM340 | −0.2184 | −0.1394 | −0.1823 |
| SSM344 | −0.0442 | −0.0268 | −0.0697 |
| SSM347 | −0.2465 | −0.1871 | −0.23 |
| SSM352 | −0.2441 | −0.1676 | −0.2105 |
| SSM353 | −0.0581 | 0.0184 | −0.0245 |
| SSM356 | −0.0341 | 0.0423 | −0.0006 |
| SSM358 | −0.1291 | −0.0527 | −0.0956 |
| SSM365 | −0.0197 | 0.0103 | −0.0327 |
| SSM367 | −0.0481 | 0.0283 | −0.0146 |
| SSM369 | −0.4421 | −0.3657 | −0.4086 |
| SSM370 | −0.0611 | 0.0154 | −0.0276 |
| SSM371 | −0.2242 | −0.1478 | −0.1907 |
| SSM372 | −0.0467 | 0.0298 | −0.0131 |
| SSM378 | 0.0277 | 0.0076 | −0.0353 |
| SSM379 | −0.0863 | −0.061 | −0.104 |
| SSM390 | −0.1222 | −0.0457 | −0.0886 |
| SSM391 | −0.1815 | −0.105 | −0.1479 |
| SSM394 | −0.0664 | 0.01 | −0.0329 |
| SSM395 | −0.174 | −0.0975 | −0.1404 |
| SSM398 | −0.0997 | −0.3813 | −0.4242 |
| SSM402 | −0.1267 | −0.0502 | −0.0931 |
| SSM408 | −0.1509 | −0.0744 | −0.1173 |
| SSM441 | −0.2075 | −0.131 | −0.1739 |
| SSM442 | −0.1856 | −0.1091 | −0.152 |
| SSM447 | −0.1324 | −0.056 | −0.0989 |
| SSM456 | −0.1116 | −0.1233 | −0.1663 |
| SSM460 | −0.2149 | −0.1384 | −0.1814 |
| wTC002 | 0.0169 | 0.0934 | 0.0505 |
| wTC003 | 0.0309 | 0.0617 | 0.0188 |
| wTC004 | −0.1869 | −0.1105 | −0.1534 |
| wTC008 | −0.0988 | −0.0224 | −0.0653 |
| wTC009 | −0.0978 | −0.0213 | −0.0642 |
| wTC013 | −0.0165 | 0.0145 | −0.0285 |
| wTC015 | 0.0926 | 0.0442 | 0.0013 |
| wTC018 | −0.0599 | 0.0166 | −0.0263 |
| wTC024 | 0.0127 | 0.0849 | 0.042 |
| wTC035 | 0.0412 | 0.0496 | 0.0067 |
| wTC041 | −0.0907 | −0.0142 | −0.0571 |
| wTC042 | −0.1933 | −0.1168 | −0.1597 |
| wTC043 | 0.0346 | 0.111 | 0.0681 |
| wTC044 | 0.0002 | 0.0767 | 0.0338 |
| wTC046 | −0.0493 | −0.0247 | −0.0676 |
| wTC048 | 0.0957 | 0.0775 | 0.0346 |
| wTC054 | −0.1355 | −0.059 | −0.1019 |
| wTC057 | −0.069 | 0.0074 | −0.0355 |
| wTC059 | 0.0016 | 0.0079 | −0.035 |
| wTC063 | −0.0467 | 0.0169 | −0.0261 |
| wTC067 | −0.099 | −0.0226 | −0.0655 |
| wTC069 | −0.0388 | −0.0496 | −0.0926 |
| wTC071 | −0.0577 | 0.0188 | −0.0242 |
| wTC072 | −0.0041 | −0.0217 | −0.0646 |
| wTC078 | −0.1811 | −0.1046 | −0.1475 |
| wTC079 | −0.1951 | −0.1187 | −0.1616 |
| wTC080 | −0.036 | 0.0105 | −0.0325 |
| wTC081 | −0.071 | −0.084 | −0.1269 |
| wTC082 | 0.0058 | 0.0458 | 0.0029 |
| wTC083 | −0.1466 | −0.0702 | −0.1131 |
| wTC086 | −0.2013 | −0.2373 | −0.2802 |
| wTC087 | −0.1977 | −0.1212 | −0.1641 |
| wTC093 | −0.0735 | −0.0645 | −0.1074 |
| wTC095 | −0.1144 | −0.156 | −0.1989 |
| wTC096 | −0.1567 | −0.0802 | −0.1231 |
| wTC100 | 0.0151 | −0.0288 | −0.0718 |
| wTC102 | −0.0349 | −0.122 | −0.1649 |
| wTC103 | −0.0854 | −0.0898 | −0.1327 |
| wTC105 | 0.0043 | −0.0786 | −0.1215 |
| wTC106 | −0.0504 | −0.0374 | −0.0803 |
| wTC107 | −0.1036 | −0.1065 | −0.1494 |
| wTC108 | −0.1953 | −0.1188 | −0.1617 |
| wTC109 | −0.06 | 0.0164 | −0.0265 |
| wTC111 | −0.0956 | −0.0191 | −0.062 |
| wTC112 | −0.0661 | −0.061 | −0.1039 |
| wTC114 | −0.1255 | −0.0491 | −0.092 |
| wTC115 | −0.2099 | −0.1335 | −0.1764 |
| wTC116 | −0.1075 | −0.0697 | −0.1126 |
| wTC127 | −0.1476 | −0.0711 | −0.114 |
| wTC128 | −0.0113 | −0.0657 | −0.1086 |
| wTC129 | 0.0278 | 0.0637 | 0.0208 |
| wTC141 | −0.0019 | 0.0668 | 0.0239 |
| wTC142 | −0.1471 | −0.0706 | −0.1135 |
| wTC156 | −0.1086 | −0.0321 | −0.075 |

### Validated Variants.

Original lines of the Selected Variants were successfully competed against the YFP common competitor in turbidostats as part of the screening process. These data were combined with regenerated line 1:1 competition data for validation determination. 28 variants had average Δs values that were greater than the wild type complemented in both original and regenerated line turbidostat competitions. These 28 lines are considered validated. Additionally, 5 of the 28 validated variants had average Δs values greater than the SE0050 wild type control. Those lines were wTC002, wTC024, wTC043, wTC044, and wTC048.

A one-sample, one-sided t-test was applied to both datasets by calculating a 95% confidence interval (CI, α=0.025) from the standard deviation followed by comparison of this CI to the average. Any Δs measurements with a CI less than the average were determined to be statistically greater than zero.

TABLE 40

| | Δs_{avg} > wt comp | All 3 Δsᵣₑₚ > 0 | Significant |
|---|---|---|---|
| Original Lines | 91 | 49 | 7 |
| Regenerated Lines | 28 | 4 | 2 |

The two regenerated lines determined to be significantly greater than zero were not significant in the original lines. However, the four regenerated lines with all Δs replicates greater than zero were also all greater than zero in the original lines. Those lines are SSM356, wTC002, wTC043, and wTC044.

### Characterization Assays.

Microplate Growth Rate Assay.

The 96 original Selected Variants were analyzed in microtiter plates as described previously to test whether mutants that have high selection coefficients in turbidostats have improved growth characteristics in another format. RuBisCO variants in changing temperature and CO₂ conditions were assayed to provide an indication that changes to the RuBisCO protein are responsible for increased yield over wild type. As carbon fixation and overall Rubisco function are known to be impacted by temperature, growth performance of the Selected Variants was assayed at two temperatures to give an indication of the potential temperature dependence of any predicted yield increase.

The OD₇₅₀ versus time data were not suitable for logistic curve fitting for all wells. Therefore, an exponential analysis was performed in order to calculate growth rates. With this type of analysis, the OD₇₅₀ data were plotted with time. Then, the linear region of these data was selected to define the log phase growth region of the curve. The most difficult part of this type of analysis was to determine which data represent “the linear region.” This experiment studied clones having different growth profiles; therefore a subjective time range to analyze was not suitable. In order to overcome this challenge, an algorithm for selecting the linear region of the OD₇₅₀ versus time data was developed and programmed into MS Excel VBA to analyze the data.

The linear selection algorithm uses a two phase process. Phase one of the algorithm steps through all the transformed data using all possible starting points and between 4 and 7 consecutive points to calculate the Slope, R², and the t value of the slope. Any slopes failing the t-test were rejected, α=0.05 confidence level (Kachigan. Multivariate Statistical Analysis, 2^{nd} Ed. (1991) ISBN 0-942154-91-6; p178). Of the slopes which had a significant value by the t-test, the one having the maximum product of Slope*R² was selected as representing the linear region. The slope of this linear region was used to score the growth rates of the clone. Growth rate for each well was determined independently. These resulting growth rates were then analyzed in JMP.

Microplate growth rate assays were carried out in two medias (HSM and mHSM) at three different conditions. The control growth condition was 26° C. supplemented with 5% CO₂. The second condition, elevated temperature, was 32° C. supplemented with 5% CO₂. The third condition, low CO₂, was 26° C. supplemented with 1% CO₂. Growth rates for a subset of variants were greater and statistically significant in five of the six conditions when compared to the wild type complemented strain. This is summarized in the following Table 41.

TABLE 41

| | Temperature | | Lines > wt comp |
|---|---|---|---|
| Media | (° C.) | % CO₂ | (sig) |
| HSM | 26.0 | 5.0 | 45 |
| HSM | 32.0 | 5.0 | 3 |
| HSM | 26.0 | 1.0 | 15 |
| mHSM | 26.0 | 5.0 | 2 |
| mHSM | 32.0 | 5.0 | 0 |
| mHSM | 26.0 | 1.0 | 6 |

To investigate any improvement in temperature tolerance for the variants, combinatorial difference measures between 26° C. and 32° C. were analyzed. These differences were compared to the wt-complemented strain, and resulted in 7 strains in HSM and 8 strains in mHSM with a statistically significant high temperature improvement. A list of variants that exhibit significant growth improvement at 32° C. vs. 26° C. is summarized in Table 42. Two of the variants (wTC035 and wTC069) showed improvement in both media.

TABLE 42

| | |
|---|---|
| HSM | mHSM |

| Sapphire ID | Δ(32-26)_{avg} | Sapphire ID | Δ(32-26)_{avg} |
|---|---|---|---|
| SSM315 | 0.00565 | SSM335 | 0.00271 |
| SSM352 | 0.00575 | SSM347 | 0.00538 |
| SSM390 | 0.00809 | SSM356 | 0.00262 |
| SSM394 | 0.00607 | wTC009 | 0.00365 |
| SSM441 | 0.00560 | wTC018 | 0.00499 |
| wTC035 | 0.01062 | wTC035 | 0.00924 |
| wTC069 | 0.00575 | wTC069 | 0.00984 |
| wt-comp | 0.00306 | wTC127 | 0.00509 |
| | | wt-comp | −0.00179 |

A summary of growth rates is presented in Tables 43 and 44. Cells highlighted in bold text indicate those lines with a growth rate that is statistically significant when compared to the wild type complemented strain.

TABLE 43

| | HSM - control | | HSM - 32° C. | | HSM - low CO₂ | |
|---|---|---|---|---|---|---|
| Sapphire ID | r (avg) | STDEV | r (avg) | STDEV | r (avg) | STDEV |
| SE0050 | 0.0131 | 0.0028 | 0.0148 | 0.0011 | 0.0091 | 0.5002 |
| 179comp | 0.0107 | 0.0013 | 0.0145 | 0.0015 | 0.0088 | 0.5031 |
| SSM094 | 0.0171 | 0.0011 | 0.0158 | 0.0011 | 0.0079 | 0.5061 |
| SSM315 | 0.0115 | 0.0021 | 0.0172 | 0.0007 | 0.0100 | 0.0007 |
| SSM318 | 0.0152 | 0.0032 | 0.0164 | 0.0009 | 0.0089 | 0.0003 |
| SSM320 | 0.0117 | 0.0007 | 0.0042 | 0.0052 | 0.0038 | 0.0021 |
| SSM324 | 0.0135 | 0.0018 | 0.0185 | 0.0004 | 0.0089 | 0.0004 |
| SSM326 | 0.0182 | 0.0020 | 0.0177 | 0.0009 | 0.0086 | 0.0005 |
| SSM331 | 0.0143 | 0.0036 | 0.0162 | 0.0016 | 0.0098 | 0.0007 |
| SSM332 | 0.0147 | 0.0016 | 0.0165 | 0.0008 | 0.0092 | 0.0006 |
| SSM333 | 0.0174 | 0.0013 | 0.0167 | 0.0015 | 0.0093 | 0.0002 |
| SSM334 | 0.0154 | 0.0019 | 0.0172 | 0.0011 | 0.0092 | 0.0004 |
| SSM335 | 0.0218 | 0.0015 | 0.0175 | 0.0005 | 0.0094 | 0.0009 |
| SSM336 | 0.0159 | 0.0013 | 0.0163 | 0.0023 | 0.0086 | 0.0007 |
| SSM337 | 0.0158 | 0.0009 | 0.0159 | 0.0007 | 0.0086 | 0.0005 |
| SSM338 | 0.0181 | 0.0012 | 0.0176 | 0.0010 | 0.0099 | 0.0008 |
| SSM339 | 0.0140 | 0.0027 | 0.0156 | 0.0028 | 0.0093 | 0.0002 |
| SSM340 | 0.0188 | 0.0016 | 0.0170 | 0.0013 | 0.0086 | 0.0005 |
| SSM344 | 0.0162 | 0.0010 | 0.0171 | 0.0014 | 0.0093 | 0.0007 |
| SSM347 | 0.0156 | 0.0008 | 0.0103 | 0.0025 | 0.0067 | 0.0010 |
| SSM352 | 0.0122 | 0.0028 | 0.0179 | 0.0059 | 0.0092 | 0.0005 |
| SSM353 | 0.0110 | 0.0015 | 0.0163 | 0.0024 | 0.0092 | 0.0006 |
| SSM356 | 0.0144 | 0.0017 | 0.0139 | 0.0016 | 0.0095 | 0.0006 |
| SSM358 | 0.0171 | 0.0013 | 0.0175 | 0.0010 | 0.0093 | 0.0008 |
| SSM365 | 0.0162 | 0.0029 | 0.0131 | 0.0022 | 0.0098 | 0.0004 |
| SSM367 | 0.0137 | 0.0010 | 0.0168 | 0.0005 | 0.0097 | 0.0007 |
| SSM369 | 0.0144 | 0.0017 | 0.0130 | 0.0021 | 0.0093 | 0.0003 |
| SSM370 | 0.0114 | 0.0021 | 0.0140 | 0.0027 | 0.0094 | 0.0004 |
| SSM371 | 0.0135 | 0.0020 | 0.0159 | 0.0032 | 0.0090 | 0.0007 |
| SSM372 | 0.0173 | 0.0029 | 0.0165 | 0.0010 | 0.0087 | 0.0005 |
| SSM378 | 0.0113 | 0.0018 | 0.0160 | 0.0016 | 0.0091 | 0.0005 |
| SSM379 | 0.0111 | 0.0021 | 0.0143 | 0.0035 | 0.0085 | 0.0008 |
| SSM390 | 0.0088 | 0.0020 | 0.0169 | 0.0026 | 0.0091 | 0.0006 |
| SSM391 | 0.0108 | 0.0011 | 0.0137 | 0.0033 | 0.0100 | 0.0008 |
| SSM394 | 0.0114 | 0.0020 | 0.0175 | 0.0011 | 0.0100 | 0.0015 |
| SSM395 | 0.0149 | 0.0033 | 0.0184 | 0.0012 | 0.0108 | 0.0014 |
| SSM398 | 0.0156 | 0.0016 | 0.0159 | 0.0010 | 0.0085 | 0.0009 |
| SSM402 | 0.0144 | 0.0019 | 0.0135 | 0.0016 | 0.0098 | 0.0007 |
| SSM408 | 0.0168 | 0.0017 | 0.0172 | 0.0007 | 0.0097 | 0.0012 |
| SSM441 | 0.0109 | 0.0018 | 0.0165 | 0.0012 | 0.0110 | 0.0007 |
| SSM442 | 0.0132 | 0.0032 | 0.0162 | 0.0012 | 0.0088 | 0.0008 |
| SSM447 | 0.0211 | 0.0011 | 0.0160 | 0.0003 | 0.0123 | 0.0005 |
| SSM456 | 0.0213 | 0.0017 | 0.0169 | 0.0005 | 0.0109 | 0.0011 |
| SSM460 | 0.0193 | 0.0016 | 0.0164 | 0.0016 | 0.0110 | 0.0006 |
| wTC002 | 0.0131 | 0.0020 | 0.0167 | 0.0009 | 0.0115 | 0.0021 |
| wTC003 | 0.0190 | 0.0013 | 0.0161 | 0.0005 | 0.0110 | 0.0017 |
| wTC004 | 0.0172 | 0.0025 | 0.0164 | 0.0006 | 0.0105 | 0.0012 |
| wTC008 | 0.0197 | 0.0026 | 0.0166 | 0.0018 | 0.0107 | 0.0015 |
| wTC009 | 0.0228 | 0.0035 | 0.0168 | 0.0005 | 0.0091 | 0.0006 |
| wTC013 | 0.0177 | 0.0027 | 0.0160 | 0.0005 | 0.0098 | 0.0008 |
| wTC015 | 0.0145 | 0.0032 | 0.0150 | 0.0012 | 0.0109 | 0.0010 |
| wTC018 | 0.0202 | 0.0028 | 0.0167 | 0.0008 | 0.0122 | 0.0009 |
| wTC024 | 0.0096 | 0.0020 | 0.0034 | 0.0026 | 0.0071 | 0.0012 |
| wTC035 | 0.0040 | 0.0005 | 0.0141 | 0.0020 | 0.0091 | 0.0012 |
| wTC041 | 0.0169 | 0.0016 | 0.0162 | 0.0015 | 0.0084 | 0.0013 |
| wTC042 | 0.0144 | 0.0020 | 0.0146 | 0.0012 | 0.0115 | 0.0010 |
| wTC043 | 0.0170 | 0.0016 | 0.0167 | 0.0008 | 0.0109 | 0.0018 |
| wTC044 | 0.0151 | 0.0052 | 0.0157 | 0.0010 | 0.0086 | 0.0007 |
| wTC046 | 0.0190 | 0.0018 | 0.0183 | 0.0009 | 0.0089 | 0.0007 |
| wTC048 | 0.0137 | 0.0016 | 0.0152 | 0.0011 | 0.0090 | 0.0012 |
| wTC054 | 0.0183 | 0.0023 | 0.0133 | 0.0027 | 0.0096 | 0.0006 |
| wTC057 | 0.0159 | 0.0019 | 0.0149 | 0.0026 | 0.0090 | 0.0012 |
| wTC059 | 0.0159 | 0.0016 | 0.0165 | 0.0008 | 0.0101 | 0.0005 |
| wTC063 | 0.0112 | 0.0025 | 0.0144 | 0.0006 | 0.0083 | 0.0005 |
| wTC067 | 0.0116 | 0.0019 | 0.0160 | 0.0007 | 0.0086 | 0.0010 |
| wTC069 | 0.0039 | 0.0007 | 0.0096 | 0.0038 | 0.0093 | 0.0008 |
| wTC071 | 0.0123 | 0.0013 | 0.0163 | 0.0011 | 0.0085 | 0.0006 |
| wTC072 | 0.0134 | 0.0021 | 0.0157 | 0.0013 | 0.0088 | 0.0006 |
| wTC078 | 0.0163 | 0.0028 | 0.0145 | 0.0027 | 0.0097 | 0.0005 |
| wTC079 | 0.0139 | 0.0010 | 0.0148 | 0.0009 | 0.0096 | 0.0006 |
| wTC080 | 0.0129 | 0.0022 | 0.0164 | 0.0012 | 0.0090 | 0.0007 |
| wTC081 | 0.0139 | 0.0025 | 0.0158 | 0.0007 | 0.0081 | 0.0007 |
| wTC082 | 0.0120 | 0.0017 | 0.0166 | 0.0005 | 0.0092 | 0.0006 |
| wTC083 | 0.0177 | 0.0017 | 0.0176 | 0.0009 | 0.0109 | 0.0017 |
| wTC086 | 0.0143 | 0.0053 | 0.0144 | 0.0012 | 0.0096 | 0.0011 |
| wTC087 | 0.0114 | 0.0016 | 0.0124 | 0.0012 | 0.0098 | 0.0014 |
| wTC093 | 0.0140 | 0.0004 | 0.0133 | 0.0040 | 0.0096 | 0.0007 |
| wTC095 | 0.0139 | 0.0019 | 0.0157 | 0.0016 | 0.0090 | 0.0007 |
| wTC096 | 0.0147 | 0.0012 | 0.0141 | 0.0022 | 0.0091 | 0.0005 |
| wTC100 | 0.0149 | 0.0014 | 0.0153 | 0.0015 | 0.0105 | 0.0004 |
| wTC102 | 0.0151 | 0.0018 | 0.0137 | 0.0015 | 0.0112 | 0.0007 |
| wTC103 | 0.0112 | 0.0020 | 0.0143 | 0.0014 | 0.0094 | 0.0002 |
| wTC105 | 0.0162 | 0.0016 | 0.0161 | 0.0005 | 0.0089 | 0.0010 |
| wTC106 | 0.0151 | 0.0007 | 0.0156 | 0.0009 | 0.0093 | 0.0006 |
| wTC107 | 0.0152 | 0.0015 | 0.0119 | 0.0023 | 0.0101 | 0.0006 |
| wTC108 | 0.0180 | 0.0072 | 0.0132 | 0.0021 | 0.0101 | 0.0006 |
| wTC109 | 0.0127 | 0.0018 | 0.0150 | 0.0023 | 0.0102 | 0.0006 |
| wTC110 | 0.0202 | 0.0027 | 0.0155 | 0.0007 | 0.0086 | 0.0006 |
| wTC111 | 0.0108 | 0.0014 | 0.0146 | 0.0009 | 0.0101 | 0.0016 |
| wTC112 | 0.0132 | 0.0022 | 0.0158 | 0.0010 | 0.0093 | 0.0010 |
| wTC114 | 0.0102 | 0.0015 | 0.0156 | 0.0005 | 0.0099 | 0.0006 |
| wTC115 | 0.0143 | 0.0015 | 0.0148 | 0.0016 | 0.0100 | 0.0011 |
| wTC116 | 0.0104 | 0.0016 | 0.0155 | 0.0009 | 0.0101 | 0.0006 |
| wTC127 | 0.0100 | 0.0010 | 0.0139 | 0.0019 | 0.0095 | 0.0011 |
| wTC128 | 0.0144 | 0.0020 | 0.0129 | 0.0023 | 0.0102 | 0.0009 |
| wTC129 | 0.0173 | 0.0017 | 0.0151 | 0.0015 | 0.0103 | 0.0013 |
| wTC141 | 0.0131 | 0.0017 | 0.0173 | 0.0006 | 0.0092 | 0.0005 |
| wTC142 | 0.0140 | 0.0039 | 0.0147 | 0.0019 | 0.0093 | 0.0003 |
| wTC156 | 0.0125 | 0.0009 | 0.0168 | 0.0005 | 0.0098 | 0.0004 |

TABLE 44

| | mHSM - control | | mHSM - 32° C. | | mHSM - low CO₂ | |
|---|---|---|---|---|---|---|
| Sapphire ID | r (avg) | STDEV | r (avg) | STDEV | r (avg) | STDEV |
| SE0050 | 0.0342 | 0.0040 | 0.0223 | 0.0025 | 0.0090 | 0.0011 |
| 179comp | 0.0257 | 0.0016 | 0.0178 | 0.0097 | 0.0066 | 0.0035 |
| SSM094 | 0.0258 | 0.0015 | 0.0167 | 0.0064 | 0.0079 | 0.0029 |
| SSM315 | 0.0248 | 0.0017 | 0.0225 | 0.0878 | 0.0086 | 0.0003 |
| SSM318 | 0.0274 | 0.0025 | 0.0249 | 0.0274 | 0.0094 | 0.0007 |
| SSM320 | 0.0185 | 0.0015 | 0.0178 | 0.0106 | 0.0076 | 0.0010 |
| SSM324 | 0.0283 | 0.0016 | 0.0240 | 0.0195 | 0.0085 | 0.0002 |
| SSM326 | 0.0286 | 0.0020 | 0.0270 | 0.0214 | 0.0088 | 0.0009 |
| SSM331 | 0.0269 | 0.0020 | 0.0232 | 0.0181 | 0.0088 | 0.0008 |
| SSM332 | 0.0260 | 0.0012 | 0.0230 | 0.0176 | 0.0088 | 0.0008 |
| SSM333 | 0.0265 | 0.0018 | 0.0236 | 0.0337 | 0.0088 | 0.0005 |
| SSM334 | 0.0258 | 0.0015 | 0.0216 | 0.0340 | 0.0084 | 0.0003 |
| SSM335 | 0.0209 | 0.0024 | 0.0236 | 0.0238 | 0.0083 | 0.0004 |
| SSM336 | 0.0281 | 0.0013 | 0.0237 | 0.0380 | 0.0115 | 0.0082 |
| SSM337 | 0.0255 | 0.0014 | 0.0211 | 0.0097 | 0.0092 | 0.0008 |
| SSM338 | 0.0263 | 0.0013 | 0.0189 | 0.0242 | 0.0103 | 0.0032 |
| SSM339 | 0.0281 | 0.0028 | 0.0247 | 0.0308 | 0.0080 | 0.0004 |
| SSM340 | 0.0223 | 0.0025 | 0.0239 | 0.0394 | 0.0084 | 0.0001 |
| SSM344 | 0.0258 | 0.0035 | 0.0243 | 0.0334 | 0.0092 | 0.0018 |
| SSM347 | 0.0151 | 0.0009 | 0.0205 | 0.0304 | 0.0076 | 0.0006 |
| SSM352 | 0.0264 | 0.0011 | 0.0203 | 0.0199 | 0.0080 | 0.0007 |
| SSM353 | 0.0283 | 0.0028 | 0.0221 | 0.0199 | 0.0089 | 0.0003 |
| SSM356 | 0.0217 | 0.0013 | 0.0243 | 0.0692 | 0.0082 | 0.0005 |
| SSM358 | 0.0245 | 0.0048 | 0.0224 | 0.0468 | 0.0078 | 0.0002 |
| SSM365 | 0.0257 | 0.0004 | 0.0177 | 0.0395 | 0.0086 | 0.0010 |
| SSM367 | 0.0279 | 0.0027 | 0.0249 | 0.0206 | 0.0082 | 0.0004 |
| SSM369 | 0.0255 | 0.0027 | 0.0230 | 0.0111 | 0.0089 | 0.0010 |
| SSM370 | 0.0271 | 0.0007 | 0.0230 | 0.0234 | 0.0131 | 0.0091 |
| SSM371 | 0.0289 | 0.0016 | 0.0209 | 0.0387 | 0.0096 | 0.0027 |
| SSM372 | 0.0210 | 0.0017 | 0.0196 | 0.0420 | 0.0081 | 0.0007 |
| SSM378 | 0.0282 | 0.0034 | 0.0255 | 0.0169 | 0.0079 | 0.0005 |
| SSM379 | 0.0195 | 0.0018 | 0.0177 | 0.0099 | 0.0105 | 0.0015 |
| SSM390 | 0.0285 | 0.0046 | 0.0166 | 0.0207 | 0.0100 | 0.0007 |
| SSM391 | 0.0233 | 0.0029 | 0.0178 | 0.0144 | 0.0126 | 0.0036 |
| SSM394 | 0.0315 | 0.0055 | 0.0222 | 0.0197 | 0.0083 | 0.0003 |
| SSM395 | 0.0307 | 0.0034 | 0.0205 | 0.0303 | 0.0091 | 0.0010 |
| SSM398 | 0.0290 | 0.0056 | 0.0176 | 0.0207 | 0.0090 | 0.0009 |
| SSM402 | 0.0297 | 0.0036 | 0.0237 | 0.0344 | 0.0081 | 0.0005 |
| SSM408 | 0.0256 | 0.0010 | 0.0181 | 0.0049 | 0.0105 | 0.0053 |
| SSM441 | 0.0274 | 0.0039 | 0.0213 | 0.0259 | 0.0083 | 0.0003 |
| SSM442 | 0.0247 | 0.0023 | 0.0220 | 0.0099 | 0.0110 | 0.0061 |
| SSM447 | 0.0241 | 0.0014 | 0.0240 | 0.0277 | 0.0085 | 0.0005 |
| SSM456 | 0.0205 | 0.0016 | 0.0215 | 0.0410 | 0.0086 | 0.0004 |
| SSM460 | 0.0251 | 0.0027 | 0.0223 | 0.0204 | 0.0086 | 0.0006 |
| wTC002 | 0.0261 | 0.0017 | 0.0209 | 0.0226 | 0.0091 | 0.0011 |
| wTC003 | 0.0232 | 0.0020 | 0.0182 | 0.0530 | 0.0086 | 0.0006 |
| wTC004 | 0.0214 | 0.0049 | 0.0203 | 0.0254 | 0.0092 | 0.0004 |
| wTC008 | 0.0234 | 0.0020 | 0.0216 | 0.0266 | 0.0085 | 0.0007 |
| wTC009 | 0.0202 | 0.0017 | 0.0239 | 0.0129 | 0.0103 | 0.0006 |
| wTC013 | 0.0260 | 0.0008 | 0.0192 | 0.0283 | 0.0085 | 0.0005 |
| wTC015 | 0.0272 | 0.0015 | 0.0227 | 0.0154 | 0.0082 | 0.0006 |
| wTC018 | 0.0187 | 0.0016 | 0.0237 | 0.0419 | 0.0081 | 0.0005 |
| wTC024 | 0.0268 | 0.0023 | 0.0170 | 0.0092 | 0.0099 | 0.0006 |
| wTC035 | 0.0073 | 0.0010 | 0.0165 | 0.0504 | 0.0073 | 0.0010 |
| wTC041 | 0.0217 | 0.0050 | 0.0223 | 0.0130 | 0.0090 | 0.0007 |
| wTC042 | 0.0286 | 0.0015 | 0.0220 | 0.0540 | 0.0089 | 0.0008 |
| wTC043 | 0.0240 | 0.0040 | 0.0212 | 0.0223 | 0.0100 | 0.0007 |
| wTC044 | 0.0239 | 0.0063 | 0.0222 | 0.0102 | 0.0111 | 0.0027 |
| wTC046 | 0.0239 | 0.0025 | 0.0231 | 0.0273 | 0.0085 | 0.0005 |
| wTC048 | 0.0290 | 0.0021 | 0.0209 | 0.0389 | 0.0092 | 0.0007 |
| wTC054 | 0.0226 | 0.0043 | 0.0237 | 0.0098 | 0.0103 | 0.0004 |
| wTC057 | 0.0241 | 0.0067 | 0.0217 | 0.0550 | 0.0098 | 0.0008 |
| wTC059 | 0.0201 | 0.0015 | 0.0197 | 0.0306 | 0.0086 | 0.0009 |
| wTC063 | 0.0289 | 0.0053 | 0.0233 | 0.0367 | 0.0085 | 0.0006 |
| wTC067 | 0.0329 | 0.0045 | 0.0218 | 0.0621 | 0.0095 | 0.0009 |
| wTC069 | 0.0086 | 0.0017 | 0.0181 | 0.0431 | 0.0079 | 0.0009 |
| wTC071 | 0.0263 | 0.0041 | 0.0231 | 0.0301 | 0.0101 | 0.0006 |
| wTC072 | 0.0282 | 0.0040 | 0.0234 | 0.0235 | 0.0095 | 0.0006 |
| wTC078 | 0.0281 | 0.0021 | 0.0215 | 0.0238 | 0.0093 | 0.0009 |
| wTC079 | 0.0271 | 0.0050 | 0.0083 | 0.0051 | 0.0092 | 0.0009 |
| wTC080 | 0.0283 | 0.0016 | 0.0216 | 0.0138 | 0.0103 | 0.0006 |
| wTC081 | 0.0270 | 0.0018 | 0.0225 | 0.0368 | 0.0091 | 0.0003 |
| wTC082 | 0.0274 | 0.0027 | 0.0241 | 0.0270 | 0.0083 | 0.0007 |
| wTC083 | 0.0214 | 0.0031 | 0.0207 | 0.0230 | 0.0082 | 0.0007 |
| wTC086 | 0.0283 | 0.0020 | 0.0238 | 0.0345 | 0.0079 | 0.0006 |
| wTC087 | 0.0289 | 0.0025 | 0.0210 | 0.0681 | 0.0065 | 0.0036 |
| wTC093 | 0.0266 | 0.0005 | 0.0245 | 0.0273 | 0.0082 | 0.0015 |
| wTC095 | 0.0276 | 0.0016 | 0.0219 | 0.0107 | 0.0094 | 0.0007 |
| wTC096 | 0.0280 | 0.0032 | 0.0233 | 0.0450 | 0.0109 | 0.0015 |
| wTC100 | 0.0263 | 0.0018 | 0.0198 | 0.0410 | 0.0079 | 0.0005 |
| wTC102 | 0.0298 | 0.0040 | 0.0222 | 0.0279 | 0.0099 | 0.0005 |
| wTC103 | 0.0271 | 0.0007 | 0.0221 | 0.0422 | 0.0096 | 0.0003 |
| wTC105 | 0.0252 | 0.0028 | 0.0250 | 0.0292 | 0.0097 | 0.0009 |
| wTC106 | 0.0266 | 0.0020 | 0.0217 | 0.0161 | 0.0101 | 0.0004 |
| wTC107 | 0.0286 | 0.0041 | 0.0153 | 0.1612 | 0.0068 | 0.0038 |
| wTC108 | 0.0291 | 0.0024 | 0.0205 | 0.0262 | 0.0088 | 0.0005 |
| wTC109 | 0.0283 | 0.0016 | 0.0247 | 0.0161 | 0.0093 | 0.0008 |
| wTC110 | 0.0222 | 0.0053 | 0.0154 | 0.0195 | 0.0085 | 0.0004 |
| wTC111 | 0.0307 | 0.0049 | 0.0239 | 0.0182 | 0.0098 | 0.0008 |
| wTC112 | 0.0306 | 0.0017 | 0.0221 | 0.0226 | 0.0100 | 0.0005 |
| wTC114 | 0.0287 | 0.0029 | 0.0225 | 0.0300 | 0.0095 | 0.0005 |
| wTC115 | 0.0302 | 0.0021 | 0.0222 | 0.0197 | 0.0104 | 0.0008 |
| wTC116 | 0.0278 | 0.0010 | 0.0217 | 0.0332 | 0.0102 | 0.0003 |
| wTC127 | 0.0176 | 0.0023 | 0.0227 | 0.0235 | 0.0111 | 0.0002 |
| wTC128 | 0.0271 | 0.0012 | 0.0232 | 0.0923 | 0.0095 | 0.0009 |
| wTC129 | 0.0260 | 0.0016 | 0.0249 | 0.0251 | 0.0091 | 0.0003 |
| wTC141 | 0.0259 | 0.0032 | 0.0203 | 0.0638 | 0.0093 | 0.0007 |
| wTC142 | 0.0258 | 0.0025 | 0.0263 | 0.0157 | 0.0091 | 0.0003 |
| wTC156 | 0.0276 | 0.0006 | 0.0181 | 0.0141 | 0.0084 | 0.0006 |

Photosynthesis Yield Assay.

Original lines of the 96 Selected Variants were screened for photosynthetic yield using the FluorCAM. All strains were tested in both HSM and mHSM. Values for photosynthetic yield are listed in Table 45. Analysis of these data resulted in lines that are statistically different than wild type, however all lines are considered to be photosynthetically healthy based on their Fᵥ/Fₘ values.

TABLE 45

| | |
|---|---|
| HSM | mHSM |

| Sapphire ID | Fv/Fm | STDEV | Fv/Fm | STDEV |
|---|---|---|---|---|
| SSM315 | 0.7600 | 0.0000 | 0.7467 | 0.0058 |
| SSM318 | 0.7600 | 0.0000 | 0.7400 | 0.0000 |
| SSM320 | 0.7533 | 0.0058 | 0.7433 | 0.0058 |
| SSM324 | 0.7367 | 0.0058 | 0.7400 | 0.0000 |
| SSM326 | 0.7500 | 0.0000 | 0.7433 | 0.0058 |
| SSM331 | 0.7633 | 0.0058 | 0.7433 | 0.0058 |
| SSM332 | 0.7633 | 0.0058 | 0.7400 | 0.0000 |
| SSM333 | 0.7667 | 0.0058 | 0.7500 | 0.0000 |
| SSM334 | 0.7600 | 0.0000 | 0.7433 | 0.0058 |
| SSM335 | 0.7600 | 0.0000 | 0.7500 | 0.0000 |
| SSM336 | 0.7633 | 0.0058 | 0.7500 | 0.0000 |
| SSM337 | 0.7600 | 0.0000 | 0.7600 | 0.0000 |
| SSM338 | 0.7600 | 0.0000 | 0.7467 | 0.0058 |
| SSM339 | 0.7600 | 0.0000 | 0.7500 | 0.0000 |
| SSM340 | 0.7400 | 0.0000 | 0.7400 | 0.0000 |
| SSM344 | 0.7600 | 0.0000 | 0.7500 | 0.0000 |
| SSM347 | 0.7667 | 0.0058 | 0.7533 | 0.0058 |
| SSM352 | 0.7600 | 0.0000 | 0.7500 | 0.0000 |
| SSM353 | 0.7633 | 0.0058 | 0.7400 | 0.0000 |
| SSM356 | 0.7700 | 0.0000 | 0.7500 | 0.0000 |
| SSM358 | 0.7633 | 0.0058 | 0.7400 | 0.0000 |
| SSM365 | 0.7667 | 0.0058 | 0.7400 | 0.0000 |
| SSM367 | 0.7167 | 0.0058 | 0.6867 | 0.0058 |
| SSM369 | 0.7700 | 0.0000 | 0.7600 | 0.0000 |
| SSM370 | 0.7600 | 0.0000 | 0.7467 | 0.0058 |
| SSM371 | 0.7567 | 0.0058 | 0.7500 | 0.0000 |
| SSM372 | 0.7400 | 0.0000 | 0.7400 | 0.0000 |
| SSM378 | 0.7500 | 0.0000 | 0.7400 | 0.0000 |
| SSM379 | 0.7600 | 0.0000 | 0.7467 | 0.0058 |
| SSM390 | 0.7400 | 0.0000 | 0.7400 | 0.0000 |
| SSM391 | 0.7600 | 0.0000 | 0.7500 | 0.0000 |
| SSM394 | 0.7600 | 0.0000 | 0.7433 | 0.0058 |
| SSM395 | 0.7500 | 0.0000 | 0.7433 | 0.0058 |
| SSM398 | 0.7633 | 0.0058 | 0.7500 | 0.0000 |
| SSM402 | 0.7633 | 0.0058 | 0.7433 | 0.0058 |
| SSM408 | 0.7633 | 0.0058 | 0.7500 | 0.0000 |
| SSM441 | 0.7500 | 0.0000 | 0.7433 | 0.0058 |
| SSM442 | 0.7500 | 0.0000 | 0.7400 | 0.0000 |
| SSM447 | 0.7500 | 0.0000 | 0.7400 | 0.0000 |
| SSM456 | 0.7500 | 0.0000 | 0.7400 | 0.0000 |
| SSM460 | 0.7567 | 0.0058 | 0.7300 | 0.0000 |
| wTC002 | 0.7567 | 0.0058 | 0.7400 | 0.0000 |
| wTC003 | 0.7567 | 0.0058 | 0.7400 | 0.0000 |
| wTC004 | 0.7600 | 0.0000 | 0.7500 | 0.0000 |
| wTC008 | 0.7600 | 0.0000 | 0.7433 | 0.0058 |
| wTC009 | 0.7533 | 0.0058 | 0.7467 | 0.0058 |
| wTC013 | 0.7633 | 0.0058 | 0.7500 | 0.0000 |
| wTC015 | 0.7567 | 0.0058 | 0.7500 | 0.0000 |
| wTC018 | 0.7533 | 0.0058 | 0.7400 | 0.0000 |
| wTC024 | 0.7433 | 0.0058 | 0.7333 | 0.0058 |
| wTC035 | 0.7633 | 0.0058 | 0.7500 | 0.0000 |
| wTC041 | 0.7433 | 0.0058 | 0.7433 | 0.0058 |
| wTC042 | 0.7600 | 0.0000 | 0.7433 | 0.0058 |
| wTC043 | 0.7633 | 0.0058 | 0.7500 | 0.0000 |
| wTC044 | 0.7567 | 0.0058 | 0.7433 | 0.0058 |
| wTC046 | 0.7567 | 0.0058 | 0.7433 | 0.0058 |
| wTC048 | 0.7633 | 0.0058 | 0.7500 | 0.0000 |
| wTC054 | 0.7600 | 0.0000 | 0.7500 | 0.0000 |
| wTC057 | 0.7700 | 0.0000 | 0.7533 | 0.0058 |
| wTC059 | 0.7500 | 0.0000 | 0.7433 | 0.0058 |
| wTC063 | 0.7467 | 0.0058 | 0.7400 | 0.0000 |
| wTC067 | 0.7533 | 0.0058 | 0.7467 | 0.0058 |
| wTC069 | 0.7600 | 0.0000 | 0.7500 | 0.0000 |
| wTC071 | 0.7500 | 0.0000 | 0.7433 | 0.0058 |
| wTC072 | 0.7500 | 0.0000 | 0.7400 | 0.0000 |
| wTC078 | 0.7600 | 0.0000 | 0.7467 | 0.0058 |
| wTC079 | 0.7533 | 0.0058 | 0.7400 | 0.0000 |
| wTC080 | 0.7633 | 0.0058 | 0.7500 | 0.0000 |
| wTC081 | 0.7600 | 0.0000 | 0.7500 | 0.0000 |
| wTC082 | 0.7700 | 0.0000 | 0.7567 | 0.0058 |
| wTC083 | 0.7700 | 0.0000 | 0.7500 | 0.0000 |
| wTC086 | 0.7500 | 0.0000 | 0.7467 | 0.0058 |
| wTC087 | 0.7600 | 0.0000 | 0.7500 | 0.0000 |
| wTC093 | 0.7600 | 0.0000 | 0.7500 | 0.0000 |
| wTC095 | 0.7600 | 0.0000 | 0.7500 | 0.0000 |
| wTC096 | 0.7567 | 0.0058 | 0.7500 | 0.0000 |
| wTC100 | 0.7500 | 0.0000 | 0.7467 | 0.0058 |
| wTC102 | 0.7600 | 0.0000 | 0.7500 | 0.0000 |
| wTC103 | 0.7600 | 0.0000 | 0.7500 | 0.0000 |
| wTC105 | 0.7600 | 0.0000 | 0.7500 | 0.0000 |
| wTC106 | 0.7600 | 0.0000 | 0.7500 | 0.0000 |
| wTC107 | 0.7600 | 0.0000 | 0.7500 | 0.0000 |
| wTC108 | 0.7633 | 0.0058 | 0.7533 | 0.0058 |
| wTC109 | 0.7500 | 0.0000 | 0.7500 | 0.0000 |
| wTC110 | 0.6967 | 0.0058 | 0.7267 | 0.0058 |
| wTC111 | 0.7533 | 0.0058 | 0.7500 | 0.0000 |
| wTC112 | 0.7533 | 0.0058 | 0.7467 | 0.0058 |
| wTC114 | 0.7500 | 0.0000 | 0.7400 | 0.0000 |
| wTC115 | 0.7500 | 0.0000 | 0.7400 | 0.0000 |
| wTC116 | 0.7500 | 0.0000 | 0.7467 | 0.0058 |
| wTC127 | 0.7500 | 0.0000 | 0.7467 | 0.0058 |
| wTC128 | 0.7500 | 0.0000 | 0.7400 | 0.0000 |
| wTC129 | 0.7600 | 0.0000 | 0.7500 | 0.0000 |
| wTC141 | 0.7433 | 0.0058 | 0.7500 | 0.0000 |
| wTC142 | 0.7500 | 0.0000 | 0.7500 | 0.0000 |
| wTC156 | 0.7600 | 0.0000 | 0.7600 | 0.0000 |
| 179comp | 0.7400 | 0.0076 | 0.7275 | 0.0046 |
| SE0050 | 0.7263 | 0.0052 | 0.7450 | 0.0053 |
| SSM094 | 0.7375 | 0.0046 | 0.7213 | 0.0035 |

ELISA.

The Selected Variants were assayed for RuBisCO protein levels by ELISA. Nine samples were run with wt-complemented, SE0050, and SSM094 on each assay plate (11 plates were assayed). 60 μg of total protein was added to each sample well and protein levels were determined with antibodies specific to RBCL and the L30 ribosomal protein. Analysis was performed by combinatorial measures of the RBCL-to-L30 ratio for each Selected Variant compared to that of the wt-complemented strain.

Although the ELISA process has been standardized to reduce variance, combinatorial analysis of the controls demonstrated plate to plate variation. Ratios of the wild type complemented strain and wild type SE0050 are above to the right. The variation may be attributed to the efficiency of lysis, overall kinetics of the enzymatic reaction, or some other unknown factor. Accurate quantitation and comparison of all samples (11 plates) as a single dataset was not feasible. An ANOVA and a Dunnett's post-test were performed for each assay plate and RBCL protein levels were evaluated as higher or lower than the RBCL level of the wt-complemented strain. Table 46 below summarizes these results and provides a ranked list of samples in each plate. Any strain that is statistically distinguishable from the control is indicated in the final column.

TABLE 46

| Plate ID | Sapphire ID | Abs(Dif)-LSD | p-Value | Hi/Lo |
|---|---|---|---|---|
| Plate 1 | SSM324 | 1.521941 | <.0001* | Hi |
| Plate 1 | SSM326 | 1.054321 | <.0001* | Hi |
| Plate 1 | SSM332 | 0.726796 | <.0001* | Hi |
| Plate 1 | SSM331 | 0.711642 | <.0001* | Hi |
| Plate 1 | SSM318 | 0.521941 | <.0001* | Hi |
| Plate 1 | SSM315 | 0.198594 | <.0001* | Hi |
| Plate 1 | wt comp | −0.23416 | 1 | |
| Plate 1 | SSM334 | −0.0201 | 0.0898 | |
| Plate 1 | SSM333 | −0.01029 | 0.0679 | |
| Plate 1 | SSM320 | 0.610618 | <.0001* | Lo |
| Plate 2 | SSM337 | −0.10324 | 0.3077 | |
| Plate 2 | SSM340 | −0.18954 | 0.7975 | |
| Plate 2 | SSM352 | −0.21799 | 0.9287 | |
| Plate 2 | SSM347 | −0.25295 | 0.9936 | |
| Plate 2 | SSM336 | −0.26895 | 0.999 | |
| Plate 2 | SSM344 | −0.29194 | 1 | |
| Plate 2 | wt comp | −0.35989 | 1 | |
| Plate 2 | SSM338 | −0.34185 | 1 | |
| Plate 2 | SSM339 | −0.15494 | 0.587 | |
| Plate 2 | SSM335 | 0.130131 | 0.0021* | Lo |
| Plate 3 | SSM369 | 2.159098 | <.0001* | Hi |
| Plate 3 | SSM370 | 0.297907 | <.0001* | Hi |
| Plate 3 | SSM372 | 0.229845 | <.0001* | Hi |
| Plate 3 | SSM367 | 0.100749 | 0.0008* | Hi |
| Plate 3 | SSM371 | 0.049483 | 0.0076* | Hi |
| Plate 3 | SSM353 | −0.02952 | 0.1289 | |
| Plate 3 | wt comp | −0.2064 | 1 | |
| Plate 3 | SSM356 | −0.08068 | 0.464 | |
| Plate 3 | SSM365 | −0.06346 | 0.3185 | |
| Plate 3 | SSM358 | 0.619999 | <.0001* | Lo |
| Plate 4 | SSM408 | 0.939581 | <.0001* | Hi |
| Plate 4 | SSM395 | 0.386877 | <.0001* | Hi |
| Plate 4 | SSM402 | 0.066177 | 0.0066* | Hi |
| Plate 4 | SSM390 | 0.033768 | 0.0186* | Hi |
| Plate 4 | SSM378 | −0.14226 | 0.7885 | |
| Plate 4 | SSM398 | −0.21802 | 0.9999 | |
| Plate 4 | wt comp | −0.25805 | 1 | |
| Plate 4 | SSM379 | −0.18581 | 0.9836 | |
| Plate 4 | SSM394 | −0.13519 | 0.7354 | |
| Plate 4 | SSM391 | 0.661473 | <.0001* | Lo |
| Plate 5 | SSM442 | 1.61559 | <.0001* | Hi |
| Plate 5 | wTC008 | 0.468685 | <.0001* | Hi |
| Plate 5 | wTC004 | 0.234678 | <.0001* | Hi |
| Plate 5 | wTC003 | 0.16205 | <.0001* | Hi |
| Plate 5 | wTC002 | −0.04087 | 0.2623 | |
| Plate 5 | wt comp | −0.15208 | 1 | |
| Plate 5 | SSM460 | 0.238172 | <.0001* | Lo |
| Plate 5 | SSM441 | 0.344224 | <.0001* | Lo |
| Plate 5 | SSM456 | 0.416474 | <.0001* | Lo |
| Plate 5 | SSM447 | 0.459042 | <.0001* | Lo |
| Plate 6 | wTC009 | 0.717497 | <.0001* | Hi |
| Plate 6 | wTC018 | 0.705026 | <.0001* | Hi |
| Plate 6 | wTC024 | 0.693562 | <.0001* | Hi |
| Plate 6 | wTC041 | 0.540787 | <.0001* | Hi |
| Plate 6 | wTC042 | 0.483163 | <.0001* | Hi |
| Plate 6 | wTC013 | 0.415857 | <.0001* | Hi |
| Plate 6 | wTC015 | 0.303678 | <.0001* | Hi |
| Plate 6 | wt comp | −0.18635 | 1 | |
| Plate 6 | wTC035 | −0.00283 | 0.0557 | |
| Plate 7 | wTC054 | 0.926364 | <.0001* | Hi |
| Plate 7 | wTC048 | 0.746328 | <.0001* | Hi |
| Plate 7 | wTC059 | 0.578019 | <.0001* | Hi |
| Plate 7 | wTC067 | 0.484626 | <.0001* | Hi |
| Plate 7 | wTC044 | 0.45267 | <.0001* | Hi |
| Plate 7 | wTC063 | 0.430623 | <.0001* | Hi |
| Plate 7 | wTC057 | 0.405139 | <.0001* | Hi |
| Plate 7 | wTC046 | 0.256509 | <.0001* | Hi |
| Plate 7 | wTC043 | −0.07706 | 0.4044 | |
| Plate 7 | wt comp | −0.21475 | 1 | |
| Plate 8 | wTC071 | 0.285401 | <.0001* | Hi |
| Plate 8 | wTC083 | 0.16103 | <.0001* | Hi |
| Plate 8 | wTC080 | 0.159917 | <.0001* | Hi |
| Plate 8 | wTC081 | 0.135286 | 0.0002* | Hi |
| Plate 8 | wTC079 | 0.063903 | 0.0052* | Hi |
| Plate 8 | wTC069 | 0.046261 | 0.0102* | Hi |
| Plate 8 | wTC082 | −0.10821 | 0.6445 | |
| Plate 8 | wTC072 | −0.17483 | 0.9967 | |
| Plate 8 | wt comp | −0.2256 | 1 | |
| Plate 8 | wTC078 | 0.149948 | 0.0001* | Lo |
| Plate 9 | wTC100 | 0.821747 | <.0001* | Hi |
| Plate 9 | wTC095 | 0.687942 | <.0001* | Hi |
| Plate 9 | wTC102 | 0.346744 | <.0001* | Hi |
| Plate 9 | wTC086 | 0.043961 | 0.0044* | Hi |
| Plate 9 | wTC087 | −0.07201 | 0.6717 | |
| Plate 9 | wt comp | −0.14614 | 1 | |
| Plate 9 | wTC096 | 0.265541 | <.0001* | Lo |
| Plate 9 | wTC093 | 0.559386 | <.0001* | Lo |
| Plate 9 | wTC105 | 0.697993 | <.0001* | Lo |
| Plate 9 | wTC103 | 0.750104 | <.0001* | Lo |
| Plate 10 | wTC115 | 7.401122 | <.0001* | Hi |
| Plate 10 | wTC112 | 2.832211 | <.0001* | Hi |
| Plate 10 | wTC111 | 2.641663 | <.0001* | Hi |
| Plate 10 | wTC114 | 0.819469 | <.0001* | Hi |
| Plate 10 | wTC116 | −0.08818 | 0.118 | |
| Plate 10 | wTC108 | −0.60532 | 1 | |
| Plate 10 | wt comp | −0.68506 | 1 | |
| Plate 10 | wTC107 | −0.21087 | 0.319 | |
| Plate 10 | wTC109 | 1.25378 | <.0001* | Lo |
| Plate 10 | wTC106 | 1.832846 | <.0001* | Lo |
| Plate 11 | wTC128 | 2.022101 | <.0001* | Hi |
| Plate 11 | wTC141 | −0.46743 | 0.9406 | |
| Plate 11 | wt comp | −0.68657 | 1 | |
| Plate 11 | wTC156 | 0.527348 | <.0001* | Lo |
| Plate 11 | wTC127 | 0.586254 | <.0001* | Lo |
| Plate 11 | wTC142 | 0.824274 | <.0001* | Lo |
| Plate 11 | wTC129 | 1.299957 | <.0001* | Lo |

### Validated RuBisCO Variants

Based on the process of wild type competition and regeneration of transgenic lines, 28 Selected Variants were validated as having a competitive growth advantage due to expression of the RuBisCO variant. Additional confirmatory data was generated from physiological and biochemical assays of the Selected Variants. The validated variants and a summary of the data are listed in the Table 47.

TABLE 47

| Sapphire | | Original | | EM | | | |
|---|---|---|---|---|---|---|---|
| ID | Variant | Δs | Regen Δs | Winner | MGRA | r (Δ32° C.) | ELISA |
| SSM326 | V30L-R83H-T200S | 0.2126 | 0.0087 | | YES | | Hi |
| SSM353 | R83H-P89G-T200S | 0.1705 | 0.0184 | | | | |
| SSM356 | R83H-P89S-T200S | 0.2791 | 0.0423 | | | YES | |
| SSM365 | R83H-D94H-T200S | 0.0678 | 0.0102 | | YES | | |
| SSM367 | R83H-D94P-T200S | 0.2024 | 0.0283 | YES | | | Hi |
| SSM370 | R83H-N95H-T200S | 0.0688 | 0.0154 | YES | YES | | Hi |
| SSM372 | R83H-N95Q-T200S | 0.1195 | 0.0298 | YES | YES | | Hi |
| SSM378 | R83H-A102S- | 0.1359 | 0.0076 | | | | |
| | T200S | | | | | | |
| SSM394 | R83H-T200S- | 0.0048 | 0.0100 | | YES | YES | |
| | E249D | | | | | | |
| wTC002 | M1M-E6G-N95Q | 0.0706 | 0.0934 | | YES | | |
| wTC003 | M1M-E6G-V145G | 0.4045 | 0.0617 | | YES | | Hi |
| wTC013 | M1M-I36L-N95Q | 0.0236 | 0.0298 | | YES | | Hi |
| wTC015 | M1M-P46Q-P89G | 0.0036 | 0.0442 | | YES | | Hi |
| wTC018 | M1M-R83Q-D367S | 0.2503 | 0.0166 | | YES | YES | Hi |
| wTC024 | M1M-P89G-S359G | 0.0009 | 0.0849 | | | | Hi |
| wTC035 | M1M-E355P-S359G | 0.1514 | 0.0496 | YES | | YES | |
| wTC043 | E6G-P46Q-P89G | 0.0786 | 0.1110 | | YES | | |
| wTC044 | E6G-R83Q-I87L | 0.1527 | 0.0767 | | YES | | Hi |
| wTC048 | E6G-G272S-E355P | 0.0804 | 0.0775 | | | | Hi |
| wTC057 | A11L-R83H-E355P | 0.0220 | 0.0075 | | YES | | Hi |
| wTC059 | A11L-I87Y-E355P | 0.4308 | 0.0079 | YES | YES | | Hi |
| wTC063 | A11L-E93H-S359L | 0.0386 | 0.0168 | | | | Hi |
| wTC071 | A11S-S359G- | 0.0795 | 0.0188 | | | | Hi |
| | D367Q | | | | | | |
| wTC080 | P46Q-N95Q-E355Y | 0.0442 | 0.0104 | | | | Hi |
| wTC082 | R83H-T200S- | 0.1726 | 0.0458 | | | | |
| | E355S | | | | | | |
| wTC109 | M1M-P46Q-S359L | 0.0923 | 0.0164 | | | | Lo |
| wTC129 | I87L-P89G-E355Q | 0.2600 | 0.0637 | YES | YES | | Lo |
| wTC141 | M1M-E93H-N95Q | 0.0541 | 0.0668 | | | | |

KEY
Original Δs = original line Δs, wt-comp in 1:1 competition
Regen Δs = regenerated line Δs, wt-comp in 1:1 competition
EM Winner = en masse winner, significantly greater than zero
MGRA = significantly greater than wt-comp in any one of the MGRA conditions
r (Δ32° C.) = combinatorial difference measure between 32° C. and 26° C.; significantly > wt-comp
ELISA = statistically higher (Hi) or lower (Lo) than wt-comp

What is claimed is:

1. A transformed photosynthetic organism comprising an exogenous polynucleotide encoding a modified rbcL protein of any one of SEQ ID NO: 1; wherein said modification consists of at least one amino acid substitution in the loop at positions 25-35, the β-sheet at positions 83-89, the α-helix at positions 310-321 and the loop-helix-loop at positions 355-365; and wherein the transformed photosynthetic organism expresses the modified rbcL protein and produces increased biomass as compared to an untransformed photosynthetic organism of the same species.

2. A transformed photosynthetic organism comprising an exogenous polynucleotide encoding a modified rbcL protein of any one of SEQ ID NOs 2 to 79; wherein the transformed photosynthetic organism expresses the modified rbcL protein and produces increased biomass as compared to an untransformed photosynthetic organism of the same species.

3. A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein comprises at least one of the following modifications:
a) a change from D to H at position 28;
b) a change from V to K at position 31;
c) a change from T to H at position 34;
d) a change from I to L at position 36;
e) a change from T to Q at position 68;
f) a change from T to S at position 68;
g) a change from R to Q at position 83;
h) a change from R to H at position 83;
i) a change from E to S at position 88;
j) a change from I to Y at position 87;
k) a change from R to K at position 312;
l) a change from A to G at position 317;
m) a change from M to L at position 320;
n) a change from E to P at position 355;
o) a change from R to Q at position 358;
p) a change from T to S at position 365;
q) a change from A to S at position 315;
r) a change from A to T at position 317;
s) a change from R to H at position 83 and a change from E to G at position 6;
t) a change from R to H at position 83 and a change from K to Y at position 8;
u) a change from R to H at position 83 and a change from A to L at position 11;
v) a change from R to H at position 83 and a change from I to L at position 36;
w) a change from R to H at position 83 and a change from C to L at position 53;
x) a change from R to H at position 83 and a change from I to L at position 87;
y) a change from R to H at position 83 and a change from P to G at position 89;
z) a change from R to H at position 83 and a change from E to H at position 93;
aa) a change from R to H at position 83 and a change from N to Q at position 95;
ab) a change from R to H at position 83 and a change from V to G at position 145;
ac) a change from R to H at position 83 and a change from G to P at position 168;
ad) a change from R to H at position 83 and a change from T to S at position 200;
ae) a change from R to H at position 83 and a change from A to Y at position 230;
af) a change from R to H at position 83 and a change from V to Q at position 341;
ag) a change from R to H at position 83 and a change from E to L at position 355;
ah) a change from R to H at position 83 and a change from E to Y at position 355;
ai) a change from R to H at position 83 and a change from E to Q at position 355;
aj) a change from R to H at position 83 and a change from E to S at position 355;
ak) a change from R to H at position 83 and a change from S to L at position 359;
al) a change from R to H at position 83 and a change from S to G at position 359;
am) a change from R to H at position 83 and a change from D to Q at position 367;
an) a change from R to H at position 83 and a change from D to S at position 367;
ao) a change from R to H at position 83 and a change from E to D at position 392;
ap) a change from R to H at position 83 and a change from R to Y at position 439;
aq) a change from R to H at position 83 and a change from K to Y at position 450;
ar) a change from R to H at position 83 and a change from E to Q at position 460;
as) a change from I to Y at position 87 and a change from P to Q at position 46;
at) a change from I to Y at position 87 and a change from P to G at position 89;
au) a change from I to Y at position 87 and a change from V to K at position 255;
av) a change from I to Y at position 87 and a change from G to S at position 272;
aw) a change from R to K at position 312 and a change from A to S at position 11;
ax) a change from R to K at position 312 and a change from E to P at position 355;
ay) a change from V to L at position 30, a change from R to H at position 83 and a change from T to S at position 200;
az) a change from R to H at position 83, a change from P to G at position 89 and a change from T to S at position 200;
ba) a change from a change from R to H at position 83, a change from P to S at position 89 and a change from T to S at position 200;
bb) a change from R to H at position 83, a change from D to H at position 94 and a change from T to S at position 200;
bc) a change from R to H at position 83, a change from D to P at position 94 and a change from T to S at position 200;
bd) a change from R to H at position 83, a change from N to H at position 95 and a change from T to S at position 200;
be) a change from R to H at position 83, a change from N to Q at position 95 and a change from T to S at position 200;
bf) a change from R to H at position 83, a change from A to S at position 102 and a change from T to S at position 200;
bg) a change from R to H at position 83, a change from T to S at position 200 and a change from E to D at position 249;
bh) a change from E to G at position 6 and a change from N to Q at position 95;
bi) a change from E to G at position 6 and a change from V to G at position 145;
bj) a change from I to L at position 36 and a change from N to Q at position 95;
bk) a change from P to Q at position 46 and a change from P to G at position 89;
bl) a change from R to Q at position 83 and a change from D to S at position 367;
bm) a change from P to G at position 89 and a change from S to G at position 359;
bn) a change from E to P at position 355 and a change from S to G at position 359;
bo) a change from E to G at position 6, a change from P to Q at position 46 and a change from P to G at position 89;
bp) a change from E to G at position 6, a change from R to Q at position 83 and a change from I to L at position 87;
bq) a change from a change from E to G at position 6, a change from G to S at position 272 and a change from E to P at position 355;
br) a change from A to L at position 11, a change from R to H at position 83 and a change from E to P at position 355;
bs) a change from A to L at position 11, a change from I to Y at position 87 and a change from E to P at position 355;
bt) a change from A to L a position 11, a change from E to H at position 93 and change from S to L at position 359;
bu) a change from A to S at position 11, a change from S to G at position 359 and a change from D to Q at position 367;
bv) a change from P to Q at position 46, a change from N to Q at position 95 and a change from E to Y at position 355;
bw) a change from R to H at position 83, a change from T to S at position 200 and a change from E to S at position 355;
bx) a change from P to G at position 46 and a change from S to L at position 359;
by) a change from I to L at position 87, a change from P to G at position 89, and a change from E to Q at position 355; and
bz) a change from E to H at position 93 and a change from N to Q at position 95.

4. A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein consists of a modification selected from:
a) a change from D to H at position 28;
b) a change from V to K at position 31;
c) a change from T to H at position 34;
d) a change from I to L at position 36;
e) a change from T to Q at position 68;
f) a change from T to S at position 68;
g) a change from R to Q at position 83;
h) a change from R to H at position 83;
i) a change from E to S at position 88;
j) a change from I to Y at position 87;
k) a change from R to K at position 312;
l) a change from A to G at position 317;
m) a change from M to L at position 320;
n) a change from E to P at position 355;
o) a change from R to Q at position 358;
p) a change from T to S at position 365;
q) a change from A to S at position 315;
r) a change from A to T at position 317;
s) a change from R to H at position 83 and a change from E to G at position 6;
t) a change from R to H at position 83 and a change from K to Y at position 8;
u) a change from R to H at position 83 and a change from A to L at position 11;
v) a change from R to H at position 83 and a change from I to L at position 36;
w) a change from R to H at position 83 and a change from C to L at position 53;
x) a change from R to H at position 83 and a change from I to L at position 87;
y) a change from R to H at position 83 and a change from P to G at position 89;
z) a change from R to H at position 83 and a change from E to H at position 93;
aa) a change from R to H at position 83 and a change from N to Q at position 95;
ab) a change from R to H at position 83 and a change from V to G at position 145;
ac) a change from R to H at position 83 and a change from G to P at position 168;
ad) a change from R to H at position 83 and a change from T to S at position 200;
ae) a change from R to H at position 83 and a change from A to Y at position 230;
af) a change from R to H at position 83 and a change from V to Q at position 341;
ag) a change from R to H at position 83 and a change from E to L at position 355;
ah) a change from R to H at position 83 and a change from E to Y at position 355;
ai) a change from R to H at position 83 and a change from E to Q at position 355;
aj) a change from R to H at position 83 and a change from E to S at position 355;
ak) a change from R to H at position 83 and a change from S to L at position 359;
al) a change from R to H at position 83 and a change from S to G at position 359;
am) a change from R to H at position 83 and a change from D to Q at position 367;
an) a change from R to H at position 83 and a change from D to S at position 367;
ao) a change from R to H at position 83 and a change from E to D at position 392;
ap) a change from R to H at position 83 and a change from R to Y at position 439;
aq) a change from R to H at position 83 and a change from K to Y at position 450;
ar) a change from R to H at position 83 and a change from E to Q at position 460;
as) a change from I to Y at position 87 and a change from P to Q at position 46;
at) a change from I to Y at position 87 and a change from P to G at position 89;
au) a change from I to Y at position 87 and a change from V to K at position 255;
av) a change from I to Y at position 87 and a change from G to S at position 272;
aw) a change from R to K at position 312 and a change from A to S at position 11;
ax) a change from R to K at position 312 and a change from E to P at position 355;
ay) a change from V to L at position 30, a change from R to H at position 83 and a change from T to S at position 200;
az) a change from R to H at position 83, a change from P to G at position 89 and a change from T to S at position 200;
ba) a change from a change from R to H at position 83, a change from P to S at position 89 and a change from T to S at position 200;
bb) a change from R to H at position 83, a change from D to H at position 94 and a change from T to S at position 200;
bc) a change from R to H at position 83, a change from D to P at position 94 and a change from T to S at position 200;
bd) a change from R to H at position 83, a change from N to H at position 95 and a change from T to S at position 200;
be) a change from R to H at position 83, a change from N to Q at position 95 and a change from T to S at position 200;
bf) a change from R to H at position 83, a change from A to S at position 102 and a change from T to S at position 200;
bg) a change from R to H at position 83, a change from T to S at position 200 and a change from E to D at position 249;
bh) a change from E to G at position 6 and a change from N to Q at position 95;
bi) a change from E to G at position 6 and a change from V to G at position 145;
bj) a change from I to L at position 36 and a change from N to Q at position 95;
bk) a change from P to Q at position 46 and a change from P to G at position 89;
bl) a change from R to Q at position 83 and a change from D to S at position 367;
bm) a change from P to G at position 89 and a change from S to G at position 359;
bn) a change from E to P at position 355 and a change from S to G at position 359;
bo) a change from E to G at position 6, a change from P to Q at position 46 and a change from P to G at position 89;
bp) a change from E to G at position 6, a change from R to Q at position 83 and a change from I to L at position 87;
bq) a change from a change from E to G at position 6, a change from G to S at position 272 and a change from E to P at position 355;
br) a change from A to L at position 11, a change from R to H at position 83 and a change from E to P at position 355;
bs) a change from A to L at position 11, a change from I to Y at position 87 and a change from E to P at position 355;
bt) a change from A to L a position 11, a change from E to H at position 93 and change from S to L at position 359;
bu) a change from A to S at position 11, a change from S to G at position 359 and a change from D to Q at position 367;
bv) a change from P to Q at position 46, a change from N to Q at position 95 and a change from E to Y at position 355;
bw) a change from R to H at position 83, a change from T to S at position 200 and a change from E to S at position 355;
bx) a change from P to G at position 46 and a change from S to L at position 359;
by) a change from I to L at position 87, a change from P to G at position 89, and a change from E to Q at position 355; or
bz) a change from E to H at position 93 and a change from N to Q at position 95.

5. A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein comprises at least one of the following:
a) an H at position 28;
b) a K at position 31;
c) an H at position 34;
d) an L at position 36;
e) a Q at position 68;
f) an S at position 68;
g) a Q at position 83;
h) an H at position 83;
i) an S at position 88;
j) a Y at position 87;
k) a K at position 312;
l) a G at position 317;
m) an L at position 320;
n) a P at position 355;
o) a Q at position 358;
p) an S at position 365;
q) an S at position 315;
r) a Tat position 317;
s) an H at position 83 and a G at position 6;
t) an H at position 83 and a Y at position 8;
u) an H at position 83 and an L at position 11;
v) an H at position 83 and an L at position 36;
w) an H at position 83 and an L at position 53;
x) an H at position 83 and an L at position 87;
y) an H at position 83 and a G at position 89;
z) an H at position 83 and an H at position 93;
aa) an H at position 83 and a Q at position 95;
ab) an H at position 83 and a G at position 145;
ac) an H at position 83 and a P at position 168;
ad) an H at position 83 and an S at position 200;
ae) an H at position 83 and a Y at position 230;
af) an H at position 83 and Q at position 341;
ag) an H at position 83 and an L at position 355;
ah) an H at position 83 and a Y at position 355;
al) an H at position 83 and a Q at position 355;
aj) an H at position 83 and an S at position 355;
ak) an H at position 83 and an L at position 359;
al) an R to H at position 83 and a G at position 359;
am) an H at position 83 and a Q at position 367;
an) an H at position 83 and an S at position 367;
ao) an H at position 83 and a D at position 392;
ap) an H at position 83 and a Y at position 439;
aq) an H at position 83 and a Y at position 450;
ar) an H at position 83 and a Q at position 460;
as) a Y at position 87 and a Q at position 46;
at) a Y at position 87 and a G at position 89;
au) a Y at position 87 and a K at position 255;
av) a Y at position 87 and an S at position 272;
aw) a K at position 312 and an S at position 11;
ax) a K at position 312 and a P at position 355;
ay) an L at position 30, an H at position 83 and an S at position 200;
az) an H at position 83, a G at position 89 and an S at position 200;
ba) an H at position 83, an S at position 89 and an S at position 200;
bb) an H at position 83, an H at position 94 and an S at position 200;
bc) an H at position 83, a P at position 94 and an S at position 200;
bd) an H at position 83, an H at position 95 and an S at position 200;
be) an H at position 83, a Q at position 95 and an S at position 200;
bf) an H at position 83, an S at position 102 and an S at position 200;
bg) an H at position 83, an S at position 200 and a D at position 249;
bh) a G at position 6 and a Q at position 95;
bi) a G at position 6 and G at position 145;
bj) an L at position 36 and a Q at position 95;
bk) a Q at position 46 and a G at position 89;
bl) a Q at position 83 and an S at position 367;
bm) a G at position 89 and a G at position 359;
bn) a P at position 355 and a G at position 359;
bo) a G at position 6, a Q at position 46 and a G at position 89;
bp) a G at position 6, a Q at position 83 and an L at position 87;
bq) a G at position 6, an S at position 272 and a P at position 355;
br) an L at position 11, an H at position 83 and a P at position 355;
bs) an L at position 11, a Y at position 87 and a P at position 355;
bt) an L a position 11, an H at position 93 and an L at position 359;
bu) an S at position 11, a G at position 359 and a Q at position 367;
bv) a Q at position 46, a Q at position 95 and a Y at position 355;
bw) an H at position 83, an S at position 200 and an S at position 355;
bx) a G at position 46 and an L at position 359;
by) an L at position 87, a G at position 89, and a Q at position 355; and
bz) an H at position 93 and a Q at position 95.

6. A transformed photosynthetic organism comprising an exogenous polynucleotide encoding a modified rbcL protein of SEQ ID NO. 1 or a protein having at least 95% sequence identity to SEQ ID NO. 1; wherein the modified rbcL protein consists of:
a) an H at position 28;
b) a K at position 31;
c) an H at position 34;
d) an L at position 36;
e) a Q at position 68;
f) an S at position 68;
g) a Q at position 83;
h) an H at position 83;
i) an S at position 88;
j) a Y at position 87;
k) a K at position 312;
l) a G at position 317;
m) an L at position 320;
n) a P at position 355;
o) a Q at position 358;
p) an S at position 365;
q) an S at position 315;
r) a Tat position 317;
S) an H at position 83 and a G at position 6;
t) an H at position 83 and a Y at position 8;
u) an H at position 83 and an L at position 11;
v) an H at position 83 and an L at position 36;
w) an H at position 83 and an L at position 53;
x) an H at position 83 and an L at position 87;
y) an H at position 83 and a G at position 89;
z) an H at position 83 and an H at position 93;
aa) an H at position 83 and a Q at position 95;
ab) an H at position 83 and a G at position 145;
ac) an H at position 83 and a P at position 168;
ad) an H at position 83 and an S at position 200;
ae) an H at position 83 and a Y at position 230;
af) an H at position 83 and Q at position 341;
ag) an H at position 83 and an L at position 355;
ah) an H at position 83 and a Y at position 355;
ai) an H at position 83 and a Q at position 355;
aj) an H at position 83 and an S at position 355;
ak) an H at position 83 and an L at position 359;
al) an R to H at position 83 and a G at position 359;
am) an H at position 83 and a Q at position 367;
an) an H at position 83 and an S at position 367;
ao) an H at position 83 and a D at position 392;
ap) an H at position 83 and a Y at position 439;
aq) an H at position 83 and a Y at position 450;
ar) an H at position 83 and a Q at position 460;
as) a Y at position 87 and a Q at position 46;
at) a Y at position 87 and a G at position 89;
au) a Y at position 87 and a K at position 255;
av) a Y at position 87 and an S at position 272;
aw) a K at position 312 and an S at position 11;
ax) a K at position 312 and a P at position 355;
ay) an L at position 30, an H at position 83 and an S at position 200;
az) an H at position 83, a G at position 89 and an S at position 200;
ba) an H at position 83, an S at position 89 and an S at position 200;
bb) an H at position 83, an H at position 94 and an S at position 200;
bc) an H at position 83, a P at position 94 and an S at position 200;
bd) an H at position 83, an H at position 95 and an S at position 200;
be) an H at position 83, a Q at position 95 and an S at position 200;
bf) an H at position 83, an S at position 102 and an S at position 200;
bg) an H at position 83, an S at position 200 and a D at position 249;
bh) a G at position 6 and a Q at position 95;
bi) a G at position 6 and G at position 145;
bj) an L at position 36 and a Q at position 95;
bk) a Q at position 46 and a G at position 89;
bl) a Q at position 83 and an S at position 367;
bm) a G at position 89 and a G at position 359;
bn) a P at position 355 and a G at position 359;
bo) a G at position 6, a Q at position 46 and a G at position 89;
bp) a G at position 6, a Q at position 83 and an L at position 87;
bq) a G at position 6, an S at position 272 and a P at position 355;
br) an L at position 11, an H at position 83 and a P at position 355;
bs) an L at position 11, a Y at position 87 and a P at position 355;
bt) an L a position 11, an H at position 93 and an L at position 359;
bu) an S at position 11, a G at position 359 and a Q at position 367;
bv) a Q at position 46, a Q at position 95 and a Y at position 355;
bw) an H at position 83, an S at position 200 and an S at position 355;
bx) a G at position 46 and an L at position 359;
by) an L at position 87, a G at position 89, and a Q at position 355; or
bz) an H at position 93 and a Q at position 95.

7. The transformed photosynthetic organism of any one of 3 to 6, wherein the transformed photosynthetic organism expresses the modified rbcL protein and produces increased biomass as compared to an untransformed photosynthetic organism of the same species.

8. The transformed photosynthetic organism of 1, 2 or 7 wherein the increase in biomass is measured by at least one of a competition assay, growth rate, carrying capacity, productivity or cell proliferation.

9. The transformed photosynthetic organism of 8, wherein the increase in biomass is measured by a competition assay.

10. The transformed photosynthetic organism of 9, wherein the competition assay is performed in a turbidostat.

11. The transformed photosynthetic organism of 1, 2 or 7, wherein the increase is shown by the transformed photosynthetic organism having a positive selection coefficient as compared to an untransformed organism of the same species.

12. The transformed photosynthetic organism of 11, wherein the positive selection coefficient is from 0.05 to 0.10, from 0.10 to 0.5, from 0.5 to 0.75, from 0.75 to 1.0, from 1.0 to 1.5, from 1.5 to 2.0, or from 2.0 to 3.0.

13. The transformed photosynthetic organism of 8, wherein the increased biomass is measured by growth rate.

14. The transformed photosynthetic organism of 13, wherein the transformed photosynthetic organism has an increased growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%.

15. The transformed photosynthetic organism of 8, wherein the increased biomass is measured by carrying capacity.

16. The transformed photosynthetic organism of 15, wherein the units of carrying capacity are mass per unit of volume or mass per unit of area.

17. The transformed photosynthetic organism of 8, where the increase biomass is measured by increased productivity.

18. The transformed photosynthetic organism of 17, wherein the units of productivity are grams per meter squared per day, mass per unit area such as tons per acre/hectare, or volume per unit area such as bushels per acre/hectare.

19. The transformed photosynthetic organism of 17 wherein the transformed photosynthetic organism has an increase in productivity as compared to an untransformed photosynthetic organism of the same species of from 5% to 25%, or from 25% to 50%, of from 50% to 75%, of from 75% to 100%, of from 100% to 150%, of from 150% to 200%, for from 200% to 300% or from 300% to 400%.

20. The transformed photosynthetic organism of any one of 1 to 6, wherein the transformed photosynthetic organism is a bacterium.

21. The transformed photosynthetic organism of 20, wherein the bacterium is a cyanobacterium.

22. The transformed photosynthetic organism of any one of 1 to 6, wherein the transformed photosynthetic organism is an alga

23. The transformed photosynthetic organism of 22, wherein the alga is a microalga.

24. The transformed photosynthetic organism of 23, wherein the microalga is at least one of Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

25. The transformed photosynthetic organism of 24, wherein the microalga is at least one of Chlamydomonas. reinhardtii, N. oceanica, N. salina, Dunaliella. salina, H. pluvalis, S. dimorphus, Dunaliella viridis, N. oculata, Dunaliella tertiolecta, S. Maximus, or A. Fusiformus.

26. The transformed photosynthetic organism of one of 1 to 6, wherein the transformed photosynthetic organism is a vascular plant.

27. The transformed photosynthetic organism of 26, wherein the vascular plant is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, peanut (Arachis hypogaea), Arabidopsis sp., tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops or fruits.

28. The transformed photosynthetic organism of 2, wherein said exogenous polynucleotide is selected from the group consisting of SEQ ID NOs. 81-159.

29. A method for increasing biomass production in a photosynthetic organism comprising transforming the photosynthetic organism with an exogenous polynucleotide encoding a modified rbcL protein of SEQ ID NO: 1 to produce a transformed photosynthetic organism; wherein said modification to SEQ ID NO 1 comprises at least one amino acid substitution in the loop at positions 25-35, the β-sheet at positions 83-89, the α-helix at positions 310-321 and the loop-helix-loop at positions 355-365; and wherein the transformed photosynthetic organism expresses the modified rbcL protein and produces increased biomass as compared to an untransformed photosynthetic organism of the same species.

30. A method for increasing biomass production by a photosynthetic organism comprising transforming the photosynthetic organism with an exogenous polynucleotide encoding a modified rbcL protein of any one of SEQ ID NOs 2 to 79 to produce a transformed photosynthetic organism; wherein the transformed photosynthetic organism expresses the modified rbcL protein and produces increased biomass as compared to an untransformed photosynthetic organism of the same species.

31. A method for increasing biomass production by a photosynthetic organism comprising transforming the photosynthetic organism with an exogenous polynucleotide encoding a modified rbcL protein of SEQ ID NO. 1 or a protein having at least 95% sequence identity to SEQ ID NO. 1; wherein the transformed photosynthetic organism expresses the modified rbcL protein and produces increased biomass as compared to an untransformed photosynthetic organism of the same species; and wherein the modified rbcL protein comprises at least one of the following modifications:
a) a change from D to H at position 28;
b) a change from V to K at position 31;
c) a change from T to H at position 34;
d) a change from I to L at position 36;
e) a change from T to Q at position 68;
f) a change from T to S at position 68;
g) a change from R to Q at position 83;
h) a change from R to H at position 83;
i) a change from E to S at position 88;
j) a change from I to Y at position 87;
k) a change from R to K at position 312;
l) a change from A to G at position 317;
m) a change from M to L at position 320;
n) a change from E to P at position 355;
o) a change from R to Q at position 358;
p) a change from T to S at position 365;
q) a change from A to S at position 315;
r) a change from A to T at position 317;
s) a change from R to H at position 83 and a change from E to G at position 6;
t) a change from R to H at position 83 and a change from K to Y at position 8;
u) a change from R to H at position 83 and a change from A to L at position 11;
v) a change from R to H at position 83 and a change from I to L at position 36;
w) a change from R to H at position 83 and a change from C to L at position 53;
x) a change from R to H at position 83 and a change from I to L at position 87;
y) a change from R to H at position 83 and a change from P to G at position 89;
z) a change from R to H at position 83 and a change from E to H at position 93;
aa) a change from R to H at position 83 and a change from N to Q at position 95;
ab) a change from R to H at position 83 and a change from V to G at position 145;
ac) a change from R to H at position 83 and a change from G to P at position 168;
ad) a change from R to H at position 83 and a change from T to S at position 200;
ae) a change from R to H at position 83 and a change from A to Y at position 230;
af) a change from R to H at position 83 and a change from V to Q at position 341;
ag) a change from R to H at position 83 and a change from E to L at position 355;
ah) a change from R to H at position 83 and a change from E to Y at position 355;
ai) a change from R to H at position 83 and a change from E to Q at position 355;
aj) a change from R to H at position 83 and a change from E to S at position 355;
ak) a change from R to H at position 83 and a change from S to L at position 359;
al) a change from R to H at position 83 and a change from S to G at position 359;
am) a change from R to H at position 83 and a change from D to Q at position 367;
an) a change from R to H at position 83 and a change from D to S at position 367;
ao) a change from R to H at position 83 and a change from E to D at position 392;
ap) a change from R to H at position 83 and a change from R to Y at position 439;
aq) a change from R to H at position 83 and a change from K to Y at position 450;
ar) a change from R to H at position 83 and a change from E to Q at position 460;
as) a change from I to Y at position 87 and a change from P to Q at position 46;
at) a change from I to Y at position 87 and a change from P to G at position 89;
au) a change from I to Y at position 87 and a change from V to K at position 255;
av) a change from I to Y at position 87 and a change from G to S at position 272;
aw) a change from R to K at position 312 and a change from A to S at position 11;
ax) a change from R to K at position 312 and a change from E to P at position 355;
ay) a change from V to L at position 30, a change from R to H at position 83 and a change from T to S at position 200;
az) a change from R to H at position 83, a change from P to G at position 89 and a change from T to S at position 200;
ba) a change from a change from R to H at position 83, a change from P to S at position 89 and a change from T to S at position 200;
bb) a change from R to H at position 83, a change from D to H at position 94 and a change from T to S at position 200;
bc) a change from R to H at position 83, a change from D to P at position 94 and a change from T to S at position 200;
bd) a change from R to H at position 83, a change from N to H at position 95 and a change from T to S at position 200;
be) a change from R to H at position 83, a change from N to Q at position 95 and a change from T to S at position 200;
bf) a change from R to H at position 83, a change from A to S at position 102 and a change from T to S at position 200;
bg) a change from R to H at position 83, a change from T to S at position 200 and a change from E to D at position 249;
bh) a change from E to G at position 6 and a change from N to Q at position 95;
bi) a change from E to G at position 6 and a change from V to G at position 145;
bj) a change from I to L at position 36 and a change from N to Q at position 95;
bk) a change from P to Q at position 46 and a change from P to G at position 89;
bi) a change from R to Q at position 83 and a change from D to S at position 367;
bm) a change from P to G at position 89 and a change from S to G at position 359;
bn) a change from E to P at position 355 and a change from S to G at position 359;
bo) a change from E to G at position 6, a change from P to Q at position 46 and a change from P to G at position 89;
bp) a change from E to G at position 6, a change from R to Q at position 83 and a change from I to L at position 87;
bq) a change from a change from E to G at position 6, a change from G to S at position 272 and a change from E to P at position 355;
br) a change from A to L at position 11, a change from R to H at position 83 and a change from E to P at position 355;
bs) a change from A to L at position 11, a change from I to Y at position 87 and a change from E to P at position 355;
bt) a change from A to L a position 11, a change from E to H at position 93 and change from S to L at position 359;
bu) a change from A to S at position 11, a change from S to G at position 359 and a change from D to Q at position 367;
bv) a change from P to Q at position 46, a change from N to Q at position 95 and a change from E to Y at position 355;
bw) a change from R to H at position 83, a change from T to S at position 200 and a change from E to S at position 355;
bx) a change from P to G at position 46 and a change from S to L at position 359;
by) a change from I to L at position 87, a change from P to G at position 89, and a change from E to Q at position 355; and
bz) a change from E to H at position 93 and a change from N to Q at position 95.

32. A method for increasing biomass production by a photosynthetic organism comprising transforming the photosynthetic organism with an exogenous polynucleotide encoding a modified rbcL protein of SEQ ID NO. 1 or a protein having at least 95% sequence identity to SEQ ID NO. 1 to produce a transformed photosynthetic organism; wherein the transformed photosynthetic organism expresses the modified rbcL protein and produces increased biomass as compared to an untransformed photosynthetic organism of the same species; and wherein the modified rbcL protein consists of a modification selected from:
a) a change from D to H at position 28;
b) a change from V to K at position 31;
c) a change from T to H at position 34;
d) a change from I to L at position 36;
e) a change from T to Q at position 68;
f) a change from T to S at position 68;
g) a change from R to Q at position 83;
h) a change from R to H at position 83;
i) a change from E to S at position 88;
j) a change from I to Y at position 87;
k) a change from R to K at position 312;
l) a change from A to G at position 317;
m) a change from M to L at position 320;
n) a change from E to P at position 355;
o) a change from R to Q at position 358;
p) a change from T to S at position 365;
q) a change from A to S at position 315;
r) a change from A to T at position 317;
s) a change from R to H at position 83 and a change from E to G at position 6;
t) a change from R to H at position 83 and a change from K to Y at position 8;
u) a change from R to H at position 83 and a change from A to L at position 11;
v) a change from R to H at position 83 and a change from I to L at position 36;
w) a change from R to H at position 83 and a change from C to L at position 53;
x) a change from R to H at position 83 and a change from I to L at position 87;
y) a change from R to H at position 83 and a change from P to G at position 89;
z) a change from R to H at position 83 and a change from E to H at position 93;
aa) a change from R to H at position 83 and a change from N to Q at position 95;
ab) a change from R to H at position 83 and a change from V to G at position 145;
ac) a change from R to H at position 83 and a change from G to P at position 168;
ad) a change from R to H at position 83 and a change from T to S at position 200;
ae) a change from R to H at position 83 and a change from A to Y at position 230;
af) a change from R to H at position 83 and a change from V to Q at position 341;
ag) a change from R to H at position 83 and a change from E to L at position 355;
ah) a change from R to H at position 83 and a change from E to Y at position 355;
ai) a change from R to H at position 83 and a change from E to Q at position 355;
aj) a change from R to H at position 83 and a change from E to S at position 355;
ak) a change from R to H at position 83 and a change from S to L at position 359;
al) a change from R to H at position 83 and a change from S to G at position 359;
am) a change from R to H at position 83 and a change from D to Q at position 367;
an) a change from R to H at position 83 and a change from D to S at position 367;
ao) a change from R to H at position 83 and a change from E to D at position 392;
ap) a change from R to H at position 83 and a change from R to Y at position 439;
aq) a change from R to H at position 83 and a change from K to Y at position 450;
ar) a change from R to H at position 83 and a change from E to Q at position 460;
as) a change from I to Y at position 87 and a change from P to Q at position 46;
at) a change from I to Y at position 87 and a change from P to G at position 89;
au) a change from I to Y at position 87 and a change from V to K at position 255;
av) a change from I to Y at position 87 and a change from G to S at position 272;
aw) a change from R to K at position 312 and a change from A to S at position 11;
ax) a change from R to K at position 312 and a change from E to P at position 355;
ay) a change from V to L at position 30, a change from R to H at position 83 and a change from T to S at position 200;
az) a change from R to H at position 83, a change from P to G at position 89 and a change from T to S at position 200;
ba) a change from a change from R to H at position 83, a change from P to S at position 89 and a change from T to S at position 200;
bb) a change from R to H at position 83, a change from D to H at position 94 and a change from T to S at position 200;
bc) a change from R to H at position 83, a change from D to P at position 94 and a change from T to S at position 200;
bd) a change from R to H at position 83, a change from N to H at position 95 and a change from T to S at position 200;
be) a change from R to H at position 83, a change from N to Q at position 95 and a change from T to S at position 200;
bf) a change from R to H at position 83, a change from A to S at position 102 and a change from T to S at position 200;
bg) a change from R to H at position 83, a change from T to S at position 200 and a change from E to D at position 249;
bh) a change from E to G at position 6 and a change from N to Q at position 95;
bi) a change from E to G at position 6 and a change from V to G at position 145;
bj) a change from I to L at position 36 and a change from N to Q at position 95;
bk) a change from P to Q at position 46 and a change from P to G at position 89;
bl) a change from R to Q at position 83 and a change from D to S at position 367;
bm) a change from P to G at position 89 and a change from S to G at position 359;
bn) a change from E to P at position 355 and a change from S to G at position 359;
bo) a change from E to G at position 6, a change from P to Q at position 46 and a change from P to G at position 89;
bp) a change from E to G at position 6, a change from R to Q at position 83 and a change from I to L at position 87;
bq) a change from a change from E to G at position 6, a change from G to S at position 272 and a change from E to P at position 355;
br) a change from A to L at position 11, a change from R to H at position 83 and a change from E to P at position 355;
bs) a change from A to L at position 11, a change from I to Y at position 87 and a change from E to P at position 355;
bt) a change from A to L a position 11, a change from E to H at position 93 and change from S to L at position 359;
bu) a change from A to S at position 11, a change from S to G at position 359 and a change from D to Q at position 367;
bv) a change from P to Q at position 46, a change from N to Q at position 95 and a change from E to Y at position 355;
bw) a change from R to H at position 83, a change from T to S at position 200 and a change from E to S at position 355;
bx) a change from P to G at position 46 and a change from S to L at position 359;
by) a change from I to L at position 87, a change from P to G at position 89, and a change from E to Q at position 355; or
bz) a change from E to H at position 93 and a change from N to Q at position 95.

33. A method for increasing biomass production by a photosynthetic organism comprising transforming the photosynthetic organism with an exogenous polynucleotide encoding a modified rbcL protein of SEQ ID NO. 1 or a protein having at least 95% sequence identity to SEQ ID NO. 1 to produce a transformed photosynthetic organism; wherein the transformed photosynthetic organism expresses the modified rbcL protein and produces increased biomass as compared to an untransformed photosynthetic organism of the same species; and wherein the modified rbcL protein comprises at least one of the following:
a) an H at position 28;
b) a K at position 31;
c) an H at position 34;
d) an L at position 36;
e) a Q at position 68;
f) an S at position 68;
g) a Q at position 83;
h) an H at position 83;
i) an S at position 88;
j) a Y at position 87;
k) a K at position 312;
l) a G at position 317;
m) an L at position 320;
n) a P at position 355;
o) a Q at position 358;
p) an S at position 365;
q) an S at position 315;
r) a T at position 317;
s) an H at position 83 and a G at position 6;
t) an H at position 83 and a Y at position 8;
u) an H at position 83 and an L at position 11;
v) an H at position 83 and an L at position 36;
w) an H at position 83 and an L at position 53;
x) an H at position 83 and an L at position 87;
y) an H at position 83 and a G at position 89;
z) an H at position 83 and an H at position 93;
aa) an H at position 83 and a Q at position 95;
ab) an H at position 83 and a G at position 145;
ac) an H at position 83 and a P at position 168;
ad) an H at position 83 and an S at position 200;
ae) an H at position 83 and a Y at position 230;
af) an H at position 83 and Q at position 341;
ag) an H at position 83 and an L at position 355;
ah) an H at position 83 and a Y at position 355;
ai) an H at position 83 and a Q at position 355;
aj) an H at position 83 and an S at position 355;
ak) an H at position 83 and an L at position 359;
al) an R to H at position 83 and a G at position 359;
am) an H at position 83 and a Q at position 367;
an) an H at position 83 and an S at position 367;
ao) an H at position 83 and a D at position 392;
ap) an H at position 83 and a Y at position 439;
aq) an H at position 83 and a Y at position 450;
ar) an H at position 83 and a Q at position 460;
as) a Y at position 87 and a Q at position 46;
at) a Y at position 87 and a G at position 89;
au) a Y at position 87 and a K at position 255;
av) a Y at position 87 and an S at position 272;
aw) a K at position 312 and an S at position 11;
ax) a K at position 312 and a P at position 355;
ay) an L at position 30, an H at position 83 and an S at position 200;
az) an H at position 83, a G at position 89 and an S at position 200;
ba) an H at position 83, an S at position 89 and an S at position 200;
bb) an H at position 83, an H at position 94 and an S at position 200;
bc) an H at position 83, a P at position 94 and an S at position 200;
bd) an H at position 83, an H at position 95 and an S at position 200;
be) an H at position 83, a Q at position 95 and an S at position 200;
bf) an H at position 83, an S at position 102 and an S at position 200;
bg) an H at position 83, an S at position 200 and a D at position 249;
bh) a G at position 6 and a Q at position 95;
bi) a G at position 6 and G at position 145;
bj) an L at position 36 and a Q at position 95;
bk) a Q at position 46 and a G at position 89;
bl) a Q at position 83 and an S at position 367;
bm) a G at position 89 and a G at position 359;
bn) a P at position 355 and a G at position 359;
bo) a G at position 6, a Q at position 46 and a G at position 89;
bp) a G at position 6, a Q at position 83 and an L at position 87;
bq) a G at position 6, an S at position 272 and a P at position 355;
br) an L at position 11, an H at position 83 and a P at position 355;
bs) an L at position 11, a Y at position 87 and a P at position 355;
bt) an L a position 11, an H at position 93 and an L at position 359;
bu) an S at position 11, a G at position 359 and a Q at position 367;
bv) a Q at position 46, a Q at position 95 and a Y at position 355;
bw) an H at position 83, an S at position 200 and an S at position 355;
bx) a G at position 46 and an L at position 359;
by) an L at position 87, a G at position 89, and a Q at position 355; and
bz) an H at position 93 and a Q at position 95.

34. A method for increasing biomass production by a photosynthetic organism comprising transforming said photosynthetic organism with an exogenous polynucleotide encoding a modified rbcL protein of SEQ ID NO. 1 or a protein having at least 95% sequence identity to SEQ ID NO. 1 to produce a transformed photosynthetic organism; wherein the transformed photosynthetic organism expresses the modified rbcL protein and produces increased biomass as compared to an untransformed photosynthetic organism of the same species; and wherein the modified rbcL protein consists of:
a) an H at position 28;
b) a K at position 31;
c) an H at position 34;
d) an L at position 36;
e) a Q at position 68;
f) an S at position 68;
g) a Q at position 83;
h) an H at position 83;
i) an S at position 88;
j) a Y at position 87;
k) a K at position 312;
l) a G at position 317;
m) an L at position 320;
n) a P at position 355;
o) a Q at position 358;
p) an S at position 365;
q) an S at position 315;
r) a Tat position 317;
s) an H at position 83 and a G at position 6;
t) an H at position 83 and a Y at position 8;
u) an H at position 83 and an L at position 11;
v) an H at position 83 and an L at position 36;
w) an H at position 83 and an L at position 53;
x) an H at position 83 and an L at position 87;
y) an H at position 83 and a G at position 89;
z) an H at position 83 and an H at position 93;
aa) an H at position 83 and a Q at position 95;
ab) an H at position 83 and a G at position 145;
ac) an H at position 83 and a P at position 168;
ad) an H at position 83 and an S at position 200;
ae) an H at position 83 and a Y at position 230;
af) an H at position 83 and Q at position 341;
ag) an H at position 83 and an L at position 355;
ah) an H at position 83 and a Y at position 355;
ai) an H at position 83 and a Q at position 355;
aj) an H at position 83 and an S at position 355;
ak) an H at position 83 and an L at position 359;
al) an R to H at position 83 and a G at position 359;
am) an H at position 83 and a Q at position 367;
an) an H at position 83 and an S at position 367;
ao) an H at position 83 and a D at position 392;
ap) an H at position 83 and a Y at position 439;
aq) an H at position 83 and a Y at position 450;
ar) an H at position 83 and a Q at position 460;
as) a Y at position 87 and a Q at position 46;
at) a Y at position 87 and a G at position 89;
au) a Y at position 87 and a K at position 255;
av) a Y at position 87 and an S at position 272;
aw) a K at position 312 and an S at position 11;
ax) a K at position 312 and a P at position 355;
ay) an L at position 30, an H at position 83 and an S at position 200;
az) an H at position 83, a G at position 89 and an S at position 200;
ba) an H at position 83, an S at position 89 and an S at position 200;
bb) an H at position 83, an H at position 94 and an S at position 200;
bc) an H at position 83, a P at position 94 and an S at position 200;
bd) an H at position 83, an H at position 95 and an S at position 200;
be) an H at position 83, a Q at position 95 and an S at position 200;
bf) an H at position 83, an S at position 102 and an S at position 200;
bg) an H at position 83, an S at position 200 and a D at position 249;
bh) a G at position 6 and a Q at position 95;
bi) a G at position 6 and G at position 145;
bj) an L at position 36 and a Q at position 95;
bk) a Q at position 46 and a G at position 89;
bl) a Q at position 83 and an S at position 367;
bm) a G at position 89 and a G at position 359;
bn) a P at position 355 and a G at position 359;
bo) a G at position 6, a Q at position 46 and a G at position 89;
bp) a G at position 6, a Q at position 83 and an L at position 87;
bq) a G at position 6, an S at position 272 and a P at position 355;
br) an L at position 11, an H at position 83 and a P at position 355;
bs) an L at position 11, a Y at position 87 and a P at position 355;
bt) an L a position 11, an H at position 93 and an L at position 359;
bu) an S at position 11, a G at position 359 and a Q at position 367;
bv) a Q at position 46, a Q at position 95 and a Y at position 355;
bw) an H at position 83, an S at position 200 and an S at position 355;
bx) a G at position 46 and an L at position 359;
by) an L at position 87, a G at position 89, and a Q at position 355; or
bz) an H at position 93 and a Q at position 95.

35. The method of any one of 29 to 34, wherein the increase in biomass production is measured by at least one of a competition assay, growth rate, carrying capacity, productivity or cell proliferation.

36. The method of 35, wherein the increase in biomass production is measured by a competition assay.

37. The method of 36, wherein the competition assay is performed in a turbidostat.

38. The method of any one of 29 to 34, wherein the increase in biomass production is shown by the transformed photosynthetic organism having a positive selection coefficient as compared to an untransformed photosynthetic organism of the same species.

39. The method of 38, wherein the positive selection coefficient is from 0.05 to 0.10, from 0.10 to 0.5, from 0.5 to 0.75, from 0.75 to 1.0, from 1.0 to 1.5, from 1.5 to 2.0, or from 2.0 to 3.0.

40. The method of 35, wherein the increase in biomass production is measured by growth rate.

41. The method of 40, wherein the transformed photosynthetic organism has an increased growth rate as compared to an untransformed photosynthetic organism of the same species of from 5% to 10%, from 10% to 15%, from 15% to 25%, from 25% to 50%, from 50% to 75%, from 75% to 100%, from 100% to 150%, from 150% to 200%, from 200% to 300%, or from 300% to 400%,

42. The method of 35, wherein the increase in biomass production is measured by carrying capacity.

43. The method of 42, wherein the units of carrying capacity are mass per unit of volume or mass per unit of area.

44. The method of 35, wherein the increase in biomass production is measured by increased productivity.

45. The method of 44, wherein the units of productivity are grams per meter squared per day, mass per unit area such as tons per acre/hectare, or volume per unit area such as bushels per acre/hectare.

46. The method of 4, wherein the transformed photosynthetic organism has an increase in productivity as compared to an untransformed photosynthetic organism of the same species of from 5% to 25%, or from 25% to 50%, of from 50% to 75%, of from 75% to 100%, of from 100% to 150%, of from 150% to 200%, for from 200% to 300% or from 300% to 400%.

47. The method of any one of 29 to 34, wherein the transformed photosynthetic organism is a bacterium.

48. The method of 47, wherein the bacterium is a cyanobacterium.

49. The method of any one of 29 to 34, wherein the transformed photosynthetic organism is an alga

50. The method of 49, wherein the alga is a microalga.

51. The method of 50, wherein the microalga is at least one of Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

52. The method of 51, wherein the microalga is at least one of Chlamydomonas. reinhardtii, N. oceanica, N. salina, Dunaliella. salina, H. pluvalis, S. dimorphus, Dunaliella viridis, N. oculata, Dunaliella tertiolecta, S. Maximus, or A. Fusiformus.

53. The method of one of 29 to 34, wherein the transformed photosynthetic organism is a vascular plant.

54. The method of 53, wherein the vascular plant is Brassica (e.g., Brassica nigra, Brassica napus, Brassica hirta, Brassica rapa, Brassica campestris, Brassica carinata, and Brassica juncea), soybean (Glycine max), castor bean (Ricinus communis), cotton, safflower (Carthamus tinctorius), sunflower (Helianthus annuus), flax (Linum usitatissimum), corn (Zea mays), coconut (Cocos nucifera), palm (Elaeis guineensis), oil nut trees such as olive (Olea europaea), sesame, peanut (Arachis hypogaea), Arabidopsis sp., tobacco, wheat, sugarcane, sugar beet, barley, oats, amaranth, potato, rice, tomato, legumes (e.g., peas, beans, lentils, alfalfa, etc.), grasses (e.g. Miscanthus, switchgrass, energy cane), vegetable crops or fruits.

55. A modified rbcL protein of SEQ ID NO: 1 said modification to SEQ ID NO 1 comprising at least one amino acid substitution in the loop at positions 25-35, the β-sheet at positions 83-89, the α-helix at positions 310-321 and the loop-helix-loop at positions 355-365.

56. A modified rbcL protein comprising any one of SEQ ID NOs 2 to 79.

57. A modified rbcL protein comprising SEQ ID NO. 1 or a protein having at least 95% sequence identity to SEQ ID NO. 1; said modification comprising at least one of the following:
a) a change from D to H at position 28;
b) a change from V to K at position 31;
c) a change from T to H at position 34;
d) a change from I to L at position 36;
e) a change from T to Q at position 68;
f) a change from T to S at position 68;
g) a change from R to Q at position 83;
h) a change from R to H at position 83;
i) a change from E to S at position 88;
j) a change from I to Y at position 87;
k) a change from R to K at position 312;
l) a change from A to G at position 317;
m) a change from M to L at position 320;
n) a change from E to P at position 355;
o) a change from R to Q at position 358;
p) a change from T to S at position 365;
q) a change from A to S at position 315;
r) a change from A to T at position 317;
s) a change from R to H at position 83 and a change from E to G at position 6;
t) a change from R to H at position 83 and a change from K to Y at position 8;
u) a change from R to H at position 83 and a change from A to L at position 11;
v) a change from R to H at position 83 and a change from I to L at position 36;
w) a change from R to H at position 83 and a change from C to L at position 53;
x) a change from R to H at position 83 and a change from I to L at position 87;
y) a change from R to H at position 83 and a change from P to G at position 89;
z) a change from R to H at position 83 and a change from E to H at position 93;
aa) a change from R to H at position 83 and a change from N to Q at position 95;
ab) a change from R to H at position 83 and a change from V to G at position 145;
ac) a change from R to H at position 83 and a change from G to P at position 168;
ad) a change from R to H at position 83 and a change from T to S at position 200;
ae) a change from R to H at position 83 and a change from A to Y at position 230;
af) a change from R to H at position 83 and a change from V to Q at position 341;
ag) a change from R to H at position 83 and a change from E to L at position 355;
ah) a change from R to H at position 83 and a change from E to Y at position 355;
ai) a change from R to H at position 83 and a change from E to Q at position 355;
aj) a change from R to H at position 83 and a change from E to S at position 355;
ak) a change from R to H at position 83 and a change from S to L at position 359;
al) a change from R to H at position 83 and a change from S to G at position 359;
am) a change from R to H at position 83 and a change from D to Q at position 367;
an) a change from R to H at position 83 and a change from D to S at position 367;
ao) a change from R to H at position 83 and a change from E to D at position 392;
ap) a change from R to H at position 83 and a change from R to Y at position 439;
aq) a change from R to H at position 83 and a change from K to Y at position 450;
ar) a change from R to H at position 83 and a change from E to Q at position 460;
as) a change from I to Y at position 87 and a change from P to Q at position 46;
at) a change from I to Y at position 87 and a change from P to G at position 89;
au) a change from I to Y at position 87 and a change from V to K at position 255;
av) a change from I to Y at position 87 and a change from G to S at position 272;
aw) a change from R to K at position 312 and a change from A to S at position 11;
ax) a change from R to K at position 312 and a change from E to P at position 355;
ay) a change from V to L at position 30, a change from R to H at position 83 and a change from T to S at position 200;
az) a change from R to H at position 83, a change from P to G at position 89 and a change from T to S at position 200;
ba) a change from a change from R to H at position 83, a change from P to S at position 89 and a change from T to S at position 200;
bb) a change from R to H at position 83, a change from D to H at position 94 and a change from T to S at position 200;
bc) a change from R to H at position 83, a change from D to P at position 94 and a change from T to S at position 200;
bd) a change from R to H at position 83, a change from N to H at position 95 and a change from T to S at position 200;
be) a change from R to H at position 83, a change from N to Q at position 95 and a change from T to S at position 200;
bf) a change from R to H at position 83, a change from A to S at position 102 and a change from T to S at position 200;
bg) a change from R to H at position 83, a change from T to S at position 200 and a change from E to D at position 249;
bh) a change from E to G at position 6 and a change from N to Q at position 95;
bi) a change from E to G at position 6 and a change from V to G at position 145;
bj) a change from I to L at position 36 and a change from N to Q at position 95;
bk) a change from P to Q at position 46 and a change from P to G at position 89;
bl) a change from R to Q at position 83 and a change from D to S at position 367;
bm) a change from P to G at position 89 and a change from S to G at position 359;
bn) a change from E to P at position 355 and a change from S to G at position 359;
bo) a change from E to G at position 6, a change from P to Q at position 46 and a change from P to G at position 89;
bp) a change from E to G at position 6, a change from R to Q at position 83 and a change from I to L at position 87;
bq) a change from a change from E to G at position 6, a change from G to S at position 272 and a change from E to P at position 355;
br) a change from A to L at position 11, a change from R to H at position 83 and a change from E to P at position 355;
bs) a change from A to L at position 11, a change from I to Y at position 87 and a change from E to P at position 355;
bt) a change from A to L a position 11, a change from E to H at position 93 and change from S to L at position 359;
bu) a change from A to S at position 11, a change from S to G at position 359 and a change from D to Q at position 367;
bv) a change from P to Q at position 46, a change from N to Q at position 95 and a change from E to Y at position 355;
bw) a change from R to H at position 83, a change from T to S at position 200 and a change from E to S at position 355;
bx) a change from P to G at position 46 and a change from S to L at position 359;
by) a change from I to L at position 87, a change from P to G at position 89, and a change from E to Q at position 355; and
bz) a change from E to H at position 93 and a change from N to Q at position 95.

58. A modified rbcL protein comprising SEQ ID NO. 1 or a protein having at least 95% sequence identity to SEQ ID NO. 1; wherein the modification consists of:
a) a change from D to H at position 28;
b) a change from V to K at position 31;
c) a change from T to H at position 34;
d) a change from I to L at position 36;
e) a change from T to Q at position 68;
f) a change from T to S at position 68;
g) a change from R to Q at position 83;
h) a change from R to H at position 83;
i) a change from E to S at position 88;
j) a change from I to Y at position 87;
k) a change from R to K at position 312;
l) a change from A to G at position 317;
m) a change from M to L at position 320;
n) a change from E to P at position 355;
o) a change from R to Q at position 358;
p) a change from T to S at position 365;
q) a change from A to S at position 315;
r) a change from A to T at position 317;
s) a change from R to H at position 83 and a change from E to G at position 6;
t) a change from R to H at position 83 and a change from K to Y at position 8;
u) a change from R to H at position 83 and a change from A to L at position 11;
v) a change from R to H at position 83 and a change from I to L at position 36;
w) a change from R to H at position 83 and a change from C to L at position 53;
x) a change from R to H at position 83 and a change from I to L at position 87;
y) a change from R to H at position 83 and a change from P to G at position 89;
z) a change from R to H at position 83 and a change from E to H at position 93;
aa) a change from R to H at position 83 and a change from N to Q at position 95;
ab) a change from R to H at position 83 and a change from V to G at position 145;
ac) a change from R to H at position 83 and a change from G to P at position 168;
ad) a change from R to H at position 83 and a change from T to S at position 200;
ae) a change from R to H at position 83 and a change from A to Y at position 230;
af) a change from R to H at position 83 and a change from V to Q at position 341;
ag) a change from R to H at position 83 and a change from E to L at position 355;
ah) a change from R to H at position 83 and a change from E to Y at position 355;
ai) a change from R to H at position 83 and a change from E to Q at position 355;
aj) a change from R to H at position 83 and a change from E to S at position 355;
ak) a change from R to H at position 83 and a change from S to L at position 359;
al) a change from R to H at position 83 and a change from S to G at position 359;
am) a change from R to H at position 83 and a change from D to Q at position 367;
an) a change from R to H at position 83 and a change from D to S at position 367;
ao) a change from R to H at position 83 and a change from E to D at position 392;
ap) a change from R to H at position 83 and a change from R to Y at position 439;
aq) a change from R to H at position 83 and a change from K to Y at position 450;
ar) a change from R to H at position 83 and a change from E to Q at position 460;
as) a change from I to Y at position 87 and a change from P to Q at position 46;
at) a change from I to Y at position 87 and a change from P to G at position 89;
au) a change from I to Y at position 87 and a change from V to K at position 255;
av) a change from I to Y at position 87 and a change from G to S at position 272;
aw) a change from R to K at position 312 and a change from A to S at position 11;
ax) a change from R to K at position 312 and a change from E to P at position 355;
ay) a change from V to L at position 30, a change from R to H at position 83 and a change from T to S at position 200;
az) a change from R to H at position 83, a change from P to G at position 89 and a change from T to S at position 200;
ba) a change from a change from R to H at position 83, a change from P to S at position 89 and a change from T to S at position 200;
bb) a change from R to H at position 83, a change from D to H at position 94 and a change from T to S at position 200;
bc) a change from R to H at position 83, a change from D to P at position 94 and a change from T to S at position 200;
bd) a change from R to H at position 83, a change from N to H at position 95 and a change from T to S at position 200;
be) a change from R to H at position 83, a change from N to Q at position 95 and a change from T to S at position 200;
bf) a change from R to H at position 83, a change from A to S at position 102 and a change from T to S at position 200;
bg) a change from R to H at position 83, a change from T to S at position 200 and a change from E to D at position 249;
bh) a change from E to G at position 6 and a change from N to Q at position 95;
bi) a change from E to G at position 6 and a change from V to G at position 145;
bj) a change from I to L at position 36 and a change from N to Q at position 95;
bk) a change from P to Q at position 46 and a change from P to G at position 89;
bl) a change from R to Q at position 83 and a change from D to S at position 367;
bm) a change from P to G at position 89 and a change from S to G at position 359;
bn) a change from E to P at position 355 and a change from S to G at position 359;
bo) a change from E to G at position 6, a change from P to Q at position 46 and a change from P to G at position 89;
bp) a change from E to G at position 6, a change from R to Q at position 83 and a change from I to L at position 87;
bq) a change from a change from E to G at position 6, a change from G to S at position 272 and a change from E to P at position 355;
br) a change from A to L at position 11, a change from R to H at position 83 and a change from E to P at position 355;
bs) a change from A to L at position 11, a change from I to Y at position 87 and a change from E to P at position 355;
bt) a change from A to L a position 11, a change from E to H at position 93 and change from S to L at position 359;
bu) a change from A to S at position 11, a change from S to G at position 359 and a change from D to Q at position 367;
bv) a change from P to Q at position 46, a change from N to Q at position 95 and a change from E to Y at position 355;
bw) a change from R to H at position 83, a change from T to S at position 200 and a change from E to S at position 355;
bx) a change from P to G at position 46 and a change from S to L at position 359;
by) a change from I to L at position 87, a change from P to G at position 89, and a change from E to Q at position 355; or
bz) a change from E to H at position 93 and a change from N to Q at position 95.

59. A modified rbcL protein comprising SEQ ID NO. 1 or a protein having at least 95% sequence identity to SEQ ID NO. 1; wherein the modification to the rbcL protein comprises at least one of the following:
a) an H at position 28;
b) a K at position 31;
c) an H at position 34;
d) an L at position 36;
e) a Q at position 68;
f) an S at position 68;
g) a Q at position 83;
h) an H at position 83;
i) an S at position 88;
j) a Y at position 87;
k) a K at position 312;
l) a G at position 317;
m) an L at position 320;
n) a P at position 355;
o) a Q at position 358;
p) an S at position 365;
q) an S at position 315;
r) a T at position 317;
s) an H at position 83 and a G at position 6;
t) an H at position 83 and a Y at position 8;
u) an H at position 83 and an L at position 11;
v) an H at position 83 and an L at position 36;
w) an H at position 83 and an L at position 53;
x) an H at position 83 and an L at position 87;
y) an H at position 83 and a G at position 89;
z) an H at position 83 and an H at position 93;
aa) an H at position 83 and a Q at position 95;
ab) an H at position 83 and a G at position 145;
ac) an H at position 83 and a P at position 168;
ad) an H at position 83 and an S at position 200;
ae) an H at position 83 and a Y at position 230;
af) an H at position 83 and Q at position 341;
ag) an H at position 83 and an L at position 355;
ah) an H at position 83 and a Y at position 355;
ai) an H at position 83 and a Q at position 355;
aj) an H at position 83 and an S at position 355;
ak) an H at position 83 and an L at position 359;
al) an R to H at position 83 and a G at position 359;
am) an H at position 83 and a Q at position 367;
an) an H at position 83 and an S at position 367;
ao) an H at position 83 and a D at position 392;
ap) an H at position 83 and a Y at position 439;
aq) an H at position 83 and a Y at position 450;
ar) an H at position 83 and a Q at position 460;
as) a Y at position 87 and a Q at position 46;
at) a Y at position 87 and a G at position 89;
au) a Y at position 87 and a K at position 255;
av) a Y at position 87 and an S at position 272;
aw) a K at position 312 and an S at position 11;
ax) a K at position 312 and a P at position 355;
ay) an L at position 30, an H at position 83 and an S at position 200;
az) an H at position 83, a G at position 89 and an S at position 200;
ba) an H at position 83, an S at position 89 and an S at position 200;
bb) an H at position 83, an H at position 94 and an S at position 200;
bc) an H at position 83, a P at position 94 and an S at position 200;
bd) an H at position 83, an H at position 95 and an S at position 200;
be) an H at position 83, a Q at position 95 and an S at position 200;
bf) an H at position 83, an S at position 102 and an S at position 200;
bg) an H at position 83, an S at position 200 and a D at position 249;
bh) a G at position 6 and a Q at position 95;
bi) a G at position 6 and G at position 145;
bj) an L at position 36 and a Q at position 95;
bk) a Q at position 46 and a G at position 89;
bl) a Q at position 83 and an S at position 367;
bm) a G at position 89 and a G at position 359;
bn) a P at position 355 and a G at position 359;
bo) a G at position 6, a Q at position 46 and a G at position 89;
bp) a G at position 6, a Q at position 83 and an L at position 87;
bq) a G at position 6, an S at position 272 and a P at position 355;
br) an L at position 11, an H at position 83 and a P at position 355;
bs) an L at position 11, a Y at position 87 and a P at position 355;
bt) an L a position 11, an H at position 93 and an L at position 359;
bu) an S at position 11, a G at position 359 and a Q at position 367;
bv) a Q at position 46, a Q at position 95 and a Y at position 355;
bw) an H at position 83, an S at position 200 and an S at position 355;
bx) a G at position 46 and an L at position 359;
by) an L at position 87, a G at position 89, and a Q at position 355; and
bz) an H at position 93 and a Q at position 95.

60. A modified rbcL protein comprising SEQ ID NO. 1 or a protein having at least 95% sequence identity to SEQ ID NO. 1; wherein the modification to the rbcL protein consists of:
a) an H at position 28;
b) a K at position 31;
c) an H at position 34;
d) an L at position 36;
e) a Q at position 68;
f) an S at position 68;
g) a Q at position 83;
h) an H at position 83;
i) an S at position 88;
j) a Y at position 87;
k) a K at position 312;
l) a G at position 317;
m) an L at position 320;
n) a P at position 355;
o) a Q at position 358;
p) an S at position 365;
q) an S at position 315;
r) a T at position 317;
s) an H at position 83 and a G at position 6;
t) an H at position 83 and a Y at position 8;
u) an H at position 83 and an L at position 11;
v) an H at position 83 and an L at position 36;
w) an H at position 83 and an L at position 53;
x) an H at position 83 and an L at position 87;
y) an H at position 83 and a G at position 89;
z) an H at position 83 and an H at position 93;
aa) an H at position 83 and a Q at position 95;
ab) an H at position 83 and a G at position 145;
ac) an H at position 83 and a P at position 168;
ad) an H at position 83 and an S at position 200;
ae) an H at position 83 and a Y at position 230;
af) an H at position 83 and Q at position 341;
ag) an H at position 83 and an L at position 355;
ah) an H at position 83 and a Y at position 355;
ai) an H at position 83 and a Q at position 355;
aj) an H at position 83 and an S at position 355;
ak) an H at position 83 and an L at position 359;
al) an R to H at position 83 and a G at position 359;
am) an H at position 83 and a Q at position 367;
an) an H at position 83 and an S at position 367;
ao) an H at position 83 and a D at position 392;
ap) an H at position 83 and a Y at position 439;
aq) an H at position 83 and a Y at position 450;
ar) an H at position 83 and a Q at position 460;
as) a Y at position 87 and a Q at position 46;
at) a Y at position 87 and a G at position 89;
au) a Y at position 87 and a K at position 255;
av) a Y at position 87 and an S at position 272;
aw) a K at position 312 and an S at position 11;
ax) a K at position 312 and a P at position 355;
ay) an L at position 30, an H at position 83 and an S at position 200;
az) an H at position 83, a G at position 89 and an S at position 200;
ba) an H at position 83, an S at position 89 and an S at position 200;
bb) an H at position 83, an H at position 94 and an S at position 200;
bc) an H at position 83, a P at position 94 and an S at position 200;
bd) an H at position 83, an H at position 95 and an S at position 200;
be) an H at position 83, a Q at position 95 and an S at position 200;
bf) an H at position 83, an S at position 102 and an S at position 200;
bg) an H at position 83, an S at position 200 and a D at position 249;
bh) a G at position 6 and a Q at position 95;
bi) a G at position 6 and G at position 145;
bj) an L at position 36 and a Q at position 95;
bk) a Q at position 46 and a G at position 89;
bl) a Q at position 83 and an S at position 367;
bm) a G at position 89 and a G at position 359;
bn) a P at position 355 and a G at position 359;
bo) a G at position 6, a Q at position 46 and a G at position 89;
bp) a G at position 6, a Q at position 83 and an L at position 87;
bq) a G at position 6, an S at position 272 and a P at position 355;
br) an L at position 11, an H at position 83 and a P at position 355;
bs) an L at position 11, a Y at position 87 and a P at position 355;
bt) an L a position 11, an H at position 93 and an L at position 359;
bu) an S at position 11, a G at position 359 and a Q at position 367;
bv) a Q at position 46, a Q at position 95 and a Y at position 355;
bw) an H at position 83, an S at position 200 and an S at position 355;
bx) a G at position 46 and an L at position 359;
by) an L at position 87, a G at position 89, and a Q at position 355; or
bz) an H at position 93 and a Q at position 95.

61. An isolated polynucleotide encoding any one of proteins SEQ ID NO. 2 to 79.

62. The isolated polynucleotide of 61, wherein said polynucleotide is any one of SEQ ID NO. 81 to 159.
